(19) United States
(12) Patent Application Publication
(10) Pub. No.: US 2025/0049948 A1
(43) Pub. Date: Feb. 13, 2025

(54) NOVEL IONIZABLE LIPIDS AND LIPID NANOPARTICLES AND METHODS OF USING THE SAME

(71) Applicant: Sail Biomedicines, Inc., Cambridge, MA (US)

(72) Inventors: Alessandra BARTOLOZZI, Cambridge, MA (US); John PROUDFOOT, Cambridge, MA (US); Roman ERDMANN, Cambridge, MA (US); Siddharth PATEL, Cambridge, MA (US); Alaina HOWE, Cambridge, MA (US); Dominick SALERNO, Cambridge, MA (US); Sanmit ADHIKARI, Cambridge, MA (US); Roman BOGORAD, Cambridge, MA (US); Arijit ADHIKARI, Cambridge, MA (US)

(21) Appl. No.: 18/710,109

(22) PCT Filed: Nov. 16, 2022
(86) PCT No.: PCT/US22/50111

§ 371 Date: May 14, 2024

Related U.S. Application Data
(60) Provisional application No. 63/264,149, filed on Nov. 16, 2021.

(51) Int. Cl. A61K 48/00; A61K 47/18; A61K 47/20; A61K 47/22; C07C 229/16; C07C 237/12; C07C 275/16; C07C 333/04; C07D 203/12; C07D 295/108; C07D 295/13; C07D 295/15

(52) U.S. Cl. CPC A61K 48/0033; A61K 47/183; A61K 47/20; A61K 47/22; C07C 229/16; C07C 237/12; C07C 275/16; C07C 333/04; C07D 203/12; C07D 295/108; C07D 295/13; C07D 295/15

(57) ABSTRACT

Novel ionizable lipids and lipid nanoparticles that can be used in the delivery of therapeutic cargos are disclosed.

### CROSS-REFERENCE TO RELATED APPLICATIONS

This application claims benefit of priority to U.S. Provisional Application No. 63/264,149 filed Nov. 16, 2021, which is herein incorporated by reference in its entirety.

### BACKGROUND

Lipid nanoparticles (“LNPs”) formed from ionizable amine-containing lipids can serve as therapeutic cargo vehicles for delivery of biologically active agents, such as coding RNAs (i.e., messenger RNAs (mRNAs), guide RNAs) and non-coding RNAs (i.e. antisense, siRNA), into cells. LNPs can facilitate delivery of oligonucleotide agents across cell membranes and can be used to introduce components and compositions into living cells.

Biologically active agents that are particularly difficult to deliver to cells include proteins, nucleic acid-based drugs, and derivatives thereof, particularly drugs that include relatively large oligonucleotides, such as mRNA or guide RNA. Compositions for delivery of promising mRNA therapy or editing technologies into cells, such as for delivery of CRISPR/Cas9 system components, have become of particular interest.

With the advent of the recent pandemic, messenger RNA therapy has become an increasingly important option for treatment of various diseases, including for viral infectious diseases and for those associated with deficiency of one or more proteins. Compositions with useful properties for in vitro and in vivo delivery that can stabilize and/or deliver RNA components, have also become of particular interest.

There thus continues to be a need in the art for novel lipid compounds to develop lipid nanoparticles or other lipid delivery mechanisms for therapeutics delivery. This invention answers that need.

### SUMMARY OF THE INVENTION

Disclosed herein are novel ionizable lipids that can be used in combination with at least one other lipid component, such as neutral lipids, cholesterol, and polymer conjugated lipids, to form lipid nanoparticle compositions. The lipid nanoparticle compositions may be used to facilitate the intracellular delivery of therapeutic nucleic acids in vitro and/or in vivo.

Disclosed herein are ionizable amine-containing lipids useful for formation of lipid nanoparticle compositions. Such LNP compositions may have properties advantageous for delivery of nucleic acid cargo, such as delivery of coding and non-coding RNAs to cells. Methods for treatment of various diseases or conditions, such as those caused by infectious entities and/or insufficiency of a protein, using the disclosed lipid nanoparticles are also provided.

Disclosed below are ionizable lipids of Formulas (IO)-(VIIO) and Formulas (I)-(VIID).

In some embodiments, disclosed are ionizable lipids of Formula (IO):

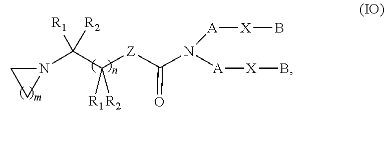

pharmaceutically acceptable salts thereof, and stereoisomers of any of the foregoing, wherein:
each R₁ and each R₂ is independently H, C₁-C₃ branched or unbranched alkyl, C₂-C₃ branched or unbranched alkenyl, OH, halogen, SH, or NR₁₀R₁₁, or
   R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
m is 1, 2, 3, 4, 5, 6, 7 or 8;
n is 0, 1, 2, 3 or 4;
Z is absent, O, S, or NR₁₂, wherein R₁₂ is H, C₁-C₇ branched or unbranched alkyl, C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally substituted with heteroatom or optionally substituted with OH, SH, or halogen;
each B is each independently C₁-C₁₆ branched or unbranched alkyl, C₂-C₁₆ branched or unbranched alkenyl, optionally substituted with heteroatom or optionally substituted with OH, SH, or halogen; and
each X is independently a biodegradable moiety.

In some embodiments, X is —OCO—, —COO—, —NHCO—, —CONH—, —C(O—R₁₃)—O—, —COO(CH₂)S—, —CONH(CH₂)S—, —C(O—R₁₃)—O—(CH₂)ₛ—, wherein R₁₃ is C₃-C₁₀ alkyl and s is 1, 2, 3, 4, or 5. In some embodiments, X is —OCO— or —COO—.

In some embodiments, disclosed are ionizable lipids of Formula (I) or (IA):

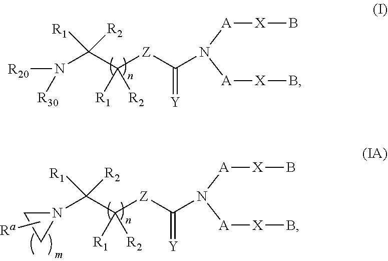

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or R₁ and R₂ are taken together to form a cyclic ring;
each R₁₀ and R₁₁ is independently H, C₁-C₃ branched or unbranched alkyl, C₂-C₃ branched or unbranched alkenyl, or R₁₀ and R₁₁ are taken together to form a heterocyclic ring;
m is 1, 2, 3, 4, 5, 6, 7 or 8;
n is 0, 1, 2, 3 or 4;
Y is O or S;
Z is absent, O, S, or N(R₁₂)(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, C₂-C₁₆ branched or unbranched alkenyl, optionally interrupted with one or more heteroatoms or optionally substituted with OH, SH, or halogen; and
each X is independently a biodegradable moiety.

In some embodiments, disclosed are ionizable lipids of the following formulas:

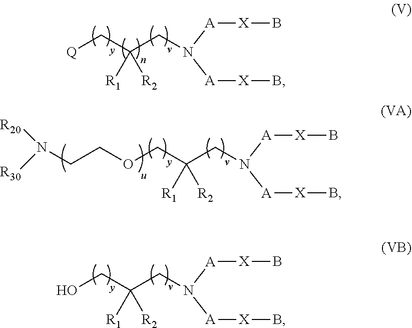

pharmaceutically acceptable salts thereof, and stereoisomers of any of the foregoing, wherein:
R₁ is H, C₁-C₃ branched or unbranched alkyl, C₂-C₃ branched or unbranched alkenyl, OH, halogen, SH, or NR₁₀R₁₁, and
R₂ is H, OH, halogen, SH, or NR₁₀R₁₁, or
R₁ and R₂ are taken together to form a cyclic ring;
R₁₀ and R₁₁ are each independently H or C₁-C₃ alkyl, or R₁₀ and R₁₁ are taken together to form a heterocyclic ring;
Q is OH or —(OCH₂CH₂)ᵤNR₂₀R₃₀,
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring optionally substituted with R^{a};
R^{a} is H, C₁-C₃ branched or unbranched alkyl, C₂-C₃ branched or unbranched alkenyl, halogen, OH, or SH;
u is 0, 1, 2, 3, 4, 5, 6, 7, or 8;
v is 0, 1, 2, 3, or 4;
y is 0, 1, 2, 3, or 4;
each A is independently C₁-C₁₆ branched or unbranched alkyl or C₁-C₁₆ branched or unbranched alkenyl, optionally interrupted with one or more heteroatoms or optionally substituted with OH, SH, or halogen;
each B is each independently C₁-C₁₆ branched or unbranched alkyl or C₂-C₁₆ branched or unbranched alkenyl, optionally interrupted with one or more heteroatoms or optionally substituted with OH, SH, or halogen; and
each X is independently a biodegradable moiety.

In some embodiments, in each of the above formulas, X is —OC(O)—, —C(O)O—, —N(R⁷)C(O)—, —C(O)N(R⁷)—, —C(O—R₁₃)—O—, —C(O)O(CH₂)S—, —OC(O)(CH₂)S—, —C(O)N(R⁷)(CH₂)S—, —N(R⁷)C(O)(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and each s is independently 0-16.

Also disclosed herein are pharmaceutical compositions comprising one or more compounds chosen from the ionizable lipid compounds in the formulas disclosed below, and a therapeutic agent. In some embodiments, the pharmaceutical compositions further comprise one or more components selected from neutral lipids, charged lipids, steroids, and polymer conjugated lipids. Such compositions may be useful for formation of lipid nanoparticles for delivery of a therapeutic agent.

In some embodiments, the present disclosure provides methods for delivering a therapeutic agent to a patient in need thereof, comprising administering to said patient a lipid nanoparticle composition comprising the ionizable lipid compound in the formulas disclosed below, a pharmaceutically acceptable salt thereof, and/or a stereoisomer of any of the foregoing and the therapeutic agent. In some embodiments, the method further comprises preparing a lipid nanoparticle composition comprising the ionizable lipid compound in the formulas disclosed below, a pharmaceutically acceptable salt thereof, and/or a stereoisomer of any of the foregoing and a therapeutic agent.

These and other aspects of the disclosure will be apparent upon reference to the following detailed description.

### BRIEF DESCRIPTION OF DRAWINGS

FIG. 1 represents the average radiance (p/s/cm2/sr) of various compounds in different body organs and areas in mice.

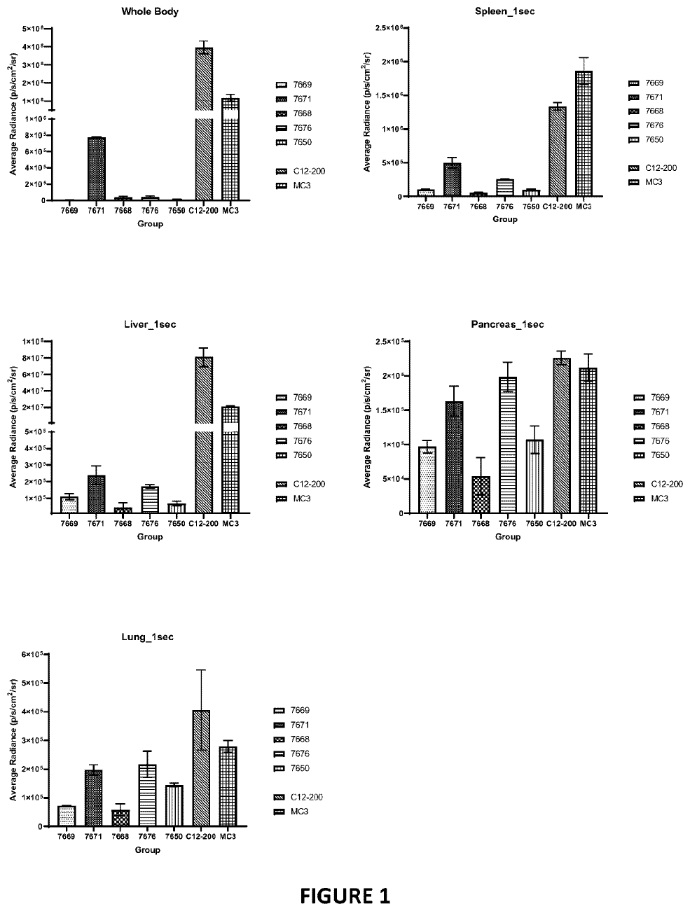

FIG. 2 shows bioluminescent images in mice liver (1 second after), spleen (1 second and 1 minute after) following administration of various compounds, for various lipids.

FIG. 3 show bioluminescent images in mice after administration of lipid compounds No. 7669 (left) and No. 7671 (right), respectively.

FIG. 4 show bioluminescent images in mice after administration of lipid compounds No. 7668 (left) and No. 7676 (right), respectively.

FIG. 5 show bioluminescent image in mice after administration of lipid compound No. 7650.

FIG. 6 show bioluminescent images in mice after administration of lipids C12-200 (left) and MC3 (right), respectively.

### DETAILED DESCRIPTION OF THE INVENTION

### Definitions

As used herein, the following terms have the meanings ascribed to them unless specified otherwise.

Unless defined otherwise, all technical and scientific terms used herein have the same meaning as is commonly understood by one of skill in the art to which this disclosure belongs.

As used in the specification and claims, the singular form “a”, “an” and “the” include plural references unless the context clearly dictates otherwise.

Unless the context requires otherwise, throughout the present specification and claims, the word “comprise” and variations thereof, such as, “comprises” and “comprising” are to be construed in an open and inclusive sense, that is, as “including, but not limited to”.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g., a mammal such as a human or an animal) model such as a rodent (e.g., mouse) or a non-human primate (e.g., monkey) model is contacted with a nucleic acid (e.g., nucleic acid in combination with a lipid of the present disclosure). Expression of the desired protein in the test sample or test animal is compared to expression of the desired protein in a control sample (e.g., a sample of cells in culture expressing the desired protein) or a control mammal (e.g., a mammal such as a human or an animal) model such as a rodent (e.g., mouse) or non-human primate (e.g., monkey) model that is not contacted with or administered the nucleic acid. When the desired protein is present in a control sample or a control mammal, the expression of a desired protein in a control sample or a control mammal may be assigned a value of 1.0. In some embodiments, inducing expression of a desired protein is achieved when the ratio of desired protein expression in the test sample or the test mammal to the level of desired protein expression in the control sample or the control mammal is greater than 1, for example, about 1.1, 1.5, 2.0, 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g., a sample of cells in culture expressing the target gene) or a test mammal (e.g., a mammal such as a human or an animal) model such as a rodent (e.g., mouse) or a non-human primate (e.g., monkey) model is contacted with a nucleic acid that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal) model such as a rodent (e.g., mouse) or non-human primate (e.g., monkey) model that is not contacted with or administered the nucleic acid. The expression of the target gene in a control sample or a control mammal may be assigned a value of 100%. In some embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

An “effective amount” or “therapeutically effective amount” of an active agent or therapeutic agent such as a therapeutic nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%), 15%), 10%), 5%), or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Sol. Cell. Probes, 8:91-98 (1994)). “Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipids” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

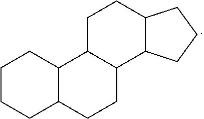 [SMILES: C1CCC2C(C1)CCC1C3CCCC3CCC21]

A non-limiting example of a steroid is cholesterol.

As used herein, “ionizable lipid” refers to a lipid capable of being charged. In some embodiments, an ionizable lipid includes one or more positively charged amine groups. In some embodiments, ionizable lipids are ionizable such that they can exist in a positively charged or neutral form depending on pH. The ionization of an ionizable lipid affects the surface charge of a lipid nanoparticle comprising the ionizable lipid tinder different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids. In some embodiments, ionizable lipids include those that are generally neutral, e.g., at physiological pH (e.g., pH about 7), but can carry net charge(s) at an acidic pH or basic pH. In one embodiment, ionizable lipids include those that are generally neutral at pH about 7, but can carry net charge(s) at an acidic pH. In one embodiment, ionizable lipids include those that are generally neutral at pH about 7, but can carry net charge(s) at a basic pH. In some embodiments, ionizable lipids do not include those cationic lipids or anionic lipids that generally carry net charge(s) at physiological pH (e.g., pH about 7).

The term “N:P ratio” refers to the molar ratio of the ionizable (in the physiological pH range) nitrogen atoms in a lipid to the phosphate groups in a nucleic acid (e.g., an RNA), e.g., in a lipid nanoparticle composition including lipid components and a nucleic acid (e.g., an RNA).

The term “polymer conjugated lipid” refers to a molecule comprising both a lipid portion and a polymer portion. A non-limiting example of a polymer conjugated lipid is a pegylated lipid. The term “pegylated lipid” refers to a molecule comprising both a lipid portion and a polyethylene glycol portion. Pegylated lipids are known in the art and include, for example, 1-(monomethoxy-polyethyleneglycol)-2,3-dimyristoylglycerol (PEG-D G) and the like.

The term “neutral lipid” refers to any lipid that exists either in an uncharged or neutral zwitterionic form at a selected pH. At physiological pH, such lipids include, but are not limited to, phosphotidylcholines such as 1,2-distearoyl-sn-glycero-3-phosphocholine (DSPC), 1,2-dipalmitoyl-5n-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, and steroids such as sterols and their derivatives. Neutral lipids may be synthetic or naturally derived.

The term “PEG lipid” or “PEGylated lipid” refers to a lipid conjugate comprising a polyethylene glycol (PEG) component.

The term “phospholipid” refers to a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

The term “lipid nanoparticle” refers to a particle having at least one dimension on the order of nanometers (e.g., 1-1,000 nm) and comprising one or more ionizable lipid compounds disclosed herein. In some embodiments, lipid nanoparticles comprising one or more ionizable lipid compounds disclosed herein, pharmaceutically acceptable salts thereof, and/or stereoisomers of any of the foregoing are included in a composition that can be used to deliver a therapeutic agent, such as a nucleic acid (e.g., mRNA), to a target site of interest (e.g., cell, tissue, organ, tumor, and the like). In some embodiments, lipid nanoparticles comprise one or more ionizable lipid compounds disclosed herein, pharmaceutically acceptable salts thereof, and/or stereoisomers of any of the foregoing, and a nucleic acid. In some embodiments, lipid nanoparticles comprise one or more ionizable lipid compounds disclosed herein, pharmaceutically acceptable salts thereof, and/or stereoisomers of any of the foregoing, and a nucleic acid. Such lipid nanoparticles typically comprise one or more ionizable lipid compounds disclosed herein, and one or more other lipids selected from neutral lipids, charged lipids, steroids, and polymer conjugated lipids. In some embodiments, the therapeutic agent, such as a nucleic acid, may be encapsulated in a lipid portion of the lipid nanoparticle or an aqueous space enveloped by some or all of a lipid portion of the lipid nanoparticle, thereby protecting it from enzymatic degradation or other undesirable effects induced by the mechanisms of the host organism or cells, e.g., an adverse immune response.

In some embodiments, the lipid nanoparticles have a mean diameter of from about 30 urn to about 150 urn, from about 40 nm to about 150 nm, from about 50 nm to about 150 un, from about 60 urn to about 130 nm, from about 70 nm to about 110 urn, from about 70 nm to about 100 un, from about 80 un to about 100 nm, from about 90 nm to about 100 nm, from about 70 to about 90 nm, from about 80 nm to about 90 un, from about 70 urn to about 80 nm, or about 30 nm, 35 nm, 40 nm, 45 nm, 50 nm, 55 nm, 60 nm, 65 nm, 70 urn, 75 urn, 80 urn, 85 nm, 90 nm, 95 nm, 100 nm, 105 nm, 110 nm, 115 nm, 120 nm, 125 nm, 130 nm, 135 nm, 140 nm, 145 urn, or 150 nm, and are substantially non-toxic. In some embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548. the full disclosures of which are herein incorporated by reference in their entirety for all purposes.

The term “polydispersity index” or “PDI” refers to a ratio that describes the homogeneity of the particle size distribution of a system, e.g., a lipid nanoparticle composition. A small value, e.g., less than 0.3, indicates a narrow particle size distribution.

As used herein, “encapsulated” by a lipid refers a therapeutic agent, such as a nucleic acid (e.g., mRNA), that is fully or partially encapsulated by a lipid nanoparticle. In some embodiments, the therapeutic agent such as a nucleic acid (e.g., mRNA) is fully encapsulated in a lipid nanoparticle.

“Serum-stable” in relation to nucleic acid-lipid nanoparticles means that the nucleic acid is not significantly degraded after exposure to a serum or nuclease assay that would significantly degrade free DNA or RNA. Suitable assays include, for example, a standard serum assay, a DNAse assay, or an RNAse assay.

Some techniques of administration can lead to systemic delivery of certain agents but not others. “Systemic delivery” means that a useful, such as a therapeutic, amount of an agent is delivered to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

“Alkyl” refers to a straight or branched hydrocarbon chain radical consisting solely of carbon and hydrogen atoms, which is saturated or unsaturated (i.e., contains one or more double (alkenyl) and/or triple bonds (alkynyl)), having, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n-propyl, 1-methylethyl (isopropyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group is optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain.

The term “substituted” used herein means any of the above groups (e.g., alkyl, alkylene, cycloalkyl or cycloalkylene) wherein at least one hydrogen atom is replaced by a bond to a non-hydrogen atom such as, but not limited to: a halogen atom such as F, Cl, Br, or I; oxo groups (=O); hydroxyl groups (—H); C₁-C₁₂ alkyl groups; cycloalkyl groups; —(C=O)OR; —O(C═O)R; —C(═O)R; —OR; —S(O)ₓR; —S—SR; —C(═O)SR; —SC(═O)R; —NRR′; —R′C(═O)R; —C(═O)RR′; —RC(═O)R′R″; —OC(═O)RR′; —RC(═O)OR′; —R′S(O)_{X}R″R; —R′S(O)_{X}R; and —S(O)ₓRR′, wherein: R, R′, and R″ is, at each occurrence, independently H, C₁-C₁₅ alkyl or cycloalkyl, and x is 0, 1 or 2. In some embodiments, the substituent is a C₁-C₁₂ alkyl group. In some embodiments, the substituent is a cycloalkyl group. In some embodiments, the substituent is a halo group, such as fluoro. In some embodiments, the substituent is an oxo group. In some embodiments, the substituent is a hydroxyl group. In some embodiments, the substituent is an alkoxy group (—OR). In some embodiments, the substituent is a carboxyl group. In some embodiments, the substituent is an amine group (—NRR′).

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

The present disclosure is also meant to encompass all pharmaceutically acceptable compounds of the ionizable lipid compounds in the formulas disclosed herein, being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ^{U}C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These isotopically-labelled compounds could be useful to help determine or measure the effectiveness of the compounds, by characterizing, for example, the site or mode of action, or binding affinity to pharmacologically important site of action. Certain isotopically-labelled lipid compounds, for example, those incorporating a radioactive isotope, are useful in drug and/or substrate tissue distribution studies. The radioactive isotopes tritium, i.e., ³H, and carbon-14, i.e., ¹⁴C, may be useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be useful in some circumstances.

Substitution with positron emitting isotopes, such as ^{U}C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

The present disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments of the disclosure include compounds produced by a process comprising administering an ionizable lipid of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Non-limiting examples of inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethyl aminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Non-limiting examples of organic bases are isopropylamine, diethylamine, ethanolamine, trimethylamine, dicyclohexylamine, choline and caffeine.

Crystallization of ionizable lipid(s) disclosed herein may produce a solvate of the ionizable lipid(s). As used herein, the term “solvate” refers to an aggregate that comprises one or more molecules of an ionizable lipid compound of the disclosure with one or more molecules of solvent. The solvent may be water, in which case the solvate may be a hydrate. Alternatively, the solvent may be an organic solvent. Thus, the lipid compounds of the present disclosure may exist as a hydrate, including a monohydrate, dihydrate, hemihydrate, sesquihydrate, trihydrate, tetrahydrate and the like, as well as the corresponding solvated forms. Solvates of the lipid compound of the disclosure may be true solvates, while in other cases, the lipid compound of the disclosure may merely retain adventitious water or be a mixture of water plus some adventitious solvent.

A “pharmaceutical composition” refers to a composition which may comprise an ionizable lipid compound of the disclosure and a medium generally accepted in the art for the delivery of the biologically active compound to mammals, e.g., humans. Such a medium includes pharmaceutically acceptable carriers, diluents or excipients therefor.

“Effective amount” or “therapeutically effective amount” refers to that amount of an ionizable lipid compound of the disclosure which, when administered to a mammal, such as a human, is sufficient to effect treatment in the mammal, such as a human. The amount of an ionizable lipid compound of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, such as a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The ionizable lipid compounds of the disclosure, or their pharmaceutically acceptable salts may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the ionizable lipid compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

In the following description, certain specific details are set forth to provide a thorough understanding of various embodiments of the disclosure. However, one of ordinary skill in the art will understand that the disclosure may be practiced without these details.

### Ionizable Lipid Compounds

In some embodiments, disclosed are ionizable lipids of Formula (IO):

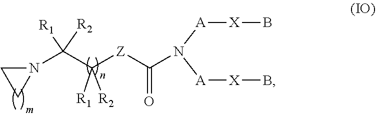

pharmaceutically acceptable salts thereof, and stereoisomers of any of the foregoing, wherein:
each R₁ and each R₂ is independently H, C₁-C₃ branched or unbranched alkyl, C₂-C₃ branched or unbranched alkenyl, OH, halogen, SH, or NR₁₀R₁₁, or
   R₁ and R₂ are taken together to form a cyclic ring;
each R₁₀ and R₁₁ is independently H, C₁-C₃ branched or unbranched alkyl, C₂-C₃ branched or unbranched alkenyl, or
R₁₀ and R₁₁ are taken together to form a heterocyclic ring;
m is 1, 2, 3, 4, 5, 6, 7 or 8;
n is 0, 1, 2, 3 or 4;
Z is absent, O, S, or NR₁₂, wherein R₁₂ is H or C₁-C₇ branched or unbranched alkyl, provided that when Z is not absent, the adjacent R₁ and R₂ cannot be OH, NR₁₀R₁₁, or SH;
each A is each independently C₁-C₁₆ branched or unbranched alkyl, or C₁-C₁₆ branched or unbranched alkenyl, optionally substituted with heteroatom or optionally substituted with OH, SH, or halogen;
each B is each independently C₁-C₁₆ branched or unbranched alkyl, or C₂-C₁₆ branched or unbranched alkenyl, optionally substituted with a heteroatom or optionally substituted with OH, SH, or halogen; and
each X is independently a biodegradable moiety.

In some embodiments, in formula (IO), X is —OCO—, —COO—, —NHCO—, —CONH—, —C(O—R₁₃)—O—, —COO(CH₂)S—, —CONH(CH₂)S—, —C(O—R₁₃)—O—(CH₂)ₛ—, wherein R₁₃ is C₃-C₁₀ alkyl and s is 1, 2, 3, 4, or 5. In some embodiments, X is —OCO— or —COO—.

In some embodiments, disclosed are ionizable lipids of Formula (I):

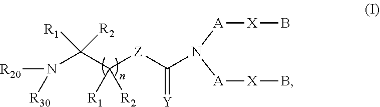

pharmaceutically acceptable salts thereof, and stereoisomers of any of the foregoing, wherein:
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring, optionally substituted with R^{a};
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or
R₁₀ and R₁₁ are taken together to form a heterocyclic ring;
n is 0, 1, 2, 3 or 4;
Y is O or S;
Z is absent, O, S, or N(R₁₂)(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen;
each X is independently a biodegradable moiety.

In some embodiments, R₂₀ and R₃₀ are each independently H or C₁-C₃ branched or unbranched alkyl.

In some embodiments, R₂₀ and R₃₀ together with the adjacent N atom form a 3 to 7 membered cyclic ring, optionally substituted with R^{a}. In some embodiments, R^{a} is H, C₁-C₃ branched or unbranched alkyl or OH.

In some embodiments, disclosed are ionizable lipids of Formula (IA):

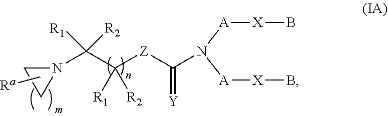

pharmaceutically acceptable salts thereof, and stereoisomers of any of the foregoing, wherein:
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, or
R₁ and R₂ are taken together to form a cyclic ring;
each R₁₀ and R₁₁ is independently H, C₁-C₃ branched or unbranched alkyl, C₂-C₃ branched or unbranched alkenyl, or
R₁₀ and R₁₁ are taken together to form a heterocyclic ring;
m is 1, 2, 3, 4, 5, 6, 7 or 8;
n is 0, 1, 2, 3 or 4;
Y is O or S;
Z is absent, O, S, or N(R₁₂)(R₁₂), wherein each R₁₂ is independently H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally interrupted with one or more heteroatoms or optionally substituted with OH, SH, or halogen;
each B is each independently C₁-C₁₆ branched or unbranched alkyl, or C₂-C₁₆ branched or unbranched alkenyl, optionally interrupted with one or more heteroatoms or optionally substituted with OH, SH, or halogen;
each X is independently a biodegradable moiety.

In some embodiments, disclosed are ionizable lipids of Formula (IIO):

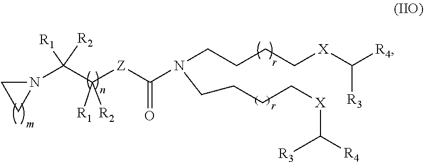

pharmaceutically acceptable salts thereof, and stereoisomers of any of the foregoing, wherein:
each R₁ and each R₂ is independently H, C₁-C₃ branched or unbranched alkyl, C₂-C₃ branched or unbranched alkenyl, OH, halogen, SH, or NR₁₀R₁₁, wherein each R₁₀ and R₁₁ is independently H, C₁-C₃ branched or unbranched alkyl, C₂-C₃ branched or unbranched alkenyl, or R₁₀ and R₁₁ are taken together to form a heterocyclic ring, or
R₁ and R₂ are taken together to form a cyclic ring;
m is 1, 2, 3, 4, 5, 6, 7 or 8;
n is 0, 1, 2, 3 or 4;
each r is independently 0, 1, 2, 3, 4, 5, 6, 7 or 8;
each R₃ and each R₄ is independently H, C₃-C₁₀ branched or unbranched alkyl, or C₃-C₁₀ branched or unbranched alkenyl, provided that at least one of R₃ and R₄ is not H;
Z is absent, O, S, or NR₁₂; wherein R₁₂ is C₁-C₇ alkyl;
X is a biodegradable moiety.

In some embodiments, in formula (IIO), X is —OCO—, —COO—, —NHCO—, —CONH—, —C(O—R₁₃)—O—, —COO(CH₂)S—, —CONH(CH₂)S—, —C(O—R₁₃)—O—(CH₂)ₛ—, wherein R₁₃ is C₃-C₁₀ alkyl and s is 1, 2, 3, 4, or 5. In some embodiments, X is —OCO— or —COO—.

In some embodiments, disclosed are ionizable lipids of Formula (IIA) or (IIB):

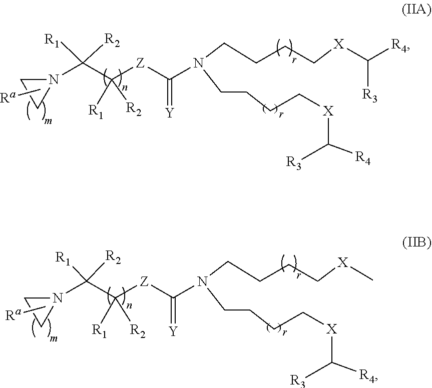

pharmaceutically acceptable salts thereof, and stereoisomers of any of the foregoing, wherein:
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, wherein each R₁₀ and R₁₁ is independently H, C₁-C₃ branched or unbranched alkyl, C₂-C₃ branched or unbranched alkenyl, or R₁₀ and R₁₁ are taken together to form a heterocyclic ring, or
R₁ and R₂ are taken together to form a cyclic ring;
m is 1, 2, 3, 4, 5, 6, 7 or 8;
n is 0, 1, 2, 3 or 4;
each r is independently 0, 1, 2, 3, 4, 5, 6, 7 or 8;
each R₃ and each R₄ is independently H, C₃-C₁₀ branched or unbranched alkyl, or C₃-C₁₀ branched or unbranched alkenyl (optionally, at least one of R₃ and R₄ is not H);
Y is O or S;
Z is absent, O, S, or (NR₁₂)(R₁₂), wherein R₁₂ is independently H or C₁-C₇ alkyl;
each X is independently a biodegradable moiety.

In some embodiments, disclosed are ionizable lipids of Formula (IIC) or (IID):

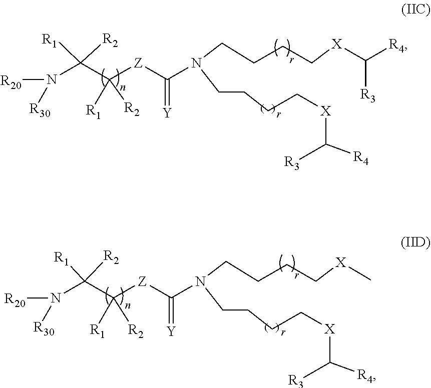

pharmaceutically acceptable salts thereof, and stereoisomers of any of the foregoing. R₂₀ and R₃₀ are each independently H or C₁-C₃ branched or unbranched alkyl. The definitions of other variables in this formula are the same as the definitions of the variables in Formula (IIA).

In some embodiments, disclosed are ionizable lipids of Formula (IIO):

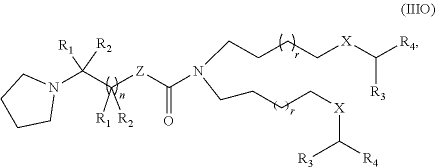

pharmaceutically acceptable salts thereof, and stereoisomers of any of the foregoing, wherein:
each R₁ and each R₂ is independently H, C₁-C₃ branched or unbranched alkyl, C₂-C₃ branched or unbranched alkenyl, OH, halogen, SH, or NR₁₀R₁₁, wherein each R₁₀ and R₁₁ is independently H, C₁-C₃ branched or unbranched alkyl, C₂-C₃ branched or unbranched alkenyl, or R₁₀ and R₁₁ are taken together to form a heterocyclic ring, or
   R₁ and R₂ are taken together to form a cyclic ring;
n is 0, 1, 2, 3 or 4;
each r is independently 0, 1, 2, 3, 4, 5, 6, 7 or 8;
each R₃ and each R₄ is independently H, C₃-C₁₀ branched or unbranched alkyl, or C₃-C₁₀ branched or unbranched alkenyl, provided that at least one of R₃ and R₄ is not H;
Z is absent, O, S, or NR₁₂; wherein R₁₂ is C₁-C₇ alkyl;
X is a biodegradable moiety.

In some embodiments, in formula (IIIO), X is —OCO—, —COO—, —NHCO—, —CONH—, —C(O—R₁₃)—O-(acetal), —COO(CH₂)ₛ—, —CONH(CH₂)ₛ—, —C(O—R₁₃)—O—(CH₂)ₛ—; wherein R₁₃ is C₃-C₁₀ alkyl.

In some embodiments, disclosed are ionizable lipids of Formula (IIIA) or (IIIB):

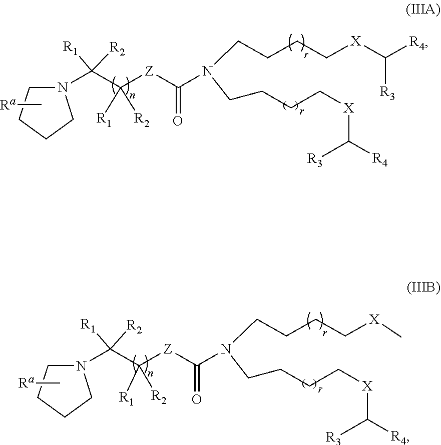

pharmaceutically acceptable salts thereof, and stereoisomers of any of the foregoing, wherein:
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁, wherein each R₁₀ and R₁₁ is independently H, C₁-C₃ branched or unbranched alkyl, C₂-C₃ branched or unbranched alkenyl, or R₁₀ and R₁₁ are taken together to form a heterocyclic ring, or
R₁ and R₂ are taken together to form a cyclic ring;
n is 0, 1, 2, 3 or 4;
each r is independently 0, 1, 2, 3, 4, 5, 6, 7 or 8;
each R₃ and each R₄ is independently H, C₃-C₁₀ branched or unbranched alkyl, or C₃-C₁₀ branched or unbranched alkenyl, provided that at least one of R₃ and R₄ is not H;
Z is absent, O, S, or N(R₁₂)(R₁₂), wherein R₁₂ is independently H or C₁-C₇ alkyl;
each X is independently a biodegradable moiety.

In some embodiments, in each of the above formulas, X is —OC(O)—, —C(O)O—, —N(R⁷)C(O)—, —C(O)N(R⁷)—, —C(O—R₁₃)—O—, —C(O)O(CH₂)ₛ—, —OC(O)(CH₂)ₛ—, —C(O)N(R⁷)(CH₂)ₛ—, —N(R⁷)C(O)(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and each s is independently 0-16.

In some embodiments, in each of the above formulas, X is —OC(O)—, —C(O)O—, —C(O)O(CH₂)ₛ—, or —OC(O)(CH₂)ₛ—. In some embodiments, s is 0, 1, 2, 3, 4, 5, 6, 7, 8, 9, or 10.

In some embodiments, in each of the above formulas, X is —C(O)N(R⁷)—, —N(R⁷)C(O)—, —C(O)N(R⁷)(CH₂)ₛ—, or —N(R⁷)C(O)(CH₂)ₛ—, wherein R⁷ is independently H, alkyl, alkenyl, or cycloalkyl. In some embodiments, s is 0, 1, 2, 3, 4, 5, 6, 7, 8, 9, or 10.

In some embodiments, in each of the above formulas, X is —C(O—R₁₃)—O-(acetal) or —C(O—R₁₃)—O—(CH₂)ₛ—, wherein R₁₃ is C₃-C₁₀ alkyl.

In some embodiments, in each of the above formulas, R^{a} is H, C₁-C₃ branched or unbranched alkyl or OH.

In some embodiments, R^{a} is H, methyl, ethyl, propyl, or OH.

In one embodiment, R^{a} is H or OH.

In some embodiments, Z is absent, S, O, or NH. In one embodiment, Z is absent. In one embodiment Z is NH. In one embodiment Z is S. In one embodiment Z is O.

In some embodiments, m is 1, 2, 3, or 4.

In some embodiments, n is 0, 1, or 2.

In some embodiments, in each of the above formulas, the two r variables in the same formula are the same.

In some embodiments, in each of the above formulas, the two r variables in the same formula are different.

In some embodiments, in each of the above formulas, the two X variables in the same formula are the same.

In some embodiments, in each of the above formulas, the two X variables in the same formula are different,

In some embodiments, in each of the above formulas, the two R₃ variables in the same formula are the same. In some embodiments, in each of the above formulas, the two R₄ variables in the same formula are the same.

In some embodiments, in each of the above formulas, the two R₃ variables in the same formula are different. In some embodiments, in each of the above formulas, the two R₄ variables in the same formula are different.

In some embodiments, in each of the above formulas, R₁ and R₂ are each H. In some embodiments, in each of the above formulas, each R₁ is H, and one of the R₂ variables is OH.

In some embodiments, disclosed are ionizable lipids of Formula (IVO):

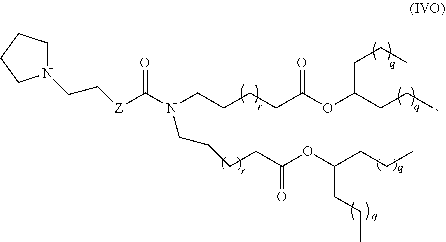

pharmaceutically acceptable salts, thereof, and stereoisomers of any of the foregoing, wherein:
each r is independently 0, 1, 2, 3, 4, 5, 6, 7 or 8;
each q is independently 1, 2, 3, 4, 5, 6, 7 or 8, 9, or 10; and
Z is absent, O, S, or NR₁₂, wherein R₁₂ is H or C₁-C₇ branched or unbranched alkyl.

In some embodiments, disclosed are ionizable lipids of one of the following formulas:

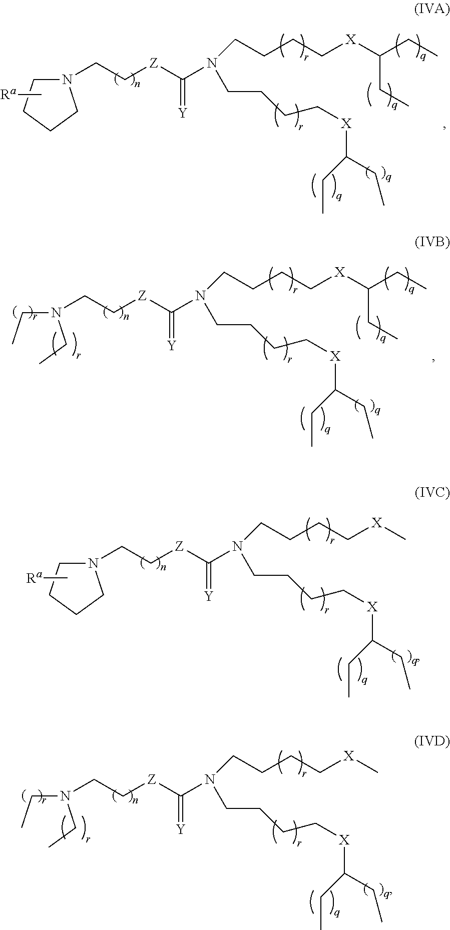

pharmaceutically acceptable salts, thereof, and stereoisomers of any of the foregoing, wherein:
R^{a} is H, C₁-C₃ branched or unbranched alkyl or OH;
each n is independently 0, 1, 2, 3, or 4;
each r is independently 0, 1, 2, 3, 4, 5, 6, 7 or 8;
each q is independently 1, 2, 3, 4, 5, 6, 7 or 8, 9, or 10;
X is —OC(O)—, —C(O)O—, —N(R⁷)C(O)—, —C(O)N(R⁷)—, —C(O—R₁₃)—O—, —C(O)O(CH₂)ₛ—, —OC(O)(CH₂)ₛ—, —C(O)N(R⁷)(CH₂)ₛ—, —N(R⁷)C(O)(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and each s is independently 0-16; and
Z is absent, O, S, or NR₁₂, wherein R₁₂ is H or C₁-C₇ branched or unbranched alkyl.

In some embodiments, in each of the above formulas, R^{a} is H, methyl, ethyl, propyl, or OH.

In some embodiments, in each of the above formulas, Z is absent.

In some embodiments, in each of the above formulas, Z is S.

In some embodiments, in each of the above formulas, Z is O.

In some embodiments, in each of the above formulas, Z is NH.

In some embodiments, in each of the above formulas, r is 2.

In some embodiments, in each of the above formulas, r is 3.

In some embodiments, in each of the above formulas, r is 4.

In some embodiments, in each of the above formulas, q is 3.

In some embodiments, in each of the above formulas, q is 4.

In some embodiments, in each of the above formulas, Z is absent, r is 4 and q is 4.

In some embodiments, in each of the above formulas, X is —OC(O)—, —C(O)O—, —C(O)O(CH₂)ₛ—, or —OC(O)(CH₂)ₛ—. In some embodiments, s is 0, 1, 2, 3, 4, 5, 6, 7, 8, 9, or 10.

In some embodiments, X is —C(O)N(R⁷)—, —N(R⁷)C(O)—, —C(O)N(R⁷)(CH₂)ₛ—, or —N(R⁷)C(O)(CH₂)ₛ—, wherein R⁷ is independently H, alkyl, alkenyl, or cycloalkyl. In some embodiments, s is 0, 1, 2, 3, 4, 5, 6, 7, 8, 9, or 10.

In some embodiments, X is —C(O—R₁₃)—O-(acetal) or —C(O—R₁₃)—O—(CH₂)ₛ—, wherein R₁₃ is C₃-C₁₀ alkyl.

In some embodiments, the disclosure relates to ionizable lipids of Formula (VO):

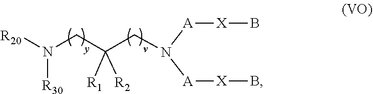

pharmaceutically acceptable salts thereof, and stereoisomers of any of the foregoing, wherein:
R₁ is H, C₁-C₃ alkyl, OH, halogen, SH, or NR₁₀R₁₁ and R₂ is OH, halogen, SH, or NR₁₀R₁₁, wherein R₁₀ and R₁₁ are each independently H or C₁-C₃ alkyl or R₁₀ and R₁₁ are taken together to form a heterocyclic ring, or
R₁ and R₂ are taken together to form a cyclic ring;
R₂₀ and R₃₀ are each independently H, C₁-C₅ branched or unbranched alkyl, or C₁-C₅ branched or unbranched alkenyl, or
R₂₀ and R₃₀ are taken together to form a cyclic ring;
v is 1, 2, 3, or 4;
y is 1, 2, 3, or 4;
each A is independently C₁-C₁₆ branched or unbranched alkyl or C₁-C₁₆ branched or unbranched alkenyl, optionally substituted with heteroatom or substituted with OH, SH, or halogen;
each B is each independently C₁-C₁₆ branched or unbranched alkyl or C₁-C₁₆ branched or unbranched alkenyl, optionally substituted with heteroatom or substituted with OH, SH, or halogen; and
X is a biodegradable moiety.

In some embodiments, in formula (VO), X is —OCO—, —COO—, —NHCO—, —CONH—, —C(O—R₁₃)—O—, —COO(CH₂)ₛ—, —CONH(CH₂)ₛ—, —C(O—R₁₃)—O—(CH₂)ₛ—, wherein R₁₃ is C₃-C₁₀ alkyl and s is 1, 2, 3, 4, or 5.

In some embodiments, disclosed are ionizable lipids of Formula (V):

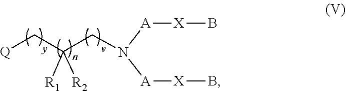

pharmaceutically acceptable salts thereof, and stereoisomers of any of the foregoing,
wherein
R₁ is H, C₁-C₃ branched or unbranched alkyl, C₂-C₃ branched or unbranched alkenyl, OH, halogen, SH, or NR₁₀R₁₁, and
R₂ is H, OH, halogen, SH, or NR₁₀R₁₁, or
R₁ and R₂ are taken together to form a cyclic ring;
R₁₀ and R₁₁ are each independently H or C₁-C₃ alkyl, or R₁₀ and R₁₁ are taken together to form a heterocyclic ring;
Q is OH or —(OCH₂CH₂)ᵤNR₂₀R₃₀,
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring optionally substituted with R^{a};
R^{a} is H, C₁-C₃ branched or unbranched alkyl, C₂-C₃ branched or unbranched alkenyl, halogen, OH, or SH;
u is 0, 1, 2, 3, 4, 5, 6, 7, or 8;
v is 0, 1, 2, 3, or 4;
y is 0, 1, 2, 3, or 4;
each A is independently C₁-C₁₆ branched or unbranched alkyl or C₁-C₁₆ branched or unbranched alkenyl, optionally interrupted with one or more heteroatoms or optionally substituted with OH, SH, or halogen;
each B is each independently C₁-C₁₆ branched or unbranched alkyl or C₂-C₁₆ branched or unbranched alkenyl, optionally interrupted with one or more heteroatoms or optionally substituted with OH, SH, or halogen; and
each X is independently a biodegradable moiety.

In some embodiments, the disclosure relates to ionizable lipids of one of the following formulas:

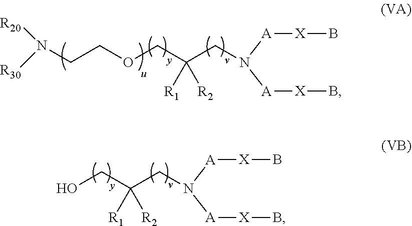

pharmaceutically acceptable salts thereof, and stereoisomers of any of the foregoing, wherein:
R₁ is H, C₁-C₃ branched or unbranched alkyl, C₂-C₃ branched or unbranched alkenyl, OH, halogen, SH, or NR₁₀R₁₁, and
R₂ is H, OH, halogen, SH, or NR₁₀R₁₁, or
R₁ and R₂ are taken together to form a cyclic ring;
R₁₀ and R₁₁ are each independently H or C₁-C₃ alkyl, or R₁₀ and R₁₁ are taken together to form a heterocyclic ring;
R₂₀ and R₃₀ are each independently H, C₁-C₅ branched or unbranched alkyl, or C₂-C₅ branched or unbranched alkenyl, or
R₂₀ and R₃₀ together with the adjacent N atom form a 3 to 7 membered cyclic ring optionally substituted with R^{a};
R^{a} is H, C₁-C₃ branched or unbranched alkyl, C₂-C₃ branched or unbranched alkenyl, halogen, OH, or SH;
u is 0, 1, 2, 3, 4, 5, 6, 7, or 8;
v is 0, 1, 2, 3, or 4;
y is 0, 1, 2, 3, or 4;
each A is independently C₁-C₁₆ branched or unbranched alkyl or C₁-C₁₆ branched or unbranched alkenyl, optionally interrupted with one or more heteroatoms or optionally substituted with OH, SH, or halogen;
each B is each independently C₁-C₁₆ branched or unbranched alkyl or C₂-C₁₆ branched or unbranched alkenyl, optionally interrupted with one or more heteroatoms or optionally substituted with OH, SH, or halogen; and
each X is independently a biodegradable moiety.

In some embodiments, the disclosure relates to ionizable lipids of Formula (VIO):

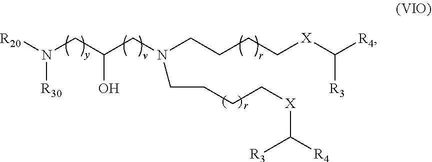

pharmaceutically acceptable salts thereof, and stereoisomers of any of the foregoing, wherein:
R₂₀ and R₃₀ are independently H, C₁-C₅ branched or unbranched alkyl, or C₂-C₅ branched or unbranched alkenyl, or
R₂₀ and R₃₀ are taken together to form a cyclic ring;
v is 1, 2, 3, or 4;
y is 1, 2, 3, or 4;
each R₃ and each R₄ is independently H, C₁-C₃ branched or unbranched alkyl, or C₂-C₃ branched or unbranched alkenyl, provided that at least one of R₃ and R₄ is not H;
each r is independently 0, 1, 2, 3, 4, 5, 6, 7 or 8; and
X is —OCO—, —COO—, —NHCO—, —CONH—, —C(O—R₁₃)—O—, —COO(CH₂)ₛ—, —CONH(CH₂)ₛ—, —C(O—R₁₃)—O—(CH₂)ₛ—, wherein R₁₃ is C₃-C₁₀ alkyl and s is 1, 2, 3, 4, or 5.

In some embodiments, the disclosure relates to ionizable lipids of one of the following formulas:

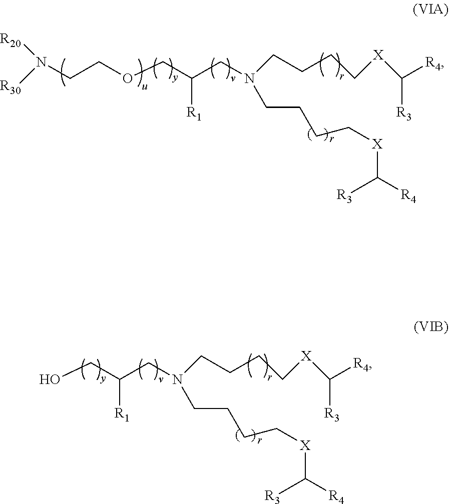

pharmaceutically acceptable salts thereof, and stereoisomers of any of the foregoing, wherein:
R₂₀ and R₃₀ are independently H, C₁-C₅ branched or unbranched alkyl, or C₂-C₅ branched or unbranched alkenyl, or
R₂₀ and R₃₀ together with the adjacent N atom form a 3 to 7 membered cyclic ring;
R₁ is independently H or OH;
u is 0, 1, 2, 3, or 4;
v is 0, 1, 2, 3, or 4;
y is 0, 1, 2, 3, or 4;
each R₃ and each R₄ is independently H, C₁-C₃ branched or unbranched alkyl, or C₂-C₃ branched or unbranched alkenyl, provided that at least one of R₃ and R₄ is not H;
each r is independently 0, 1, 2, 3, 4, 5, 6, 7 or 8; and
X is —OCO—, —COO—, —NR⁷CO—, —CONR⁷—, —C(O—R₁₃)—O—, —COO(CH₂)ₛ—, —OCO(CH₂)ₛ—, —CONR⁷(CH₂)ₛ—, —NR⁷CO(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and s is independently 0-16.

In some embodiments, in each of the above formulas, X is —OC(O)—, —C(O)O—, —C(O)O(CH₂)S—, or —OC(O)(CH₂)S—. In some embodiments, s is 0, 1, 2, 3, 4, 5, 6, 7, 8, 9, or 10.

In some embodiments, in each of the above formulas, X is —C(O)N(R⁷)—, —N(R⁷)C(O)—, —C(O)N(R⁷)(CH₂)S—, or —N(R⁷)C(O)(CH₂)S—, wherein R⁷ is independently H, alkyl, alkenyl, or cycloalkyl. In some embodiments, s is 0, 1, 2, 3, 4, 5, 6, 7, 8, 9, or 10.

In some embodiments, in each of the above formulas, X is —C(O—R₁₃)—O-(acetal) or —C(O—R₁₃)—O—(CH₂)ₛ—, wherein R₁₃ is C₃-C₁₀ alkyl.

In some embodiments, in each of the above formulas, R^{a} is H, C₁-C₃ branched or unbranched alkyl or OH. In some embodiments, R^{a} is H, methyl, ethyl, propyl, or OH.

In some embodiments, in each of the above formulas, the two r variables in the same formula are the same.

In some embodiments, in each of the above formulas, the two r variables in the same formula are different.

In some embodiments, in each of the above formulas, the two X variables in the same formula are the same.

In some embodiments, in each of the above formulas, the two X variables in the same formula are different.

In some embodiments, in each of the above formulas, the two R₃ variables in the same formula are the same. In some embodiments, in each of the above formulas, the two R₄ variables in the same formula are the same.

In some embodiments, in each of the above formulas, the two R₃ variables in the same formula are different. In some embodiments, in each of the above formulas, the two R₄ variables in the same formula are different.

In some embodiments, the disclosure relates to ionizable lipids of Formula (VIIO):

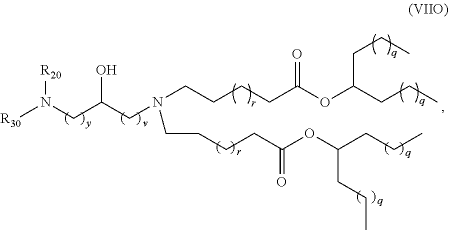

pharmaceutically acceptable salts thereof, and stereoisomers of any of the foregoing, wherein:
R₂₀ and R₃₀ are independently H, C₁-C₅ branched or unbranched alkyl, or C₂-C₅ branched or unbranched alkenyl, or
R₂₀ and R₃₀ are taken together to form a cyclic ring;
v is 1, 2, 3, or 4;
y is 1, 2, 3, or 4;
each r is independently 0, 1, 2, 3, 4, 5, 6, 7 or 8; and
each q is independently 1, 2, 3, 4, 5, 6, 7 or 8, 9, or 10.

In some embodiments, the disclosure relates to ionizable lipids of one of the following formulas:

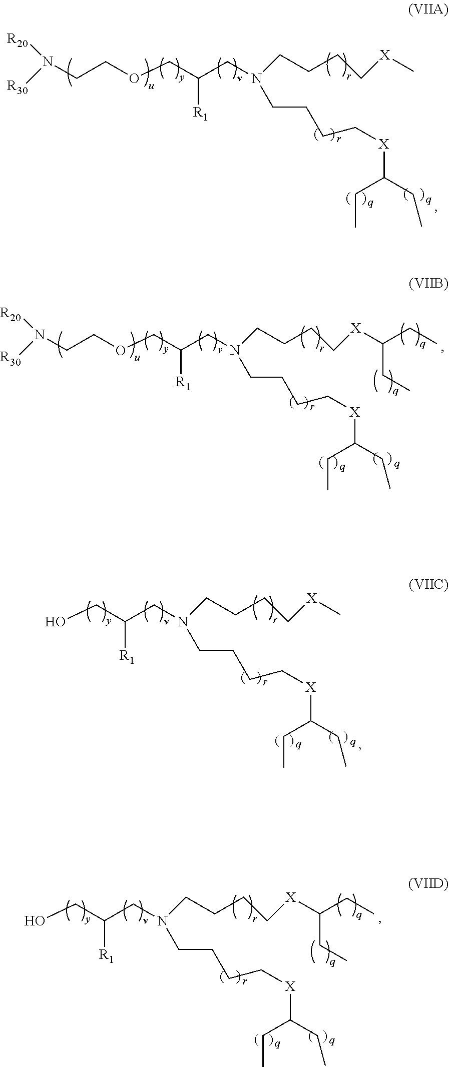

pharmaceutically acceptable salts thereof, and stereoisomers of any of the foregoing, wherein:
R₂₀ and R₃₀ are independently H, C₁-C₅ branched or unbranched alkyl, or C₂-C₅ branched or unbranched alkenyl, or
R₂₀ and R₃₀ are taken together to form a cyclic ring;
X is —OC(O)—, —C(O)O—, —N(R⁷)C(O)—, —C(O)N(R⁷)—, —C(O—R₁₃)—O—, —C(O)O(CH₂)ₛ—, —OC(O)(CH₂)ₛ—, —C(O)N(R⁷)(CH₂)ₛ—, —N(R⁷)C(O)(CH₂)ₛ—, —C(O—R₁₃)—O—(CH₂)ₛ—, wherein each R⁷ is independently H, alkyl, alkenyl, cycloalkyl, hydroxyalkyl, or aminoalkyl, each R₁₃ is independently C₃-C₁₀ alkyl, and each s is independently 0-16;
u is 0, 1, 2, 3, or 4;
v is 0, 1, 2, 3, or 4;
y is 0, 1, 2, 3, or 4;
each r is independently 0, 1, 2, 3, 4, 5, 6, 7 or 8; and
each q is independently 1, 2, 3, 4, 5, 6, 7 or 8, 9, or 10.

In some embodiments, in each of the above formulas, X is —OC(O)—, —C(O)O—, —C(O)O(CH₂)ₛ—, or —OC(O)(CH₂)ₛ—. In some embodiments, s is 0, 1, 2, 3, 4, 5, 6, 7, 8, 9, or 10.

In some embodiments, in each of the above formulas, X is —C(O)N(R⁷)—, —N(R⁷)C(O)—, —C(O)N(R⁷)(CH₂)ₛ—, or —N(R⁷)C(O)(CH₂)ₛ—, wherein R⁷ is independently H, alkyl, alkenyl, or cycloalkyl. In some embodiments, s is 0, 1, 2, 3, 4, 5, 6, 7, 8, 9, or 10.

In some embodiments, in each of the above formulas, X is —C(O—R₁₃)—O-(acetal) or —C(O—R₁₃)—O—(CH₂)ₛ—, wherein R₁₃ is C₃-C₁₀ alkyl.

In some embodiments, in each of the above formulas, r is 2.

In some embodiments, in each of the above formulas, r is 3.

In some embodiments, in each of the above formulas, r is 4.

In some embodiments, in each of the above formulas, q is 3.

In some embodiments, in each of the above formulas, q is 4.

In some embodiments, in each of the above formulas, r is 4 and q is 4.

In some embodiments, in each of the above formulas, B or

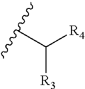

is selected from:

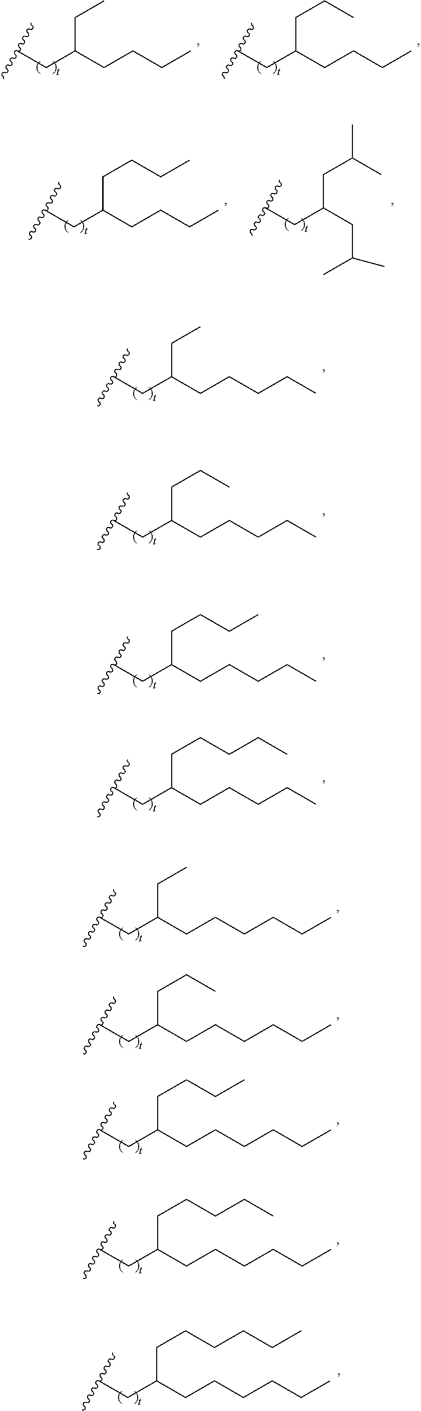

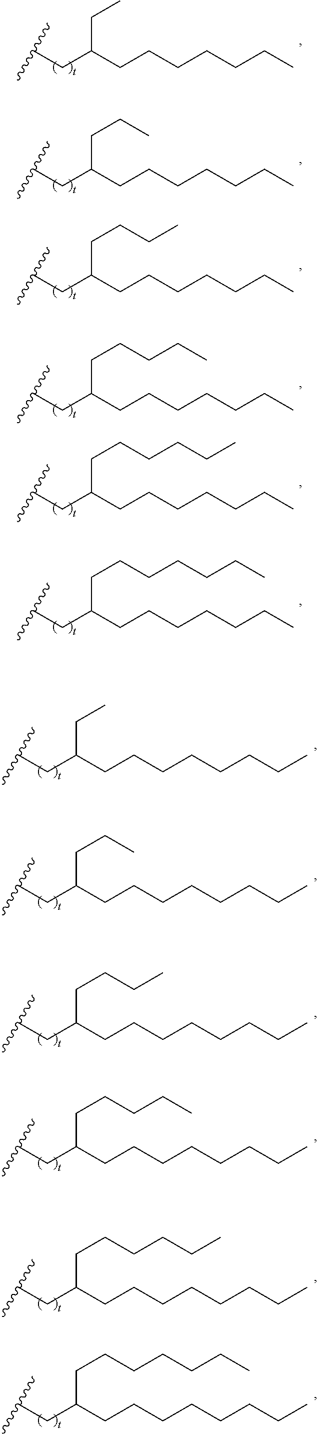

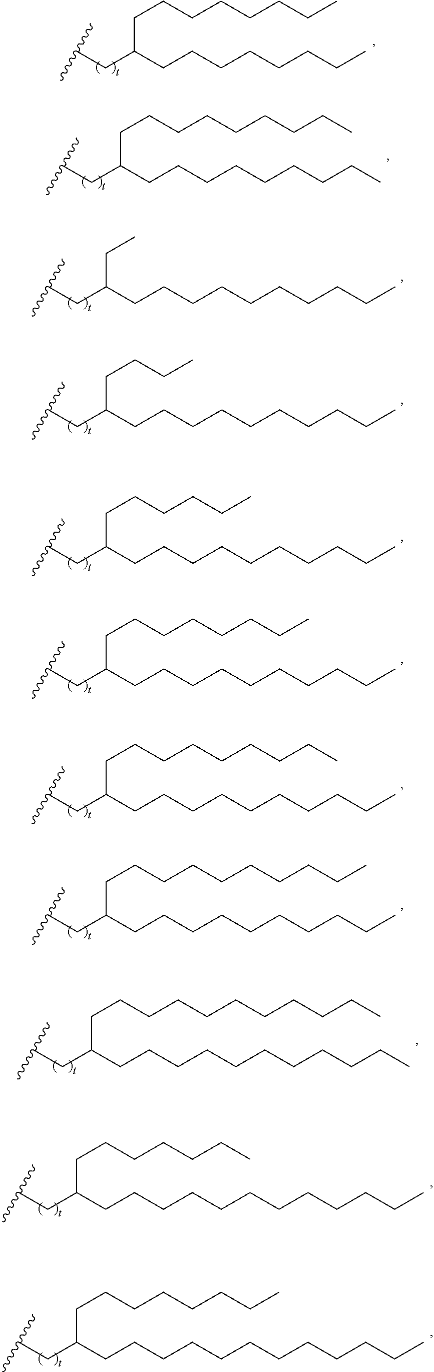

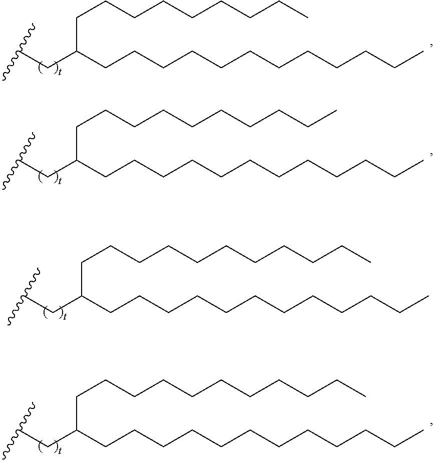

where t is 0, 1, 2, 3, 4, or 5.

In some embodiments, the pKa of the protonated form of the ionizable lipid compound described herein is about 5.1 to about 8.0, for example about 5.7 to about 6.5, about 5.7 to about 6.4, or from about 5.8 to about 6.2. In some embodiments, the pKa of the protonated form of the compound is about 5.5 to about 6.0. In some embodiments, the pKa of the protonated form of the compound is about 6.1 to about 6.3.

Non-limiting examples of ionizable lipid compounds disclosed here are set forth below.

| Lipid | | |
|---|---|---|
| No. | Structure | IUPAC Name |
| 7677 (2140) | 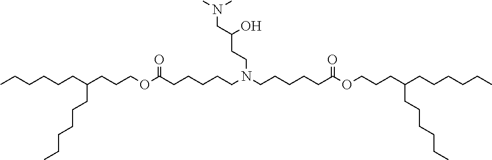 [SMILES: CCCCCCC(CCCCCC)CCCOC(=O)CCCCCN(CCCCCC(=O)OCCCC(CCCCCC)CCCCCC)CCC(O)CN(C)C] | bis(4-hexyldecyl) 6.6′-((4- (dimethylamino)- 3- hydroxybutyl) azanediyl)dihexanoate |
| 7676 (2139) | 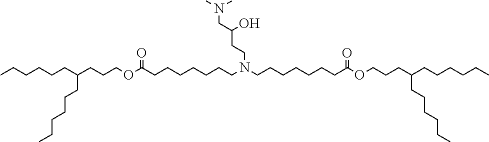 [SMILES: CCCCCCC(CCCCCC)CCCOC(=O)CCCCCCCN(CCCCCCCC(=O)OCCCC(CCCCCC)CCCCCC)CCC(O)CN(C)C] | bis(4-hexyldecyl) 8.8′-((4- (dimethylamino)- 3- hydroxybutyl) azanediyl) dioctanoate |
| 7675 (2138) | 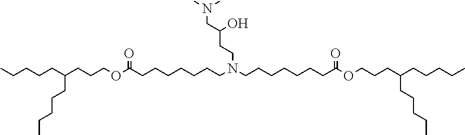 [SMILES: CCCCCC(CCCCC)CCCOC(=O)CCCCCCCN(CCCCCCCC(=O)OCCCC(CCCCC)CCCCC)CCC(O)CN(C)C] | bis(4-pentylnonyl) 8,8′-((4- (dimethylamino)- 3-hydroxybutyl) azanediyl) dioctanoate |
| 7671 (2142) | 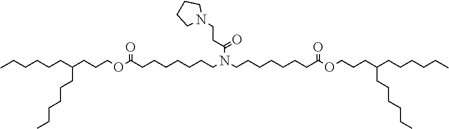 [SMILES: CCCCCCC(CCCCCC)CCCOC(=O)CCCCCCCN(CCCCCCCC(=O)OCCCC(CCCCCC)CCCCCC)C(=O)CCN1CCCC1] | bis(4-hexyldecyl) 8,8′-((3- (pyrrolidin-1- yl)propanoyl)azanediyl) dioctanoate |
| 7670 (2146) | 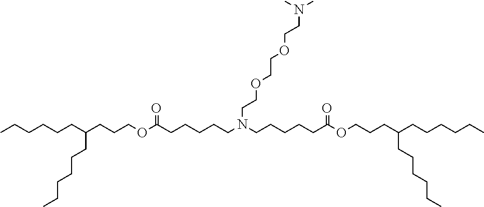 [SMILES: CCCCCCC(CCCCCC)CCCOC(=O)CCCCCN(CCCCCC(=O)OCCCC(CCCCCC)CCCCCC)CCOCCOCCN(C)C] | 4-hexyldecyl 11- (6-((4- hexyldecyl)oxy)-6- oxohexyl)-2- methyl-5,8-dioxa- 2,11- diazaheptadecan- 17-oate |
| 7669 (2145) | 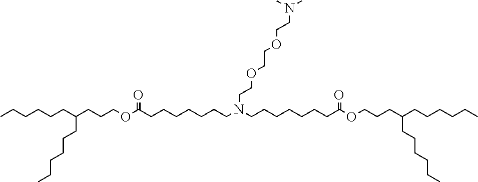 [SMILES: CCCCCCC(CCCCCC)CCCOC(=O)CCCCCCCN(CCCCCCCC(=O)OCCCC(CCCCCC)CCCCCC)CCOCCOCCN(C)C] | 4-hexyldecyl 11- (8-((4- hexyldecyl)oxy)-8- oxooctyl)-2- methyl-5,8-dioxa- 2,11- diazanonadecan- 19-oate |
| 7668 (2133) | 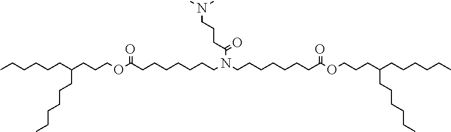 [SMILES: CCCCCCC(CCCCCC)CCCOC(=O)CCCCCCCN(CCCCCCCC(=O)OCCCC(CCCCCC)CCCCCC)C(=O)CCCN(C)C] | bis(4-hexyldecyl) 8,8′-((4- (dimethylamino) butanoyl)azanediyl) dioctanoate |
| 7667 (2137) | 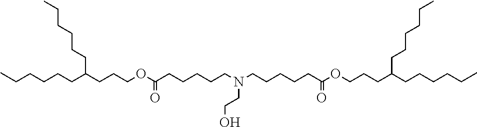 [SMILES: CCCCCCC(CCCCCC)CCCOC(=O)CCCCCN(CCO)CCCCCC(=O)OCCCC(CCCCCC)CCCCCC] | bis(4-hexyldecyl) 6,6′-((2- hydroxyethyl)azanediyl) dihexanoate |
| 7651 (2131) | 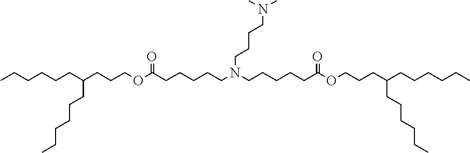 [SMILES: CCCCCCC(CCCCCC)CCCOC(=O)CCCCCN(CCCCCC(=O)OCCCC(CCCCCC)CCCCCC)CCCCN(C)C] | bis(4-hexyldecyl) 6,6′-((4- (dimethylamino) butyl)azanediyl) dihexanoate |
| 7650 (2130) | 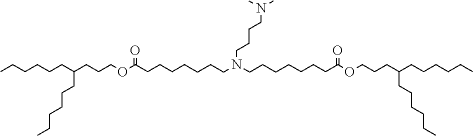 [SMILES: CCCCCCC(CCCCCC)CCCOC(=O)CCCCCCCN(CCCCCCCC(=O)OCCCC(CCCCCC)CCCCCC)CCCCN(C)C] | bis(4-hexyldecyl) 8,8′-((4- (dimethylamino) butyl)azanediyl) dioctanoate |
| 7649 (2129) | 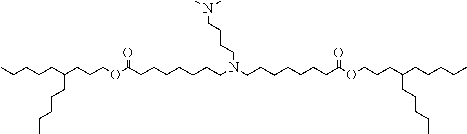 [SMILES: CCCCCC(CCCCC)CCCOC(=O)CCCCCCCN(CCCCCCCC(=O)OCCCC(CCCCC)CCCCC)CCCCN(C)C] | bis(4-pentylnonyl) 8,8′-((4- (dimethylamino) butyl)azanediyl) dioctanoate |
| 7633 (2144) | 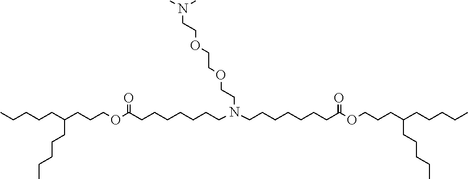 [SMILES: CCCCCC(CCCCC)CCCOC(=O)CCCCCCCN(CCCCCCCC(=O)OCCCC(CCCCC)CCCCC)CCOCCOCCN(C)C] | 4-pentylnonyl 2- methyl-11-(8-oxo- 8-((4- pentylnonyl)oxy) octyl)-5,8-dioxa- 2,11- diazanonadecan- 19-oate |
| 7632 (2143) | 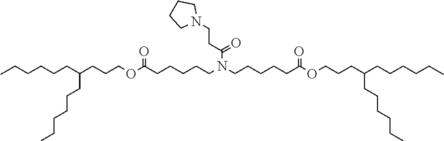 [SMILES: CCCCCCC(CCCCCC)CCCOC(=O)CCCCCN(CCCCCC(=O)OCCCC(CCCCCC)CCCCCC)C(=O)CCN1CCCC1] | bis(4-hexyldecyl) 6,6′-((3- (pyrrolidin-1- yl)propanoyl) azanediyl)dihexanoate |
| 7631 (2134) | 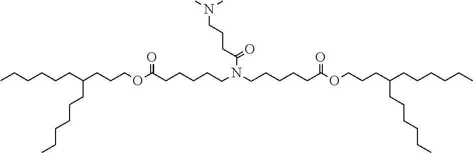 [SMILES: CCCCCCC(CCCCCC)CCCOC(=O)CCCCCN(CCCCCC(=O)OCCCC(CCCCCC)CCCCCC)C(=O)CCCN(C)C] | bis(4-hexyldecyl) 6,6′-((4- (dimethylamino) butanoyl)azanediyl) dihexanoate |
| 7608 (2135) | 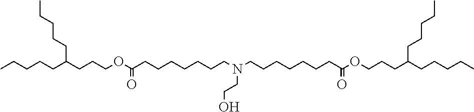 [SMILES: CCCCCC(CCCCC)CCCOC(=O)CCCCCCCN(CCO)CCCCCCCC(=O)OCCCC(CCCCC)CCCCC] | bis(4-pentylnonyl) 8,8′-((2- hydroxyethyl) azanediyl)dioctanoate |
| 7607 (2136) | 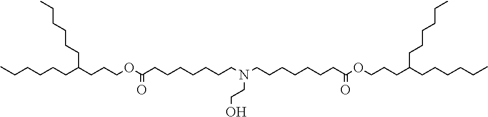 [SMILES: CCCCCCC(CCCCCC)CCCOC(=O)CCCCCCCN(CCO)CCCCCCCC(=O)OCCCC(CCCCCC)CCCCCC] | bis(4-hexyldecyl) 8,8′-((2- hydroxyethyl) azanediyl)dioctanoate |
| 7596 (2141) | 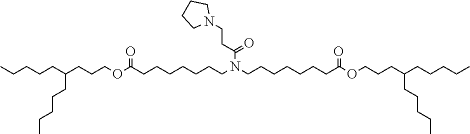 [SMILES: CCCCCC(CCCCC)CCCOC(=O)CCCCCCCN(CCCCCCCC(=O)OCCCC(CCCCC)CCCCC)C(=O)CCN1CCCC1] | bis(4-pentylnonyl) 8,8′-((3- (pyrrolidin-1- yl)propanoyl) azanediyl)dioctanoate |
| 7593 (2132) | 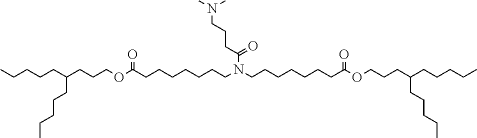 [SMILES: CCCCCC(CCCCC)CCCOC(=O)CCCCCCCN(CCCCCCCC(=O)OCCCC(CCCCC)CCCCC)C(=O)CCCN(C)C] | bis(4-pentylnonyl) 8,8′-((4- (dimethylamino) butanoyl)azanediyl) dioctanoate |
| 2229 | 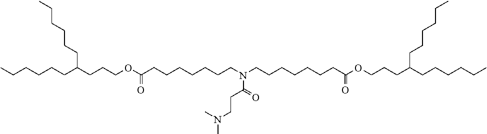 [SMILES: CCCCCCC(CCCCCC)CCCOC(=O)CCCCCCCN(CCCCCCCC(=O)OCCCC(CCCCCC)CCCCCC)C(=O)CCN(C)C] | bis(4-hexyldecyl) 8,8′-((3- (dimethylamino) propanoyl)azanediyl) dioctanoate |
| 2228 | 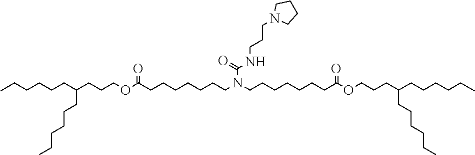 [SMILES: CCCCCCC(CCCCCC)CCCOC(=O)CCCCCCCN(CCCCCCCC(=O)OCCCC(CCCCCC)CCCCCC)C(=O)NCCCN1CCCC1] | bis(4-hexyldecyl) 8,8′-(((3- (pyrrolidin-1- yl)propyl)carbamoyl) azanediyl)dioctanoate |
| 2227 | 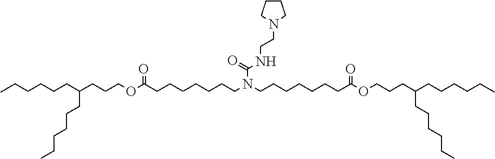 [SMILES: CCCCCCC(CCCCCC)CCCOC(=O)CCCCCCCN(CCCCCCCC(=O)OCCCC(CCCCCC)CCCCCC)C(=O)NCCN1CCCC1] | bis(4-hexyldecyl) 8,8′-(((2- (pyrrolidin-1- yl)ethyl)carbamoyl) azanediyl)dioctanoate |
| 2226 | 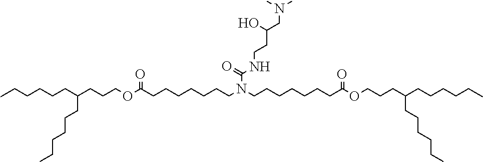 [SMILES: CCCCCCC(CCCCCC)CCCOC(=O)CCCCCCCN(CCCCCCCC(=O)OCCCC(CCCCCC)CCCCCC)C(=O)NCCC(O)CN(C)C] | bis(4-hexyldecyl) 8,8′-(((4- (dimethylamino)-3- hydroxybutyl) carbamoyl) azanediyl)dioctanoate |
| 2225 | 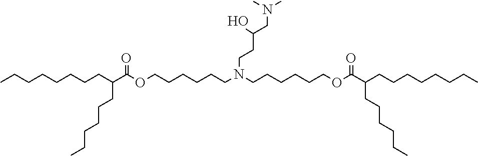 [SMILES: CCCCCCCCC(CCCCCC)C(=O)OCCCCCCN(CCCCCCOC(=O)C(CCCCCC)CCCCCCCC)CCC(O)CN(C)C] | ((4-(dimethylamino)-3- hydroxybutyl) azanediyl) bis(hexane- 6,1-diyl) bis(2- hexyldecanoate) |
| 2216 | 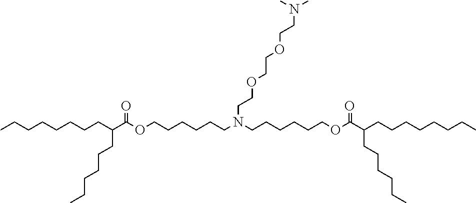 [SMILES: CCCCCCCCC(CCCCCC)C(=O)OCCCCCCN(CCCCCCOC(=O)C(CCCCCC)CCCCCCCC)CCOCCOCCN(C)C] | 11-(6-((2- hexyldecanoyl)oxy) hexyl)-2-methyl- 5,8-dioxa-2,11- diazaheptadecan- 17-yl 2- hexyldecanoate |
| 2215 | 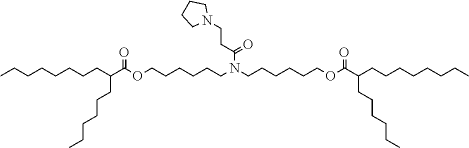 [SMILES: CCCCCCCCC(CCCCCC)C(=O)OCCCCCCN(CCCCCCOC(=O)C(CCCCCC)CCCCCCCC)C(=O)CCN1CCCC1] | ((3-(pyrrolidin-1- yl)propanoyl) azanediyl)bis (hexane- 6,1-diyl) bis(2- hexyldecanoate) |
| | 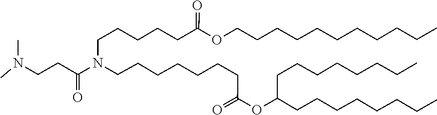 [SMILES: CCCCCCCCCCCOC(=O)CCCCCN(CCCCCCCC(=O)OC(CCCCCCCC)CCCCCCCC)C(=O)CCN(C)C] | |
| | 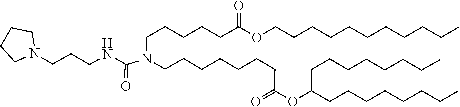 [SMILES: CCCCCCCCCCCOC(=O)CCCCCN(CCCCCCCC(=O)OC(CCCCCCCC)CCCCCCCC)C(=O)NCCCN1CCCC1] | |
| | 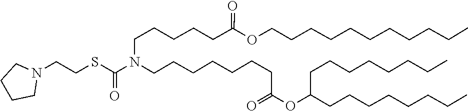 [SMILES: CCCCCCCCCCCOC(=O)CCCCCN(CCCCCCCC(=O)OC(CCCCCCCC)CCCCCCCC)C(=O)SCCN1CCCC1] | |
| | 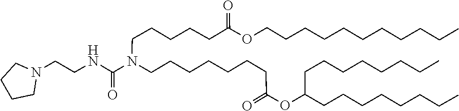 [SMILES: CCCCCCCCCCCOC(=O)CCCCCN(CCCCCCCC(=O)OC(CCCCCCCC)CCCCCCCC)C(=O)NCCN1CCCC1] | |
| | 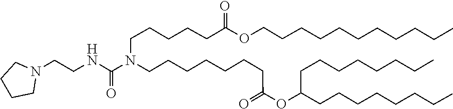 [SMILES: CCCCCCCCCCCOC(=O)CCCCCN(CCCCCCCC(=O)OC(CCCCCCCC)CCCCCCCC)C(=O)NCCN1CCCC1] | |
| | 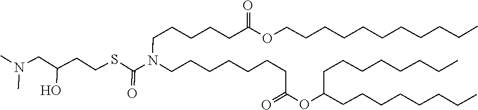 [SMILES: CCCCCCCCCCCOC(=O)CCCCCN(CCCCCCCC(=O)OC(CCCCCCCC)CCCCCCCC)C(=O)SCCC(O)CN(C)C] | |
| | 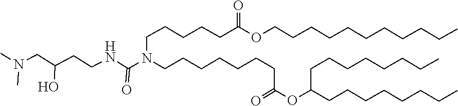 [SMILES: CCCCCCCCCCCOC(=O)CCCCCN(CCCCCCCC(=O)OC(CCCCCCCC)CCCCCCCC)C(=O)NCCC(O)CN(C)C] | |
| | 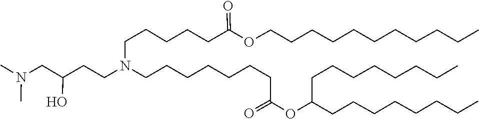 [SMILES: CCCCCCCCCCCOC(=O)CCCCCN(CCCCCCCC(=O)OC(CCCCCCCC)CCCCCCCC)CCC(O)CN(C)C] | |
| | 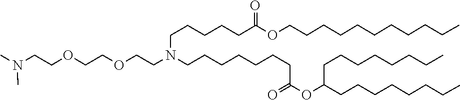 [SMILES: CCCCCCCCCCCOC(=O)CCCCCN(CCCCCCCC(=O)OC(CCCCCCCC)CCCCCCCC)CCOCCOCCN(C)C] | |
| | 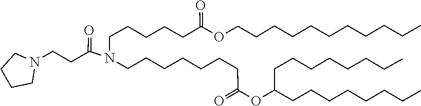 [SMILES: CCCCCCCCCCCOC(=O)CCCCCN(CCCCCCCC(=O)OC(CCCCCCCC)CCCCCCCC)C(=O)CCN1CCCC1] | |
| | 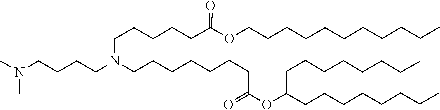 [SMILES: CCCCCCCCCCCOC(=O)CCCCCN(CCCCCCCC(=O)OC(CCCCCCCC)CCCCCCCC)CCCCN(C)C] | |
| | 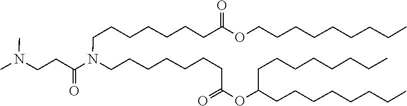 [SMILES: CCCCCCCCCOC(=O)CCCCCCCN(CCCCCCCC(=O)OC(CCCCCCCC)CCCCCCCC)C(=O)CCN(C)C] | |
| | 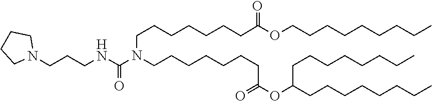 [SMILES: CCCCCCCCCOC(=O)CCCCCCCN(CCCCCCCC(=O)OC(CCCCCCCC)CCCCCCCC)C(=O)NCCCN1CCCC1] | |
| | 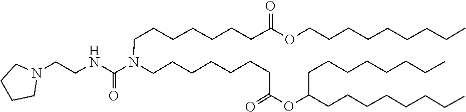 [SMILES: CCCCCCCCCOC(=O)CCCCCCCN(CCCCCCCC(=O)OC(CCCCCCCC)CCCCCCCC)C(=O)NCCN1CCCC1] | |
| | 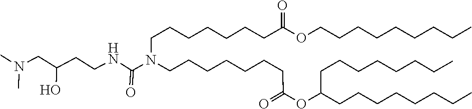 [SMILES: CCCCCCCCCOC(=O)CCCCCCCN(CCCCCCCC(=O)OC(CCCCCCCC)CCCCCCCC)C(=O)NCCC(O)CN(C)C] | |
| | 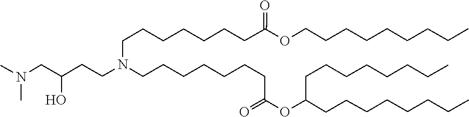 [SMILES: CCCCCCCCCOC(=O)CCCCCCCN(CCCCCCCC(=O)OC(CCCCCCCC)CCCCCCCC)CCC(O)CN(C)C] | |
| | 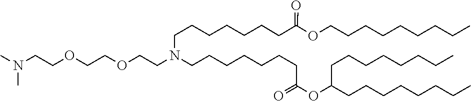 [SMILES: CCCCCCCCCOC(=O)CCCCCCCN(CCCCCCCC(=O)OC(CCCCCCCC)CCCCCCCC)CCOCCOCCN(C)C] | |
| | 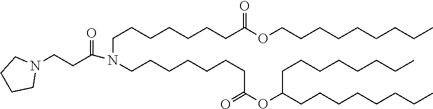 [SMILES: CCCCCCCCCOC(=O)CCCCCCCN(CCCCCCCC(=O)OC(CCCCCCCC)CCCCCCCC)C(=O)CCN1CCCC1] | |
| | 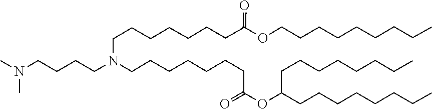 [SMILES: CCCCCCCCCOC(=O)CCCCCCCN(CCCCCCCC(=O)OC(CCCCCCCC)CCCCCCCC)CCCCN(C)C] | |
| | 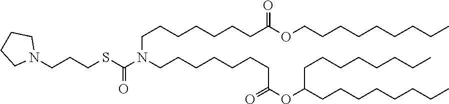 [SMILES: CCCCCCCCCOC(=O)CCCCCCCN(CCCCCCCC(=O)OC(CCCCCCCC)CCCCCCCC)C(=O)SCCCN1CCCC1] | |
| | 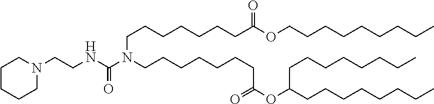 [SMILES: CCCCCCCCCOC(=O)CCCCCCCN(CCCCCCCC(=O)OC(CCCCCCCC)CCCCCCCC)C(=O)NCCN1CCCCC1] | |
| | 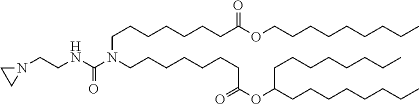 [SMILES: CCCCCCCCCOC(=O)CCCCCCCN(CCCCCCCC(=O)OC(CCCCCCCC)CCCCCCCC)C(=O)NCCN1CC1] | |
| | 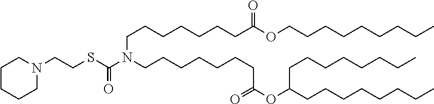 [SMILES: CCCCCCCCCOC(=O)CCCCCCCN(CCCCCCCC(=O)OC(CCCCCCCC)CCCCCCCC)C(=O)SCCN1CCCCC1] | |
| | 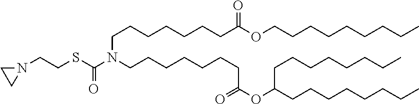 [SMILES: CCCCCCCCCOC(=O)CCCCCCCN(CCCCCCCC(=O)OC(CCCCCCCC)CCCCCCCC)C(=O)SCCN1CC1] | |
| | 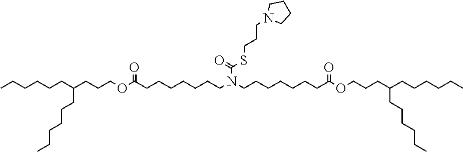 [SMILES: CCCCCCC(CCCCCC)CCCOC(=O)CCCCCCCN(CCCCCCCC(=O)OCCCC(CCCCCC)CCCCCC)C(=O)SCCCN1CCCC1] | |
| | 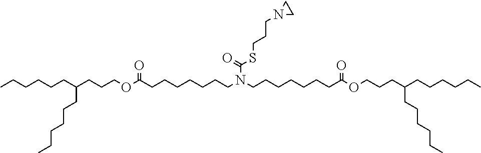 [SMILES: CCCCCCC(CCCCCC)CCCOC(=O)CCCCCCCN(CCCCCCCC(=O)OCCCC(CCCCCC)CCCCCC)C(=O)SCCCN1CC1] | |
| | 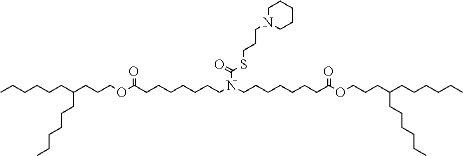 [SMILES: CCCCCCC(CCCCCC)CCCOC(=O)CCCCCCCN(CCCCCCCC(=O)OCCCC(CCCCCC)CCCCCC)C(=O)SCCCN1CCCCC1] | |
| | 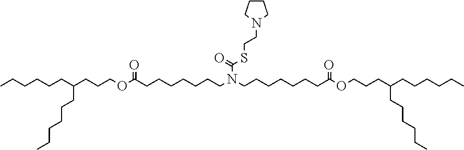 [SMILES: CCCCCCC(CCCCCC)CCCOC(=O)CCCCCCCN(CCCCCCCC(=O)OCCCC(CCCCCC)CCCCCC)C(=O)SCCN1CCCC1] | |
| | 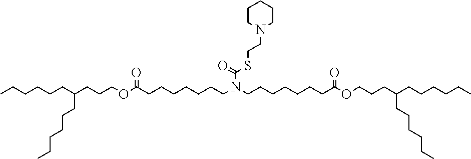 [SMILES: CCCCCCC(CCCCCC)CCCOC(=O)CCCCCCCN(CCCCCCCC(=O)OCCCC(CCCCCC)CCCCCC)C(=O)SCCN1CCCCC1] | |
| | 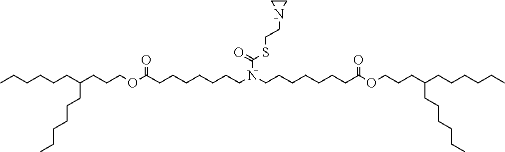 [SMILES: CCCCCCC(CCCCCC)CCCOC(=O)CCCCCCCN(CCCCCCCC(=O)OCCCC(CCCCCC)CCCCCC)C(=O)SCCN1CC1] | |
| 2233 | 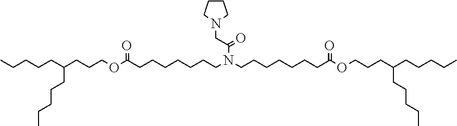 [SMILES: CCCCCC(CCCCC)CCCOC(=O)CCCCCCCN(CCCCCCCC(=O)OCCCC(CCCCC)CCCCC)C(=O)CN1CCCC1] | bis(4-pentylnonyl) 8,8′-((2- (pyrrolidin-1- yl)acetyl)azanediyl) dioctanoate |
| 2234 | 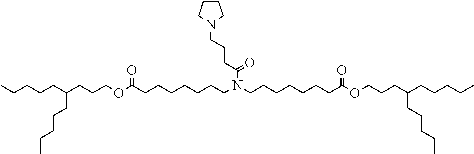 [SMILES: CCCCCC(CCCCC)CCCOC(=O)CCCCCCCN(CCCCCCCC(=O)OCCCC(CCCCC)CCCCC)C(=O)CCCN1CCCC1] | bis(4-pentylnonyl) 8,8′-((4- (pyrrolidin-1- yl)butanoyl)azanediyl) dioctanoate |
| 2235 | 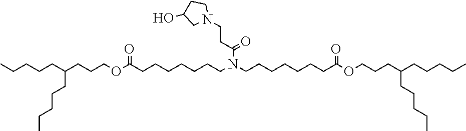 [SMILES: CCCCCC(CCCCC)CCCOC(=O)CCCCCCCN(CCCCCCCC(=O)OCCCC(CCCCC)CCCCC)C(=O)CCN1CCC(O)C1] | bis(4-pentylnonyl) 8,8′-((3-(3- hydroxypyrrolidin-1- yl)propanoyl) azanediyl)dioctanoate |
| 2236 | 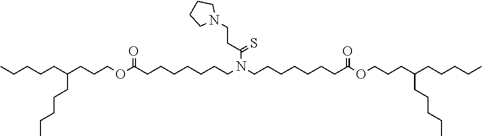 [SMILES: CCCCCC(CCCCC)CCCOC(=O)CCCCCCCN(CCCCCCCC(=O)OCCCC(CCCCC)CCCCC)C(=S)CCN1CCCC1] | bis(4-pentylnonyl) 8,8′-((3-(pyrrolidin-1- yl)propanethioyl) azanediyl)dioctanoate |
| 2237 | 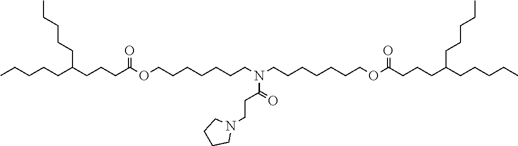 [SMILES: CCCCCC(CCCCC)CCCC(=O)OCCCCCCCN(CCCCCCCOC(=O)CCCC(CCCCC)CCCCC)C(=O)CCN1CCCC1] | ((3-(pyrrolidin-1- yl)propanoyl) azanediyl)bis(heptane- 7,1-diyl) bis(5- pentyldecanoate) |
| 2238 | 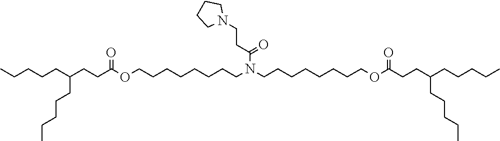 [SMILES: CCCCCC(CCCCC)CCC(=O)OCCCCCCCCN(CCCCCCCCOC(=O)CCC(CCCCC)CCCCC)C(=O)CCN1CCCC1] | ((3-(pyrrolidin-1- yl)propanoyl) azanediyl)bis(octane- 8,1-diyl) bis(4- pentylnonanoate) |
| 2239 | 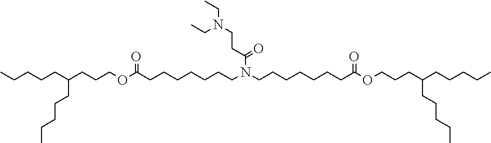 [SMILES: CCCCCC(CCCCC)CCCOC(=O)CCCCCCCN(CCCCCCCC(=O)OCCCC(CCCCC)CCCCC)C(=O)CCN(CC)CC] | bis(4-pentylnonyl) 8,8′-((3- (diethylamino) propanoyl)azanediyl) dioctanoate |
| 2241 | 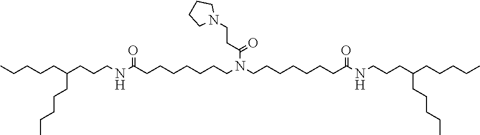 [SMILES: CCCCCC(CCCCC)CCCNC(=O)CCCCCCCN(CCCCCCCC(=O)NCCCC(CCCCC)CCCCC)C(=O)CCN1CCCC1] | 8-(N-(8-oxo-8-((4- pentylnonyl)amino) octyl)-3- (pyrrolidin-1- yl)propanamido)- N-(4- pentylnonyl) octanamide |
| 2242 | 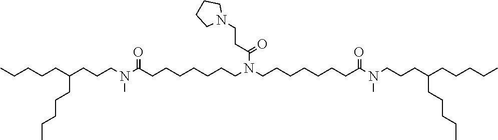 [SMILES: CCCCCC(CCCCC)CCCN(C)C(=O)CCCCCCCN(CCCCCCCC(=O)N(C)CCCC(CCCCC)CCCCC)C(=O)CCN1CCCC1] | N-methyl-8-(N-(8- (methyl(4- pentylnonyl)amino)- 8-oxooctyl)-3- (pyrrolidin-1- yl)propanamido)-N-(4- pentylnonyl)octanamide |
| 2244 | 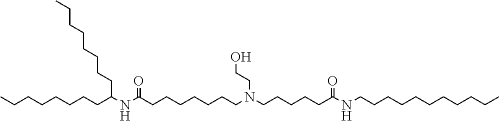 [SMILES: CCCCCCCCCCCNC(=O)CCCCCN(CCO)CCCCCCCC(=O)NC(CCCCCCCC)CCCCCCCC] | N-(heptadecan-9- yl)-8-((2- hydroxyethyl)(6- oxo-6- (undecylamino)hexyl) amino)octanamide |
| 2245 | 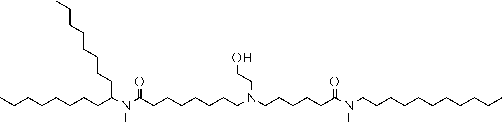 [SMILES: CCCCCCCCCCCN(C)C(=O)CCCCCN(CCO)CCCCCCCC(=O)N(C)C(CCCCCCCC)CCCCCCCC] | N-(heptadecan-9- yl)-8-((2- hydroxyethyl)(6-(methyl (undecyl)amino)-6- oxohexyl)amino)-N- methyloctanamide |
| 2249 | 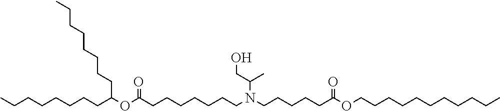 [SMILES: CCCCCCCCCCCOC(=O)CCCCCN(CCCCCCCC(=O)OC(CCCCCCCC)CCCCCCCC)C(C)CO] | heptadecan-9-yl 8- ((1- hydroxypropan-2- yl)(6-oxo-6- (undecyloxy)hexyl) amino)octanoate |
| 2250 | 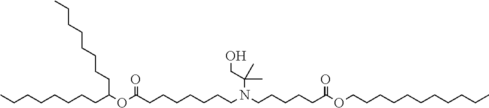 [SMILES: CCCCCCCCCCCOC(=O)CCCCCN(CCCCCCCC(=O)OC(CCCCCCCC)CCCCCCCC)C(C)(C)CO] | heptadecan-9-yl 8- ((1-hydroxy-2- methylpropan-2- yl)(6-oxo-6- (undecyloxy)hexyl) amino)octanoate |
| 2276 | 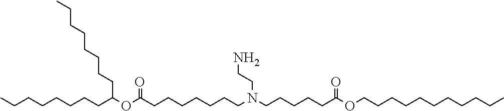 [SMILES: CCCCCCCCCCCOC(=O)CCCCCN(CCN)CCCCCCCC(=O)OC(CCCCCCCC)CCCCCCCC] | heptadecan-9-yl 8- ((2-aminoethyl)(6- oxo-6- (undecyloxy)hexyl) amino)octanoate |
| 2277 | 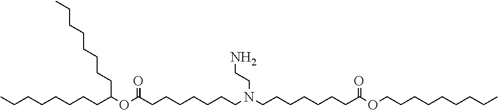 [SMILES: CCCCCCCCCOC(=O)CCCCCCCN(CCN)CCCCCCCC(=O)OC(CCCCCCCC)CCCCCCCC] | heptadecan-9-yl 8- ((2-aminoethyl)(8- (nonyloxy)-8- oxooctyl)amino) octanoate |
| 2300 | 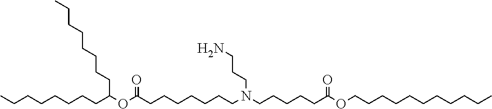 [SMILES: CCCCCCCCCCCOC(=O)CCCCCN(CCCN)CCCCCCCC(=O)OC(CCCCCCCC)CCCCCCCC] | heptadecan-9-yl 8- ((3- aminopropyl)(6- oxo-6- (undecyloxy)hexyl) amino)octanoate |
| 2301 | 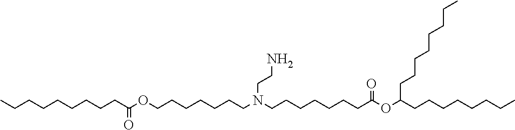 [SMILES: CCCCCCCCCC(=O)OCCCCCCCN(CCN)CCCCCCCC(=O)OC(CCCCCCCC)CCCCCCCC] | 7-((2- aminoethyl)(8- (heptadecan-9- yloxy)-8- oxooctyl)amino) heptyl decanoate |
| 2312 | 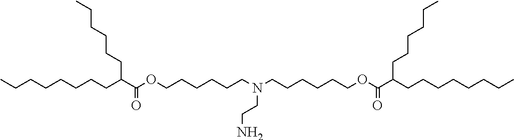 [SMILES: CCCCCCCCC(CCCCCC)C(=O)OCCCCCCN(CCN)CCCCCCOC(=O)C(CCCCCC)CCCCCCCC] | ((2- aminoethyl)azanediyl) bis(hexane-6,1- diyl) bis(2- hexyldecanoate) |
| 2313 | 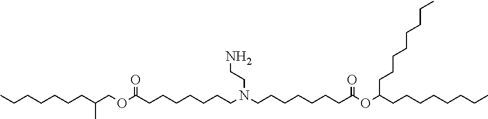 [SMILES: CCCCCCCCC(CCCCCCCC)OC(=O)CCCCCCCN(CCN)CCCCCCCC(=O)OCC(C)CCCCCCC] | heptadecan-9-yl 8- ((2-aminoethyl)(8-((2- methylnonyl)oxy)-8- oxooctyl)amino) octanoate |
| 2314 | 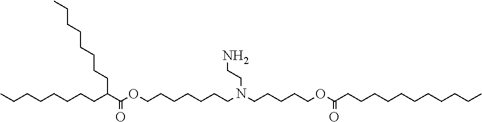 [SMILES: CCCCCCCCCCCC(=O)OCCCCCN(CCN)CCCCCCCOC(=O)C(CCCCCCCC)CCCCCCCC] | 5-((2- aminoethyl)(7-((2- octyldecanoyl)oxy) heptyl)amino)pentyl dodecanoate |
| | 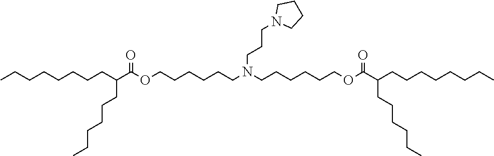 [SMILES: CCCCCCCCC(CCCCCC)C(=O)OCCCCCCN(CCCCCCOC(=O)C(CCCCCC)CCCCCCCC)CCCN1CCCC1] | ((3-(pyrrolidin-1- yl)propyl)azanediyl) bis(hexane-6,1- diyl) bis(2- hexyldecanoate) |
| | 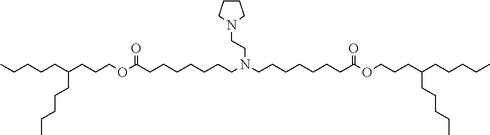 [SMILES: CCCCCC(CCCCC)CCCOC(=O)CCCCCCCN(CCCCCCCC(=O)OCCCC(CCCCC)CCCCC)CCN1CCCC1] | bis(4-pentylnonyl) 8,8′-((2- (pyrrolidin-1- yl)ethyl)azanediyl) dioctanoate |
| | 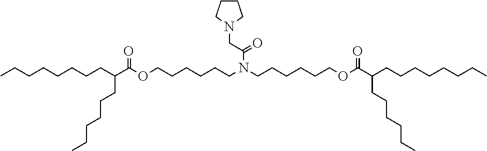 [SMILES: CCCCCCCCC(CCCCCC)C(=O)OCCCCCCN(CCCCCCOC(=O)C(CCCCCC)CCCCCCCC)C(=O)CN1CCCC1] | ((2-(pyrrolidin-1- yl)acetyl)azanediyl) bis(hexane-6,1- diyl) bis(2- hexyldecanoate) |
| | 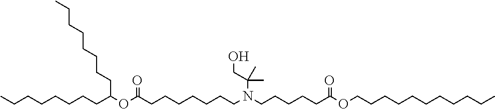 [SMILES: CCCCCCCCCCCOC(=O)CCCCCN(CCCCCCCC(=O)OC(CCCCCCCC)CCCCCCCC)C(C)(C)CO] | heptadecan-9-yl 8- ((1-hydroxy-2- methylpropan-2- yl)(6-oxo-6- (undecyloxy)hexyl) amino)octanoate |
| | 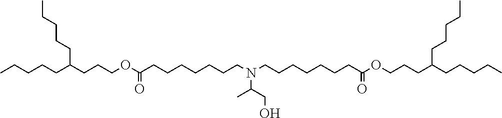 [SMILES: CCCCCC(CCCCC)CCCOC(=O)CCCCCCCN(CCCCCCCC(=O)OCCCC(CCCCC)CCCCC)C(C)CO] | bis(4-pentylnonyl) 8,8′-((1- hydroxypropan-2- yl)azanediyl) dioctanoate |
| | 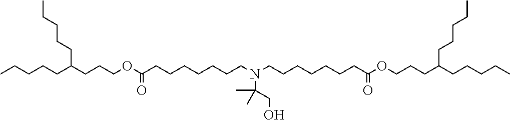 [SMILES: CCCCCC(CCCCC)CCCOC(=O)CCCCCCCN(CCCCCCCC(=O)OCCCC(CCCCC)CCCCC)C(C)(C)CO] | bis(4-pentylnonyl) 8,8′-((1-hydroxy-2- methylpropan-2- yl)azanediyl) dioctanoate |
| | 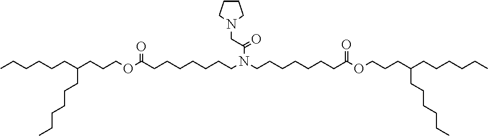 [SMILES: CCCCCCC(CCCCCC)CCCOC(=O)CCCCCCCN(CCCCCCCC(=O)OCCCC(CCCCCC)CCCCCC)C(=O)CN1CCCC1] | bis(4-hexyldecyl) 8,8′-((2- (pyrrolidin-1- yl)acetyl)azanediyl) dioctanoate |
| | 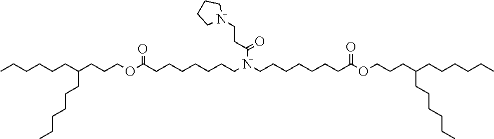 [SMILES: CCCCCCC(CCCCCC)CCCOC(=O)CCCCCCCN(CCCCCCCC(=O)OCCCC(CCCCCC)CCCCCC)C(=O)CCN1CCCC1] | bis(4-hexyldecyl) 8,8′-((3- (pyrrolidin-1- yl)propanoyl) azanediyl)dioctanoate |
| | 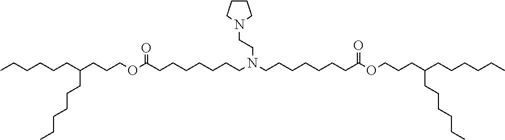 [SMILES: CCCCCCC(CCCCCC)CCCOC(=O)CCCCCCCN(CCCCCCCC(=O)OCCCC(CCCCCC)CCCCCC)CCN1CCCC1] | bis(4-hexyldecyl) 8,8′-((2- (pyrrolidin-1- yl)ethyl)azanediyl) dioctanoate |
| | 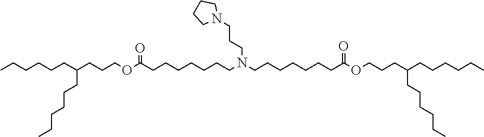 [SMILES: CCCCCCC(CCCCCC)CCCOC(=O)CCCCCCCN(CCCCCCCC(=O)OCCCC(CCCCCC)CCCCCC)CCCN1CCCC1] | bis(4-hexyldecyl) 8,8′-((3- (pyrrolidin-1- yl)propyl)azanediyl) dioctanoate |
| | 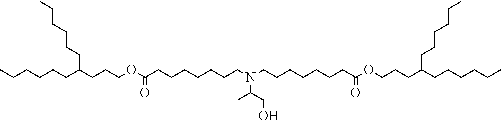 [SMILES: CCCCCCC(CCCCCC)CCCOC(=O)CCCCCCCN(CCCCCCCC(=O)OCCCC(CCCCCC)CCCCCC)C(C)CO] | bis(4-hexyldecyl) 8,8′-((1- hydroxypropan-2- yl)azanediyl) dioctanoate |
| | 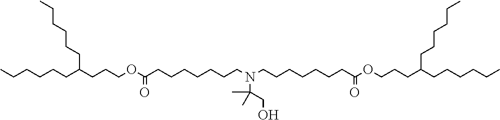 [SMILES: CCCCCCC(CCCCCC)CCCOC(=O)CCCCCCCN(CCCCCCCC(=O)OCCCC(CCCCCC)CCCCCC)C(C)(C)CO] | bis(4-hexyldecyl) 8,8′-((1-hydroxy-2. methylpropan-2- yl)azanediyl) dioctanoate |
| | 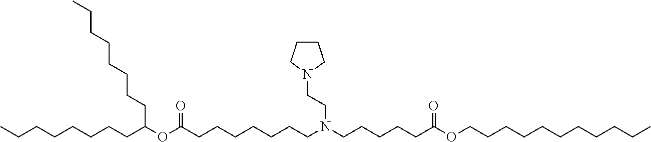 [SMILES: CCCCCCCCCCCOC(=O)CCCCCN(CCCCCCCC(=O)OC(CCCCCCCC)CCCCCCCC)CCN1CCCC1] | heptadecan-9-yl 8- ((6-oxo-6- (undecyloxy)hexyl) (2-(pyrrolidin-1- yl)ethyl) amino)octanoate |
| | 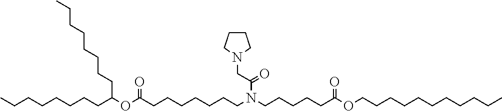 [SMILES: CCCCCCCCCCCOC(=O)CCCCCN(CCCCCCCC(=O)OC(CCCCCCCC)CCCCCCCC)C(=O)CN1CCCC1] | heptadecan-9-yl 8- (N-(6-oxo-6- (undecyloxy)hexyl)- 2-(pyrrolidin-1- yl)acetamido)octanoate |
| | 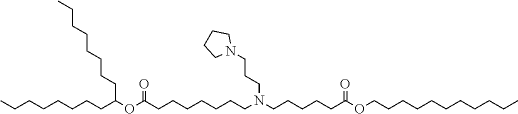 [SMILES: CCCCCCCCCCCOC(=O)CCCCCN(CCCCCCCC(=O)OC(CCCCCCCC)CCCCCCCC)CCCN1CCCC1] | heptadecan-9-yl 8- ((6-oxo-6- (undecyloxy)hexyl) (3-(pyrrolidin-1- yl)propyl)amino) octanoate |
| | 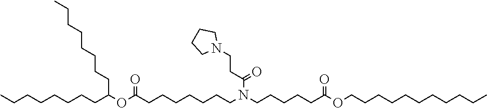 [SMILES: CCCCCCCCCCCOC(=O)CCCCCN(CCCCCCCC(=O)OC(CCCCCCCC)CCCCCCCC)C(=O)CCN1CCCC1] | heptadecan-9-yl 8- (N-(6-oxo-6- (undecyloxy)hexyl)- 3-(pyrrolidin-1- yl)propanamido) octanoate |
| | 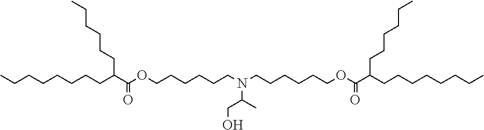 [SMILES: CCCCCCCCC(CCCCCC)C(=O)OCCCCCCN(CCCCCCOC(=O)C(CCCCCC)CCCCCCCC)C(C)CO] | ((1- hydroxypropan-2- yl)azanediyl)bis (hexane-6,1-diyl) bis(2- hexyldecanoate) |
| | 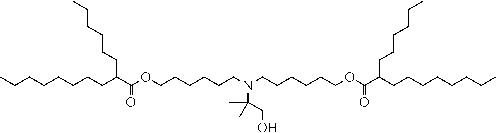 [SMILES: CCCCCCCCC(CCCCCC)C(=O)OCCCCCCN(CCCCCCOC(=O)C(CCCCCC)CCCCCCCC)C(C)(C)CO] | ((1-hydroxy-2- methylpropan-2- yl)azanediyl)bis (hexane-6,1-diyl) bis(2- hexyldecanoate) |
| | 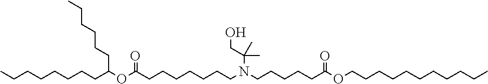 [SMILES: CCCCCCCCCCCOC(=O)CCCCCN(CCCCCCCC(=O)OC(CCCCCC)CCCCCCCC)C(C)(C)CO] | pentadecan-7-yl 8- ((1-hydroxy-2- methylpropan-2- yl)(6-oxo-6- (undecyloxy)hexyl) amino)octanoate |
| | 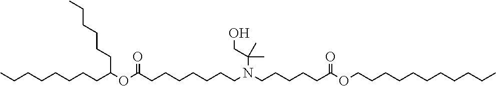 [SMILES: CCCCCCCCCCCOC(=O)CCCCCN(CCCCCCCC(=O)OC(CCCCCC)CCCCCCCC)C(C)(C)CO] | tridecan-7-yl 8-((1- hydroxy-2- methylpropan-2- yl)(6-oxo-6- (undecyloxy)hexyl) amino)octanoate |
| | 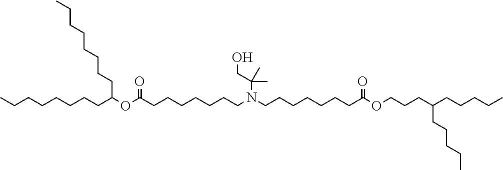 [SMILES: CCCCCCCCC(CCCCCCCC)OC(=O)CCCCCCCN(CCCCCCCC(=O)OCCCC(CCCCC)CCCCC)C(C)(C)CO] | heptadecan-9-yl 8- ((1-hydroxy-2- methylpropan-2- yl)(8-oxo-8-((4- pentylnonyl)oxy) octyl)amino)octanoate |
| | 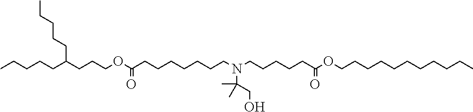 [SMILES: CCCCCCCCCCCOC(=O)CCCCCN(CCCCCCCC(=O)OCCCC(CCCCC)CCCCC)C(C)(C)CO] | 4-pentylnonyl 8- ((1-hydroxy-2- methylpropan-2- yl)(6-oxo-6- (undecyloxy)hexyl) amino)octanoate |
| | 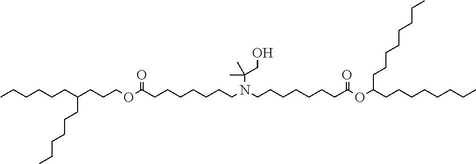 [SMILES: CCCCCCCCC(CCCCCCCC)OC(=O)CCCCCCCN(CCCCCCCC(=O)OCCCC(CCCCCC)CCCCCC)C(C)(C)CO] | heptadecan-9-yl 8- ((8-((4- hexyldecyl)oxy)-8- oxooctyl)(1- hydroxy-2- methylpropan-2- yl)amino)octanoate |
| | 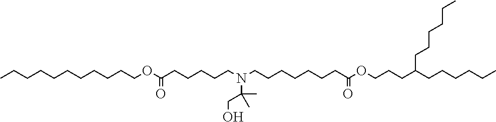 [SMILES: CCCCCCCCCCCOC(=O)CCCCCN(CCCCCCCC(=O)OCCCC(CCCCCC)CCCCCC)C(C)(C)CO] | 4-hexyldecyl 8- ((1-hydroxy-2- methylpropan-2- yl)(6-oxo-6- (undecyloxy)hexyl) amino)octanoate |
| | 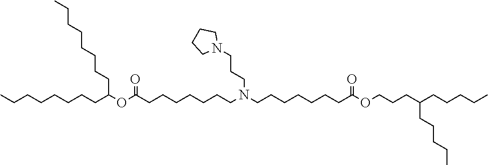 [SMILES: CCCCCCCCC(CCCCCCCC)OC(=O)CCCCCCCN(CCCCCCCC(=O)OCCCC(CCCCC)CCCCC)CCCN1CCCC1] | heptadecan-9-yl 8- ((8-oxo-8-((4- pentylnonyl)oxy) octyl)(3-(pyrrolidin-1- yl)propyl)amino) octanoate |
| | 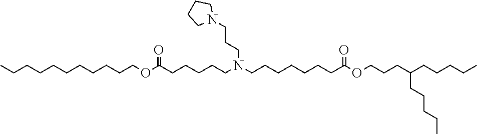 [SMILES: CCCCCCCCCCCOC(=O)CCCCCN(CCCCCCCC(=O)OCCCC(CCCCC)CCCCC)CCCN1CCCC1] | 4-pentylnonyl 8- ((6-oxo-6- (undecyloxy)hexyl) (3-(pyrrolidin-1- yl)propyl)amino) octanoate |
| | 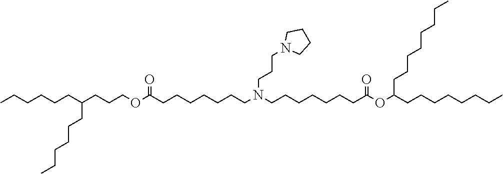 [SMILES: CCCCCCCCC(CCCCCCCC)OC(=O)CCCCCCCN(CCCCCCCC(=O)OCCCC(CCCCCC)CCCCCC)CCCN1CCCC1] | heptadecan-9-yl 8- ((8-((4- hexyldecyl)oxy)-8- oxooctyl)(3- (pyrrolidin-1- yl)propyl)amino) octanoate |
| | 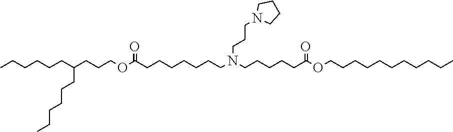 [SMILES: CCCCCCCCCCCOC(=O)CCCCCN(CCCCCCCC(=O)OCCCC(CCCCCC)CCCCCC)CCCN1CCCC1] | 4-hexyldecyl 8- ((6-oxo-6- (undecyloxy)hexyl) (3-(pyrrolidin-1- yl)propyl)amino) octanoate |
| | 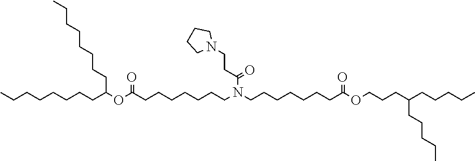 [SMILES: CCCCCCCCC(CCCCCCCC)OC(=O)CCCCCCCN(CCCCCCCC(=O)OCCCC(CCCCC)CCCCC)C(=O)CCN1CCCC1] | heptadecan-9-yl 8- (N-(8-oxo-8-((4- pentylnonyl)oxy) octyl)-3-(pyrrolidin-1- yl)propanamido) octanoate |
| | 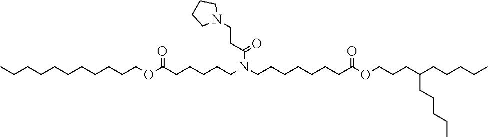 [SMILES: CCCCCCCCCCCOC(=O)CCCCCN(CCCCCCCC(=O)OCCCC(CCCCC)CCCCC)C(=O)CCN1CCCC1] | 4-pentylnonyl 8- (N-(6-oxo-6- (undecyloxy)hexyl)- 3-(pyrrolidin-1- yl)propanamido) octanoate |
| | 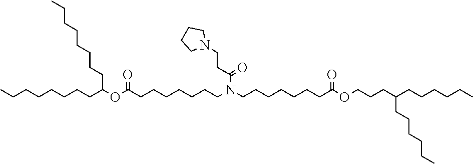 [SMILES: CCCCCCCCC(CCCCCCCC)OC(=O)CCCCCCCN(CCCCCCCC(=O)OCCCC(CCCCCC)CCCCCC)C(=O)CCN1CCCC1] | heptadecan-9-yl 8- (N-(8-((4- hexyldecyl)oxy)-8- oxooctyl)-3- (pyrrolidin-1- yl)propanamido) octanoate |
| | 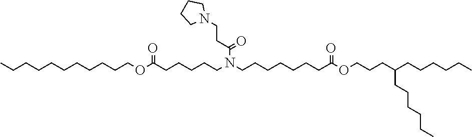 [SMILES: CCCCCCCCCCCOC(=O)CCCCCN(CCCCCCCC(=O)OCCCC(CCCCCC)CCCCCC)C(=O)CCN1CCCC1] | 4-hexyldecyl 8-(N- (6-oxo-6- (undecyloxy)hexyl)- 3-(pyrrolidin-1- yl)propanamido) octanoate |
| | 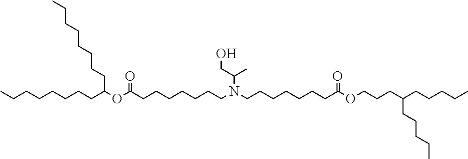 [SMILES: CCCCCCCCC(CCCCCCCC)OC(=O)CCCCCCCN(CCCCCCCC(=O)OCCCC(CCCCC)CCCCC)C(C)CO] | heptadecan-9-yl 8- ((1- hydroxypropan-2- yl)(8-oxo-8-((4- pentylnonyl)oxy) octyl)amino)octanoate |
| | 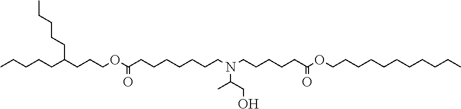 [SMILES: CCCCCCCCCCCOC(=O)CCCCCN(CCCCCCCC(=O)OCCCC(CCCCC)CCCCC)C(C)CO] | 4-pentylnonyl 8- ((1- hydroxypropan-2- yl)(6-oxo-6- (undecyloxy)hexyl) amino)octanoate |
| | 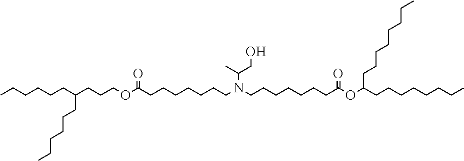 [SMILES: CCCCCCCCC(CCCCCCCC)OC(=O)CCCCCCCN(CCCCCCCC(=O)OCCCC(CCCCCC)CCCCCC)C(C)CO] | heptadecan-9-yl 8- ((8-((4- hexyldecyl)oxy)-8- oxooctyl)(1- hydroxypropan-2- yl)amino)octanoate |
| | 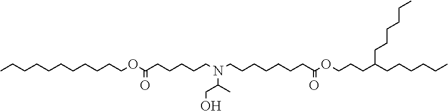 [SMILES: CCCCCCCCCCCOC(=O)CCCCCN(CCCCCCCC(=O)OCCCC(CCCCCC)CCCCCC)C(C)CO] | 4-hexyldecyl 8- ((1- hydroxypropan-2- yl)(6-oxo-6- (undecyloxy)hexyl) amino)octanoate |
| | 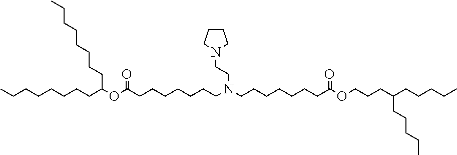 [SMILES: CCCCCCCCC(CCCCCCCC)OC(=O)CCCCCCCN(CCCCCCCC(=O)OCCCC(CCCCC)CCCCC)CCN1CCCC1] | heptadecan-9-yl 8- ((8-oxo-8-((4- pentylnonyl)oxy) octyl)(2-(pyrrolidin-1- yl)ethyl)amino) octanoate |
| | 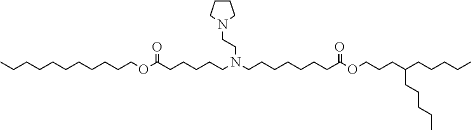 [SMILES: CCCCCCCCCCCOC(=O)CCCCCN(CCCCCCCC(=O)OCCCC(CCCCC)CCCCC)CCN1CCCC1] | 4-pentylnonyl 8- ((6-oxo-6- (undecyloxy)hexyl) (2-(pyrrolidin-1- yl)ethyl)amino) octanoate |
| | 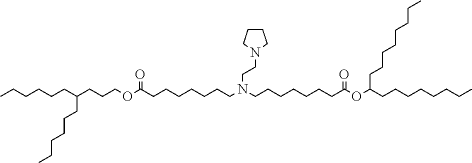 [SMILES: CCCCCCCCC(CCCCCCCC)OC(=O)CCCCCCCN(CCCCCCCC(=O)OCCCC(CCCCCC)CCCCCC)CCN1CCCC1] | heptadecan-9-yl 8- ((8-((4- hexyldecyl)oxy)-8- oxooctyl)(2- (pyrrolidin-1- yl)ethyl)amino) octanoate |
| | 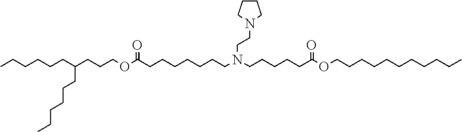 [SMILES: CCCCCCCCCCCOC(=O)CCCCCN(CCCCCCCC(=O)OCCCC(CCCCCC)CCCCCC)CCN1CCCC1] | 4-hexyldecyl 8- ((6-oxo-6- (undecyloxy)hexyl) (2-(pyrrolidin-1- yl)ethyl)amino) octanoate |
| | 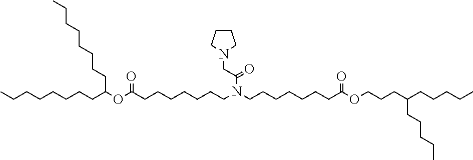 [SMILES: CCCCCCCCC(CCCCCCCC)OC(=O)CCCCCCCN(CCCCCCCC(=O)OCCCC(CCCCC)CCCCC)C(=O)CN1CCCC1] | heptadecan-9-yl 8- (N-(8-oxo-8-((4- pentylnonyl)oxy) octyl)-2-(pyrrolidin-1- yl)acetamido)octanoate |
| | 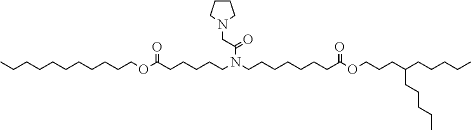 [SMILES: CCCCCCCCCCCOC(=O)CCCCCN(CCCCCCCC(=O)OCCCC(CCCCC)CCCCC)C(=O)CN1CCCC1] | 4-pentylnonyl 8- (N-(6-oxo-6- (undecyloxy)hexyl)- 2-(pyrrolidin-1- yl)acetamido)octanoate |
| | 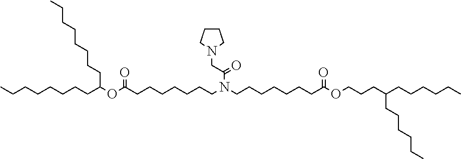 [SMILES: CCCCCCCCC(CCCCCCCC)OC(=O)CCCCCCCN(CCCCCCCC(=O)OCCCC(CCCCCC)CCCCCC)C(=O)CN1CCCC1] | heptadecan-9-yl 8- (N-(8-((4- hexyldecyl)oxy)-8- oxooctyl)-2- (pyrrolidin-1- yl)acetamido) octanoate |
| | 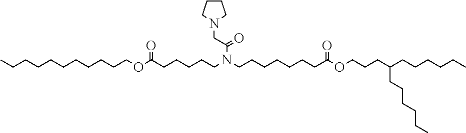 [SMILES: CCCCCCCCCCCOC(=O)CCCCCN(CCCCCCCC(=O)OCCCC(CCCCCC)CCCCCC)C(=O)CN1CCCC1] | 4-hexyldecyl 8-(N- (6-oxo-6- (undecyloxy)hexyl)- 2-(pyrrolidin-1- yl)acetamido) octanoate |

### Lipid Nanoparticle Composition

Ionizable lipids disclosed herein may be used to form lipid nanoparticle compositions. In some embodiments, the lipid nanoparticle composition further comprises one or more therapeutic agents. In some embodiments, the lipid nanoparticle in the composition encapsulates or is associated with the one or more therapeutic agents.

In some embodiments, the LNP composition has an N/P ratio of about 3 to about 10, for example the N/P ratio is about 6±1, or the N/P ratio is about 6±0.5. In some embodiments, the N/P ratio is about 6.

In some embodiments, the disclosure relates to a combination comprising (i) one or more compounds chosen from the ionizable lipids of Formula (I)—(VII), pharmaceutically acceptable salts thereof, and stereoisomers of any of the foregoing and (ii) a lipid component. In some embodiments, the combination comprises 5%, 10%, 15%, 20%, 25%, 30%, 35%, 40%, 45%, 50%, 55%, 60%, 65%, 70%, 75%, 80%, 85%, 90%, or 95% of the one or more compounds of (i). In some embodiments, the combination comprises about a 1:1 ratio of the compounds of (i) and the lipid component (ii). In some embodiments, the combination is a lipid nanoparticle (LNP) composition.

In some embodiments, the disclosure relates to a lipid nanoparticle composition comprising (i) one or more ionizable lipid compounds as described herein and (ii) one or more lipid components.

In some embodiments, the one or more lipid components in the LNP composition comprise one or more helper lipids and one or more PEG lipids. In some embodiments, the lipid component(s) comprise(s) one or more helper lipids, one or more PEG lipids, and one or more neutral lipids.

### The Non-Ionizable Lipid Components

### Neutral Lipids

In some embodiments, the lipid components comprise one or more neutral lipids. The neutral lipids may be one or more phospholipids, such as one or more (poly)unsaturated lipids.

Phospholipids may assemble into one or more lipid bilayers. In general, phospholipids may include a phospholipid moiety and one or more fatty acid moieties. For example, a phospholipid may be a lipid according to formula:

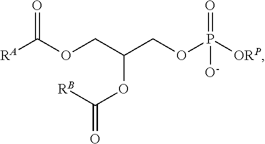

wherein R^{p} represents a phospholipid moiety, and R^{A} and R^{B} represent fatty acid moieties with or without unsaturation that may be the same or different. A phospholipid moiety may be a phosphatidyl choline, phosphatidyl ethanolamine, phosphatidyl glycerol, phosphatidyl serine, phosphatidic acid, 2-lysophosphatidyl choline, or a sphingomyelin. A fatty acid moiety may be a lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, or docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a lipid nanoparticle to facilitate membrane permeation or cellular recognition or in conjugating a lipid nanoparticle to a useful component such as a targeting or imaging moiety (e.g. a dye).

In some embodiments, the neutral lipids may be phospholipids such as distearoyl-sn-glycero-3-phosphocholine (DSPC), 1,2-dioleoyl-sn-glycero-3-phosphoethanolamine (DOPE), 1,2-dilinolenoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC) 1-palmitoyl-2-oleoyl-sn-glycol-3-phosphocholine (POPC), 1,2-di-O-octadecenyl-sn-glycero-3-phosphocholine (18:0 diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinolenoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or mixtures thereof.

Additional non-limiting examples of non-ionizable lipids also include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids may be acyl groups derived from fatty acids having C₁₀-C₂₄ carbon chains, e.g., lauroyl, myristoyl, palmitoyl, stearoyl, or oleoyl.

### Steroids and Other Non-Ionizable Lipid Components

In some embodiments, the lipid components comprise one or more steroids or analogues thereof.

In some embodiments, the lipid components comprise sterols such as cholesterol, sisterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In some embodiments, the cholesterol derivative is a polar analogue such as cholesteryl-(4′-hydroxy)-butyl ether.

In some embodiments, the non-ionizable lipid components comprise or consist of a mixture of one or more phospholipids and cholesterol or a derivative thereof. In some embodiments, the non-ionizable lipid components present comprise or consist of one or more phospholipids, e.g., a cholesterol-free lipid particle formulation. In some embodiments, the non-ionizable lipid components present comprise or consist of cholesterol or a derivative thereof, e.g., a phospholipid-free lipid particle formulation.

In some embodiments, the LNP composition comprises a phytosterol or a combination of a phytosterol and cholesterol. In some embodiments, the phytosterol is selected from the group consisting of b-sitosterol, stigmasterol, b-sitostanol, campesterol, brassicasterol, and combinations thereof. In some embodiments, the phytosterol is selected from the group consisting of b-sitosterol, b-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In some embodiments, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In some embodiments, the phytosterol is a combination of Compound S-141, Compound S-140, Compound S-143 and Compound S-148. In some embodiments, the phytosterol comprises a sitosterol or a salt or an ester thereof. In some embodiments, the phytosterol comprises a stigmasterol or a salt or an ester thereof. In some embodiments, the phytosterol is beta-sitosterol,

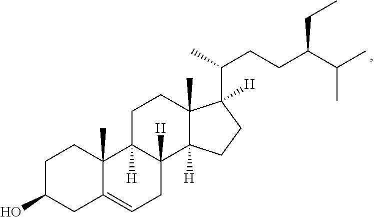

a salt thereof, or an ester thereof.

In some embodiments, the LNP composition comprises a phytosterol, or a salt or ester thereof, and cholesterol or a salt thereof.

In some embodiments, the target cell is a cell described herein e.g., a liver cell or a splenic cell), and the phytosterol or a salt or ester thereof is selected from the group consisting of b-sitosterol, b-sitostanol, campesterol, and brassicasterol, and combinations thereof. In some embodiments, the phytosterol is b-sitosterol. In some embodiments, the phytosterol is b-sitostanol. In some embodiments, the phytosterol is campesterol. In some embodiments, the phytosterol is brassicasterol.

In some embodiments, the target cell is a cell described herein (e.g., a liver cell or a splenic cell), and the phytosterol or a salt or ester thereof is selected from the group consisting of b-sitosterol, and stigmasterol, and combinations thereof. In some embodiments, the phytosterol is b-sitosterol. In some embodiments, the phytosterol is stigmasterol.

Other examples of non-ionizable lipid components include non-phosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the non-ionizable lipid components are present from 10 mol % to 60 mol %, from 20 mol % to 55 mol %, from 20 mol % to 45 mol %, 20 mol % to 40 mol %, from 25 mol % to 50 mol %, from 25 mol % to 45 mol %, from 30 mol % to 50 mol %, from 30 mol % to 45 mol %, from 30 mol % to 40 mol %, from 35 mol % to 45 mol %, from 37 mol % to 42 mol %, or 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipids present in the lipid nanoparticle composition.

In the embodiments where the lipid nanoparticle compositions contain a mixture of phospholipid and cholesterol or a cholesterol derivative, the mixture may be present up to 40 mol %, 45 mol %, 50 mol %, 55 mol %, or 60 mol % of the total lipids present in the lipid nanoparticle composition.

In some embodiments, the phospholipid component in the mixture may be present from 2 mol % to 20 mol %, from 2 mol % to 15 mol %, from 2 mol % to 12 mol %, from 4 mol % to 15 mol %, or from 4 mol % to 10 mol % (or any fraction thereof or range therein) of the total lipids present in the lipid nanoparticle composition. In some embodiments, the phospholipid component in the mixture may be present from 5 mol % to 10 mol %, from 5 mol % to 9 mol %, from 5 mol % to 8 mol %, from 6 mol % to 9 mol %, from 6 mol % to 8 mol %, or 5 mol %, 6 mol %, 7 mol %, 8 mol %, 9 mol %, or 10 mol % (or any fraction thereof or range therein) of the total lipids present in the lipid nanoparticle composition.

In some embodiments, the cholesterol component in the mixture may be present from 25 mol % to 45 mol %, from 25 mol % to 40 mol %, from 30 mol % to 45 mol %, from 30 mol % to 40 mol %, from 27 mol % to 37 mol %, from 25 mol % to 30 mol %, or from 35 mol % to 40 mol % (or any fraction thereof or range therein) of the total lipids present in the lipid nanoparticle composition. In some embodiments, the cholesterol component in the mixture may be present from 25 mol % to 35 mol %, from 27 mol % to 35 mol %, from 29 mol % to 35 mol %, from 30 mol % to 35 mol %, from 30 mol % to 34 mol %, from 31 mol % to 33 mol %, or 30 mol %, 31 mol %, 32 mol %, 33 mol %, 34 mol %, or 35 mol % (or any fraction thereof or range therein) of the total lipids present in the lipid nanoparticle composition.

In the embodiments where the lipid nanoparticle compositions are phospholipid-free, the cholesterol or derivative thereof may be present up to 25 mol %, 30 mol %, 35 mol %, 40 mol %, 45 mol %, 50 mol %, 55 mol %, or 60 mol % of the total lipid present in the lipid nanoparticle composition.

In some embodiments, the cholesterol or derivative thereof in the phospholipid-free lipid particle formulation may be present from 25 mol % to 45 mol %, from 25 mol % to 40 mol %, from 30 mol % to 45 mol %, from 30 mol % to 40 mol %, from 31 mol % to 39 mol %, from 32 mol % to 38 mol %, from 33 mol % to 37 mol %, from 35 mol % to 45 mol %, from 30 mol % to 35 mol %, from 35 mol % to 40 mol %, or 30 mol %, 31 mol %, 32 mol %, 33 mol %, 34 mol %, 35 mol %, 36 mol %, 37 mol %, 38 mol %, 39 mol %, or 40 mol % (or any fraction thereof or range therein) of the total lipids present in the lipid nanoparticle composition.

In some embodiments, the non-ionizable lipid components may be present from 5 mol % to 90 mol %, from 10 mol % to 85 mol %, from 20 mol % to 80 mol %, 10 mol % (e.g., phospholipid only), or 60 mol % (e.g., phospholipid and cholesterol or derivative thereof) (or any fraction thereof or range therein) of the total lipids present in the lipid nanoparticle composition.

The percentage of non-ionizable lipid present in the lipid nanoparticle composition is a target amount, and that the actual amount of non-ionizable lipid present may vary, for example, by 5 mol %.

### Lipid Conjugates

The lipid nanoparticle composition described herein may further comprise one or more lipid conjugates. A conjugated lipid may prevent the aggregation of particles. Non-limiting examples of conjugated lipids include PEG-lipid conjugates, cationic polymer-lipid conjugates, and mixtures thereof.

In some embodiments, the lipid conjugate is a PEG-lipid or PEG-modified lipid (alternatively referred to as PEGylated lipid). A PEG lipid is a lipid modified with polyethylene glycol. Examples of PEG-lipids include, but are not limited to, PEG coupled to dialkyloxypropyls (PEG-DAA), PEG coupled to diacylglycerol (PEG-DAG), PEG-modified dialkylamines, PEG-modified diacylglycerols (PEG-DEG), PEG coupled to phospholipids such as phosphatidylethanolamine (PEG-PE), PEG-modified phosphatidic acids, PEG conjugated to ceramides (PEG-CER), PEG conjugated to cholesterol or a derivative thereof, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In some embodiments, the PEG-lipid is selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PE G-diacylglyceride (PEG-DAG), PEG-dipalmitoyl phosphatidylethanolamine (PEG-DPPE), or PEG-1,2-dimyristyloxlpropyl-3-amine (PEG-c-DMA).

PEG is a linear, water-soluble polymer of ethylene PEG repeating units with two terminal hydroxyl groups. PEGs are classified by their molecular weights; and include the following: monomethoxypoly ethylene glycol (MePEG-OH), monomethoxypoly ethylene glycol-succinate (MePEG-S), monomethoxypoly ethylene glycol-succinimidyl succinate (MePEG-S—NHS), monomethoxypoly ethylene glycol-amine (MePEG-NH₂), monomethoxypoly ethylene glycol-tresylate (MePEG-TRES), monomethoxypoly ethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂).

The PEG moiety of the PEG-lipid conjugates described herein may comprise an average molecular weight ranging from 550 daltons to 10,000 daltons. In certain instances, the PEG moiety has an average molecular weight of from 750 daltons to 5,000 daltons (e.g., from 1,000 daltons to 5,000 daltons, from 1,500 daltons to 3,000 daltons, from 750 daltons to 3,000 daltons, from 750 daltons to 2,000 daltons). In some embodiments, the PEG moiety has an average molecular weight of 2,000 daltons or 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In some embodiments, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In some embodiments, a carbamate linker is used to couple the PEG to the lipid.

In some embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available, or can be isolated or synthesized using conventional techniques known to those of skill in the art.

In some embodiments, phosphatidylethanolamines contain saturated or unsaturated fatty acids with carbon chain lengths in the range of C10 to C20. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

The term “diacylglycerol” or “DAG” includes a compound having 2 fatty acyl chains, R1 and R2, both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (CM), palmitoyl (C16), stearoyl (C18), and icosoyl (C20). In some embodiments, R1 and R2 are the same, i.e., R1 and R2 are both myristoyl (i.e., dimyristoyl), R1 and R2 are both stearoyl (i.e., distearoyl).

The term “dialkyloxy propyl” or “DAA” includes a compound having 2 alkyl chains, R and R′, both of which have independently between 2 and 30 carbons. The alkyl groups can be saturated or have varying degrees of unsaturation.

In some embodiments, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxy propyl (C16) conjugate, or a PEG-distearyloxy propyl (C18) conjugate. In some embodiments, the PEG has an average molecular weight of 750 or 2,000 daltons. In some embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, poly gly colic acid, and derivatized celluloses such as hydroxymethylcellulose or hydroxy ethylcellulose.

In some embodiments, the PEG-lipid is a compound of formula

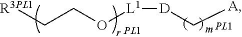

or a salt thereof, wherein:
R^{3PL1} is —OR^{OPL1};
R^{OPL1} is hydrogen, optionally substituted alkyl, or an oxygen protecting group;
r^{PL1} is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{NPL1}), S, C(O), C(O)N(R^{NPL1}) NR^{NPL1}C(O), —C(O)O, OC(O), OC(O)O, OC(O)N(R^{NPL1}), NR^{NPL1}C(O)O, or NR^{NPL1}C(O)N(R^{NPL1});
D is a moiety obtained by click chemistry or a moiety cleavable under physiological conditions; m^{PL1} is 0, 1, 2, 3, 4, 5, 6, 7, 8, 9, or 10;

A is of the formula:

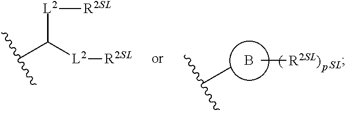

each instance of L is independently a bond or optionally substituted C₁₋₆ alkylene, wherein one methylene unit of the optionally substituted C₁₋₆ alkylene is optionally replaced with O, N(R^{NPL1}), S, C(O), C(O)N(R^{NPL1}), NR^{NPL1}C(O), C(O)O, OC(O), OC(O)O, —OC(O)N(R^{NPL1}), NR^{NPL1}C(O)O, or NR^{NPL1}C(O)N(R^{NPL1});
each instance of R^{2SL} is independently optionally substituted C₁₋₃₀ alkyl, optionally substituted C₁₋₃₀ alkenyl, or optionally substituted C₁₋₃₀ alkynyl; optionally wherein one or more methylene units of R^{2SL} are independently replaced with optionally substituted carbocyclylene, optionally substituted heterocyclylene, optionally substituted arylene, optionally substituted heteroarylene, N(R^{NPL1}), O, S, C(O), C(O)N(R^{NPL1}), NR^{NPL1}C(O), —NR^{NPL1}C(O)N(R^{NPL1}), C(O)O, OC(O), OC(O)O, OC(O)N(R^{NPL1}), NR^{NPL1}C(O)O, C(O)S, —SC(O), C(═NR^{NPL1}), C(═NR^{NPL1})N(R^{NPL1}), NR^{NPL1}C(═NR^{NPL1}), —NR^{NPL1}C(═NR^{NPL1})N(R^{NPL1}), C(S), C(S)N(R^{NPL1}), NR^{NPL1}C(S), NR^{NPL1}C(S)N(R^{NPL1}), S(O), OS(O), S(O)O, OS(O)O, OS(O)₂, S(O)₂O, OS(O)₂O, N(R^{NPL1})S(O), S(O)N(R^{NPL1}), —N(R^{NPL1})S(O)N(R^{NPL1}), OS(O)N(R^{NPL1}), N(R^{NPL1})S(O)O, S(O)₂, N(R^{NPL1})S(O)₂, —S(O)₂N(R^{NPL1}), N(R^{NPL1})S(O)₂N(R^{NPL1}), OS(O)₂N(R^{NPL1}) or N(R^{NPL1})S(O)₂O;
each instance of R^{NPL1} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p^{SL} is 1 or 2.

In some embodiments, the PEG-lipid is a compound of formula

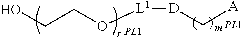

or a salt thereof, wherein r^{PL1}, L¹, D, m^{PL1}, and A are as above defined.

In some embodiments, the PEG-lipid is a compound of formula

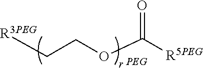

or salt or isomer thereof, wherein:
R^{3PEG} is —OR^{O};
R^{O} is hydrogen, C₁₋₆ alkyl or an oxygen protecting group;
r^{5PEG} is an integer between 1 and 100 (e.g., between 40 and 50, e.g., 45);
R^{5PEG} is C₁₀₋₄₀ alkyl (e.g., C₁₇ alkyl), C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{NPEG}), —O—, —S—, —C(O)—, —C(O)N(R^{NPEG})—, —NR^{NPEG}C(O)—, —NR^{NPEG}C(O)N(R^{NPEG})—, —C(O)O—, —OC(O)—, —OC(O)O—, —OC(O)N(R^{NPEG})—, —NR^{NPEG}C(O)O—, —C(O)S—, —SC(O)—, —C(═NR^{NPEG})—, —C(═NR^{NPEG})N(R^{NPEG})—, —NR^{NPEG}C(═NR^{NPEG})—, —NR^{NPEG}C(═NR^{NPEG})N(R^{NPEG})—, —C(S)—, —C(S)N(R^{NPEG})—, —NR^{NPEG}C(S)—, —NR^{NPEG}C(S)N(R^{NPEG})—, —S(O)—, —OS(O)—, —S(O)O—, —OS(O)O—, —OS(O)₂—, —S(O)₂O—, —OS(O)₂O—, —N(R^{NPEG})S(O)—, —S(O)N(R^{NPEG})—, —N(R^{NPEG})S(O)N(R^{NPEG})—, —OS(O)N(R^{NPEG})—, —N(R^{NPEG})S(O)O—, —S(O)₂—, —N(R^{NPEG})S(O)₂—, —S(O)₂N(R^{NPEG})—, —N(R^{NPEG})S(O)₂N(R^{NPEG})—, —OS(O)₂N(R^{NPEG})—, or —N(R^{NPEG})S(O)₂O—; and
each instance of R^{NPEG} is independently hydrogen, C₁₋₆ alkyl, or a nitrogen protecting group.

In some embodiments, the PEG-lipid is a compound of formula

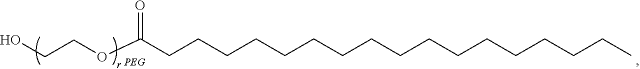

wherein r^{PEG} is an integer between 1 and 100 (e.g., between 40 an 50, e.g., 45).

In some embodiments, the PEG-lipid is a compound of formula

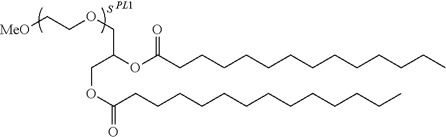

or a salt or isomer thereof, wherein s^{PL1} is an integer between 1 and 100 (e.g., between 40 and 50, e.g., 45).

In some embodiments, the PEG-lipid has the formula of

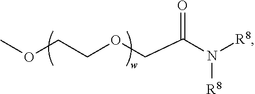

or a pharmaceutically acceptable salt, tautomer or stereoisomer thereof, wherein
R⁸ and R⁹ are each independently a straight or branched, saturated or unsaturated alkyl chain containing from 10 to 30 carbon atoms, wherein the alkyl chain is optionally interrupted by one or more ester bonds (e.g., R⁸ and R⁹ are each independently straight, saturated alkyl chains containing from 12 to 16 carbon atoms); and
w has a mean value ranging from 30 to 60 (e.g., the average w is about 49).

In some embodiments, the incorporation of any of the above-discussed PEG-lipids in the lipid nanoparticle composition can improve the pharmacokinetics and/or biodistribution of the LNP composition. For example, incorporation of any of the above-discussed PEG-lipids in the lipid nanoparticle composition can reduce the accelerated blood clearance (ABC) effect.

In some embodiments, the lipid conjugate (e.g., PEG-lipid) is present from 0.1 mol % to 2 mol %, from 0.5 mol % to 2 mol %, from 1 mol % to 2 mol %, from 0.6 mol % to 1.9 mol %, from 0.7 mol % to 1.8 mol %, from 0.8 mol % to 1.7 mol %, from 0.9 mol % to 1.6 mol %, from 0.9 mol % to 1.8 mol %, from 1 mol % to 1.8 mol %, from 1 mol % to 1.7 mol %, from 1.2 mol % to 1.8 mol %, from 1.2 mol % to 1.7 mol %, from 1.3 mol % to 1.6 mol %, or from 1.4 mol % to 1.5 mol % (or any fraction thereof or range therein) of the total lipids present in the lipid nanoparticle composition. In some embodiments, the lipid conjugate (e.g., PEG-lipid) is present from 0 mol % to 20 mol %, from 0.5 mol % to 20 mol %, from 2 mol % to 20 mol %, from 1.5 mol % to 18 mol %, from 2 mol % to 15 mol %, from 4 mol % to 15 mol %, from 2 mol % to 12 mol %, from 5 mol % to 12 mol %, or 2 mol % (or any fraction thereof or range therein) of the total lipids present in the lipid nanoparticle composition.

In some embodiments, the lipid conjugate (e.g., PEG-lipid) is present from 4 mol % to 10 mol %, from 5 mol % to 10 mol %, from 5 mol % to 9 mol %, from 5 mol % to 8 mol %, from 6 mol % to 9 mol %, from 6 mol % to 8 mol %, or 5 mol %, 6 mol %, 7 mol %, 8 mol %, 9 mol %, or 10 mol % (or any fraction thereof or range therein) of the total lipids present in the lipid nanoparticle composition.

The percentage of lipid conjugate (e.g., PEG-lipid) present in the lipid nanoparticle composition is a target amount, and the actual amount of lipid conjugate present in the composition may vary, for example, by ±2 mol %. One of ordinary skill in the art will appreciate that the concentration of the lipid conjugate can be varied depending on the lipid conjugate employed and the rate at which the lipid particle is to become fusogenic.

By controlling the composition and concentration of the lipid conjugate, one can control the rate at which the lipid conjugate exchanges out of the lipid nanoparticle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will become apparent to those of skill in the art upon reading this disclosure. Also, by controlling the composition and concentration of the lipid conjugate, one can control the lipid nanoparticle size.

In some embodiments, the lipid nanoparticle composition may comprise 30-70% ionizable lipid compound, 0-60% cholesterol, 0-30% phospholipid, and 1-10% polyethylene glycol (PEG)-lipid. In some embodiments, the LNP composition may comprise 30-40% ionizable lipid compound, 40-50% cholesterol, and 10-20% PEG-lipid. In some embodiments, the LNP composition may comprise 50-75% ionizable lipid compound, 20-40% cholesterol, 5-10% phospholipid, and 1-10% PEG-lipid. In some embodiments, the LNP composition may contain 60-70% ionizable lipid compound, 25-35% cholesterol, and 5-10% PEG-lipid.

In some embodiments, the LNP composition may contain up to 90% ionizable lipid compound and 2-15% helper lipid.

In some embodiments, the lipid nanoparticle composition may contain 8-30% ionizable lipid compound, 5-30% helper lipid, and 0-20% cholesterol. In some embodiments, the lipid nanoparticle composition contains 4-25% ionizable lipid compound, 4-25% helper lipid, 2-25% cholesterol, 10-35% cholesterol-PEG, and 5% cholesterol-amine. In some embodiments, the lipid nanoparticle composition contains 2-30% ionizable lipid compound, 2-30% helper lipid, 1-15% cholesterol, 2-35% cholesterol-PEG, and 1-20% cholesterol-amine. In some embodiments, the lipid nanoparticle composition contains up to 90% ionizable lipid compound and 2-10% helper lipids. In some embodiments, the lipid nanoparticle composition contains 100% ionizable lipid compound.

### Other Components for the LNP Composition

The lipid nanoparticle composition may include one or more components in addition to those described above. For example, a LNP composition may include one or more small hydrophobic molecules such as a vitamin (e.g., vitamin A or vitamin E) or a sterol.

The lipid nanoparticle composition may also include one or more permeability enhancer molecules, carbohydrates, polymers, surface altering agents, or other components.

Suitable carbohydrates may include simple sugars (e.g., glucose) and polysaccharides (e.g., glycogen and derivatives and analogs thereof).

A polymer may be used to encapsulate or partially encapsulate a nanoparticle composition. The polymer may be biodegradable and/or biocompatible. Suitable polymers include, but are not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Suitable surface altering agents include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase) A surface altering agent may be disposed within a lipid nanoparticle and/or on the surface of a lipid nanoparticle (e.g., by coating, adsorption, covalent linkage, or other process).

The lipid nanoparticle composition may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a lipid nanoparticle may also be conjugated with one or more useful antibodies. Functional groups and conjugates useful in targeted cell delivery, imaging, and membrane permeation are well known in the art.

The lipid nanoparticle composition may include any substance useful in pharmaceutical compositions. For example, the lipid nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included.

Suitable diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Suitable surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers. (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Suitable binding agents may be starch (e.g. cornstarch and starch paste); gelatin; sugars (e.g. sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol, inorganic calcium salts, silicic acid; polymethacrylates; waxes; water; alcohol, and combinations thereof, or any other suitable binding agent.

Suitable preservatives may include, but are not limited to, antioxidants, chelating agents, antimicrobial preservatives, antifungal preservatives, alcohol preservatives, acidic preservatives, and/or other preservatives Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisultite, sodium metabisulfite, potassium sulfite, potassium metabisultite, GLYDANT PLUS®, PHENONIEP®, methylparaben, GERMALL® 115, GERMABEN®II, NEOLONE™, KATHON™, and/or EUXYL®.

Suitable lubricating agents include, but are not limited to, magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Suitable oils include, but are not limited to, almond, apricot kernel, avocado, babassu, bergamot, black current seed, borage, cade, camomile, canola, caraway, caranuba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

In some embodiments, the lipid nanoparticle composition further comprises one or more cryoprotectants. Suitable cryoprotective agents include, but are not limited to, a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof.

In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, the lipid nanoparticle composition further comprises one or more buffers. Suitable buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the buffer is an acetate buffer, a citrate buffer, a phosphate buffer, a tris buffer, or combinations thereof.

In some embodiments, the lipid nanoparticle composition further comprises one or more nucleic acids, ionizable lipids, amphiphiles, phospholipids, cholesterol, and/or PEG-linked cholesterol.

### Therapeutic Agents

In some embodiments, the lipid nanoparticle composition further comprises one or more therapeutic and/or prophylactic agents (e.g., nucleic acid components).

In some embodiments, the therapeutic and/or prophylactic agent is a vaccine, a compound (e.g., a polynucleotide or nucleic acid molecule that encodes a protein or polypeptide or peptide or a protein or polypeptide or protein) that elicits an immune response, and/or another therapeutic and/or prophylactic. Vaccines include compounds and preparations that are capable of providing immunity against one or more conditions related to infectious diseases and can include mRNAs encoding infectious disease derived antigens and/or epitopes Vaccines also include compounds and preparations that direct an immune response against cancer cells and can include mRNAs encoding tumor cell derived antigens, epitopes, and/or neoepitopes. In some embodiments, a vaccine and/or a compound capable of eliciting an immune response is administered intramuscularly via a composition of the disclosure.

In some embodiments, the therapeutic and/or prophylactic is a protein, for example a protein needed to augment or replace a naturally-occurring protein of interest. Such proteins or polypeptides may be naturally occurring, or may be modified using methods known in the art, e.g., to increase half life. Exemplary proteins are intracellular, transmembrane, or secreted proteins, peptides, or polypeptide.

In some embodiments, the therapeutic and/or prophylactic agent comprises one or more RNA and/or DNA components. In some embodiments, the therapeutic and/or prophylactic agent comprises one or more DNA components. In some embodiments, the therapeutic and/or prophylactic agent comprises one or more RNA components.

In some embodiments, the one or more RNA components is chosen from mRNA. In some embodiments, the mRNA is a modified mRNA.

In some embodiments, the one or more RNA components comprise a gRNA nucleic acid. In some embodiments, the gRNA nucleic acid is a gRNA.

In some embodiments, the one or more RNA components comprise a Class 2 Cas nuclease mRNA and a gRNA. In some embodiments, the gRNA nucleic acid is or encodes a dual-guide RNA (dgRNA). In some embodiments, the gRNA nucleic acid is or encodes a single-guide RNA (sgRNA). In some embodiments, the gRNA is a modified gRNA. In some embodiments, the modified gRNA comprises a modification at one or more of the first five nucleotides at a 5′ end. In some embodiments, the modified gRNA comprises a modification at one or more of the last five nucleotides at a 3′ end.

In some embodiments, the one or more RNA components comprise an mRNA. In some embodiments, the one or more RNA components comprise an RNA-guided DNA-binding agent, for example a Cas nuclease mRNA (such as a Class 2 Cas nuclease mRNA) or a Cas9 nuclease mRNA.

In some embodiments, the therapeutic and/or prophylactic agent comprises one or more template nucleic acids.

In some embodiments, the therapeutic agent is chosen from one or more nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Nucleic acids may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012, which are incorporated herein by reference in their entirety).

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g. Losick, R., 1972, In vitro transcription, Ann Rev Biochem v. 41 409-46; Kamakaka, R. T. and Kraus, W. L. 2001. In Vitro Transcription. Current Protocols in Cell Biology. 2: 11.6: 11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA may be purified from the undesired components of the transcription or associated reactions (including unincorporated rNTPs, protein enzyme, salts, short RNA oligos, etc.). Techniques for the isolation of the mRNA transcripts are well known in the art. Well known procedures include, for non-limiting examples, phenol/chloroform extraction or precipitation with either alcohol (ethanol, isopropanol) in the presence of monovalent cations or lithium chloride.

Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v. 10, 889-893, which is incorporated herein by reference in its entirety), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012, which is incorporated herein by reference in its entirety). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain a number of aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scaleable HPLC purification (see, e.g., Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v. 969 (Rabinovich, P. H. Ed), 2013, which are incorporated herein by reference in their entirety). HPLC purified mRNA has been reported to be translated at much greater levels, particularly in primary cells and in vivo.

A significant variety of modifications have been described in the art which are used to alter specific properties of in vitro transcribed mRNA, and may improve its utility. These include, but are not limited to modifications to the 5′ and 3′ termini of the mRNA. Endogenous eukaryotic mRNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e., capping during in vitro transcription). For example, the Anti-Reverse Cap Analog (ARC A) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life and reduced susceptibility to 5′ endonucleases and/or reduced 5′ decapping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v. 969 (Rabinovich, P. H. Ed), 2013, which are incorporated herein by reference in their entirety).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v. I1, 611-613, which are incorporated herein by reference in their entirety).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′termini of RNA, requiring no additional manipulation of the DNA template, but results in mRNA with poly(A) tails of heterogeneous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self DNA and RNA has been shown to be based, at least in part, on structure and nucleoside modifications since most nucleic acids from natural sources contain modified nucleosides. In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see, e.g., Kariko, K. And Weissman, D. 2007, Naturally occurring nucleoside modifications suppress the immunostimulatory activity of RNA: implication for therapeutic RNA development, Curr Opin Drug Discov Devel, v. 10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013; Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v. 16, 1833-1840). The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see, e.g., US 2012/0251618, which is incorporated herein by reference in its entirety). In vitro synthesis of nucleoside-modified mRNA has been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see, e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013, which are incorporated herein by reference in their entirety).

In addition to mRNA, other nucleic acid payloads may be used for this disclosure. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with embodiments of this disclosure commonly utilizes, but is not limited to, expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selectively grow in antibiotic-containing cultures. Methods of isolating plasmid DNA are widely used and well known in the art (see, e.g., Heilig, J., Elbing, K. L. and Brent, R., (2001), Large-Scale Preparation of Plasmid DNA, Current Protocols in Molecular Biology, 41:11: 1.7: 1.7.1-1.7.16; Rozkov, A., Larsson, B., Gillstrom, S., Bjornestedt, R. and Schmidt, S. R., (2008), Large-scale production of endotoxin-free plasmids for transient expression in mammalian cell culture, Biotechnol. Bioeng., 99: 557-566; and U.S. Pat. No. 6,197,553 BI, which are incorporated herein by reference in their entirety). Plasmid isolation can be performed using a variety of commercially available kits including, but not limited to Plasmid Plus (Qiagen), GenJET plasmid MaxiPrep (Thermo) and Pure Yield MaxiPrep (Promega) kits as well as with commercially available reagents.

The amount of a therapeutic and/or prophylactic in the lipid nanoparticle composition may depend on the size, composition, desired target and/or application, or other properties of the LNP composition as well as on the properties of the therapeutic and/or prophylactic agent. For example, the amount of an RNA useful in a LNP composition may depend on the size, sequence, and other characteristics of the RNA. The relative amounts of a therapeutic and/or prophylactic agent and other elements (e.g., lipids) in a LNP composition may also vary. In some embodiments, the wt/wt ratio of the lipid component to a therapeutic and/or prophylactic agent in a LNP composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic agent may be from about 10:1 to about 40:1. In certain embodiments, the wt/wt ratio is about 20:1.

In some embodiments, the lipid nanoparticle composition includes one or more RNAs, and the one or more RNAs, lipids, and amounts thereof may be selected to provide a specific N:P ratio. The N P ratio of the LNP composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

### Production of Lipid Nanoparticle Compositions

In some embodiments, the lipid nanoparticle composition may be prepared by first combining the ionizable lipid compounds described herein with or without a helper lipid and/or other lipid components (e.g., a phospholipid (e.g., DOPE or DSPC), a PEG lipid (e.g., 1,2-dimyristoyl-sn-glycerol methoxypolyethylene glycol, also known as PEG-DMG), a structural lipid (e.g., cholesterol)) in a buffer solution and then forming the lipid nanoparticle, e.g., via nanoprecipitation.

In some embodiments, the lipid nanoparticle composition may be made according to methods described e.g., in WO 2020/160397, which is incorporated herein by reference in its entirety

### Characterization of Nanoparticle Compositions

The characteristics of the lipid nanoparticle composition may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the nanoparticle composition.

The lipid nanoparticles may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (e.g., by Malvern Instruments Ltd, Malvern, Worcestershire, UK) may also be used to measure multiple characteristics of a nanoparticle composition, such as particle size, polydispersity index, and zeta potential.

In some embodiments, the particle size, the polydispersity index (PDI) and the zeta potential of the lipid nanoparticle compositions may be determined by a zeta potential analyzer. An exemplary zeta potential analyzer is a Zetasizer Nano ZS (e.g., by Malvern Instruments Ltd, Malvern, Worcestershire, UK). The lipid nanoparticle composition can be dispersed a buffer solution for such determination. e.g., in 1×PBS for determining particle size and 15 mM PBS for determining zeta potential.

In some embodiments, the mean diameter of the lipid nanoparticle composition (e.g., an empty LNP or a therapeutic agent-loaded LNP) is between 10s of nm and 100s of nm as measured by dynamic light scattering (DLS). In some embodiments, the mean diameter of the LNP composition is from about 40 nm to about 150 nm in some embodiments, the mean diameter of the LNP composition is about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean diameter of the LNP composition is from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 150 nm, from about 70 nm to about 130 nm, from about 70 nm to about 100 nm, from about 70 nm to about 90 nm, from about 70 nm to about 80 nm, from about 80 nm to about 150 nm, from about 80 nm to about 130 nm, from about 80 nm to about 100 nm, from about 80 nm to about 90 nm, from about 90 nm to about 150 nm, from about 90 nm to about 130 nm, or from about 90 nm to about 100 nm. In certain embodiments, the mean diameter of the LNP composition is from about 70 nm to about 130 nm or from about 70 nm to about 100 nm. In some embodiments, the mean diameter of the LNP composition is about 80 nm. In some embodiments, the mean diameter of the LNP composition is about 100 nm. In some embodiments, the mean diameter of the LNP composition is about 110 nm. In some embodiments, the mean diameter of the LNP composition is about 120 nm.

In some embodiments, the polydispersity index (“PDI”) of a plurality of the lipid nanoparticles (e.g., empty LNPs or a therapeutic agent-loaded LNPs) formulated with the ionizable lipid compounds of the disclosure is less than 0.3. In some embodiments, plurality of the lipid nanoparticles formulated with the ionizable lipid compounds of the disclosure has a PDI of from about 0 to about 0.25. In some embodiments, plurality of the lipid nanoparticles formulated with the ionizable lipid compounds of the disclosure has a PDI of from about 0.10 to about 0.20.

Surface hydrophobicity of lipid nanoparticles can be measured by Generalized Polarization by Laurdan (GPL). In this method, Laurdan, a fluorescent aminonaphthalene ketone lipid, is post-inserted into the nanoparticle surface and the fluorescence spectrum of Laurdan is collected to determine the normalized Generalized Polarization (N-GP). In some embodiments, the have a surface hydrophobicity expressed as N-GP of between about 0.5 and about 1.5. For example, in some embodiments, the lipid nanoparticles formulated with the ionizable lipid compounds of the disclosure have a surface hydrophobicity expressed as N-GP of about 0.5, about 0.6, about 0.7, about 0.8, about 0.9, about 1.0, about 1.1, about 1.2, about 1.3, about 1.4, or about 1.5. In some embodiments, the lipid nanoparticles formulated with the ionizable lipid compounds of the disclosure have a surface hydrophobicity expressed as N-GP of about 1.0 or about 1.1.

The zeta potential of a lipid nanoparticle may be used to indicate the electrokinetic potential of the composition. For example, the zeta potential may describe the surface charge of a lipid nanoparticle composition. Lipid nanoparticles with relatively low charges, positive or negative, are generally desirable, as more highly charged species may interact undesirably with cells, tissues, and other elements in the body. In some embodiments, the zeta potential of the lipid nanoparticles may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The concentration of a therapeutic and/or prophylactic (e.g., RNA) in the lipid nanoparticle composition may be determined by an ultraviolet-visible spectroscopy. The lipid nanoparticle composition can be dispersed in a buffer solution and a solvent for such determination, e.g., 100 μL of the diluted formulation in 1/PBS may be added to 900 LL of a 4:1 (v/v) mixture of methanol and chloroform After mixing, the absorbance spectrum of the solution may be recorded, for example, between 230 nm and 330 nm on a DU 800 spectrophotometer (e.g., by Beckman Coulter, Beckman Coulter, Inc., Brea, CA). The concentration of the therapeutic and/or prophylactic agent in the nanoparticle composition can be calculated based on the extinction coefficient of the therapeutic and/or prophylactic agent used in the composition and on the difference between the absorbance at a wavelength of, for example, 260 nm and the baseline value at a wavelength of, for example, 330 nm.

The efficiency of the encapsulation of a therapeutic and/or prophylactic agent in a lipid nanoparticle composition describes the amount of the therapeutic and/or prophylactic agent that is encapsulated or otherwise associated with the lipid nanoparticles after preparation, relative to the initial amount provided. The encapsulation efficiency is desired to be high (e.g., close to 100%). The encapsulation efficiency may be measured, for example, by comparing the amount of the therapeutic and/or prophylactic agent in a solution containing a loaded LNP before and after breaking up the loaded LNP with one or more organic solvents or detergents. Fluorescence may be used to measure the amount of free therapeutic and/or prophylactic (e.g., RNA) in a solution.

For instance, the encapsulation efficiency may be evaluated using an assay known to one skilled in the art. In one embodiment, a QUANT-IT™ RIBOGREEN® RNA assay (e.g., by Invitrogen Corporation Carlsbad, CA) may be used. In one embodiment, the samples may be diluted to a concentration of approximately 5 μg/mL in a TE buffer solution (10 mM Tris-HCl, 1 mM EDTA, pH 7.5). 50 μL of the diluted samples may be transferred to a polystyrene 96 well plate and either 50 μL of TE buffer or 50 μL of a 2% Triton X-100 solution may be added to the wells. The plate may be incubated at a temperature of 37° C. for 15 minutes. The RIBOGREEN® reagent may be diluted 1:100 in TE buffer, and 100 μL of this solution may be added to each well. The fluorescence intensity can be measured using a fluorescence plate reader (e.g., by Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank may be subtracted from that of each of the samples and the percentage of free RNA may be determined by dividing the fluorescence intensity of the intact sample (without addition of Triton X-100) by the fluorescence value of the disrupted sample (caused by the addition of Triton X-100).

In some embodiments, for the loaded LNPs formulated with the ionizable lipid compounds of the disclosure, the encapsulation efficiency of a therapeutic and/or prophylactic agent is at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency is at least 80%. In some embodiments, the encapsulation efficiency is at least 90%.

In some embodiments, the encapsulation efficiency of the therapeutic and/or prophylactic agent is between 80% and 100%.

### Additional Exemplary LNP Formulations

The lipid nanoparticles may include a lipid component and one or more additional components, such as a therapeutic and/or prophylactic agent A lipid nanoparticle composition may be designed for one or more specific applications or targets. The elements of a lipid nanoparticle may be selected based on a particular application or target, and/or based on the efficacy, toxicity, expense, ease of use, availability, or other feature of one or more elements. Similarly, the particular formulation of a lipid nanoparticle composition may be selected for a particular application or target according to, for example, the efficacy and toxicity of particular combinations of elements.

In some embodiments, the lipid components of the lipid nanoparticle composition include one or more ionizable lipid compounds described herein, a phospholipid (such as an unsaturated lipid, e.g., DOPE or DSPC), a PEG-lipid, and a structural lipid.

In some embodiments, the lipid components of the lipid nanoparticle composition include one or more ionizable lipid compounds described herein, a phospholipid, a PEG-lipid, and a structural lipid.

In some embodiments, the LNP composition comprises one or more ionizable lipid compounds described herein, a phospholipid, a structural lipid, a PEG-lipid, and one or more therapeutic and/or prophylactic agents.

In some embodiments, the LNP composition comprises one or more ionizable lipid compounds described herein, in an amount from about 40% to about 60%.

In some embodiments, the LNP composition comprises the phospholipid in an amount from about 0% to about 20%. For example, in some embodiments, the LNP composition comprises DSPC in an amount from about 0% to about 20%.

In some embodiments, the LNP composition comprises the structural lipid in an amount from about 30% to about 50%. For example, in some embodiments, the LNP composition comprises cholesterol in an amount from about 30% to about 50%.

In some embodiments, the LNP composition comprises the PEG-lipid in an amount from about 0% to about 5% For example, in some embodiments, the LNP composition comprises PEG-1 or PEG₂ₖ-DMG in an amount from about 0% to about 5%.

In some embodiments, the lipid components of the nanoparticle composition include about 30 mol % to about 60 mol % one or more ionizable lipid compounds described herein, about 0 mol % to about 30 mol % phospholipid, about 18.5 mol % to about 48.5 mol % structural lipid, and about 0 mol % to about 10 mol % of PEG-lipid, provided that the total mol % does not exceed 100%. In some embodiments, the lipid components of the nanoparticle composition include about 35 mol % to about 55 mol % one or more ionizable lipid compounds described herein, about 5 mol % to about 25 mol % phospholipid, about 30 mol % to about 40 mol % structural lipid, and about 0 mol % to about 10 mol % of PEG-lipid. In one embodiment, the lipid components include about 50 mol % one or more ionizable lipid compounds described herein, about 10 mol % phospholipid, about 38.5 mol % structural lipid, and about 1.5 mol % of PEG-lipid. In one embodiment, the lipid components include about 40 mol % one or more ionizable lipid compounds described herein, about 20 mol % phospholipid, about 38.5 mol % structural lipid, and about 1.5 mol % of PEG-lipid. In some embodiments, the phospholipid may be DOPE or DSPC in some embodiments, the PEG-lipid may be PEG-1 or PEG₂ₖ-DMG, and/or the structural lipid may be cholesterol.

In some embodiments, the LNP composition comprises about 40 mol % to about 60 mol % of one or more ionizable lipid compounds described herein, about 0 mol % to about 20 mol % phospholipid, about 30 mol % to about 50 mol % structural lipid, and about 0 mol % to about 5 mol % PEG-lipid. In some embodiments, the LNP composition comprises comprises about 40 mol % to about 60 mol % of one or more ionizable lipid compounds described herein, about 0 mol % to about 20 mol % DSPC, about 30 mol % to about 50 mol % cholesterol, and about 0 mol % to about 5 mol % PEG-1 or PEG₂ₖ-DMG.

The lipid nanoparticles may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of the lipid nanoparticles may be altered in order to increase selectivity for particular bodily targets. For instance, particle sizes may be adjusted based on the fenestration sizes of different organs. The therapeutic and/or prophylactic agent included in a LNP composition may also be selected based on the desired delivery target or targets. For example, a therapeutic and/or prophylactic agent may be selected for a particular indication, condition, disease, or disorder and/or for delivery to a particular cell, tissue, organ, or system or group thereof (e.g., localized or specific delivery). In certain embodiments, a lipid nanoparticle composition may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver.

### In Vivo Formulation Studies

To monitor the effectiveness of the lipid nanoparticle compositions deliver therapeutic and/or prophylactics to targeted cells, different nanoparticle compositions including a particular therapeutic and/or prophylactic (for example, a modified or naturally occurring RNA such as an mRNA) may be prepared and administered to animal populations Animals (e.g., mice, rats, or non-human primates) may be intravenously, intramuscularly, intraarterially, or intratumorally administered a single dose including the LNP composition described herein and an mRNA expressing a protein, e.g., human erythropoietin (hEPO) or luciferase. A control composition including PBS may also be employed.

Upon administration of the LNP compositions to an animal, dose delivery profiles, dose responses, and toxicity of particular formulations and doses thereof can be measured by enzyme-linked immunosorbent assays (ELISA), bioluminescent imaging, or other methods. For the LNP compositions including mRNA, time courses of protein expression can also be evaluated. Samples collected from the animals for evaluation may include blood, sera, and tissue (for example, muscle tissue from the site of an intramuscular injection and internal tissue); sample collection may involve sacrifice of the animals.

In some embodiments, hEPO concentrations may be determined using an enzyme-linked lectin assay (ELLA) Simple Plex Assay (ProteinSimple) with a Human Erythroprotein cartridge Standards for this assay may be calibrated according to the 2 IRP WHO preparation.

The LNP compositions including mRNA are useful in the evaluation of the efficacy and usefulness of various formulations for the delivery of therapeutic and/or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic by a given nanoparticle composition relative to other nanoparticle compositions or the absence thereof.

In some embodiments, an in vivo expression assay may be used to assess potency of expression of the ionizable lipids of the disclosure.

In some embodiments, protein expression (e.g., hEPO) may be measured in mice following administration of the loaded LNP composition. In some embodiments, the concentration of hEPO in serum may be tested after administration (e.g., about six hours after injection).

In some embodiments, the LNP composition may be intravenously administered to mice (e.g., CD-1 mice).

In some embodiments, residual levels of the lipids in organs or tissue of the subject after administration (e.g., 6 h, 12 h, 18 h, 24 h, 36 h, or 48 h after administration) may be measured. In some embodiments, the residual levels of the lipids of the disclosure in the liver may be measured.

In some embodiments, an in vitro expression assay may be used to assess the lipids and LNP composition.

In some embodiments, cells (e.g., HeLa) may be plated in an imaging plate (e.g., poly-D-lysene coated) and cultured in serum (e.g., human serum, mouse serum, cynomolgus monkey serum or fetal bovine serum).

In some embodiments, the LNP composition comprising an mRNA expressing fluorescent protein (e.g., green fluorescent protein (GFP)) and a fluorescent lipid (e.g., rhodamine-DOPE) may be added to the plate and the plate imaged for uptake and expression. In some embodiments, expression may be evaluated by measuring fluorescence (e.g., from GFP). In some embodiments, uptake (accumulation) may be evaluated by measuring the fluorescence signal from a fluorescent lipid (e.g., rhodamine-DOPE).

### Methods for Using the LNP Composition

In some embodiments, provided herein is a method of delivering a therapeutic agent (i.e., cargo) to at least one organ chosen from the pancreas, one or both lungs, and the spleen of a subject in need thereof comprising administering to said subject a lipid nanoparticle composition comprising one or more ionizable lipid compounds disclosed herein (e.g., compounds of Formula (I)—(VII)) with a minimum amount delivered elsewhere in body, such as in the liver, of the subject.

In some embodiments, the method delivers a therapeutic agent (i.e., cargo) to the pancreas and/or one or both lungs a subject in need thereof with a minimum amount delivered elsewhere in body, such as in the liver, of the subject.

In some embodiments, less than 55%, 50%, 45%, 40%, 35%, 30%, 25%, 20%, 15%, 10%, 5% or 1% of the total therapeutic cargo administered to the subject is delivered to the liver of the subject. In some embodiments, less than 6%, 7%, 8%, 10%, 11%, 12%, 13%, 14%, 15%, 16%, 17%, 18%, 19%, or 20% of the total therapeutic cargo administered to the subject is delivered to the liver of the subject.

In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the pancreas and/or one or both lungs of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the pancreas of the subject. In some embodiments, more than 99%, 95%0, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the lungs of the subject.

As used herein, the percent amount of the total therapeutic cargo administered to the subject and delivered to a location in the subject is measured by the level of protein expression, or mRNA knockdown level.

In some embodiments, the method of delivering a therapeutic cargo disclosed above comprises administering to a subject a lipid nanoparticle composition comprising one or more ionizable lipid compounds disclosed herein, encapsulating the therapeutic cargo. In some embodiments, the lipid nanoparticles in the lipid nanoparticle composition are formed from one or more compounds chosen from the ionizable lipids of Formulas (IO)—(VIIO) and Formulas (I)—(VIID), pharmaceutically acceptable salts thereof, and stereoisomers of any of the foregoing. In some embodiments, the lipid nanoparticles are formed from one or more compounds chosen from the ionizable lipids of Formulas (IO), (I), or (IA), pharmaceutically acceptable salts thereof, and stereoisomers of any of the foregoing. In some embodiments, the lipid nanoparticles are formed from one or more compounds chosen from the ionizable lipids of Formula (IIO), (IIA), (IIB), (IIC), or (IID), pharmaceutically acceptable salts thereof, and stereoisomers of any of the foregoing. In some embodiments, the lipid nanoparticles are formed from one or more compounds chosen from the ionizable lipids of Formula (IIIO), (IIIA), or (IIIB) pharmaceutically acceptable salts thereof, and stereoisomers of any of the foregoing. In some embodiments, the lipid nanoparticles are formed from one or more compounds chosen from the ionizable lipids of Formula (IVO), (IVA), (IVB), (IVC), or (IVD), pharmaceutically acceptable salts thereof, and stereoisomers of any of the foregoing. In some embodiments, the lipid nanoparticles are formed from one or more compounds chosen from the ionizable lipids of Formula (VO), (V), (VA), or (VB), pharmaceutically acceptable salts thereof, and stereoisomers of any of the foregoing. In some embodiments, the lipid nanoparticles are formed from one or more compounds chosen from the ionizable lipids of Formula (VIO), (VIA), or (VIB), pharmaceutically acceptable salts thereof, and stereoisomers of any of the foregoing. In some embodiments, the lipid nanoparticles are formed from one or more compounds chosen from the ionizable lipids of one of Formulas (VIIO) or (VIIA)-(VIID), pharmaceutically acceptable salts thereof, and stereoisomers of any of the foregoing.

Non-limiting exemplary embodiments of the ionizable lipids of the present disclosure, lipid nanoparticles and compositions comprising the same, and their use to deliver agents (e.g., therapeutic agents, such as nucleic acids) and/or to modulate gene and/or protein expression are described in further detail below.

In some embodiments, the ionizable lipids and lipid nanoparticle compositions disclosed herein may be used for a variety of purposes, including delivery of encapsulated or associated (e.g., complexed) therapeutic agents such as nucleic acids to cells, in vitro and/or in vivo. Accordingly, in some embodiments, provided are methods of treating or preventing diseases or disorders in a subject in need thereof comprising administering to the subject the lipid nanoparticle composition described herein. In some embodiments, the lipid nanoparticle encapsulates or is associated with a suitable therapeutic agent, wherein the lipid nanoparticle comprises one or more of the novel ionizable lipids described herein, a pharmaceutically acceptable salt thereof, and/or a stereoisomer of any of the foregoing.

In some embodiments, the lipid nanoparticles of the present disclosure are useful for delivery of therapeutic cargo.

In some embodiments, disclosed herein are methods of inducing expression of a desired protein in vitro and/or in vivo by contacting cells with the lipid nanoparticle composition comprising one or more novel ionizable lipids described herein, wherein the lipid nanoparticle encapsulates or is associated with a nucleic acid that is expressed to produce a desired protein (e.g., a messenger RNA or plasmid encoding the desired protein) or inhibit processes that terminate expression of mRNA (e.g., miRNA inhibitors).

In some embodiments, disclosed herein are methods of decreasing expression of target genes and proteins in vitro and/or in vivo by contacting cells with a lipid nanoparticle comprising one or more novel ionizable lipids described herein, wherein the lipid nanoparticle encapsulates or is associated with a nucleic acid that reduces target gene expression (e.g., an antisense oligonucleotide or small interfering RNA (siRNA)).

In some embodiments, disclosed herein are methods for co-delivery of one or more nucleic acid (e.g. mRNA and plasmid DNA). separately or in combination, such as may be useful to provide an effect requiring colocalization of different nucleic acids (e.g. mRNA encoding for a suitable gene modifying enzyme and DNA segment(s) for incorporation into the host genome).

In some embodiments, the lipid nanoparticle compositions are useful for expression of protein encoded by mRNA. In some embodiments, provided herein are methods for expression of protein encoded by mRNA.

In some embodiments, the lipid nanoparticles compositions are useful for upregulation of endogenous protein expression by delivering miRNA inhibitors targeting one specific miRNA or a group of miRNA regulating one target mRNA or several mRNA. In some embodiments, provided herein are methods for upregulating endogenous protein expression comprising delivering miRNA inhibitors targeting one or more miRNA regulating one or more mRNA.

In some embodiments, the lipid nanoparticle compositions are useful for down-regulating (e.g., silencing) the protein levels and/or mRNA levels of target genes. In some embodiments, provided herein are methods for down-regulating (e.g., silencing) protein and/or mRNA levels of target genes.

In some embodiments, the lipid nanoparticles are useful for delivery of mRNA and plasmids for expression of transgenes. In some embodiments, provided herein are methods for delivering mRNA and plasmids for expression of transgenes.

In some embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antigen or antibody. In some embodiments, provided herein are methods for inducing a pharmacological effect resulting from expression of a protein, e.g., increased production of red blood cells through the delivery of a suitable erythropoietin mRNA, or protection against infection through delivery of mRNA encoding for a suitable antigen or antibody.

In some embodiments, the disclosure relates to a method of gene editing, comprising contacting a cell with an LNP. In some embodiments, the disclosure relates to any method of gene editing described herein, comprising cleaving DNA.

In some embodiments, the disclosure relates to a method of cleaving DNA, comprising contacting a cell with an LNP composition.

In some embodiments, the disclosure relates to any method of cleaving DNA described herein, wherein the cleaving step comprises introducing a single stranded DNA nick. In some embodiments, the disclosure relates to any method of cleaving DNA described herein, wherein the cleaving step comprises introducing a double-stranded DNA break. In some embodiments, the disclosure relates to any method of cleaving DNA described herein, wherein the LNP composition comprises a Class 2 Cas mRNA and a guide RNA nucleic acid.

In some embodiments, the disclosure relates to any method of cleaving DNA described herein, further comprising introducing at least one template nucleic acid into the cell. In some embodiments, the disclosure relates to any method of cleaving DNA described herein, comprising contacting the cell with an LNP composition comprising a template nucleic acid.

In some embodiments, the disclosure relates to any a method of gene editing described herein, wherein the method comprises administering the LNP composition to an animal, for example a human. In some embodiments, the disclosure relates to any method of gene editing described herein, wherein the method comprises administering the LNP composition to a cell, such as a eukaryotic cell.

In some embodiments, the disclosure relates to any method of gene editing described herein, wherein the method comprises administering the mRNA formulated in a first LNP composition and a second LNP composition comprising one or more of an mRNA, a gRNA, a gRNA nucleic acid, and a template nucleic acid. In some embodiments, the disclosure relates to any method of gene editing described herein, wherein the first and second LNP compositions are administered simultaneously. In some embodiments, the disclosure relates to any method of gene editing described herein, wherein the first and second LNP compositions are administered sequentially.

In some embodiments, the disclosure relates to any method of gene editing described herein, wherein the method comprises administering the mRNA and the guide RNA nucleic acid formulated in a single LNP composition.

In some embodiments, the disclosure relates to any method of gene editing described herein, wherein the gene editing results in a gene knockout.

In some embodiments, the disclosure relates to any method of gene editing described herein, wherein the gene editing results in a gene correction.

In some embodiments, the disclosure relates to methods for in vivo delivery of interfering RNA to the lung of a mammalian subject.

In some embodiments, relates to methods of treating a disease or disorder in a mammalian subject. In some embodiments, these methods comprise administering a therapeutically effective amount of a composition of this disclosure to a subject having a disease or disorder associated with expression or overexpression of a gene that can be reduced, decreased, downregulated, or silenced by the composition.

The compositions of this disclosure may be administered by various routes, for example, to effect systemic delivery via intravenous, parenteral, intraperitoneal, or topical routes. In some embodiments, a siRNA may be delivered intracellularly, for example, in cells of a target tissue such as lung or liver, or in inflamed tissues. In some embodiments, this disclosure provides a method for delivery of siRNA in vivo. A nucleic acid-lipid composition may be administered intravenously, subcutaneously, or intraperitoneally to a subject.

The compositions and methods of the disclosure may be administered to subjects by a variety of mucosal administration modes, including by oral, rectal, vaginal, intranasal, intrapulmonary, or transdermal or dermal delivery, or by topical delivery to the eyes, ears, skin, or other mucosal surfaces. In some aspects of this disclosure, the mucosal tissue layer includes an epithelial cell layer. The epithelial cell can be pulmonary, tracheal, bronchial, alveolar, nasal, buccal, epidermal, or gastrointestinal. Compositions of this disclosure can be administered using conventional actuators such as mechanical spray devices, as well as pressurized, electrically activated, or other types of actuators.

Compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, for example, aerosolized, atomized, or nebulized. Particles of the composition, spray, or aerosol can be in either a liquid or solid form. Non-limiting examples of systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in TRANSDERMAL SYSTEMIC MEDICATION, Y. W. Chien ed., Elsevier Publishers, New York, 1985; and in U.S. Pat. No. 4,778,810. Additional aerosol delivery forms may include, e.g., compressed air-Jet-, ultrasonic-, and piezoelectric nebulizers, which deliver the biologically active agent dissolved or suspended in a pharmaceutical solvent, e.g., water, ethanol, or mixtures thereof.

Nasal and pulmonary spray solutions of the present disclosure typically comprise the drug or drug to be delivered, optionally formulated with a surface active agent, such as a nonionic surfactant (e.g., polysorbate-80), and one or more buffers. In some embodiments of the present disclosure, the nasal spray solution further comprises a propellant. The pH of the nasal spray solution may be from pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure is a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet, or gel.

To prepare compositions for pulmonary delivery within the present disclosure, the biologically active agent can be combined with various pharmaceutically acceptable additives, as well as a base or carrier for dispersion of the active agent(s).

Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the composition, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-gly colic acid) copolymer, polyhydroxybutyric acid, poly(hydroxybutyric acid-gly colic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the biologically active agent.

Compositions for mucosal, nasal, or pulmonary delivery may contain a hydrophilic low molecular weight compound as a base or excipient. Such hydrophilic low molecular weight compounds may provide a passage medium through which a water-soluble active agent, such as a physiologically active peptide or protein, may diffuse through the base to the body surface where the active agent is absorbed. The hydrophilic low molecular weight compound may optionally absorb moisture from the mucosa or the administration atmosphere and may dissolve the water-soluble active peptide. In some embodiments, the molecular weight of the hydrophilic low molecular weight compound is less than or equal to 10,000, such as not more than 3,000. Examples of hydrophilic low molecular weight compounds include polyol compounds, such as oligo-, di- and monosaccharides including sucrose, mannitol, lactose, L-arabinose, D-erythrose, D-ribose, D-xylose, D-mannose, D-galactose, lactulose, cellobiose, gentibiose, glycerin, polyethylene glycol, and mixtures thereof. Further examples of hydrophilic low molecular weight compounds include N-methylpyrrolidone, alcohols (e.g., oligovinyl alcohol, ethanol, ethylene glycol, propylene glycol, etc.), and mixtures thereof.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the biologically active agent may be administered in a time release formulation, for example in a composition which includes a slow release polymer. The active agent can be prepared with carriers that will protect against rapid release, for example a controlled release vehicle such as a polymer, microencapsulated delivery system, or bioadhesive gel. Prolonged delivery of the active agent, in various compositions of the disclosure can be brought about by including in the composition agents that delay absorption, for example, aluminum monosterate hydrogels and gelatin.

### EXAMPLES

### Example 1. Synthesis of Various Ionizable Lipids

### Synthesis of 7596

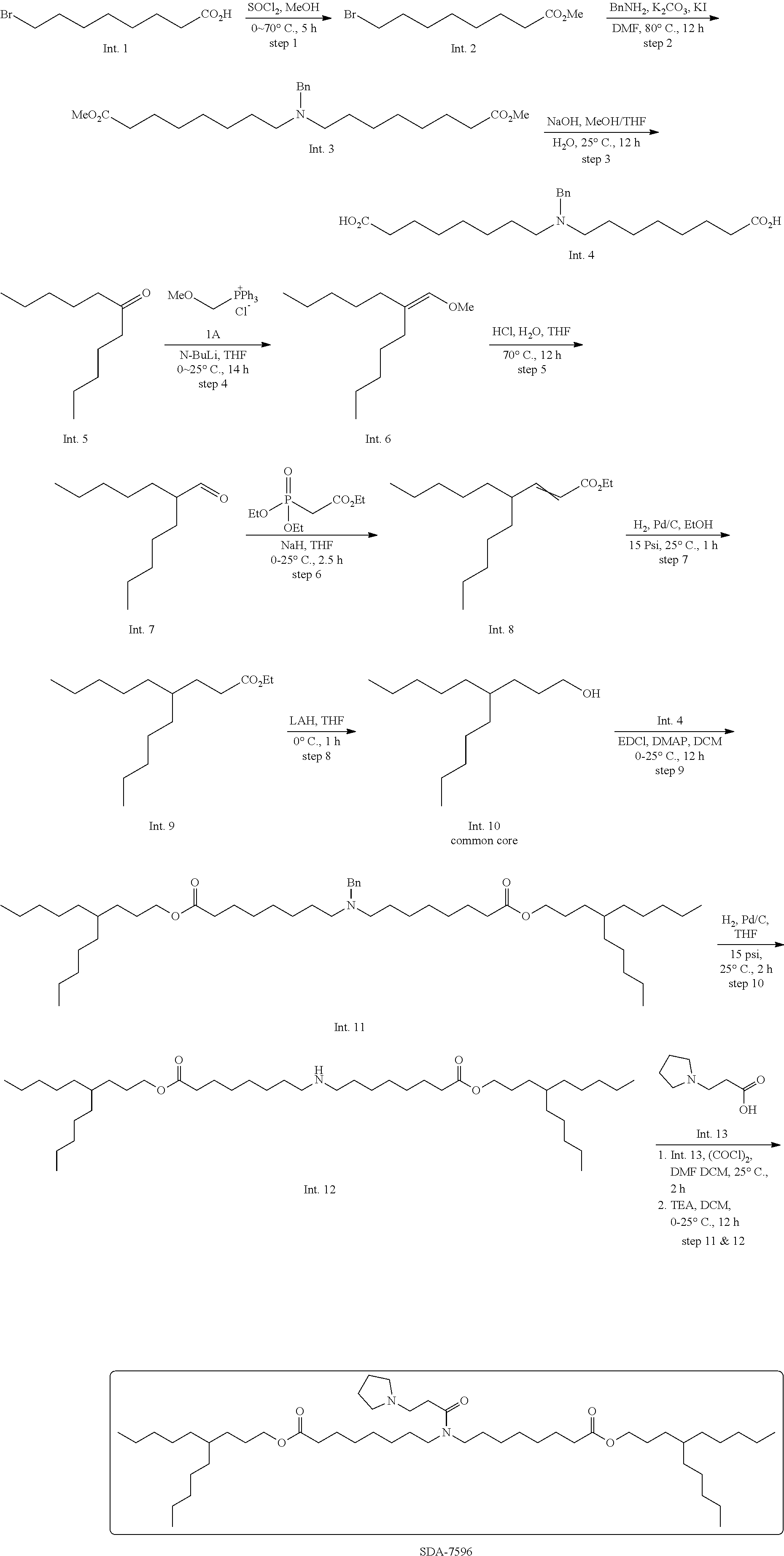

### Step 1:

To a solution of 8-bromooctanoic acid (5.0 g, 22.41 mmol, 1 eq) in MeOH (50 mL) was added dropwise SOCl₂ (5.33 g, 44.82 mmol, 3.25 mL, 2 eq) at 0° C., then the mixture was stirred at 70° C. for 5 h. The mixture was concentrated under reduced pressure to get methyl 8-bromooctanoate (4.5 g, crude) as yellow oil.

### Step 2:

To a solution of BnNH₂ (0.78 g, 7.28 mmol, 793.49 μL, 1 eq) in DMF (10 mL) was added K₂CO₃ (5.03 g, 36.40 mmol, 5 eq), KI (3.02 g, 18.20 mmol, 2.5 eq) and the solution of methyl 8-bromooctanoate (3.5 g, 14.76 mmol, 2.03 eq) in DMF (4 mL), then the mixture was stirred at 80° C. for 12 h. The mixture was filtered and the filtrate was poured into H₂O and extracted with EtOAc. The combined organic layer was washed with brine, dried over Na₂SO₄, filtered and the filtrate was concentrated under reduced pressure to give a residue. The residue was purified by silica gel chromatography to give methyl 8-[benzyl-(8-methoxy-8-oxo-octyl)amino]-octanoate (2.6 g, 6.20 mmol, 85% yield) as yellow oil.

### Step 3:

To a solution of methyl 8-[benzyl-(8-methoxy-8-oxo-octyl)amino]octanoate (2.6 g, 6.20 mmol, 1 eq) in THE (3 mL) and MeOH (10 mL) was added a solution of NaOH (845.87 mg, 21.15 mmol, 3.41 eq) in H₂O (5 mL), then the mixture was stirred at 25° C. for 12 h. The reaction mixture was concentrated under reduced pressure to get a residue. The residue was added into H₂O and extracted with EtOAc. The aqueous phase was adjusted to pH=6-7 with 1N HCl, then extracted with EtOAc. The organic layer was washed with brine, dried over Na₂SO₄, filtered and the filtrate was concentrated under reduced pressure to give 8-[benzyl(7-carboxyheptyl)amino]octanoic acid (2.0 g, 5.11 mmol, 82% yield) as colorless oil.

### Step 4:

To a solution of methoxymethyl(triphenyl)phosphonium; chloride (24.16 g, 70.47 mmol, 3 eq) in THE (360 mL) was added dropwise n-BuLi (2.5 M, 26.31 mL, 2.8 eq) at 0° C. and the mixture was stirred at 25° C. for 2 h. A solution of undecan-6-one (4.0 g, 23.49 mmol, 1 eq) in THF (120 mL) was added into the mixture at 0° C., then stirred at 25° C. for 12 h. The mixture was poured into H₂O at 0° C. and extracted with EtOAc. The combined organic layer was washed with brine, dried over Na₂SO₄, filtered and the filtrate was concentrated under reduced pressure to get a residue. The residue was purified by silica gel chromatography to give 6-(methoxymethylene)undecane (18.0 g, 90.75 mmol, 77% yield) as colorless oil.

### Step 5:

A solution of 6-(methoxymethylene)undecane (18.0 g, 90.75 mmol, 1 eq) in THE (72 mL) and HCl (3 M, 18.00 mL, 5.95e-1 eq) aq. was stirred at 70° C. for 12 h. The mixture was poured into H₂O at 0° C., extracted with EtOAc. The combined organic layer was washed with brine, dried over Na₂SO₄, filtered and the filtrate was concentrated under reduced pressure to get a residue. The residue was purified by silica gel chromatography to give 2-pentylheptanal (15.0 g, 81.38 mmol, 90% yield) as colorless oil.

### Step 6:

To a solution of NaH (3.95 g, 98.74 mmol, 7.05 mL, 60% purity, 1.3 eq) in THE (280 mL) was added dropwise ethyl 2-diethoxyphosphorylacetate (22.14 g, 98.74 mmol, 19.59 mL, 1.3 eq) at 0° C., the mixture was stirred at 25° C. for 0.5 h. A solution of 2-pentylheptanal (14.0 g, 75.96 mmol, 1 eq) in THE (70 mL) was added into the mixture at 0° C., then the mixture was warmed to 25° C. and stirred at 25° C. for 2 h. The mixture was poured into H₂O at 0° C., extracted with EtOAc. The combined organic layer was washed with brine, dried over Na₂SO₄, filtered and the filtrate was concentrated under reduced pressure to get a residue. The residue was purified by silica gel chromatography to give ethyl 4-pentylnon-2-enoate (16.0 g, 62.89 mmol, 83% yield) as colorless oil.

### Step 7:

A solution of Pd/C (2.5 g, 10% purity) and ethyl 4-pentylnon-2-enoate (5.0 g, 19.65 mmol, 1 eq) in EtOH (100 mL) was stirred at 25° C. for 1 h under H₂ (15 Psi). The mixture was filtered and the filtrate was concentrated under reduced pressure to give ethyl 4-pentylnonanoate (15.0 g, crude) as colorless oil.

### Step 8:

To a solution of LAH (1.48 g, 39.00 mmol, 7.05 mL, 2 eq) in THE (50 mL) was added a solution of ethyl 4-pentylnonanoate (5.0 g, 19.50 mmol, 1 eq) in THE (10 mL) at 0° C. and stirred at 0° C. for 1 h. The mixture was poured into H₂O at 0° C., then the mixture was filtered and the filtrate was extracted with EtOAc. The combined organic layer was washed with brine, dried over Na₂SO₄, filtered and the filtrate was concentrated under reduced pressure. The residue was purified by silica gel chromatography to give 4-pentylnonan-1-ol (10.0 g, 46.64 mmol, 80% yield) as colorless oil.

### Step 9:

To a solution of 4-pentylnonan-1-ol (1.15 g, 5.36 mmol, 2.1 eq) and 8-[benzyl(7-carboxyheptyl) amino]octanoic acid (1.0 g, 2.55 mmol, 1 eq) in DCM (10 mL) was added DMAP (156.01 mg, 1.28 mmol, 0.5 eq) and EDCI (1.47 g, 7.66 mmol, 3 eq) at 0° C., then stirred at 25° C. for 12 h. The mixture was added into H₂O and extracted with DCM. The combined organic layer was washed with brine, dried over Na₂SO₄, filtered and the filtrate was concentrated under reduced pressure to get a residue. The residue was purified by silica gel chromatography to give 4-pentylnonyl 8-[benzyl-[8-oxo-8-(4-pentylnonoxy)octyl]amino]octanoate (1.5 g, 1.91 mmol, 75% yield) as colorless oil.

### Step 10:

A solution of Pd/C (200 mg, 637.52 μmol, 10% purity, 1 eq) and 4-pentylnonyl 8-[benzyl-[8-oxo-8-(4-pentylnonoxy)octyl]amino]octanoate (500 mg, 637.52 μmol, 1 eq) in THF (20 mL) was stirred at 25° C. for 2 h under H₂ (15 Psi). The mixture was filtered and the filtrate was concentrated under reduced pressure to get a residue. The residue was purified by silica gel chromatography to give 4-pentylnonyl 8-[[8-oxo-8-(4-pentylnonoxy)octyl]amino]octanoate (900 mg, crude) as colorless oil.

### Step 11:

To a solution of 3-pyrrolidin-1-ylpropanoic acid (100 mg, 698.41 μmol, 1 eq) in DCM (5 mL) was added (COCl)₂ (443.24 mg, 3.49 mmol, 305.69 μL, 5 eq) and DMF (5.10 mg, 69.84 μmol, 5.37 μL, 0.1 eq), stirred at 25° C. for 2 h. The mixture was concentrated under reduced pressure to give 3-pyrrolidin-1-ylpropanoyl chloride (112 mg, crude) as a yellow solid. The crude was used directly.

### Step 12:

To a solution of 4-pentylnonyl 8-[[8-oxo-8-(4-pentylnonoxy)octyl]amino]octanoate (100 mg, 144.06 μmol, 1 eq) in DCM (2 mL) was added TEA (43.73 mg, 432.18 μmol, 60.15 μL, 3 eq) and 3-pyrrolidin-1-ylpropanoyl chloride (114.15 mg, 576.24 μmol, 4 eq, HCl) at 0° C., stirred at 25° C. for 12 h. The mixture was concentrated under reduced pressure to give a residue. The residue was purified by silica gel chromatography to give 4-pentylnonyl 8-[[8-oxo-8-(4-pentylnonoxy)octyl]-(3-pyrrolidin-1-ylpropanoyl)amino]octanoate (87 mg, 101.94 μmol, 71% yield) as colorless oil.

¹H NMR (400 MHz, CDCl₃), 4.03-4.07 (m, 4H), 3.15-3.40 (m, 4H), 2.85 (brs, 2H), 2.59 (brs, 6H), 2.27-2.33 (m, 4H), 1.82 (s, 4H), 1.48-1.62 (m, 12H), 1.24-1.32 (m, 50H), 0.89 (t, J=6.8 Hz, 12H)

[M+H]⁺: 819.6

### Synthesis of 7649

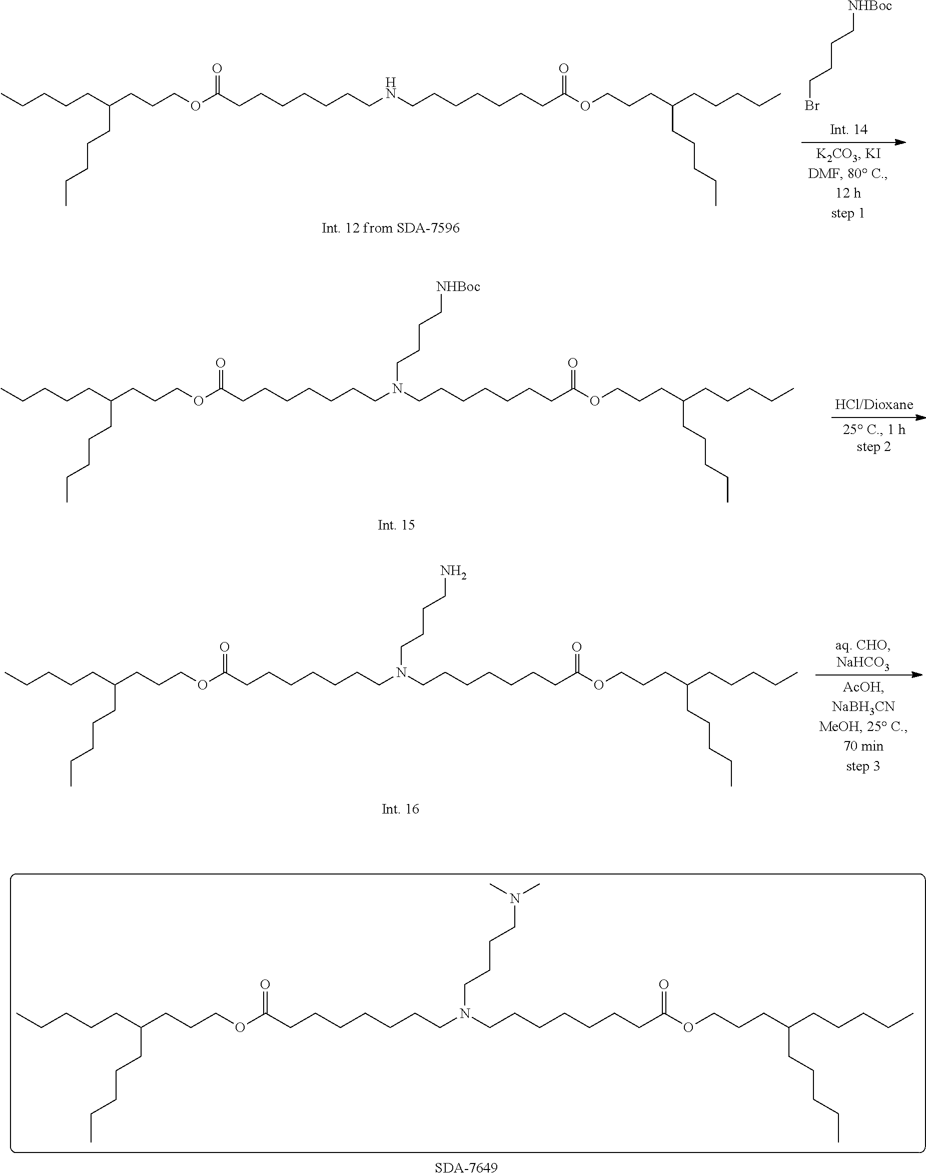

### Step 1:

To a solution of 4-pentylnonyl 8-[[8-oxo-8-(4-pentylnonoxy)octyl]amino]octanoate (150 mg, 216.09 μmol, 1 eq) in DMF (5 mL) was added K₂CO₃ (149.33 mg, 1.08 mmol, 40.10 μL, 5 eq) and KI (71.74 mg, 432.18 μmol, 2 eq), then a solution of tert-butyl N-(4-bromobutyl)carbamate (217.94 mg, 864.35 μmol, 177.19 μL, 4 eq) in DMF (2 mL) was added into the mixture and stirred at 80° C. for 12 h. The reaction mixture was filtered and concentrated under reduced pressure to get a residue. The residue was purified by silica gel chromatography to give 4-pentylnonyl8-[4-(tert-butoxycarbonylamino)butyl-[8-oxo-8-(4-pentylnonoxy)octyl]amino]octanoate (120 mg, 138.66 μmol, 64% yield) as colorless oil.

### Step 2:

A solution of 4-pentylnonyl 8-[4-(tert-butoxycarbonylamino)butyl-[8-oxo-8-(4-pentylnonoxy) octyl]amino]octanoate (120 mg, 138.66 μmol, 1 eq) in HCl/dioxane (4 M, 6.00 mL, 173.08 eq) was stirred at 25° C. for 1 h. The reaction mixture was concentrated under reduced pressure to give 4-pentylnonyl 8-[4-aminobutyl-[8-oxo-8-(4-pentylnonoxy)octyl]amino]octanoate (110 mg, crude, HCl) as a yellow solid.

### Step 3:

To a solution of 4-pentylnonyl 8-[4-aminobutyl-[8-oxo-8-(4-pentylnonoxy)octyl]amino]octanoate (110 mg, 137.20 μmol, 1 eq, HCl) and formaldehyde (1.30 g, 15.97 mmol, 1.19 mL, 37% purity, 116.42 eq) in MeOH (10 mL) was added NaHCO₃ (34.58 mg, 411.60 μmol, 16.01 uL, 3 eq), stirred at 25° C. for 10 min, then AcOH (247.17 mg, 4.12 mmol, 235.40 uL, 30 eq) and NaBH₃CN (25.87 mg, 411.60 μmol, 3 eq) was added to the mixture and stirred at 25° C. for 1 h. The reaction mixture was quenched with sat. NaHCO₃ and extracted with EtOAc. The combined organic layer was washed with brine, dried over anhydrous sodium sulfate, filtered and concentrated under reduced pressure to get a residue. The residue was purified by silica gel chromatography to give 4-pentylnonyl 8-[4-(dimethylamino)butyl-[8-oxo-8-(4-pentylnonoxy)octyl]amino]octanoate (87 mg, 109.66 μmol, 80% yield) as colorless oil.

¹H NMR (400 MHz, CDCl₃), 4.04 (t, J=6.8 Hz, 4H), 2.58-2.62 (m, 6H), 2.38 (t, J=6.4 Hz, 2H), 2.28-2.31 (m, 10H), 1.56-1.63 (m, 16H), 1.24-1.32 (m, 50H), 0.89 (t, J=7.2 Hz, 12H)

[M+H]⁺: 793.6

### Synthesis of 7593

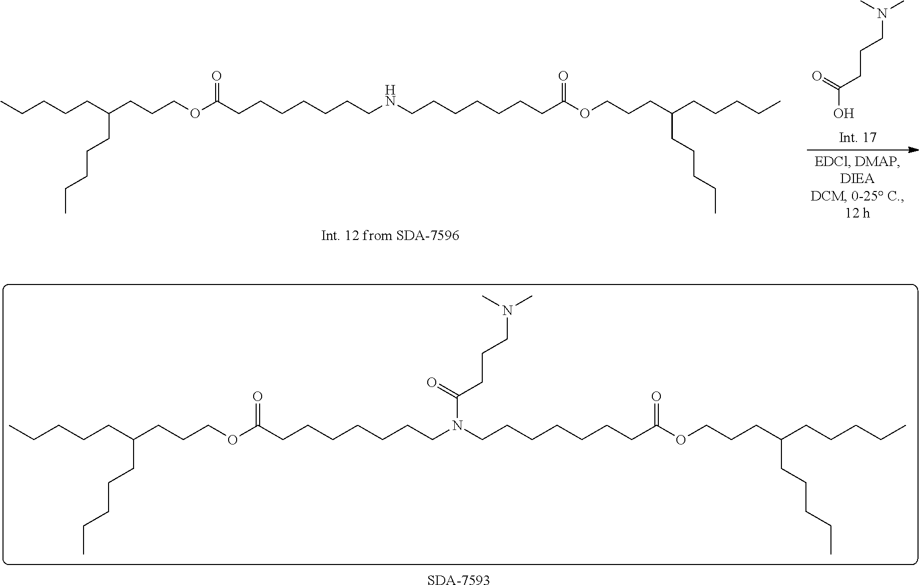

To a solution of 4-pentylnonyl 8-[[8-oxo-8-(4-pentylnonoxy)octyl]amino]octanoate 160 mg, 230.49 μmol, 1 eq) and 4-(dimethylamino)butanoic acid (60.47 mg, 360.72 μmol, 1.56 eq, HCl) in DCM (2 mL) was added DMAP (28.16 mg, 230.49 μmol, 1 eq), DIEA (59.58 mg, 460.99 μmol, 80.30 uL, 2 eq) and EDCI (132.56 mg, 691.48 μmol, 3 eq) at 0° C. and stirred at 25° C. for 12 h. The mixture was concentrated under reduced pressure. The residue was purified by prep-HPLC and desalted by sat. NaHCO₃, extracted with EtOAc, organic layer was washed with brine, dried over Na₂SO₄, filtered and the filtrate was concentrated under reduced pressure to give 4-pentylnonyl 8-[4-(dimethylamino) butanoyl-[8-oxo-8-(4-pentylnonoxy)octyl]amino]octanoate (21 mg, 26.01 μmol, 11% yield) as a white solid.

¹H NMR (400 MHz, CDCl₃), 4.04 (t, J=6.8 Hz, 4H), 3.27 (t, J=8.0 Hz, 2H), 3.20 (t, J=7.6 Hz, 2H), 2.26-2.37 (m, 14H), 1.81-1.87 (m, 2H), 1.57-1.67 (m, 8H), 1.48-1.53 (m, 4H), 1.24-1.31 (m, 50H), 0.89 (t, J=6.8 Hz, 12H)

[M+H]⁺: 807.6

### Synthesis of 7608

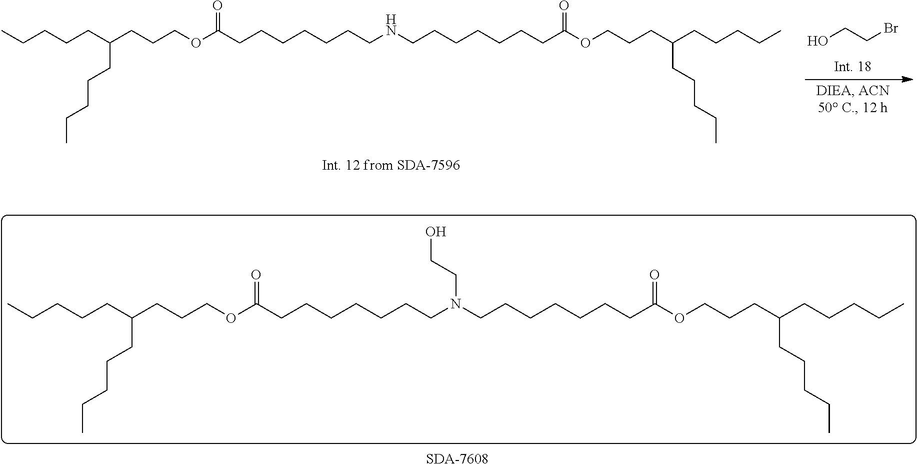

To a solution of 4-pentylnonyl 8-[[8-oxo-8-(4-pentylnonoxy)octyl]amino]octanoate (250 mg, 360.15 μmol, 1 eq) in ACN (5 mL) was added DIEA (93.09 mg, 720.29 μmol, 125.46 uL, 2 eq) and 2-bromoethanol (90.01 mg, 720.29 μmol, 51.14 μL, 2 eq). The mixture was stirred at 50° C. for 12 hrs. The reaction mixture was poured in H₂O and extracted with EtOAc. The combined organic layers were washed with brine, dried over Na₂SO₄, filtered and concentrated under reduced pressure to give a residue. The residue was purified by prep-HPLC. The compound was desalted with NaHCO₃ saturated solution and extracted with EtOAc. The combined organic layers was dried over Na₂SO₄, filtered and concentrated under reduced pressure to give 4-pentylnonyl 8-[2-hydroxyethyl-[8-oxo-8-(4-pentylnonoxy)octyl]amino]octanoate (40 mg, 54.18 μmol, 15% yield) as a yellow oil.

¹H NMR (400 MHz, CDCl₃), 4.05 (t, J=6.8 Hz 4H), 3.55 (t, J=4.8 Hz, 2H), 2.59 (t, J=5.2 Hz, 2H), 2.46 (t, J=7.2 Hz, 4H), 2.30 (t, J=7.6 Hz, 4H), 1.57-1.64 (m, 9H), 1.40-1.50 (m, 4H), 1.20-1.35 (m, 50H), 0.89 (t, J=6.8 Hz, 12H)

[M+H]⁺: 738.7

### Synthesis of 7675

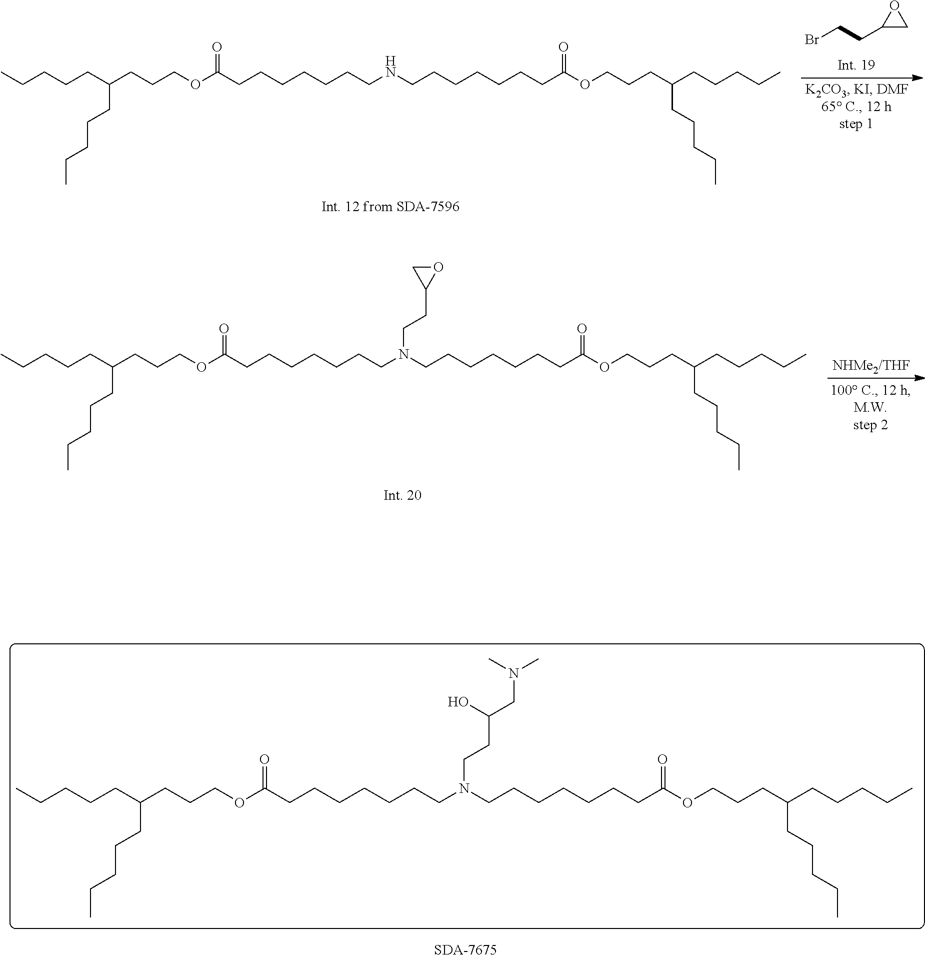

### Step 1:

To a solution of 4-pentylnonyl 8-[[8-oxo-8-(4-pentylnonoxy)octyl]amino]octanoate (500 mg, 720.29 μmol, 1 eq) in DMF (5 mL) was added K₂CO₃ (497.76 mg, 3.60 mmol, 40.10 μL, 5 eq), KI (239.14 mg, 1.44 mmol, 2 eq) and 2-(2-bromoethyl)oxirane (435.06 mg, 2.88 mmol, 58.49 μL, 4 eq). The mixture was stirred at 65° C. for 12 h. The mixture was filtered and the filtrate was added poured in H₂O, extracted with EtOAc. The organic layer was washed with brine, dried over Na₂SO₄, filtered and the filtrate was concentrated under reduced pressure. The residue was purified by silica gel chromatography to give 4-pentylnonyl 8-[2-(oxiran-2-yl)ethyl-[8-oxo-8-(4-pentylnonoxy)octyl]amino]octanoate (400 mg, 523.39 μmol, 73% yield) as colorless oil.

### Step 2:

A solution of 4-pentylnonyl 8-[2-(oxiran-2-yl)ethyl-[8-oxo-8-(4-pentylnonoxy)octyl]amino]octanoate (200 mg, 261.69 μmol, 1 eq) in Me₂NH (2 M, 20.00 mL) in THE was stirred at 100° C. for 12 h under microwave. The mixture was purified by prep-HPLC to give 4-pentylnonyl 8-[[4-(dimethylamino)-3-hydroxy-butyl]-[8-oxo-8-(4-pentylnonoxy)octyl]amino]octanoate (52 mg, 64.25 μmol, 26% yield) as yellow oil.

¹H NMR (400 MHz, CDCl₃), 4.06 (t, J=6.8 Hz 4H), 3.82-3.88 (m, 1H), 2.50-2.75 (m, 4H), 2.20-2.45 (m, 14H), 1.55-1.68 (m, 10H), 1.43-1.52 (m, 4H), 1.20-1.35 (m, 50H), 0.89 (t, J=6.8 Hz, 12H)

[M+H]⁺: 809.6

### Synthesis of 7633

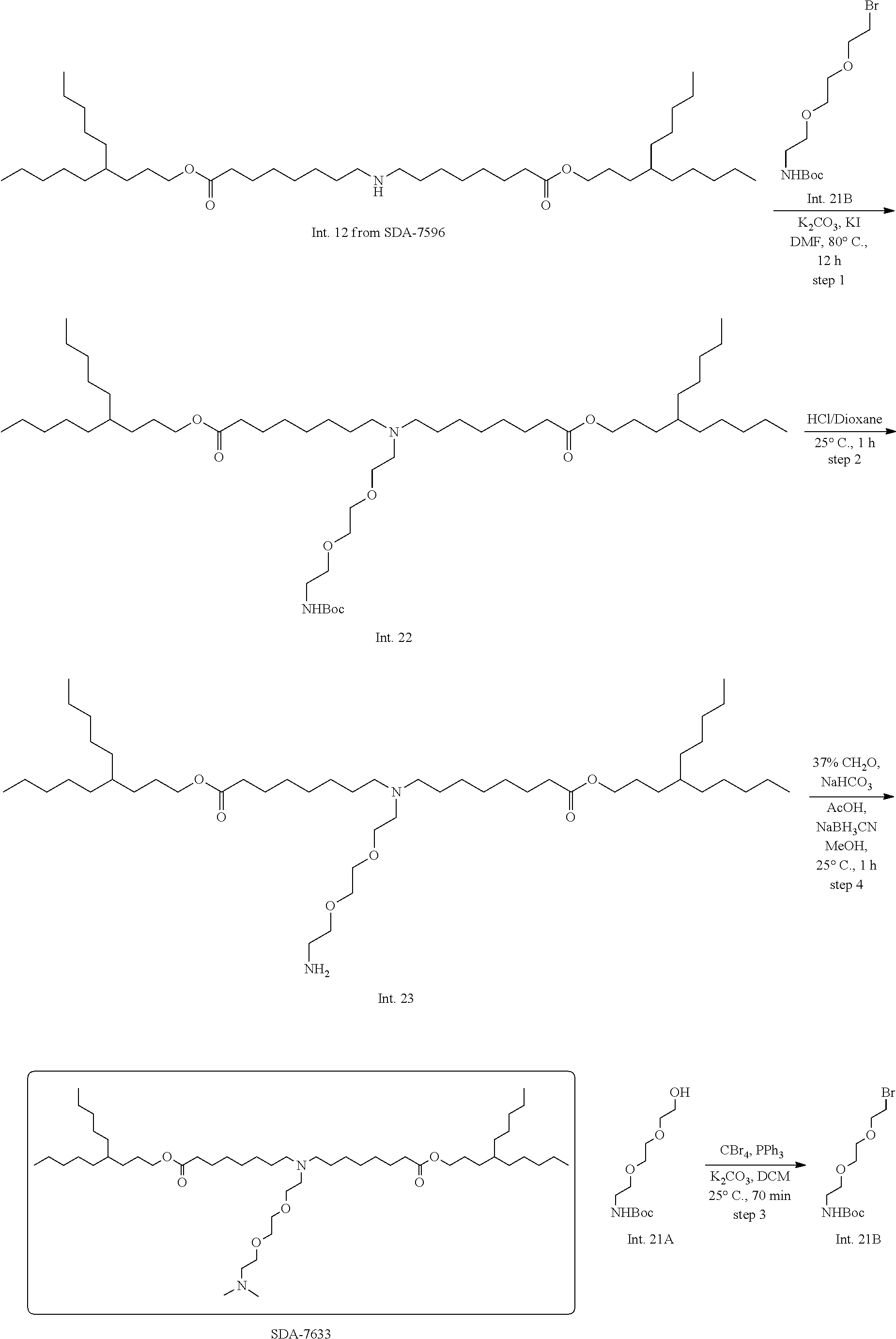

### Step 1:

To a solution of 4-pentylnonyl 8-[[8-oxo-8-(4-pentylnonoxy)octyl]amino]octanoate (300 mg, 432.18 μmol, 1 eq) in DMF (10 mL) was added K₂CO₃ (298.65 mg, 2.16 mmol, 40.10 μL, 5 eq), KI (143.48 mg, 864.35 μmol, 2 eq) and tert-butyl N-[2-[2-(2-bromoethoxy)ethoxy]ethyl]carbamate (674.63 mg, 2.16 mmol, 5 eq), stirred at 80° C. for 12 h. The reaction mixture was filtered and the filtrate was quenched with water (10 mL) and extracted with dichloromethane. The combined organic layer was washed with brine, dried over anhydrous sodium sulfate, filtered and concentrated under reduced pressure to get a residue. The residue was purified by silica gel chromatography to give 4-pentylnonyl 8-[2-[2-[2-(tert-butoxycarbonylamino)ethoxy]ethoxy]ethyl-[8-oxo-8-(4-pentylnonoxy)octyl]amino]octanoate (350 mg, 378.19 μmol, 88% yield) as yellow oil.

### Step 2:

A solution of 4-pentylnonyl 8-[2-[2-[2-(tert-butoxycarbonylamino)ethoxy]ethoxy]ethyl-[8-oxo-8-(4-pentylnonoxy)octyl]amino]octanoate (300 mg, 324.17 μmol, 1 eq) in HCl/dioxane (4 M, 6.00 mL, 74.04 eq) was stirred at 25° C. for 1 h. The reaction mixture was concentrated in vacuo to give 4-pentylnonyl 8-[2-[2-(2-aminoethoxy)ethoxy]ethyl-[8-oxo-8-(4-pentylnonoxy)octyl]amino] octanoate (300 mg, crude, HCl) as a yellow solid.

### Step 3:

To a solution of tert-butyl N-[2-[2-(2-hydroxyethoxy)ethoxy]ethyl]carbamate (2.0 g, 8.02 mmol, 1 eq) in DCM (20 mL) was added CBr₄ (3.46 g, 10.43 mmol, 1.3 eq) and K₂CO₃ (1.44 g, 10.43 mmol, 1.3 eq), then a solution of PPh₃ (3.37 g, 12.84 mmol, 1.6 eq) in DCM (40 mL), stirred at 25° C. for 1 h. The reaction mixture was filtered and concentrated under reduced pressure. The residue was purified by silica gel chromatography to give tert-butyl N-[2-[2-(2-bromoethoxy)ethoxy]ethyl]carbamate (1.2 g, 3.84 mmol, 48% yield) as colorless oil.

### Step 4:

To a solution of 4-pentylnonyl 8-[2-[2-(2-aminoethoxy)ethoxy]ethyl-[8-oxo-8-(4-pentylnonoxy) octyl]amino]octanoate (300 mg, 348.11 μmol, 1 eq, HCl) and formaldehyde (2.81 g, 34.62 mmol, 2.58 mL, 37% purity, 99.44 eq) in MeOH (5 mL) was added NaHCO₃ (87.73 mg, 1.04 mmol, 40.62 μL, 3 eq), then stirred at 25° C. for 10 min, AcOH (627.14 mg, 10.44 mmol, 597.28 μL, 30 eq) and NaBH₃CN (65.63 mg, 1.04 mmol, 3 eq) was added into the mixture and stirred at 25° C. for 1 h. The reaction mixture was quenched with sat. NaHCO₃ and extracted with EtOAc. The combined organic layer was washed with brine, dried over anhydrous sodium sulfate, filtered and concentrated under reduced pressure to get a residue. The residue was purified by silica gel chromatography to give 4-pentylnonyl8-[2-[2-[2-(dimethylamino)ethoxy]ethoxy]ethyl-[8-oxo-8-(4-pentylnonoxy)octyl]amino]octanoate (104 mg, 121.87 μmol, 35% yield) as colorless oil.

¹H NMR (400 MHz, CDCl₃), 4.05 (t, J=6.8 Hz, 4H), 3.50-3.61 (m, 8H), 2.67 (t, J=3.2 Hz, 2H), 2.53 (t, J=5.6 Hz, 2H), 2.44-2.48 (m, 4H), 2.27-2.32 (m, 10H), 1.55-1.64 (m, 8H), 1.40-1.47 (m, 4H), 1.20-1.35 (m, 50H), 0.89 (t, J=6.8 Hz, 12H)

[M+H]⁺: 853.6

### Synthesis of 7631

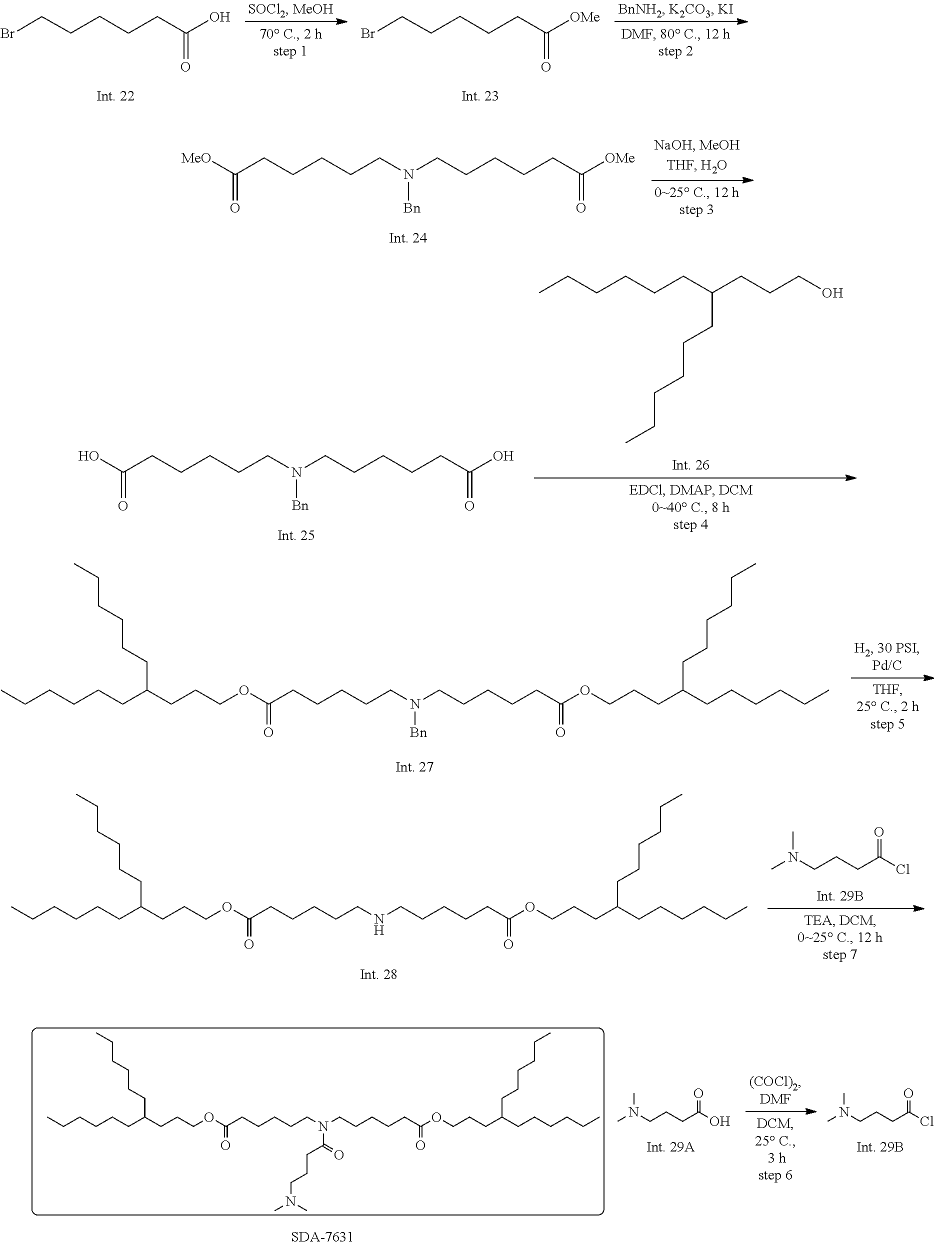

### Step 1:

To a solution of 6-bromohexanoic acid (10.0 g, 51.27 mmol, 1 eq) in MeOH (200 mL) was added SOCl₂ (12.20 g, 102.54 mmol, 7.44 mL, 2 eq). The mixture was stirred at 70° C. for 2 hr. The reaction mixture was diluted with H₂O and washed with petroleum ether, extracted with EtOAc. The combined EtOAc layers were dried over Na₂SO₄, filtered and concentrated under reduced pressure to give methyl 6-bromohexanoate (10.0 g, 47.83 mmol, 93% yield) as white solid.

### Step 2:

To a solution of BnNH₂ (1.28 g, 11.96 mmol, 1.30 mL, 1 eq) in DMF (50 mL) was added K₂CO₃ (8.26 g, 59.79 mmol, 5 eq) and KI (4.96 g, 29.89 mmol, 2.5 eq), then a solution of methyl 6-bromohexanoate (5 g, 23.91 mmol, 2 eq) in DMF (20 mL) was added to the mixture and stirred at 80° C. for 12 hr. The reaction mixture was filtered and the filtrate was diluted with EtOAc 200 mL and washed with water and brine. The combined organic layers was dried with anhydrous Na₂SO₄, filtered and concentrated under reduced pressure to give a residue. The residue was purified by silica gel chromatography to give methyl 6-[benzyl-(6-methoxy-6-oxo-hexyl) amino]hexanoate (8.5 g, 23.38 mmol, 98% yield) as white solid.

### Step 3:

To a solution of methyl 6-[benzyl-(6-methoxy-6-oxo-hexyl)amino]hexanoate (5.0 g, 13.76 mmol, 1 eq) in MeOH (20 mL), THF (6 mL) was added NaOH (1.88 g, 46.91 mmol, 3.41 eq) in H₂O (10 mL) at 0° C. The mixture was stirred at 25° C. for 12 hr. The reaction mixture was diluted with H₂O and extracted with EtOAc. The aqueous phase was freeze-dried after adjusting pH=7 with 1M HCl aqueous. The crude product was triturated with EtOH at 25° C. for 2 hr, then filtered and the filtrate was concentrated under reduced pressure to give 6-[benzyl(5-carboxypentyl)amino]hexanoic acid (4.5 g, 13.42 mmol, 98% yield) as white solid.

### Step 4:

A mixture of 6-[benzyl(5-carboxypentyl)amino]hexanoic acid (1.0 g, 2.98 mmol, 1 eq) in DCM (10 mL) was added DMAP (182.10 mg, 1.49 mmol, 0.5 eq), 4-hexyldecan-1-ol (1.48 g, 6.11 mmol, 2.05 eq), EDCI (1.71 g, 8.94 mmol, 3 eq) at 0° C. and was degassed and purged with N₂. The mixture was stirred at 40° C. for 8 hr under N₂ atmosphere. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[benzyl-[6-(4-hexyldecoxy)-6-oxo-hexyl]amino] hexanoate (0.96 g, 1.22 mmol, 41% yield) as yellow oil.

### Step 5:

A solution of 4-hexyldecyl 6-[benzyl-[6-(4-hexyldecoxy)-6-oxo-hexyl]amino]hexanoate (960 mg, 1.22 mmol, 1 eq) and Pd/C (0.6 g, 1.22 mmol, 10% purity, 1.00 eq) in THE (50 mL) was stirred under H₂ (30 psi) at 25° C. for 2 hours. The mixture was filtered and the solvent was removed under reduced pressure to give a residue. The residue was purified by silica gel chromatography to give 4-hexyldecyl 6-[[6-(4-hexyldecoxy)-6-oxo-hexyl]amino]hexanoate (650 mg, 936.38 μmol, 77% yield) as brown oil.

### Step 6:

To a solution of 4-(dimethylamino)butanoic acid hydrochloride (0.4 g, 2.39 mmol, 1 eq) and oxalyl dichloride (1.77 g, 13.97 mmol, 1.22 mL, 5 eq) in DCM (5 mL) was added two drops of DMF (20.42 mg, 279.36 μmol, 21.49 uL, 0.1 eq), and stirred at 25° C. for 3 hr under N₂ atmosphere. The reaction mixture was concentrated under reduced pressure to give 4-(dimethylamino)butanoyl chloride (0.4 g, crude) as yellow oil.

### Step 7:

To a solution of 4-hexyldecyl 6-[[6-(4-hexyldecoxy)-6-oxo-hexyl]amino]hexanoate (0.4 g, 576.23 μmol, 1 eq), 4-(dimethylamino)butanoyl chloride (344.86 mg, 2.30 mmol, 4 eq) in DCM (3 mL) was added TEA (174.93 mg, 1.73 mmol, 240.61 μL, 3 eq) at 0° C. The mixture was stirred at 25° C. for 12 hr. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[4-(dimethylamino) butanoyl-[6-(4-hexyldecoxy)-6-oxo-hexyl]amino]hexanoate (174 mg, 215.53 μmol, 37% yield) as yellow oil.

¹H NMR (400 MHz, CDCl₃), 4.01-4.07 (m, 4H), 3.20-3.31 (m, 4H), 2.28-2.35 (m, 8H), 2.24 (S, 6H), 1.80-1.84 (m, 2H), 1.57-1.70 (m, 12H), 1.24-1.34 (m, 50H), 0.89 (t, J=6.8 Hz, 12H)

[M+H]⁺: 807.6

### Synthesis of 7651

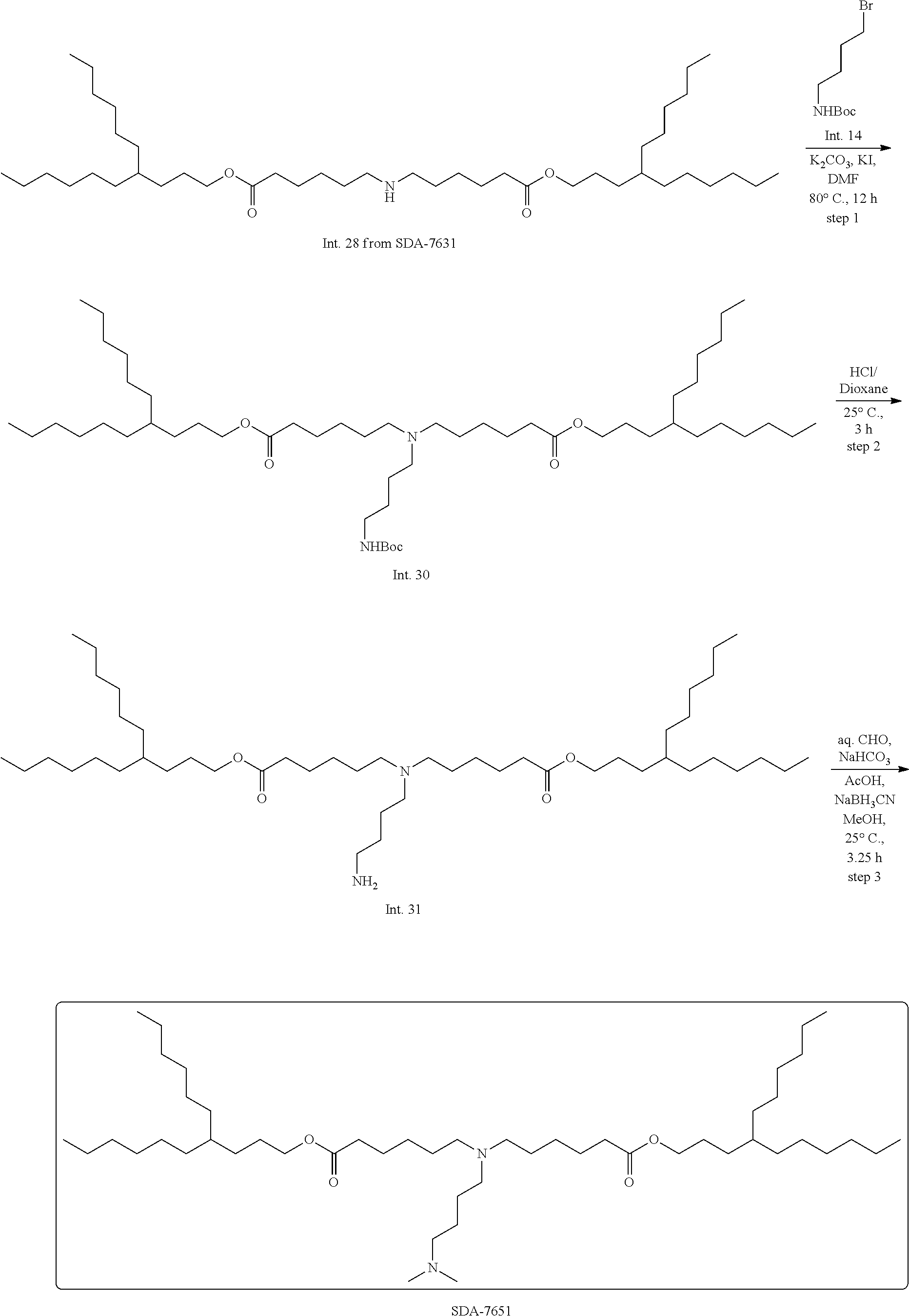

### Step 1

To a solution of 4-hexyldecyl 6-[[6-(4-hexyldecoxy)-6-oxo-hexyl]amino]hexanoate (0.3 g, 432.18 μmol, 1 eq) in DMF (3 mL) was added K₂CO₃ (298.65 mg, 2.16 mmol, 5 eq) and KI (143.48 mg, 864.35 μmol, 2 eq), tert-butyl N-(4-bromobutyl)carbamate (435.89 mg, 1.73 mmol, 354.38 μL, 4 eq). The mixture was stirred at 80° C. for 12 hr. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[4-(tert-butoxycarbonylamino)butyl-[6-(4-hexyldecoxy)-6-oxo-hexyl]amino]hexanoate (0.25 g, 288.88 μmol, 67% yield) as yellow oil.

### Step 2:

A solution of 4-hexyldecyl 6-[4-(tert-butoxycarbonylamino)butyl-[6-(4-hexyldecoxy)-6-oxo-hexyl]amino]hexanoate (0.2 g, 231.11 μmol, 1 eq) in HCl/dioxane (4 M, 4.00 mL, 69.23 eq) was stirred at 25° C. for 3 hours under N₂ atmosphere. The reaction mixture was concentrated under reduced pressure to get 4-hexyldecyl 6-[4-aminobutyl-[6-(4-hexyldecoxy)-6-oxo-hexyl]amino]hexanoate (0.1 g, crude) as yellow oil.

### Step 3:

To a solution of 4-hexyldecyl 6-[4-aminobutyl-[6-(4-hexyldecoxy)-6-oxo-hexyl]amino]hexanoate (0.1 g, 130.67 μmol, 1 eq), formaldehyde (1.09 g, 36.30 mmol, 1.00 mL, 277.81 eq) in MeOH (2 mL) was added NaHCO₃ (32.93 mg, 392.01 μmol, 15.25 uL, 3 eq) at 25° C. and stirred at 25° C. for 15 min, then AcOH (235.41 mg, 3.92 mmol, 224.20 uL, 30 eq) and NaBH₃CN (24.63 mg, 392.01 μmol, 3 eq) was added to the mixture at 25° C. The resulting mixture was stirred at 25° C. for 3 hr. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[4-(dimethylamino)butyl-[6-(4-hexyldecoxy)-6-oxo-hexyl]amino]hexanoate (38 mg, 47.90 μmol, 37% yield) as yellow oil.

¹H NMR (400 MHz, CDCl₃), 4.04 (t, J=6.8 Hz, 4H), 2.35-2.49 (m, 6H), 2.28-2.32 (m, 6H), 2.23 (s, 6H), 1.55-1.65 (m, 8H), 1.43-1.45 (m, 8H), 1.24-1.30 (m, 50H), 0.89 (t, J=6.8 Hz, 12H)

[M+H]⁺: 793.6

### Synthesis of 7667

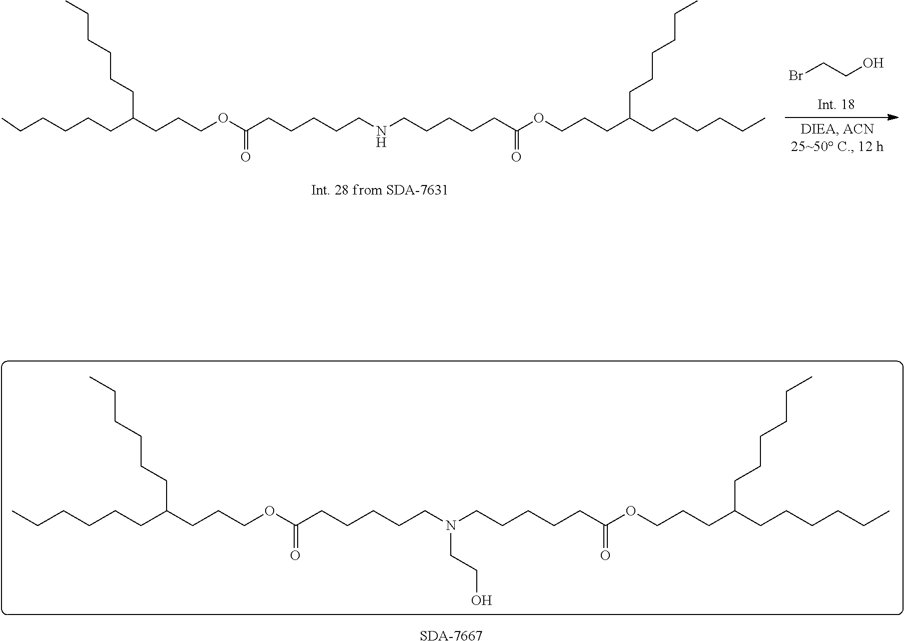

To a solution of 4-hexyldecyl 6-[[6-(4-hexyldecoxy)-6-oxo-hexyl]amino]hexanoate (0.2 g, 288.12 μmol, 1 eq) in ACN (3 mL) was added DIEA (74.47 mg, 576.23 μmol, 100.37 μL, 2 eq), 2-bromoethanol (72.01 mg, 576.23 μmol, 40.91 μL, 2 eq) at 25° C. The mixture was stirred at 50° C. for 12 hr. The reaction mixture was diluted with H₂O and extracted with EtOAc. The combined organic layers were dried over Na₂SO₄, filtered and concentrated under reduced pressure to give a residue. The residue was purified by silica gel chromatography to give the compound 4-hexyldecyl6-[[6-(4-hexyldecoxy)-6-oxo-hexyl]-(2-hydroxyethyl)amino]hexanoate (28 mg, 37.93 μmol, 13% yield) as yellow oil.

¹H NMR (400 MHz, CDCl₃), 4.04 (t, J=6.8 Hz 4H), 3.54 (t, J=5.2 Hz, 2H), 2.59 (t, J=5.2 Hz, 2H), 2.47 (t, J=7.6 Hz, 4H), 2.3 (t, J=7.6 Hz, 4H), 1.66-1.70 (m, 4H), 1.55-1.57 (m, 4H), 1.43-1.49 (m, 4H), 1.24-1.33 (m, 50H), 0.89 (t, J=6.8 Hz, 12H)

[M+H]⁺: 738.5

### Synthesis of 7677

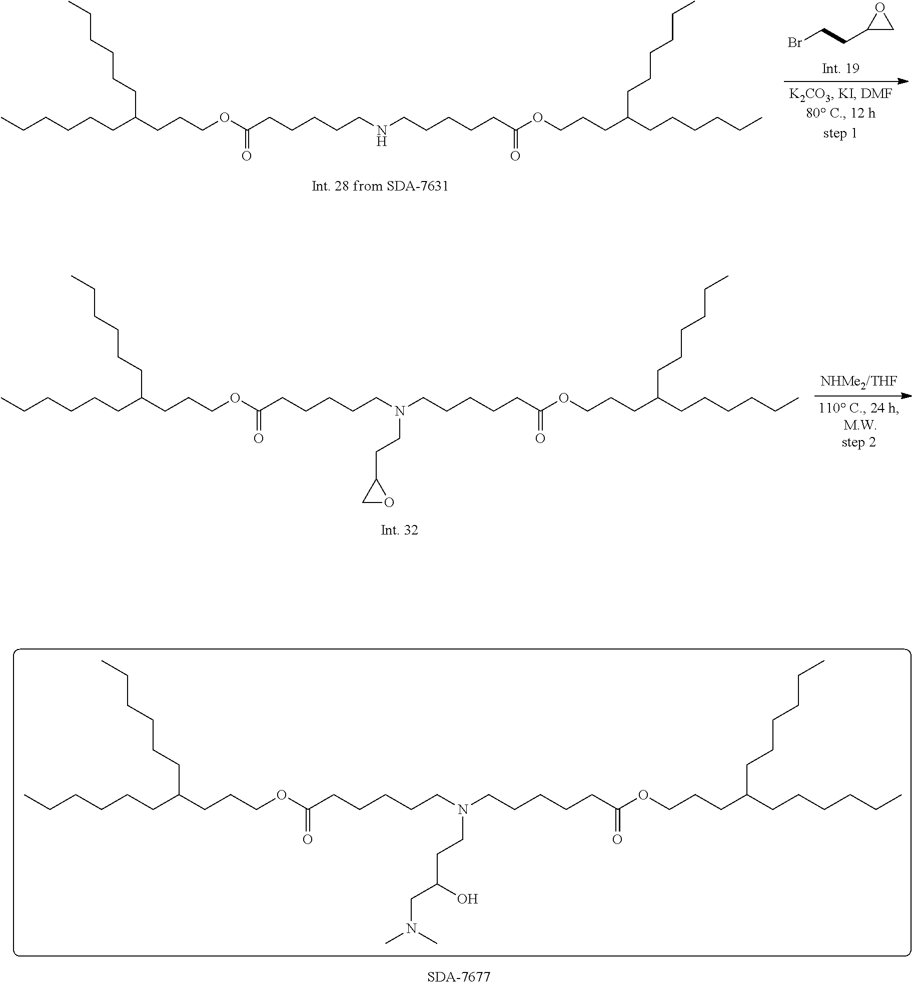

### Step 1:

To a solution of 4-hexyldecyl 6-[[6-(4-hexyldecoxy)-6-oxo-hexyl]amino]hexanoate (1 g, 1.44 mmol, 1 eq) in DMF (10 mL) was added K₂CO₃ (995.52 mg, 7.20 mmol, 5 eq) and KI (478.27 mg, 2.88 mmol, 2 eq), 2-(2-bromoethyl)oxirane (870.12 mg, 5.76 mmol, 354.38 μL, 4 eq). The mixture was stirred at 80° C. for 12 hr. TLC showed 4-hexyldecyl 6-[[6-(4-hexyldecoxy)-6-oxo-hexyl]amino]hexanoate was consumed completely and one new spot was formed. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[[6-(4-hexyldecoxy)-6-oxo-hexyl]-[2-(oxiran-2-yl)ethyl]amino]hexanoate (0.5 g, 654.23 μmol, 45% yield) as yellow oil.

### Step 2:

A mixture of 4-hexyldecyl 6-[[6-(4-hexyldecoxy)-6-oxo-hexyl]-[2-(oxiran-2-yl)ethyl]amino]hexanoate (0.2 g, 261.69 μmol, 1 eq) in Me₂NH (1 M, 261.69 μL, 1 eq) were taken up into a microwave tube. The sealed tube was heated at 110° C. for 24 hr under microwave. TLC showed 4-hexyldecyl 6-[[6-(4-hexyldecoxy)-6-oxo-hexyl]-[2-(oxiran-2-yl)ethyl]amino]hexanoate was remaining and a new spot was formed. The combined organic phase was diluted with EtOAc and washed with water and brine, dried with anhydrous Na₂SO₄, filtered and concentrated under reduced pressure to give a residue. The residue was purified by prep-HPLC to give a compound 4-hexyldecyl 6-[[4-(dimethylamino)-3-hydroxy-butyl]-[6-(4-hexyldecoxy)-6-oxo-hexyl]amino]hexanoate (42 mg, 51.89 μmol, 20% yield) as yellow oil.

¹H NMR (400 MHz, CDCl₃), 4.05 (t, J=6.8 Hz 4H), 3.65-3.88 (m, 1H), 2.25-2.61 (m, 18H), 1.52-1.70 (m, 10H), 1.42-1.50 (m, 4H), 1.20-1.35 (m, 50H), 0.89 (t, J=6.8 Hz, 12H)

[M+H]⁺: 809.6

### Synthesis of 7632

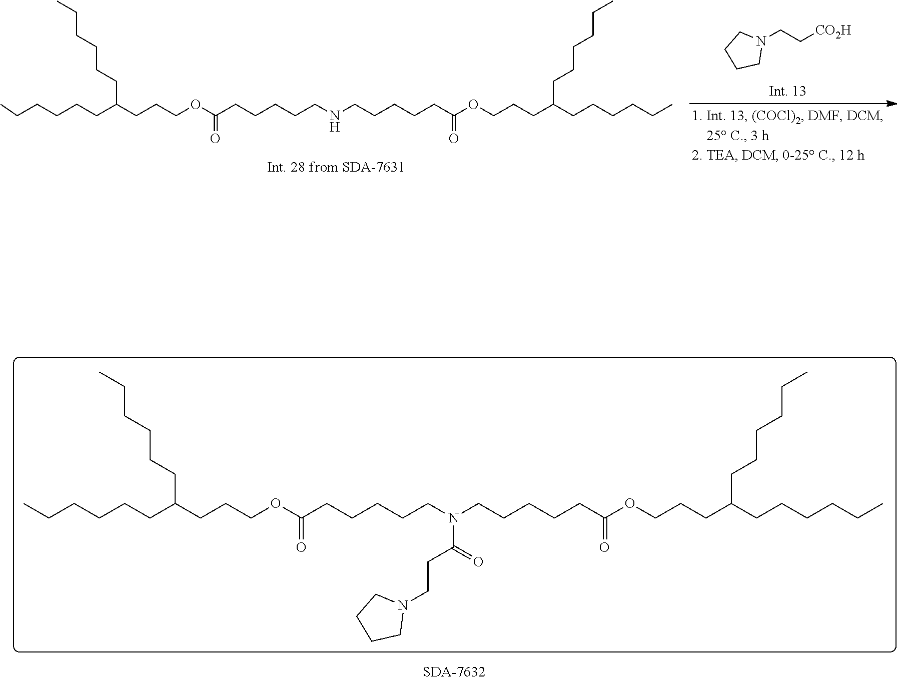

To a solution of 3-pyrrolidin-1-ylpropanoic acid (0.4 g, 2.79 mmol, 1 eq) and oxalyl dichloride (1.77 g, 13.97 mmol, 1.22 mL, 5 eq) in DCM (5 mL) was added two drops of DMF (20.42 mg, 279.36 μmol, 21.49 μL, 0.1 eq). The mixture was stirred at 25° C. for 3 hr under N₂ atmosphere. The reaction mixture was concentrated under reduced pressure to give compound 3-pyrrolidin-1-ylpropanoyl chloride (0.5 g, crude, HCl) as yellow oil. The crude was used directly.

To a solution of 4-hexyldecyl 6-[[6-(4-hexyldecoxy)-6-oxo-hexyl]amino]hexanoate (0.4 g, 576.23 μmol, 1 eq), 3-pyrrolidin-1-ylpropanoyl chloride (372.54 mg, 2.30 mmol, 4 eq) in DCM (3 mL) was added TEA (174.93 mg, 1.73 mmol, 240.61 μL, 3 eq) at 0° C. The mixture was stirred at 25° C. for 12 hr. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[[6-(4-hexyldecoxy)-6-oxo-hexyl]-(3-pyrrolidin-1-ylpropanoyl)amino]hexanoate (190 mg, 231.90 μmol, 40% yield) as yellow oil.

¹H NMR (400 MHz, CDCl₃), 3.97-4.08 (m, 4H), 3.21-331 (m, 4H), 2.89 (t, J=7.6 Hz, 2H), 2.52-2.65 (m, 6H), 2.25-2.35 (m, 4H), 1.93 (brs, 4H), 1.52-1.67 (m, 12H), 1.15-1.35 (m, 50H), 0.89 (t, J=6.4 Hz, 12H)

[M+H]⁺: 819.6

### Synthesis of 7670

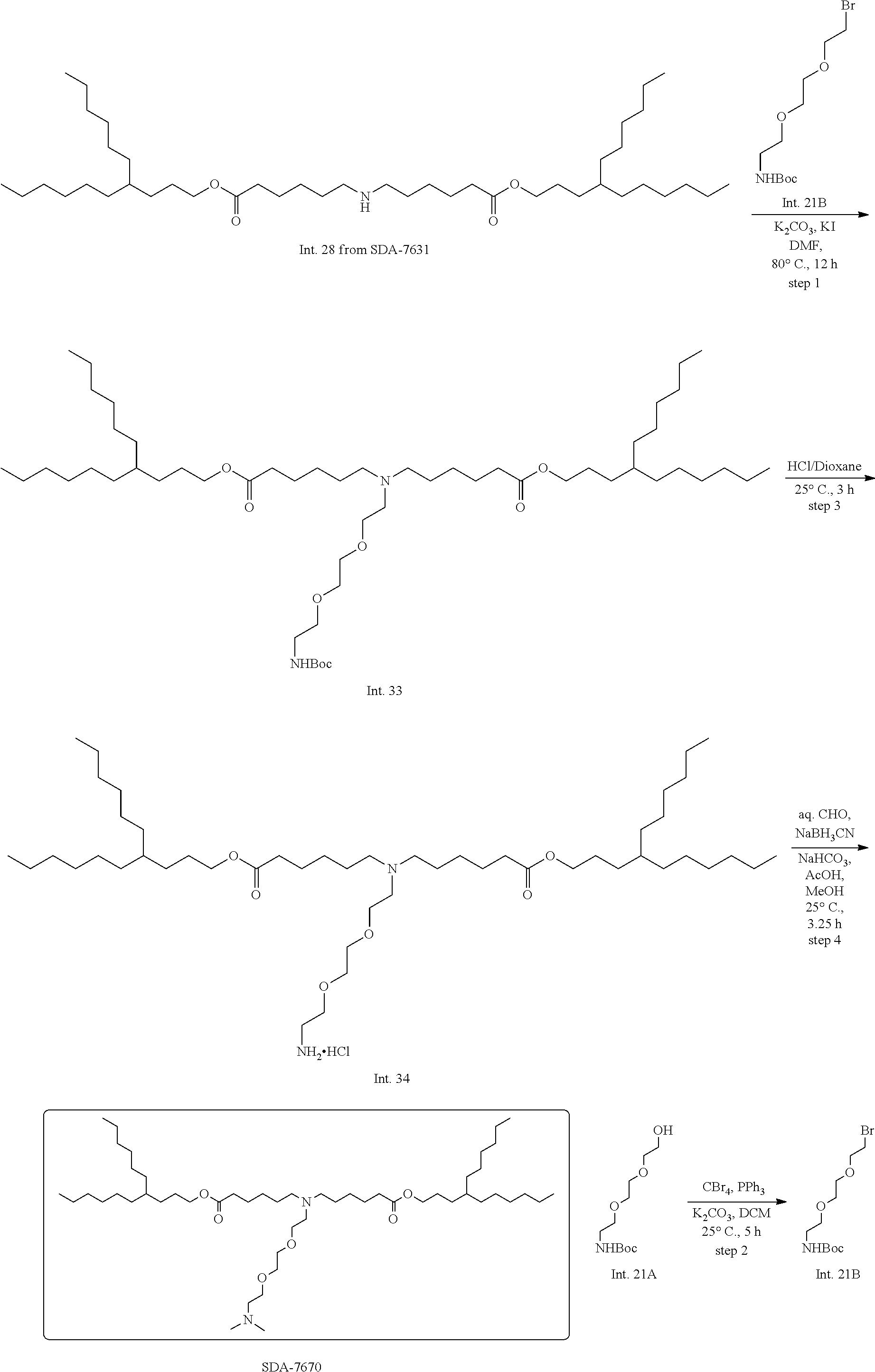

### Step 1

To a solution of 4-hexyldecyl 6-[[6-(4-hexyldecoxy)-6-oxo-hexyl]amino]hexanoate (1.0 g, 1.44 mmol, 1 eq) in DMF (3 mL) was added K₂CO₃ (995.52 mg, 7.20 mmol, 5 eq) and KI (478.28 mg, 2.88 mmol, 2 eq), tert-butyl N-[2-[2-(2-bromoethoxy)ethoxy]ethyl]carbamate (1.80 g, 5.76 mmol, 354.38 μL, 4 eq). The mixture was stirred at 80° C. for 12 hr. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[2-[2-[2-(tert-butoxycarbonylamino)ethoxy]ethoxy]ethyl-[6-(4-hexyldecoxy)-6-oxo-hexyl]amino]hexanoate (0.5 g, 540.28 μmol, 38% yield) as yellow oil.

### Step 2

To a solution of tert-butyl N-[2-[2-(2-hydroxyethoxy)ethoxy]ethyl]carbamate (1.0 g, 4.01 mmol, 1 eq) in DCM (50 mL) was added carbon tetrabromide (1.73 g, 5.21 mmol, 1.3 eq), K₂CO₃ (720.68 mg, 5.21 mmol, 1.3 eq) and PPh₃ (1.68 g, 6.42 mmol, 1.6 eq) in DCM (10 mL). The mixture was stirred at 25° C. for 5 hr. The reaction mixture was filtered and concentrated under reduced pressure to give a residue. The residue was purified by silica gel chromatography to give tert-butyl N-[2-[2-(2-bromoethoxy)ethoxy] ethyl] carbamate (2.6 g, 8.33 mmol, 42% yield) as white solid.

### Step 3:

A solution of 4-hexyldecyl 6-[2-[2-[2-(tert-butoxycarbonylamino)ethoxy]ethoxy]ethyl-[6-(4-hexyldecoxy)-6-oxo-hexyl]amino]hexanoate (0.5 g, 540.28 μmol, 1 eq) in HCl/dioxane (4 M, 9.35 mL, 69.23 eq) was stirred at 25° C. for 3 hours under N₂ atmosphere. The reaction mixture was concentrated under reduced pressure to give compound 4-hexyldecyl 6-[2-[2-(2-aminoethoxy) ethoxy]ethyl-[6-(4-hexyldecoxy)-6-oxo-hexyl]amino]hexanoate (0.55 g, crude, HCl) as yellow oil.

### Step 4:

To a solution of 4-hexyldecyl 6-[2-[2-(2-aminoethoxy)ethoxy]ethyl-[6-(4-hexyldecoxy)-6-oxo-hexyl]amino]hexanoate (0.55 g, 638.20 μmol, 1 eq, HCl), formaldehyde (5.45 g, 67.16 mmol, 5 mL, 37% purity, 105.23 eq) in MeOH (10 mL) added NaHCO₃ (160.85 mg, 1.91 mmol, 74.47 μL, 3 eq) at 25° C. and stirred at 25° C. for 15 min, then AcOH (1.16 g, 19.23 mmol, 1.10 mL, 30.14 eq), NaBH₃CN (120.31 mg, 1.91 mmol, 3 eq) was added to the mixture. The resulting mixture was stirred at 25° C. for 3 hr. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 6-[2-[2-[2-(dimethylamino)ethoxy]ethoxy]ethyl-[6-(4-hexyldecoxy)-6-oxo-hexyl]amino]hexanoate (227 mg, 266.00 μmol, 42% yield) as yellow oil.

¹H NMR (400 MHz, CDCl₃), 4.04 (t, J=7.2 Hz, 4H), 3.61 (s, 4H), 3.58 (t, J=6.4 Hz, 2H), 3.53 (t, J=6.4 Hz, 2H), 2.65 (t, J=6.4 Hz, 2H), 2.52 (t, J=5.6 Hz, 2H), 2.45 (t, J=7.2 Hz, 4H), 2.30 (t, J=7.6 Hz, 4H), 2.27 (s, 6H), 1.55-1.69 (m, 8H), 1.40-1.49 (m, 4H), 1.20-1.35 (m, 50H), 0.89 (t, J=6.4 Hz, 12H)

[M+H]⁺: 853.7

### Synthesis of 7607

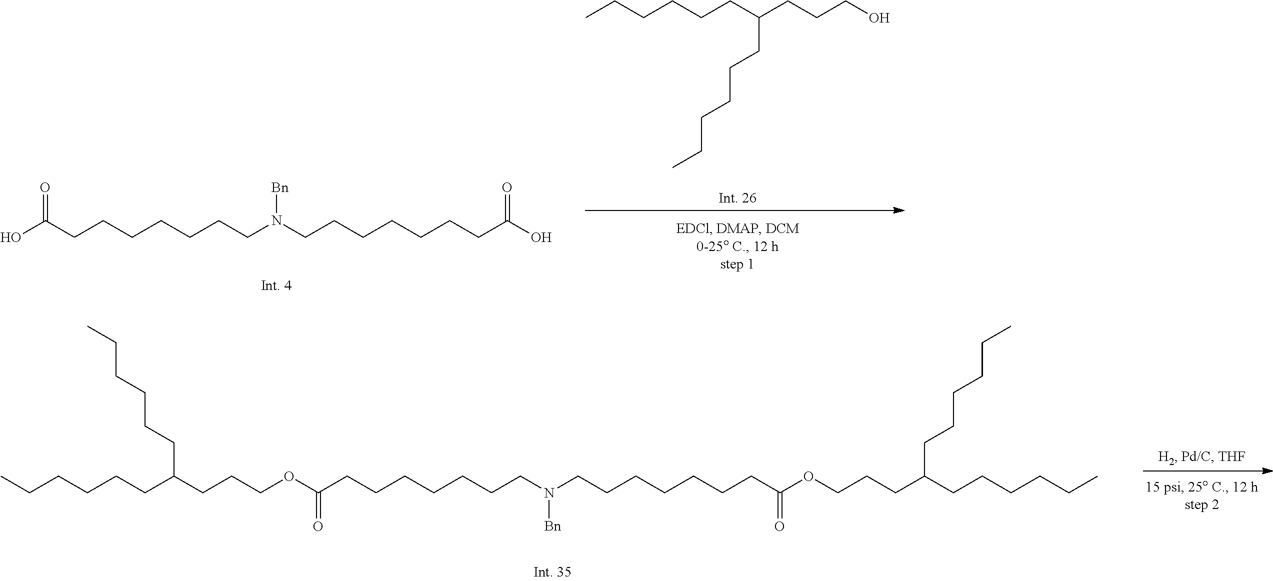 [SMILES: CCCCCCC(CCCO)CCCCCC ; O=C(O)CCCCCCCN(CCCCCCCC(=O)O)Cc1ccccc1 ; CCCCCCC(CCCCCC)CCCOC(=O)CCCCCCCN(CCCCCCCC(=O)OCCCC(CCCCCC)CCCCCC)Cc1ccccc1]

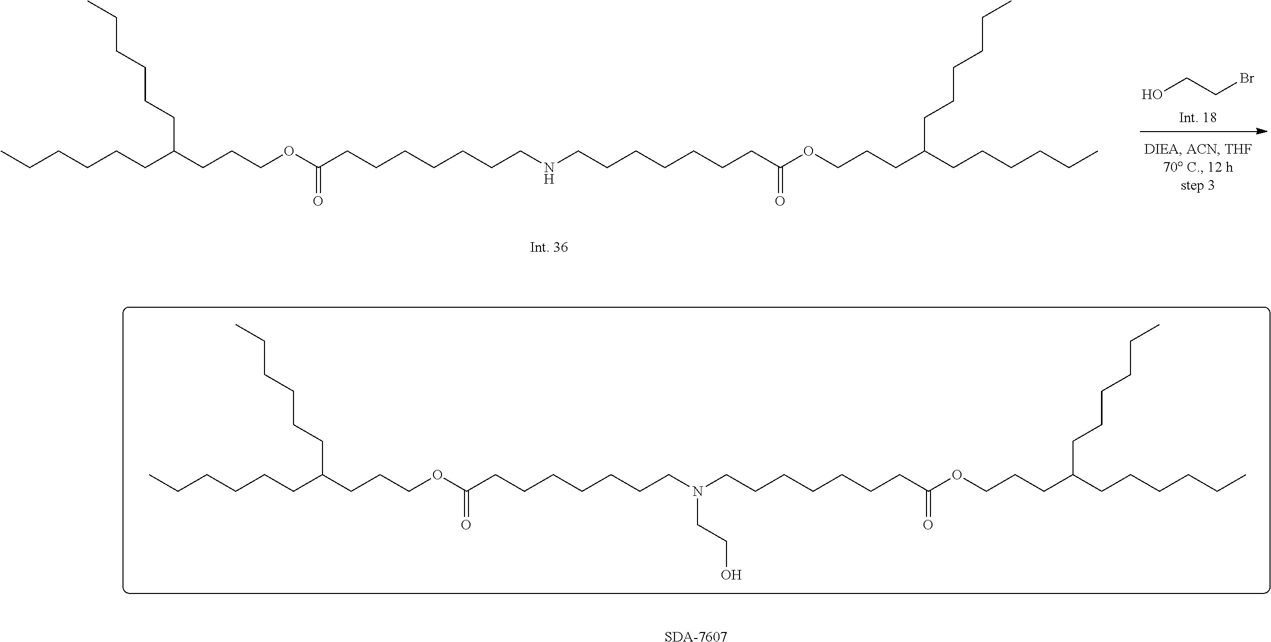

### Step 1:

To a solution of 8-[benzyl(7-carboxyheptyl)amino]octanoic acid (1.7 g, 4.34 mmol, 1 eq) in DCM (20 mL) was added DMAP (265.21 mg, 2.17 mmol, 0.5 eq), 4-hexyldecan-1-ol (2.16 g, 8.90 mmol, 2.05 eq) and EDCI (2.50 g, 13.02 mmol, 3 eq) at 0° C. and stirred at 25° C. for 12 h under N₂ atmosphere. The reaction mixture was diluted with H₂O and extracted with EtOAc. The combined organic layers were dried over Na₂SO₄, filtered and concentrated under reduced pressure to give a residue. The residue was purified by silica gel chromatography to give 4-hexyldecyl 8-[benzyl-[8-(4-hexyldecoxy)-8-oxo-octyl]amino]octanoate (2.9 g, 3.45 mmol, 80% yield) as colorless oil.

### Step 2:

A solution of Pd/C (1.0 g, 1.78 mmol, 10% purity, 1 eq) in THF (30 mL) was added 4-hexyldecyl 8-[benzyl-[8-(4-hexyldecoxy)-8-oxo-octyl]amino]octanoate (1.5 g, 1.78 mmol, 1 eq) was stirred under H₂ (15 psi) at 25° C. for 12 hr. The mixture was filtered through celite and the filtrate was removed under reduced pressure to get a residue. The residue was purified by silica gel chromatography to give 4-hexyldecyl 8-[[8-(4-hexyldecoxy)-8-oxo-octyl]amino]octanoate (700 mg, 933.00 μmol, 52% yield) as a colorless oil.

### Step 3:

To a solution of 4-hexyldecyl 8-[[8-(4-hexyldecoxy)-8-oxo-octyl]amino]octanoate (150 mg, 199.93 μmol, 1 eq) in ACN (0.5 mL) and THE (1 mL) was added DIEA (51.68 mg, 399.86 μmol, 69.65 μL, 2 eq) and then a solution of 2-bromoethanol (64.55 mg, 399.86 μmol, 36.67 uL, 2 eq, HCl) in THF (0.5 mL) was added into the mixture. The mixture was stirred at 70° C. for 12 h. The reaction mixture was diluted with H₂O and extracted with petroleum ether. The combined organic layers were washed with brine, dried over Na₂SO₄, filtered and concentrated under reduced pressure to give a residue. The residue was purified by silica gel chromatography to give 4-hexyldecyl 8-[[8-(4-hexyldecoxy)-8-oxo-octyl]-(2-hydroxyethyl)amino]octanoate (60 mg, 75.15 μmol, 38% yield) as colorless oil.

¹H NMR (400 MHz, CDCl₃), 4.05 (t, J=6.8 Hz 4H), 3.53 (t, J=5.2 Hz, 2H), 2.58 (t, J=4.8 Hz, 2H), 2.45 (t, J=7.2 Hz, 4H), 2.29 (t, J=7.6 Hz, 4H), 1.5-1.62 (m, 8H), 1.42-1.45 (m, 4H), 1.24-1.30 (m, 58H), 0.89 (t, J=6.8 Hz, 12H)

[M+H]⁺: 794.6

### Synthesis of 7650

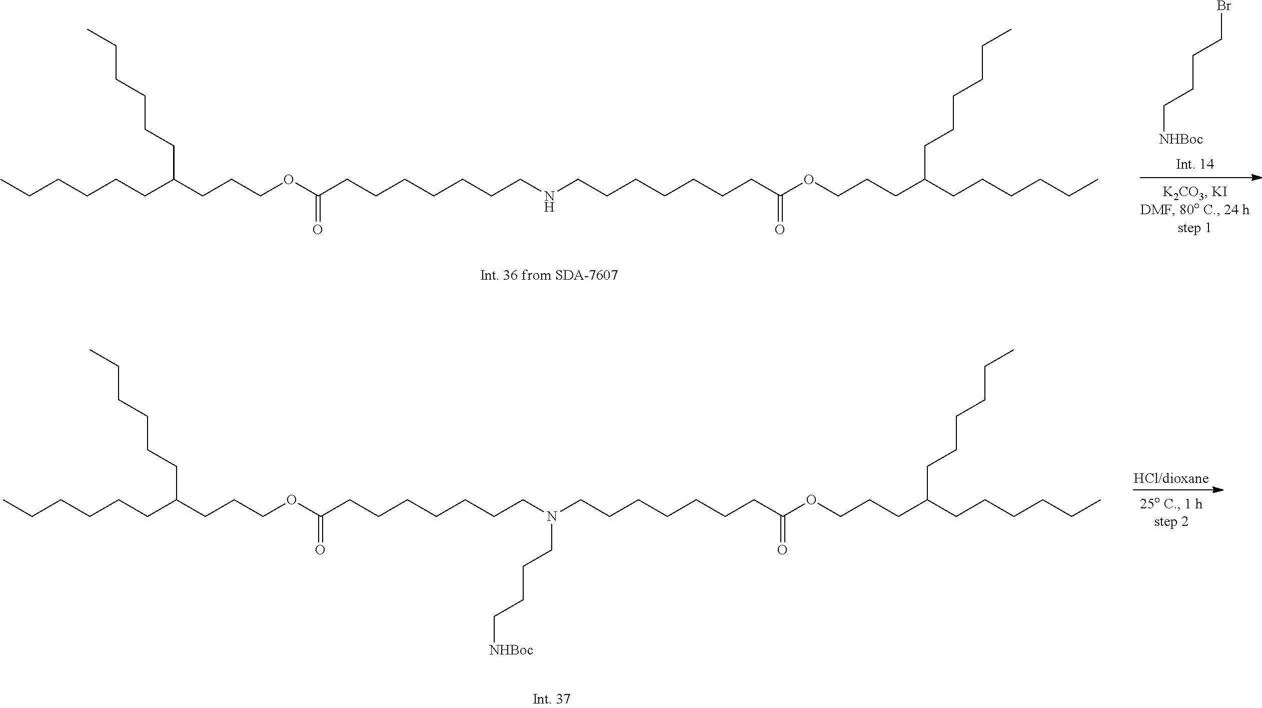

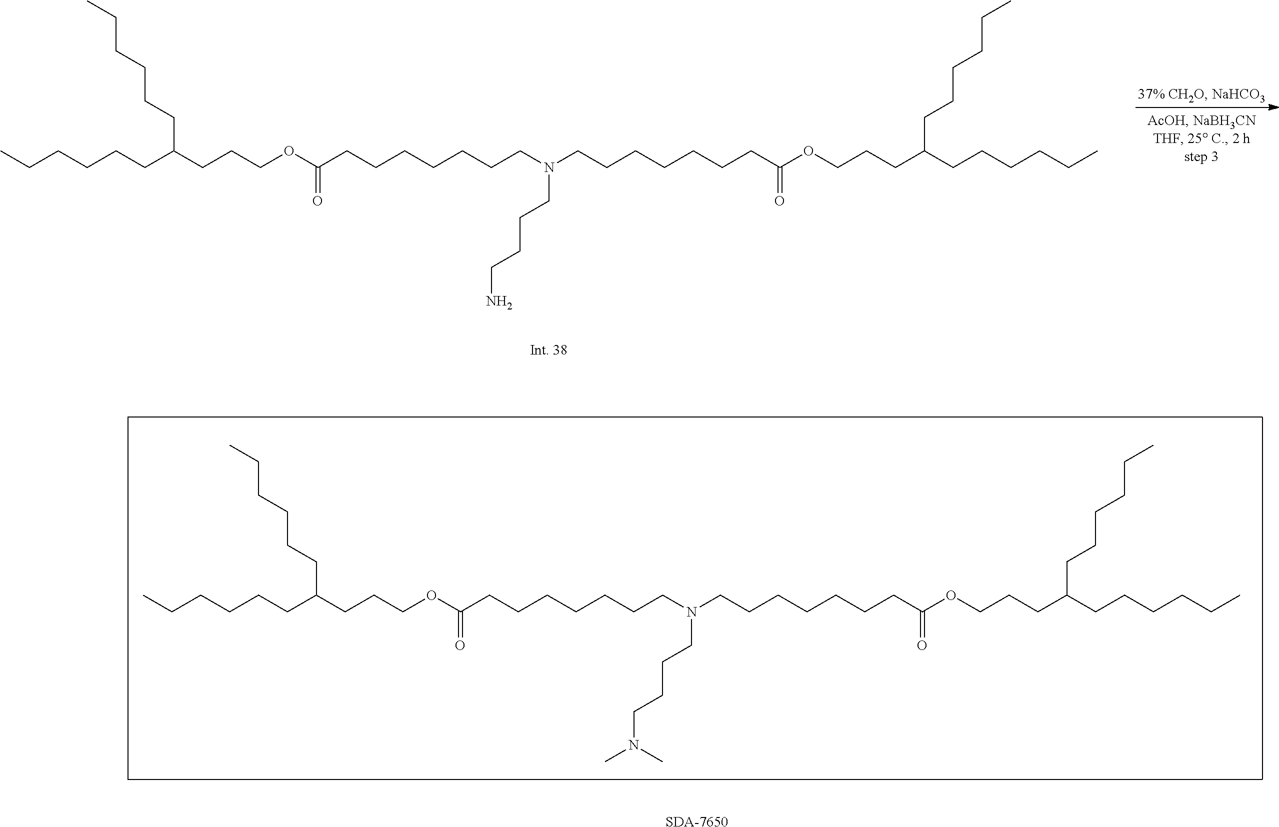

### Step 1:

To a solution of 4-hexyldecyl 8-[[8-(4-hexyldecoxy)-8-oxo-octyl]amino]octanoate (150 mg, 199.93 μmol, 1 eq) in DMF (5 mL) was added K₂CO₃ (138.16 mg, 999.64 μmol, 5 eq), KI (66.38 mg, 399.86 μmol, 2 eq) and tert-butyl N-(4-bromobutyl)carbamate (252.06 mg, 999.64 μmol, 204.93 μL, 5 eq) in DMF (2 mL) The mixture was stirred at 80° C. for 24 hr. The reaction mixture was diluted with H₂O and extracted with EtOAc. The combined organic layers were washed with brine, dried over Na₂SO₄, filtered and concentrated under reduced pressure to give a residue. The residue was purified by silica gel chromatography to give 4-hexyldecyl 8-[4-(tert-butoxycarbonylamino)butyl-[8-(4-hexyldecoxy)-8-oxo-octyl]amino]octanoate (140 mg, 151.93 μmol, 76% yield) as a white solid.

### Step 2:

A solution of 4-hexyldecyl 8-[4-(tert-butoxycarbonylamino)butyl-[8-(4-hexyldecoxy)-8-oxo-octyl]amino]octanoate (100 mg, 108.52 μmol, 1 eq) in HCl/dioxane (4 M, 4.70 mL, 173.08 eq) was stirred at 25° C. for 1 hr. The reaction mixture was concentrated under reduced pressure to give 4-hexyldecyl 8-[4-aminobutyl-[8-(4-hexyldecoxy)-8-oxo-octyl]amino]octanoate (90 mg, crude) as a colorless oil.

### Step 3:

To a solution of 4-hexyldecyl 8-[4-aminobutyl-[8-(4-hexyldecoxy)-8-oxo-octyl]amino]octanoate (125 mg, 145.71 μmol, 1 eq, HCl) and formaldehyde (509.36 mg, 16.96 mmol, 467.30 μL, 116.42 eq) in MeOH (5 mL) was added NaHCO₃ (36.72 mg, 437.14 μmol, 17.00 μL, 3 eq). The mixture was stirred at 25° C. for 10 min, then AcOH (262.51 mg, 4.37 mmol, 250.01 μL, 30 eq) and NaBH₃CN (27.47 mg, 437.14 μmol, 3 eq) was added into the mixture and stirred at 25° C. for 1 h. The reaction mixture was filtered and then diluted with aq. NaHCO₃ and extracted with EtOAc. The combined organic layers were washed with brine, dried over Na₂SO₄, and concentrated under reduced pressure to give a residue. The residue was purified by silica gel chromatography to give 4-hexyldecyl 8-[4-(dimethylamino)butyl-[8-(4-hexyldecoxy)-8-oxo-octyl]amino]octanoate (24 mg, 28.25 μmol, 19% yield) as a colorless oil.

¹H NMR (400 MHz, CDCl₃), 4.04 (t, J=6.8 Hz, 4H), 2.49-2.55 (m, 6H), 2.31-2.37 (m, 2H), 2.28-2.30 (m, 10H), 1.53-1.64 (m, 16H), 1.24-1.32 (m, 58H), 0.89 (t, J=7.2 Hz, 12H)

[M+H]⁺: 849.6

### Synthesis of 7668

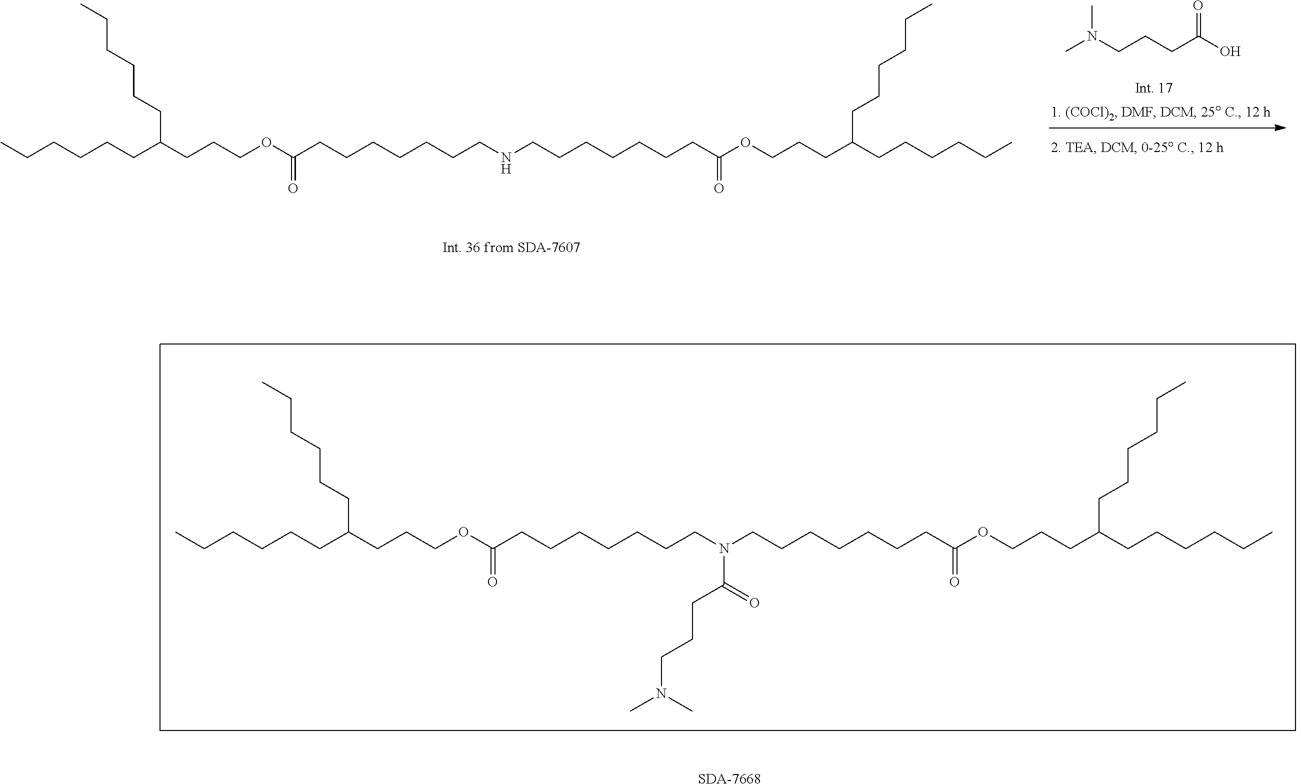

To a solution of 4-(dimethylamino)butanoic acid (100 mg, 596.54 μmol, 1 eq, HCl) in DCM (5 mL) was added oxalyl dichloride (227.15 mg, 1.79 mmol, 156.65 μL, 3 eq) and DMF (1 mL). The mixture was stirred at 25° C. for 12 hr. The reaction mixture was concentrated under reduced pressure to give 4-(dimethylamino)butanoyl chloride (110 mg, crude, HCl) as a white solid.

The 4-(dimethylamino)butanoyl chloride (49.60 mg, 266.57 μmol, 2 eq, HCl) was dropwise added to a solution of 4-hexyldecyl 8-[[8-(4-hexyldecoxy)-8-oxo-octyl]amino]octanoate (100 mg, 133.29 μmol, 1 eq) and TEA (40.46 mg, 399.86 μmol, 55.66 μL, 3 eq) in DCM (2 mL) at 0° C. and stirred at 25° C. for 12 hr. The reaction mixture was diluted with H₂O and extracted with EtOAc. The combined organic layers were washed with brine, dried over Na₂SO₄, filtered and concentrated under reduced pressure to give a residue. The residue was purified by silica gel chromatography to give 4-hexyldecyl 8-[4-(dimethylamino)butanoyl-[8-(4-hexyldecoxy)-8-oxo-octyl]amino]octanoate (36 mg, 41.11 μmol, 31% yield) as a white solid.

¹H NMR (400 MHz, CDCl₃), 4.02-4.06 (m, 4H), 3.28 (t, J=7.6 Hz, 2H), 3.20 (t, J=6.8 Hz, 2H), 2.30-2.34 (m, 8H), 2.24 (s, 6H), 1.79-1.86 (m, 2H), 1.61-1.62 (m, 8H), 1.48-1.52 (m, 4H), 1.24-1.31 (m, 58H), 0.89 (t, J=6.8 Hz, 12H)

[M+H]⁺: 863.7

### Synthesis of 7676

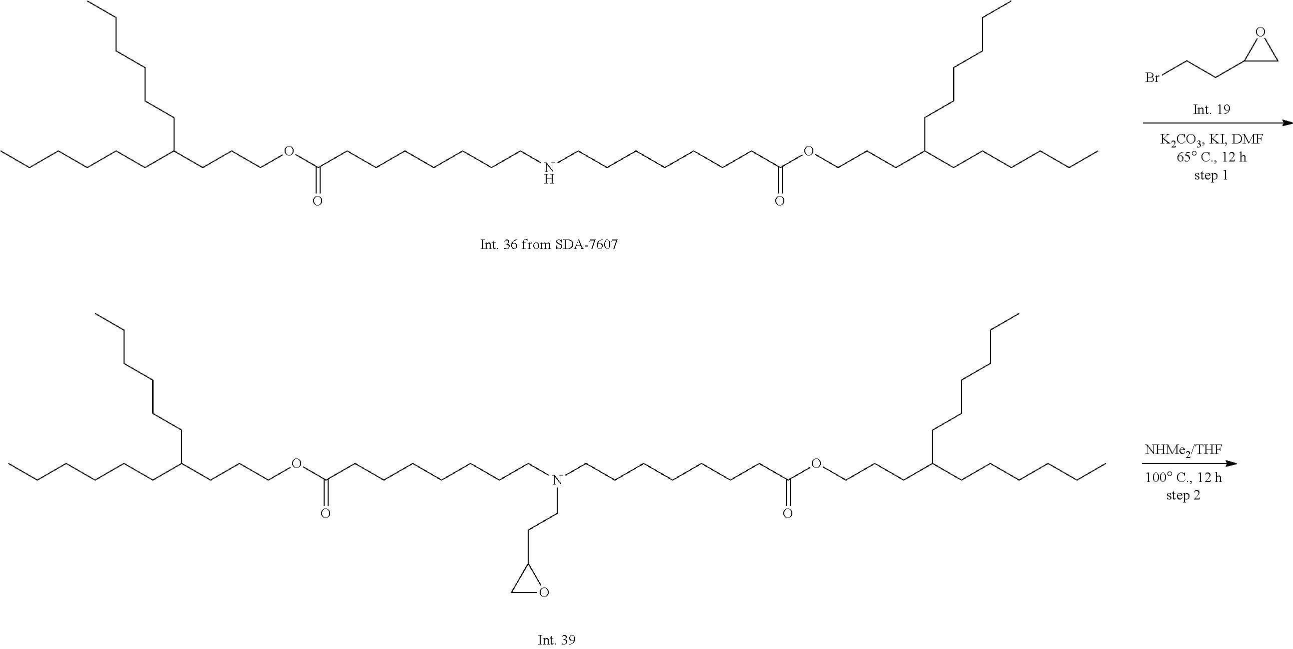

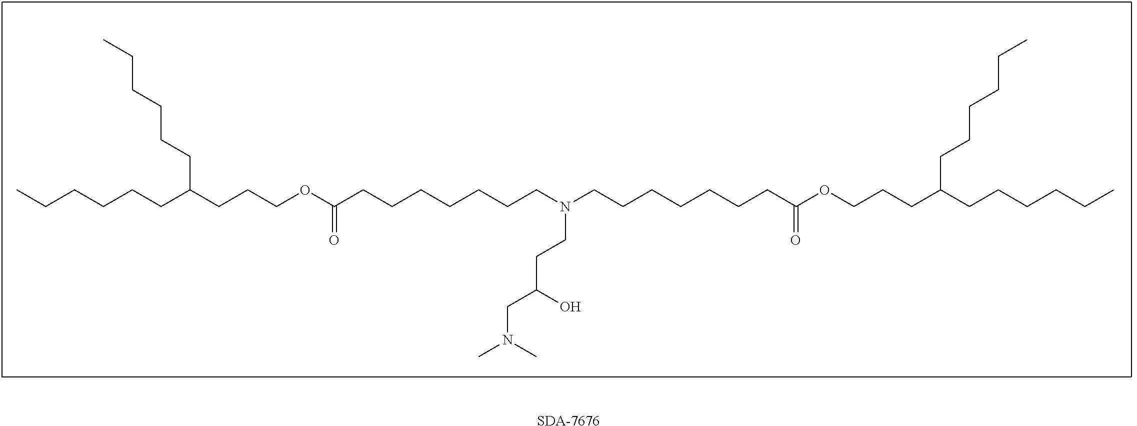

### Step 1:

To a solution of 4-hexyldecyl 8-[[8-(4-hexyldecoxy)-8-oxo-octyl]amino]octanoate (400 mg, 533.14 μmol, 1 eq) in DMF (2 mL) was added K₂CO₃ (368.42 mg, 2.67 mmol, 5 eq) and KI (177.01 mg, 1.07 mmol, 2 eq) then 2-(2-bromoethyl)oxirane (322.02 mg, 2.13 mmol, 4 eq) was added into the mixture. The mixture was stirred at 65° C. for 12 hr. The reaction mixture was filtered and the filtrate was added into H₂O and extracted with EtOAc. The combined organic layers were washed with brine, dried over Na₂SO₄, filtered and concentrated under reduced pressure to give a residue. The residue was purified by silica gel chromatography to give 4-hexyldecyl 8-[[8-(4-hexyldecoxy)-8-oxo-octyl]-[2-(oxiran-2-yl)ethyl]amino]octanoate (140 mg, 170.66 μmol, 32% yield) as a colorless oil.

### Step 2:

A solution of 4-hexyldecyl 8-[[8-(4-hexyldecoxy)-8-oxo-octyl]-[2-(oxiran-2-yl)ethyl]amino]octanoate (100 mg, 121.90 μmol, 1 eq) in N-methylmethanamine (5.48 g, 121.48 mmol, 6.15 mL, 996.61 eq, THE 2M solution) was stirred at 100° C. for 12 hr. The reaction mixture was diluted with NaHCO₃ and extracted with EtOAc. The combined organic layers were washed with brine, dried over Na₂SO₄, filtered and concentrated under reduced pressure to give a residue. The residue was purified by prep-HPLC to give 4-hexyldecyl 8-[[4-(dimethylamino)-3-hydroxy-butyl]-[8-(4-hexyldecoxy)-8-oxo-octyl]amino]octanoate (30 mg, 34.66 μmol, 28% yield) as yellow oil.

¹H NMR (400 MHz, CDCl₃), 4.05 (t, J=6.4 Hz 4H), 3.82-3.88 (m, 1H), 2.48-2.70 (m, 4H), 2.25-2.45 (m, 14H), 1.52-1.72 (m, 10H), 1.40-1.50 (m, 4H), 1.18-1.35 (m, 58H), 0.89 (t, J=6.8 Hz, 12H)

[M+H]⁺: 865.7

### Synthesis of 7671

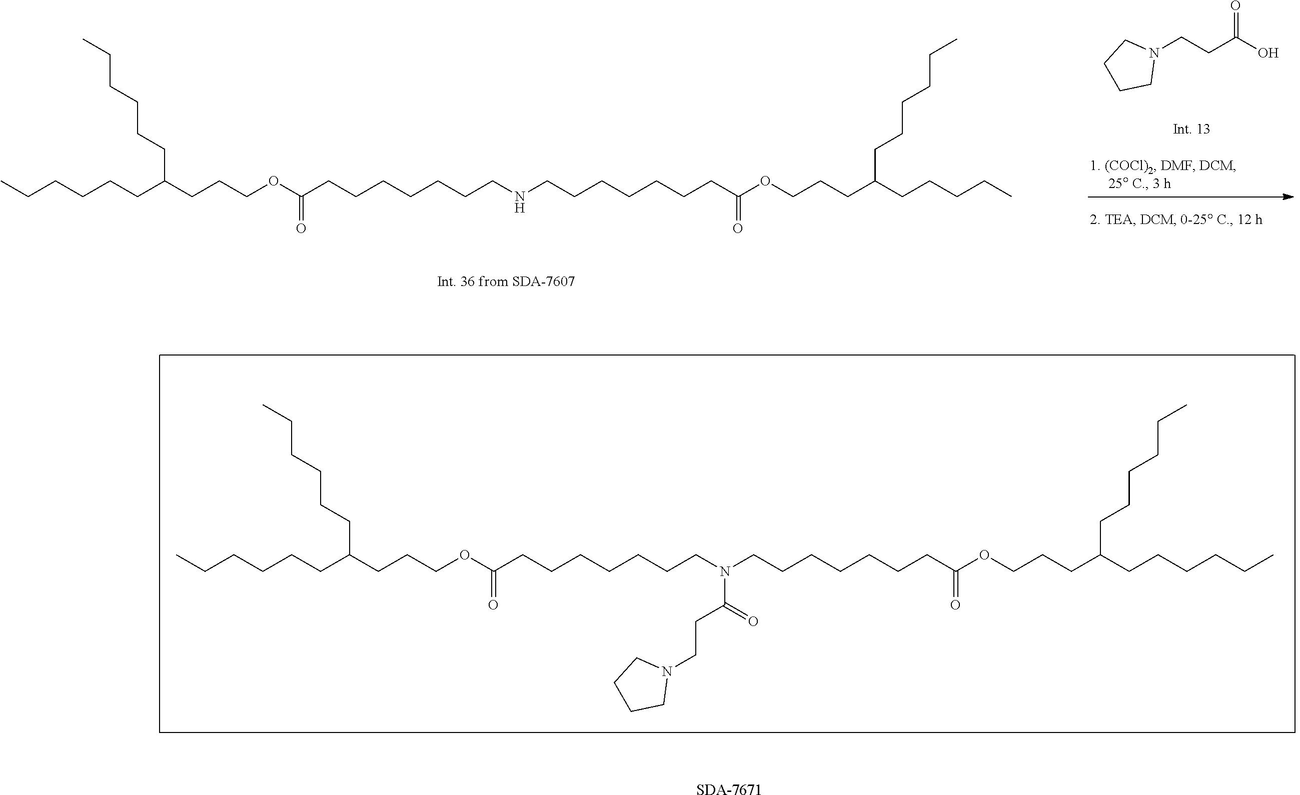

### Step 1:

To a solution of 3-pyrrolidin-1-ylpropanoic acid (450 mg, 3.14 mmol, 1 eq) in DCM (15 mL) was added (COCl)₂ (1.20 g, 9.43 mmol, 825.32 μL, 3 eq) and DMF (3 mL). The mixture was stirred at 25° C. for 3 hr. The reaction mixture was concentrated under reduced pressure to give 3-pyrrolidin-1-ylpropanoyl chloride (600 mg, crude, HCl) as a yellow solid. The 3-pyrrolidin-1-ylpropanoyl chloride (396.04 mg, 2.00 mmol, 5 eq, HCl) in DCM (4 mL) was dropwise added to a solution of 4-hexyldecyl 8-[[8-(4-hexyldecoxy)-8-oxo-octyl]amino]octanoate (300 mg, 399.86 μmol, 1 eq) and TEA (121.38 mg, 1.20 mmol, 166.96 μL, 3 eq) in DCM (3 mL) added at 0° C. The mixture was stirred at 25° C. for 12 hr. The reaction mixture was diluted with H₂O and extracted with EtOAc. The combined organic layers were washed with brine, dried over Na₂SO₄, filtered and concentrated under reduced pressure to give a residue. The residue was purified by prep-HPLC, then concentrated under reduced pressure to remove ACN, then adjusted pH=8 with aq. NaHCO₃ and extracted with EtOAc. The combined organic layers were washed with brine, dried over Na₂SO₄, and concentrated under reduced pressure to give a residue. The residue was purified by silica gel chromatography to give 4-hexyldecyl 8-[[8-(4-hexyldecoxy)-8-oxo-octyl]-(3-pyrrolidin-1-ylpropanoyl)amino]octanoate (116 mg, 128.53 μmol, 32% yield) as clear colorless oil.

¹H NMR (400 MHz, CDCl₃), 4.02-4.06 (m, 4H), 3.28 (t, J=7.6 Hz, 2H), 3.21 (t, J=7.6 Hz, 2H), 2.86 (t, J=7.6 Hz, 2H), 2.48-2.68 (m, 6H), 2.27-2.32 (m, 4H), 1.82 (brs, 4H), 1.57-1.65 (m, 8H), 1.45-1.54 (m, 4H), 1.23-1.32 (m, 58H), 0.89 (t, J=6.4 Hz, 12H)

[M+H]⁺: 875.7

### Synthesis of 7669

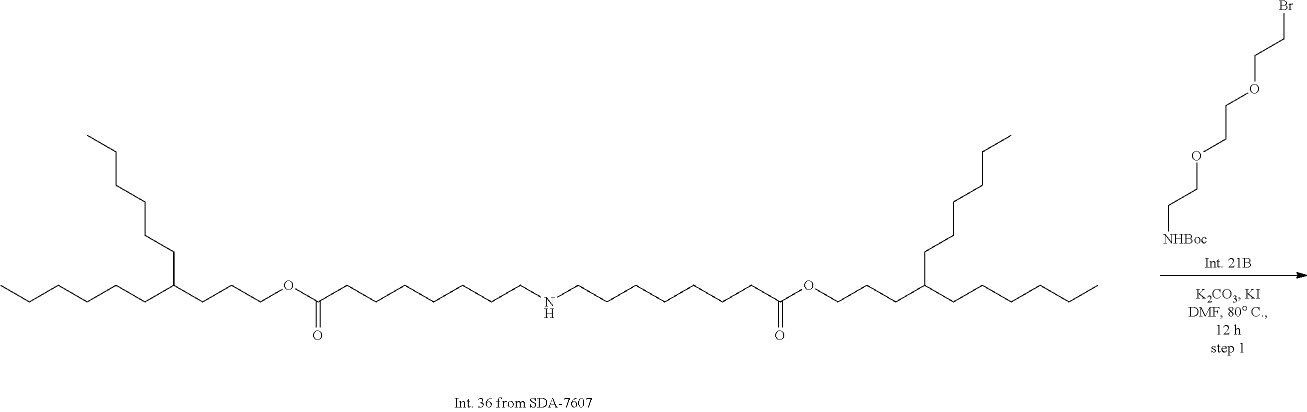 [SMILES: CCCCCCC(CCCCCC)CCCOC(=O)CCCCCCCNCCCCCCCC(=O)OCCCC(CCCCCC)CCCCCC ; CC(C)(C)OC(=O)NCCOCCOCCBr]

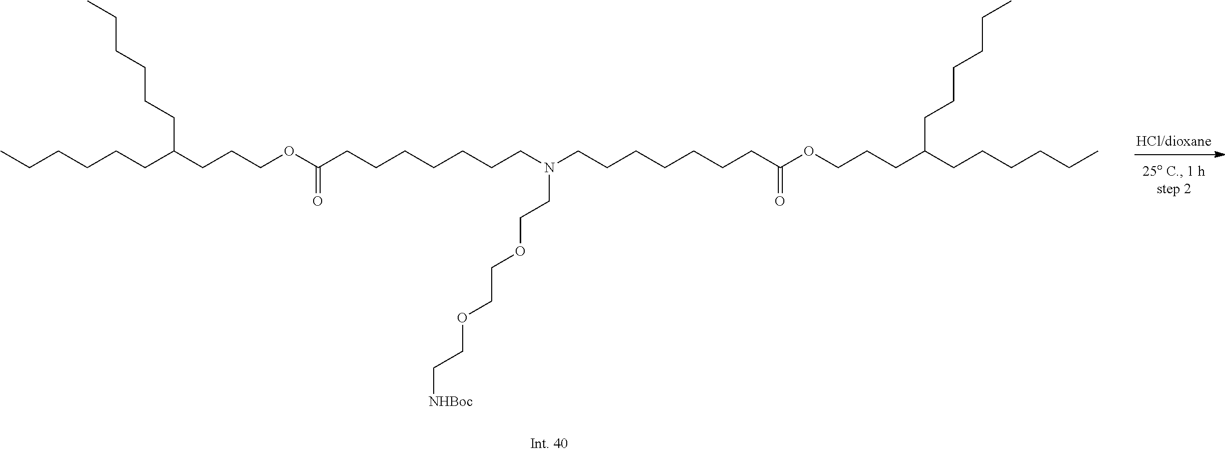 [SMILES: CCCCCCC(CCCCCC)CCCOC(=O)CCCCCCCN(CCCCCCCC(=O)OCCCC(CCCCCC)CCCCCC)CCOCCOCCNC(=O)OC(C)(C)C]

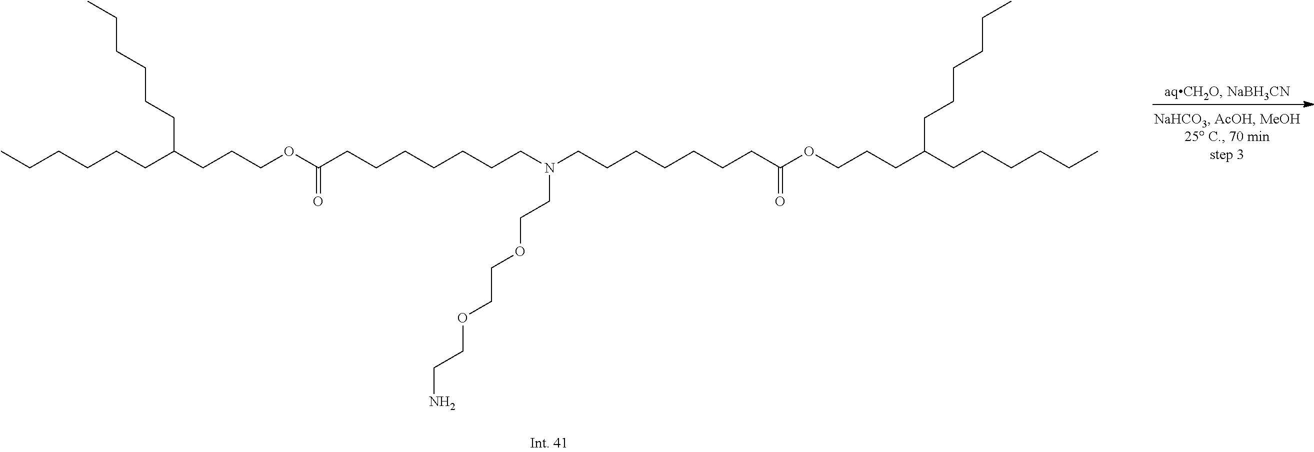 [SMILES: CCCCCCC(CCCCCC)CCCOC(=O)CCCCCCCN(CCCCCCCC(=O)OCCCC(CCCCCC)CCCCCC)CCOCCOCCN]

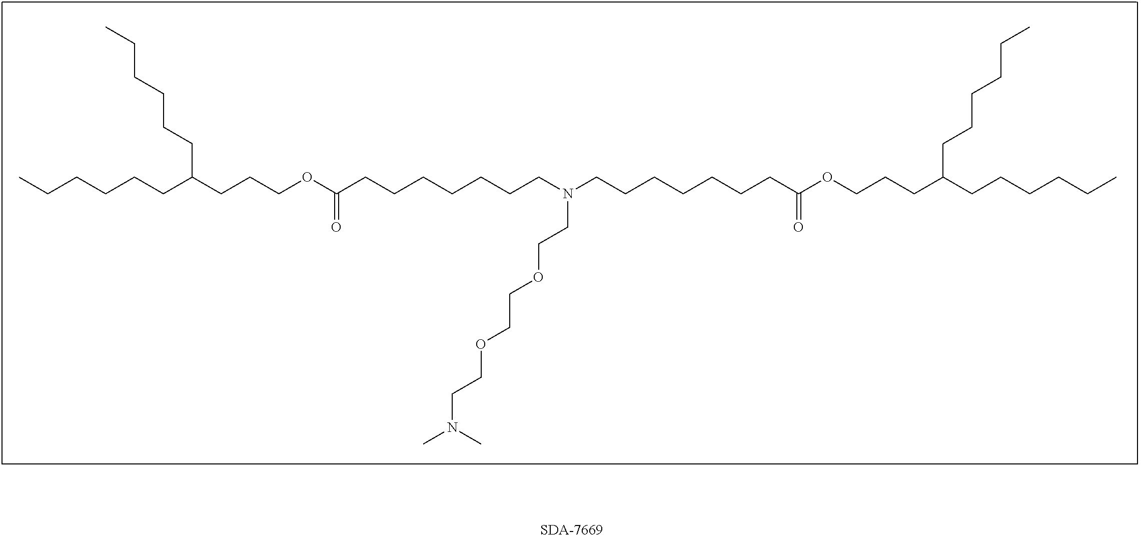

### Step 1:

To a solution of 4-hexyldecyl 8-[[8-(4-hexyldecoxy)-8-oxo-octyl]amino]octanoate (200 mg, 266.57 μmol, 1 eq) in DMF (5 mL) was added K₂CO₃ (184.21 mg, 1.33 mmol, 5 eq), KI (88.50 mg, 533.14 μmol, 2 eq) and tert-butyl N-[2-[2-(2-bromoethoxy)ethoxy]ethyl]carbamate (416.12 mg, 1.33 mmol, 5 eq) in DMF (2 mL), then the mixture was stirred at 80° C. for 12 hr. The reaction mixture was diluted with H₂O and extracted with EtOAc. The combined organic layers were washed with brine, dried over Na₂SO₄, filtered and concentrated under reduced pressure to give a residue. The residue was purified by silica gel chromatography to give 4-hexyldecyl 8-[2-[2-[2-(tert-butoxycarbonylamino)ethoxy]ethoxy]ethyl-[8-(4-hexyldecoxy)-8-oxo-octyl]amino]octanoate (150 mg, 152.82 μmol, 57% yield) as a yellow oil.

### Step 2:

A solution of 4-hexyldecyl 8-[2-[2-[2-(tert-butoxycarbonylamino)ethoxy]ethoxy]ethyl-[8-(4-hexyldecoxy)-8-oxo-octyl]amino]octanoate (140 mg, 142.63 μmol, 1 eq) in HCl/dioxane (4 M, 6.17 mL, 173.08 eq) was stirred at 25° C. for 1 hr. The reaction mixture was concentrated under reduced pressure to give 4-hexyldecyl 8-[2-[2-(2-aminoethoxy)ethoxy]ethyl-[8-(4-hexyldecoxy)-8-oxo-octyl]amino]octanoate (125 mg, crude) as a yellow oil.

### Step 3:

To a solution of 4-hexyldecyl 8-[2-[2-(2-aminoethoxy)ethoxy]ethyl-[8-(4-hexyldecoxy)-8-oxo-octyl]amino]octanoate (125 mg, 141.81 μmol, 1 eq) and formaldehyde (495.72 mg, 16.51 mmol, 454.79 μL, 116.42 eq) in MeOH (10 mL) was added NaHCO₃ (35.74 mg, 425.44 μmol, 16.55 μL, 3 eq) and stirred at 25° C. for 10 min, then AcOH (255.49 mg, 4.25 mmol, 243.32 μL, 30 eq) and NaBH₃CN (26.74 mg, 425.44 μmol, 3 eq) was added to the mixture at 25° C. for 1 hr. The reaction mixture was filtered and the filtrate was diluted with aq. NaHCO₃ and extracted with EtOAc. The combined organic layers were washed with brine, dried over Na₂SO₄, and concentrated under reduced pressure to give a residue. The residue was purified by prep-HPLC, concentrated under reduced pressure to remove ACN, then diluted with aq. NaHCO₃ and extracted with EtOAc. The combined organic layers were washed with brine, dried over Na₂SO₄, and concentrated under reduced pressure to give a residue. The residue was purified by silica gel chromatography to give 4-hexyldecyl 8-[2-[2-[2-(dimethylamino)ethoxy]ethoxy]ethyl-[8-(4-hexyldecoxy)-8-oxo-octyl]amino]octanoate (25 mg, 27.21 μmol, 19% yield) as a colorless oil.

¹H NMR (400 MHz, CDCl₃), 4.04 (t, J=7.2 Hz, 4H), 3.50-3.61 (m, 8H), 2.67 (t, J=6.4 Hz, 2H), 2.55 (t, J=6.0 Hz, 2H), 2.40-2.50 (m, 4H), 2.25-2.35 (m, 10H), 1.55-1.65 (m, 8H), 1.40-1.48 (m, 4H), 1.20-1.35 (m, 58H), 0.89 (t, J=6.8 Hz, 12H)

[M+H]⁺: 909.7

### Example 2. Preparation of Lipid Nanoparticle Compositions

C12-200 is commercially available ionizable lipid and has a chemical name of 1,1′-((2-(4-(2-((2-(bis(2-hydroxydodecyl)amino)ethyl)(2-hydroxydodecyl) amino)ethyl)piperazin-1-yl)ethyl)azanediyl)bis(dodecan-2-ol). A composition composed of ionizable lipid:structural lipid:sterol:EG-lipid (C12-200:DOPE:cholesterol:14:0 PEG2000 PE) at a molar ratio of 35:16:46.5:2.5, respectively. Lipids were solubilized in ethanol. These lipids were mixed at the above-indicated molar ratios and diluted in ethanol (organic phase) to 5.5 mM total lipid concentration. The mRNA solution (aqueous phase) was prepared with RNAse-free water and 100 mM citrate buffer pH 3 for a final concentration of 50 mM citrate buffer. The ionizable lipid to mRNA N:P ratio maintained at 15:1.

All other compositions (i.e., compositions of MC3 (a commercially available ionizable lipid having a chemical name of (6Z,9Z,28Z,31Z)-heptatriacont-6,9,28,31-tetraene-19-yl 4-(dimethylamino)butanoate, and compositions of novel ionizable lipids 7669, 7671, 7668, 767, 7650) were composed of ionizable lipid:structural lipid:sterol:PEG-lipid (SDA lipid #:DSPC:cholesterol:14:0 PEG2000 PE) at a molar ratio of 50:38.5:10:1.5, respectively. Lipids were solubilized in ethanol. Compositions were then handled as above, except the formulations were maintained at ionizable lipid to mRNA N:P ratio of 6:1. The lipid mix and mRNA solution were mixed at a 1:3 ratio by volume, respectively, on a NanoAssemblr Ignite (Precision Nanosystems) at a total flow rate of 9 mL/min. Resulting compositions were then loaded into Slide-A-Lyzer G2 dialysis cassettes (10 k MWCO) and dialyzed in 200 times sample volume of 1×PBS for 4 hrs at room temp with gentle stirring. The PBS was refreshed, and the compositions were further dialyzed for at least 14 hrs at 4° C. with gentle stirring. The dialyzed compositions were then collected and concentrated by centrifugation at 2000×g using Amicon Ultra centrifugation filters (100 k MWCO). Concentrated particles were characterized for size, polydispersity, and particle concentration using Zetasizer Ultra (Malvern Panalytical) and for mRNA encapsulation efficiency using Quant-iT RiboGreen RNA Assay Kit (ThermoFisher Scientific).

Molar ratios of the components of each composition are summarized below.

| | Molar ratio | | | | |
|---|---|---|---|---|---|
| | ionizable | | | | DMPE- |
| Lipid No. | component | cholesterol | DSPC | DOPE | PEG |
| 7596 | 50 | 38.5 | 10 | — | 1.5 |
| 7667 | 50 | 38.5 | 10 | — | 1.5 |
| 7668 | 50 | 38.5 | 10 | — | 1.5 |
| 7669 | 50 | 38.5 | 10 | — | 1.5 |
| 7670 | 50 | 38.5 | 10 | — | 1.5 |
| 7671 | 50 | 38.5 | 10 | — | 1.5 |
| 7676 | 50 | 38.5 | 10 | — | 1.5 |
| 7649 | 50 | 38.5 | 10 | — | 1.5 |
| 7650 | 50 | 38.5 | 10 | — | 1.5 |
| 7651 | 50 | 38.5 | 10 | — | 1.5 |
| 7677 | 50 | 38.5 | 10 | — | 1.5 |
| C12-200 | 35 | 46.5 | — | 16 | 2.5 |
| MC3 | 50 | 38.5 | 10 | — | 1.5 |

### Example 3. In-Vivo Bioluminescent Imaging

8-9 week old female Balb/c mice were utilized for bioluminescence-based ionizable lipid screening efforts. Mice were obtained from Jackson Laboratories (JAX Stock: 000651) and allowed to acclimate for one week prior to manipulations. Animals were placed under a heat lamp for a few minutes before introducing them to a restraining chamber. The tail was wiped with alcohol pads (Fisher Scientific) and 100 μL of a lipid nanoparticle composition described above containing 10 μg total mRNA (5 μg Fluc+5 μg EPO) was injected intravenously using a 29G insulin syringe (Covidien). Any resulting bleeding was stemmed using a sterile gauze pad (Fisher Scientific) and animals were placed back into their home cage. 4-6 hours post-dose, animals were injected with 200 μL of 15 mg/mL D-Luciferin (GoldBio) and placed in an isoflurane induction chamber set to deliver 2.5% isoflurane delivered at an oxygen flow rate of 1-2 liters per min. After 5 minutes of isoflurane exposure, mice were placed in set nose cones inside the IVIS Lumina LT imager (PerkinElmer). LivingImage software was utilized for imaging. Whole body bio-luminescence was captured at auto-exposure after which animals were removed from the IVIS and placed into a CO2 chamber for euthanasia. Cardiac puncture was performed on each animal after placing it in dorsal recumbency, and blood collection was performed using a 25G insulin syringe (BD). Blood was collected in Lithium-Heparin coated tubes (Fisher Scientific) and immediately placed on ice. Once all blood samples were collected, tubes were spun at 2000G for 10 minutes using a tabletop centrifuge and plasma was aliquoted into individual Eppendorf tubes (Fisher Scientific) and stored at −80 C for subsequent EPO quantification. EPO levels in plasma were determined using EPO MSD kit (Meso Scale Diagnostics). Results are shown below.

| Com- | | | | | | | | | | | | | | |
|---|---|---|---|---|---|---|---|---|---|---|---|---|---|---|
| pound: | | | | | | | | | | | | | | |
| Area of | | | | | | | | | | | | | | |
| Body | 7669 | | 7671 | | 7668 | | 7676 | | 7650 | | C12-200 | | MC3 | |
| Whole | 5120 | 2920 | 780000 | 772000 | 48500 | 26900 | 50900 | 30400 | 10900 | 12100 | 3.6E + | 4.32E + | 1.36E + | 96900000 |
| body | | | | | | | | | | | 08 | 08 | 08 | |
| Liver | 92400 | 129000 | 296000 | 184000 | 74000 | 14700 | 183000 | 161000 | 83100 | 56300 | 92300000 | 69700000 | 22000000 | 20300000 |
| Spleen | 112000 | 91700 | 422000 | 574000 | 56400 | 60500 | 255000 | 259000 | 108000 | 82400 | 1280000 | 1390000 | 1660000 | 2060000 |
| Pancreas | 106000 | 87800 | 141000 | 185000 | 26900 | 80800 | 177000 | 220000 | 87200 | 127000 | 236000 | 216000 | 192000 | 232000 |
| Lung | 73000 | 70100 | 215000 | 180000 | 37500 | 79000 | 262000 | 170000 | 138000 | 150000 | 545000 | 266000 | 300000 | 258000 |

As can be seen, novel compounds 7676, 7671, 7650, 7669, and 7668 selectively targeted the pancreas and lung over the whole body, liver, or spleen.

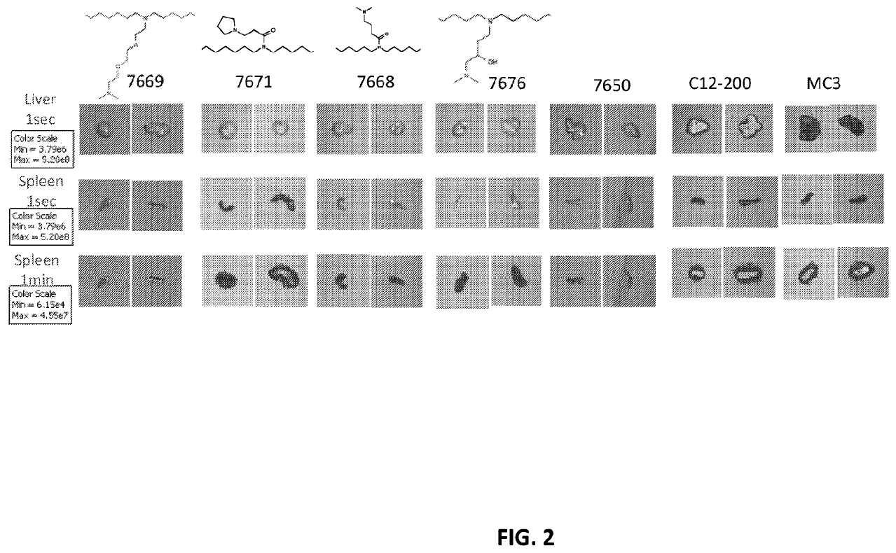

FIG. 2 contains images from bioluminescent imaging in mice liver (1 second after), spleen (1 second and 1 minute after) following administration of one of novel compounds 7669, 7671, 7668, 7676, 7650, C12-200, and MC3.

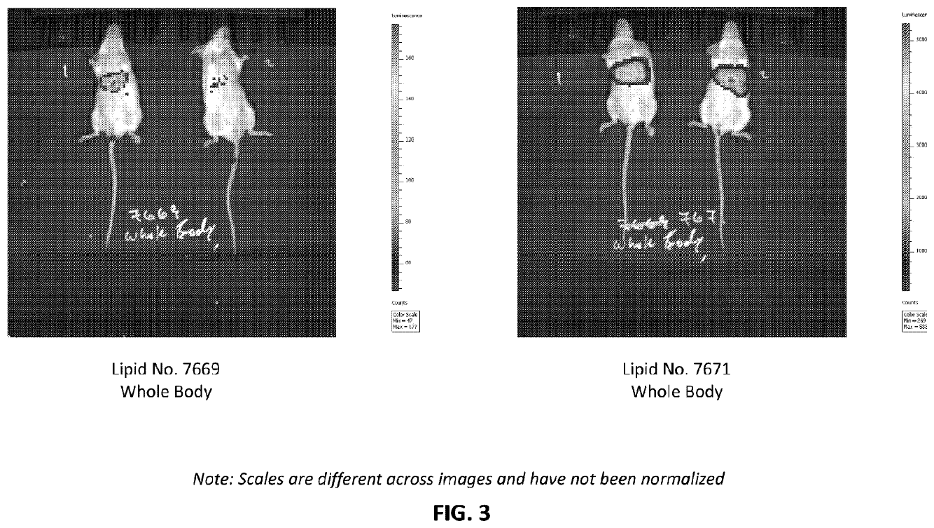

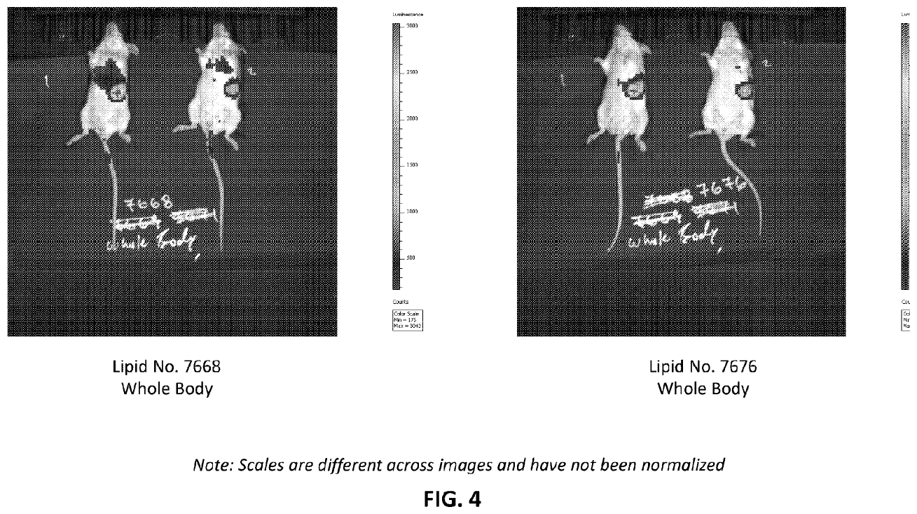

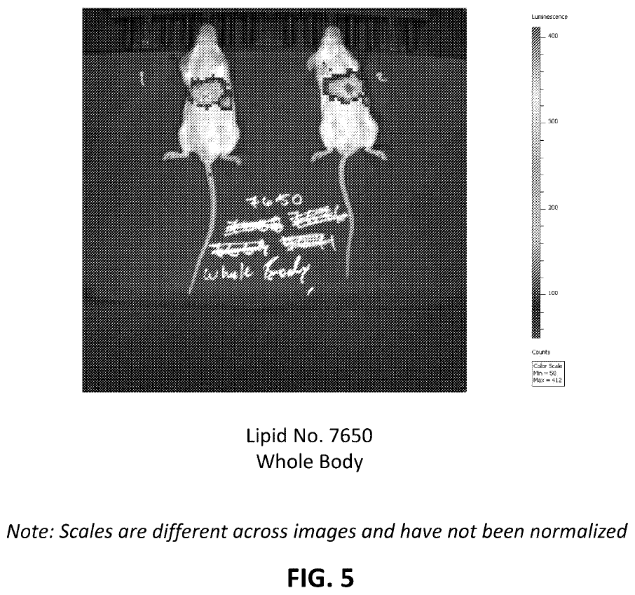

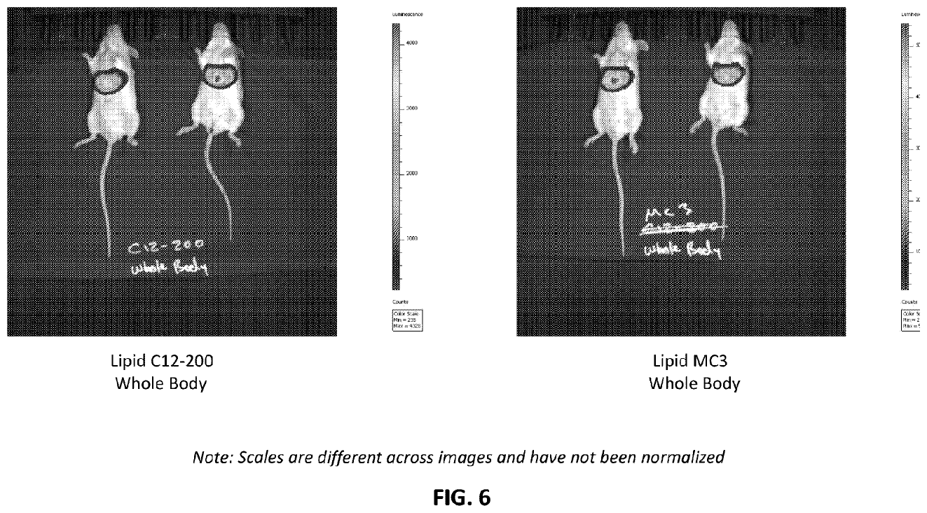

FIGS. 3-6 contain images from wholy body bioluminescent imaging in mice after administration of one of novel compounds 7669, 7671, 7668, 7676, 7650, C12-200, and MC3. The scales in FIGS. 3-6 are different across images and have not been normalized.

Accordingly, the ionizable lipid scaffolds demonstrate selective delivery of the therapeutic cargos outside the liver and, due to the lower lipid levels in the liver, lower liver toxicity is expected.

### Example 4: Synthesis of Exemplary Ionizable Lipid Compounds

### 4.1. Synthesis of Compound 2129

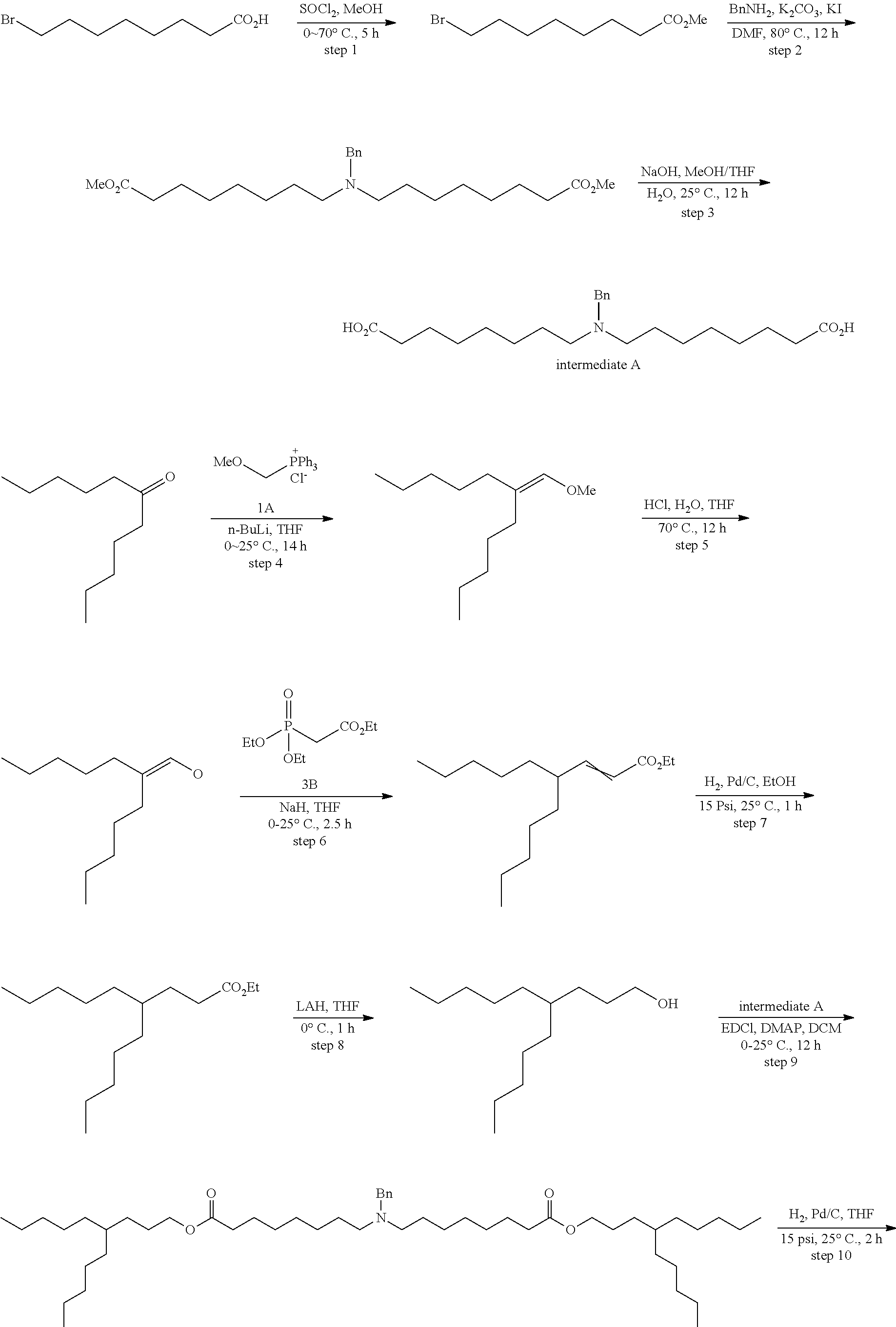

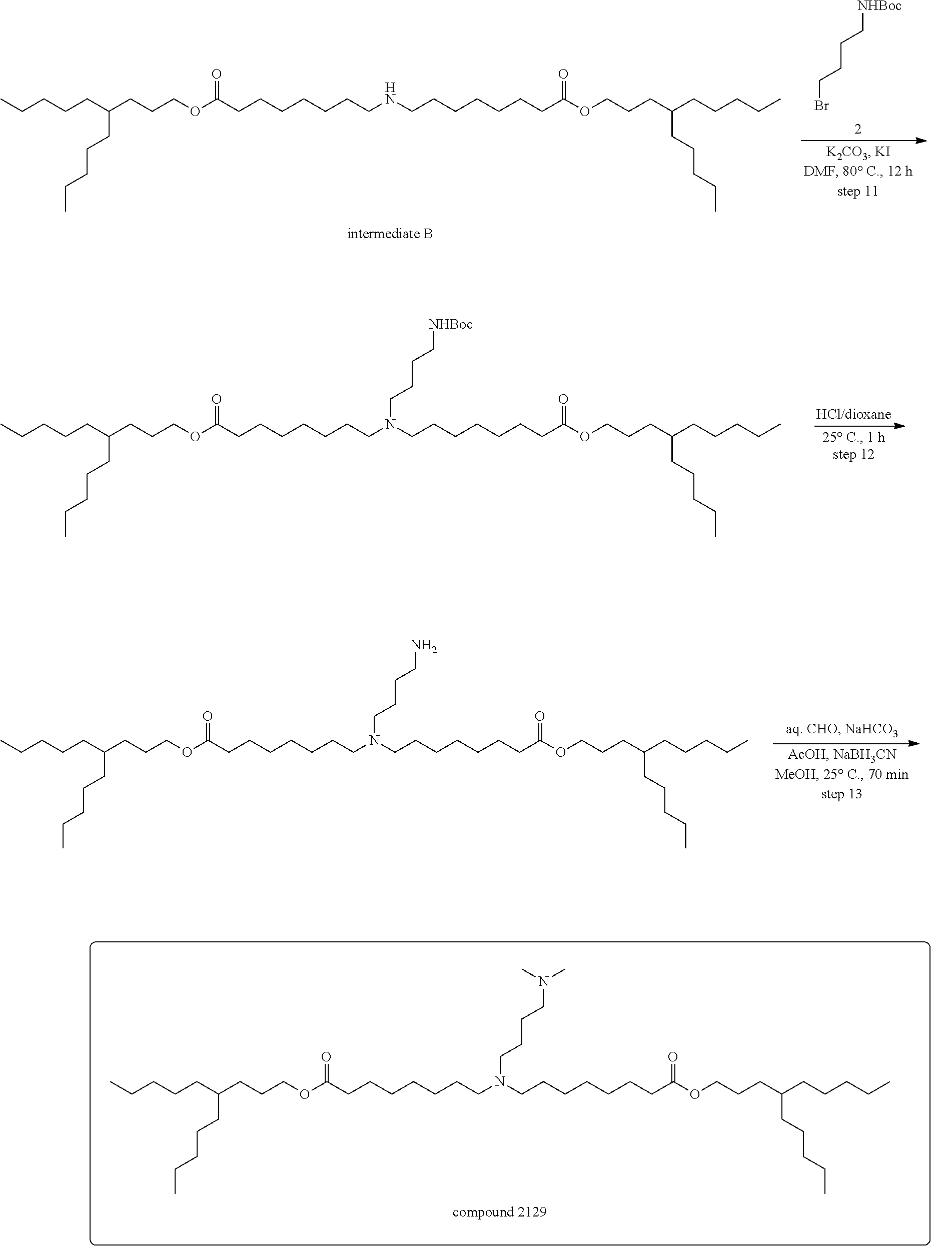

### Step 1:

To a solution of 8-bromooctanoic acid (5 g, 22.41 mmol, 1 eq) in MeOH (50 mL) was added dropwise SOCl₂ (5.33 g, 44.82 mmol, 3.25 mL, 2 eq) at 0° C., then the mixture was stirred at 70° C. for 5 hours. The mixture was concentrated under reduced pressure to get methyl 8-bromooctanoate (4.5 g, crude) as yellow oil.

### Step 2:

To a solution of BnNH₂ (0.78 g, 7.28 mmol, 793.49 uL, 1 eq) in DMF (10 mL) was added K₂CO₃ (5.03 g, 36.40 mmol, 5 eq), KI (3.02 g, 18.20 mmol, 2.5 eq) and the solution of methyl 8-bromooctanoate (3.5 g, 14.76 mmol, 2.03 eq) in DMF (4 mL), then the mixture was stirred at 80° C. for 12 hours. The mixture was filtered and the filtrate was poured into H₂O (50 mL) and extracted with EtOAc (10 mL×3). The combined organic layer was washed with brine (10 mL×2), dried over Na₂SO₄, filtered and the filtrate was concentrated under reduced pressure to give a residue. The residue was purified by column chromatography (SiO₂, Petroleum ether/Ethyl acetate=100/1 to 20/1) to give methyl 8-[benzyl-(8-methoxy-8-oxo-octyl)amino]-octanoate (2.6 g, 6.20 mmol, 85.12% yield) as yellow oil.

¹H NMR (400 MHz, CDCl₃), 7.22-7.31 (m, 5H), 3.67 (s, 6H), 3.53 (s, 4H), 2.38 (t, J=7.2 Hz, 4H), 2.30 (t, J=7.6 Hz, 4H), 1.59-1.63 (m, 6H), 1.43-1.50 (m, 4H), 1.27-1.35 (m, 14H).

### Step 3:

To a solution of methyl 8-[benzyl-(8-methoxy-8-oxo-octyl)amino]octanoate (2.6 g, 6.20 mmol, 1 eq) in THE (3 mL) and MeOH (10 mL) was added a solution of NaOH (845.87 mg, 21.15 mmol, 3.41 eq) in H₂O (5 mL), then the mixture was stirred at 25° C. for 12 hours. The reaction mixture was concentrated under reduced pressure to get a residue. The residue was added into H₂O (10 mL) and extracted with EtOAc (10 mL×3). The aqueous phase was adjusted the pH=6-7 with 1N HCl, then extracted with EtOAc (20 mL×5). The organic layer was washed with brine (10 mL×2), dried over Na₂SO₄, filtered and the filtrate was concentrated under reduced pressure to give 8-[benzyl(7-carboxyheptyl)amino]octanoic acid (2 g, 5.11 mmol, 82.43% yield, − purity) as colorless oil.

¹H NMR (400 MHz, DMSO), 7.21-7.30 (m, 5H), 3.49 (s, 2H), 2.34 (t, J=6.8 Hz, 4H), 2.16 (t, J=7.2 Hz, 4H), 1.38-1.47 (m, 8H), 1.21-1.25 (m, 12H).

### Step 4:

To a solution of methoxymethyl(triphenyl)phosphonium; chloride (24.16 g, 70.47 mmol, 3 eq) in THE (360 mL) was added dropwise n-BuLi (2.5 M, 26.31 mL, 2.8 eq) at 0° C. and the mixture was stirred at 25° C. for 2 hours. A solution of undecan-6-one (4 g, 23.49 mmol, 1 eq) in THE (120 mL) was added into the mixture at 0° C., then stirred at 25° C. for 12 hours. The mixture was poured into H₂O (200 mL) at 0° C. and extracted with EtOAc (100 mL×3). The combined organic layer was washed with brine (100 mL×2), dried over Na₂SO₄, filtered and the filtrate was concentrated under reduced pressure to get a residue. The residue was purified by column chromatography (SiO₂, Petroleum ether/Ethyl acetate=100/0 to 50/1) to give 6-(methoxymethylene)undecane (18 g, 90.75 mmol, 77.27% yield) as colorless oil.

### Step 5:

A solution of 6-(methoxymethylene)undecane (18 g, 90.75 mmol, 1 eq) in THF (72 mL) and HCl (3 M, 18.00 mL, 5.95e-1 eq) aq. was stirred at 70° C. for 12 hours. The mixture was poured into H₂O (100 mL) at 0° C., extracted with EtOAc (50 mL×3). The combined organic layer was washed with brine (50 mL×2), dried over Na₂SO₄, filtered and the filtrate was concentrated under reduced pressure to get a residue. The residue was purified by column chromatography (SiO₂, Petroleum ether/Ethyl acetate=100/1 to 20/1) to give 2-pentylheptanal (15 g, 81.38 mmol, 89.67% yield, − purity) as colorless oil.

¹H NMR (400 MHz, CDCl₃), 5.75 (s, 1H), 3.52 (s, 3H), 2.05 (t, J=7.2 Hz, 2H), 1.85 (t, J=7.2 Hz, 2H), 1.28-1.35 (m, 12H), 0.90 (t, J=7.2 Hz, 6H).

### Step 6:

To a solution of NaH (3.95 g, 98.74 mmol, 7.05 mL, 60% purity, 1.3 eq) in THE (280 mL) was added dropwise ethyl 2-diethoxyphosphorylacetate (22.14 g, 98.74 mmol, 19.59 mL, 1.3 eq) at 0° C., the mixture was stirred at 25° C. for 0.5 hour. A solution of 2-pentylheptanal (14 g, 75.96 mmol, 1 eq) in THF (70 mL) was added into the mixture at 0° C., then the mixture was warmed to 25° C. and stirred at 25° C. for 2 hours. The mixture was poured into H₂O (200 mL) at 0° C., extracted with EtOAc (100 mL×3). The combined organic layer was washed with brine (50 mL×2), dried over Na₂SO₄, filtered and the filtrate was concentrated under reduced pressure to get a residue. The residue was purified by column chromatography (SiO₂, Petroleum ether/Ethyl acetate=100/1 to 20/1) to give ethyl 4-pentylnon-2-enoate (16 g, 62.89 mmol, 82.80% yield) as colorless oil.

¹H NMR (400 MHz, CDCl₃), 9.56 (d, J=3.2, 1H), 2.24-2.25 (m, 1H), 1.43-1.61 (m, 2H), 1.29-1.34 (m, 2H), 1.26 (s, 12H), 0.90 (t, J=7.2 Hz, 6H).

### Step 7:

A solution of Pd/C (2.5 g, 10% purity) and ethyl 4-pentylnon-2-enoate (5 g, 19.65 mmol, 1 eq) in EtOH (100 mL) was stirred at 25° C. for 1 hour under H₂ (15 Psi). The mixture was filtered and the filtrate was concentrated under reduced pressure to give the compound ethyl 4-pentylnonanoate (15 g, crude) as colorless oil.

¹H NMR (400 MHz, CDCl₃), 6.74 (dd, J₁=9.2 Hz, J₂=15.6 Hz, 1H), 5.76 (d, J=15.6 Hz, 1H), 4.19 (q, J=7.2 Hz, 2H), 2.09-2.15 (m, 1H), 1.30-1.42 (m, 2H), 1.24-1.29 (m, 17H), 0.88 (t, J=7.2 Hz, 6H).

### Step 8:

To a solution of LAH (1.48 g, 39.00 mmol, 7.05 mL, 2 eq) in THE (50 mL) was added a solution of ethyl 4-pentylnonanoate (5 g, 19.50 mmol, 1 eq) in THF (10 mL) at 0° C. and stirred at 0° C. for 1 hour. The mixture was poured into H₂O (30 mL) at 0° C., then the mixture was filtered and the filtrate was extracted with EtOAc (50 mL×3). The combined organic layer was washed with brine (50 mL×2), dried over Na₂SO₄, filtered and the filtrate was concentrated under reduced pressure. The residue was purified by column chromatography (SiO₂, Petroleum ether/Ethyl acetate=100/1 to 20/1) to give 4-pentylnonan-1-ol (10 g, 46.64 mmol, 79.74% yield) as colorless oil.

¹H NMR (400 MHz, CDCl₃), 4.13 (q, J=7.2 Hz, 2H), 2.28 (t, J=8.0, 2H), 1.57-1.60 (m, 4H), 1.25-1.32 (m, 18H), 0.89 (t, J=7.2 Hz, 6H).

### Step 9:

To a solution of 4-pentylnonan-1-ol (1.15 g, 5.36 mmol, 2.1 eq) and 8-[benzyl(7-carboxyheptyl) amino]octanoic acid (1 g, 2.55 mmol, 1 eq) in DCM (10 mL) was added DMAP (156.01 mg, 1.28 mmol, 0.5 eq) and EDCI (1.47 g, 7.66 mmol, 3 eq) at 0° C., then stirred at 25° C. for 12 hours. The mixture was added into H₂O (10 mL) and extracted with DCM (10 mL×3). The combined organic layer was washed with brine (10 mL×2), dried over Na₂SO₄, filtered and the filtrate was concentrated under reduced pressure to get a residue. The residue was purified by column chromatography (SiO₂, Petroleum ether/Ethyl acetate=30/1 to 10/1) to give 4-pentylnonyl 8-[benzyl-[8-oxo-8-(4-pentylnonoxy)octyl]amino] octanoate (1.5 g, 1.91 mmol, 74.89% yield) as colorless oil.

¹H NMR (400 MHz, CDCl₃), 3.64 (q, J=6.8 Hz, 2H), 1.50-1.55 (m, 2H), 1.22-1.31 (m, 20H), 0.89 (t, J=7.2 Hz, 6H).

### Step 10:

A solution of Pd/C (200 mg, 637.52 μmol, 10% purity, 1 eq) and 4-pentylnonyl 8-[benzyl-[8-oxo-8-(4-pentylnonoxy)octyl]amino]octanoate (500 mg, 637.52 μmol, 1 eq) in THF (20 mL) was stirred at 25° C. for 2 hours under H₂ (15 Psi). The mixture was filtered and the filtrate was concentrated under reduced pressure to get a residue. The residue was purified by column chromatography (SiO₂, Petroleum ether/Ethyl acetate=30/1 to 5/1) to give 4-pentylnonyl 8-[[8-oxo-8-(4-pentylnonoxy)octyl]amino] octanoate (900 mg, crude) as colorless oil.

### Step 11:

To a solution of 4-pentylnonyl 8-[[8-oxo-8-(4-pentylnonoxy)octyl]amino]octanoate (150 mg, 216.09 μmol, 1 eq) in DMF (5 mL) was added K₂CO₃ (149.33 mg, 1.08 mmol, 40.10 uL, 5 eq) and KI (71.74 mg, 432.18 μmol, 2 eq), then a solution of tert-butyl N-(4-bromobutyl)carbamate (217.94 mg, 864.35 μmol, 177.19 μL, 4 eq) in DMF (2 mL) was added into the mixture and stirred at 80° C. for 12 hours. The reaction mixture was filtered and concentrated in vacuo to get a residue. The residue was purified by column chromatography (SiO₂, Petroleum ether/Ethyl acetate=20/1 to 3/1). 4-pentylnonyl8-[4-(tert-butoxycarbonylamino)butyl-[8-oxo-8-(4-pentylnonoxy)octyl]amino]octanoate (120 mg, 138.66 μmol, 64.17% yield, − purity) as colorless oil.

¹H NMR (400 MHz, CDCl₃), 4.05 (t, J=7.2 Hz, 4H), 2.60 (t, J=7.2 Hz, 4H), 2.30 (t, J=7.2 Hz, 4H), 1.60-1.63 (m, 12H), 1.24-1.32 (m, 50H), 0.89 (t, J=7.2 Hz, 12H).

### Step 12:

A solution of 4-pentylnonyl 8-[4-(tert-butoxycarbonylamino)butyl-[8-oxo-8-(4-pentylnonoxy) octyl]amino]octanoate (120 mg, 138.66 μmol, 1 eq) in HCl/dioxane (4 M, 6.00 mL, 173.08 eq) was stirred at 25° C. for 1 hour. The reaction mixture was concentrated in vacuo to give 4-pentylnonyl 8-[4-aminobutyl-[8-oxo-8-(4-pentylnonoxy)octyl]amino]octanoate (110 mg, crude, HCl) as a yellow solid.

### Step 13:

To a solution of 4-pentylnonyl 8-[4-aminobutyl-[8-oxo-8-(4-pentylnonoxy)octyl]amino]octanoate (110 mg, 137.20 μmol, 1 eq, HCl) and formaldehyde (1.30 g, 15.97 mmol, 1.19 mL, 37% purity, 116.42 eq) in MeOH (10 mL) was added NaHCO₃ (34.58 mg, 411.60 μmol, 16.01 μL, 3 eq), stirred at 25° C. for 10 minutes, then AcOH (247.17 mg, 4.12 mmol, 235.40 μL, 30 eq) and NaBH₃CN (25.87 mg, 411.60 μmol, 3 eq) were added to the mixture and stirred at 25° C. for 1 hour. The reaction mixture was quenched with sat.NaHCO₃ (10 mL) and extracted with EtOAc (3×10 mL). The combined organic layer was washed with brine (2×5 mL), dried over anhydrous sodium sulfate, filtered and concentrated in vacuo to get a residue. The residue was purified by column chromatography (SiO₂, Ethyl acetate:MeOH=30/1 to 1/1) to give 4-pentylnonyl 8-[4-(dimethylamino)butyl-[8-oxo-8-(4-pentylnonoxy)octyl]amino]octanoate (87 mg, 109.66 μmol, 79.93% yield, 100% purity) as colorless oil.

¹H NMR (400 MHz, CDCl₃), 4.04 (t, J=6.8 Hz, 4H), 2.58-2.62 (m, 6H), 2.38 (t, J=6.4 Hz, 2H), 2.28-2.31 (m, 10H), 1.56-1.63 (m, 16H), 1.24-1.32 (m, 50H), 0.89 (t, J=7.2 Hz, 12H).

LCMS: (M+H⁺): 793.6 @ 3.527 min.

### 4.2. Synthesis of Compound 2130

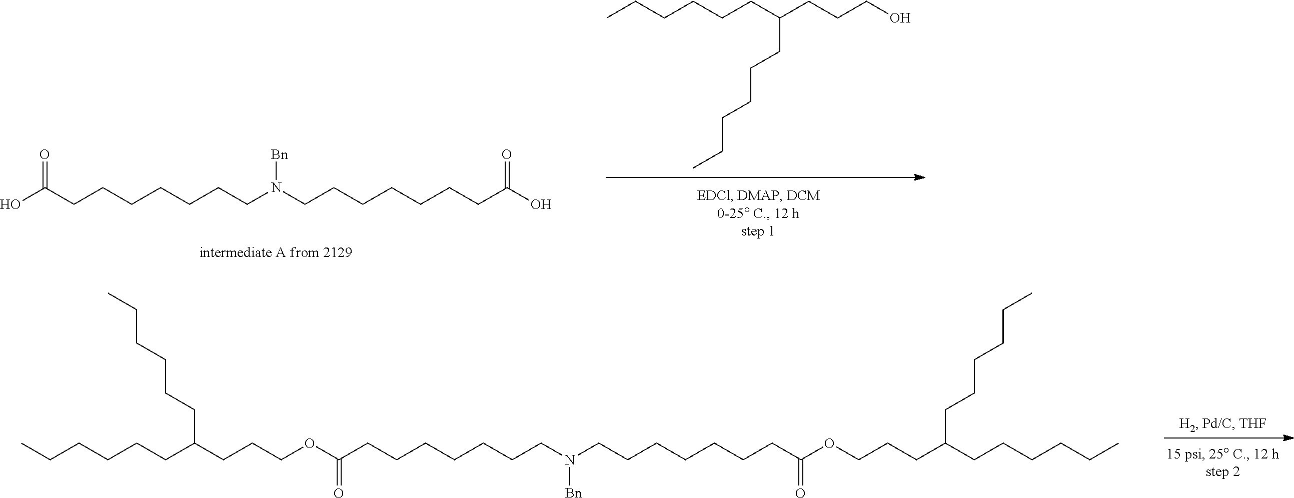 [SMILES: O=C(O)CCCCCCCN(CCCCCCCC(=O)O)Cc1ccccc1 ; CCCCCCC(CCCO)CCCCCC ; CCCCCCC(CCCCCC)CCCOC(=O)CCCCCCCN(CCCCCCCC(=O)OCCCC(CCCCCC)CCCCCC)Cc1ccccc1]

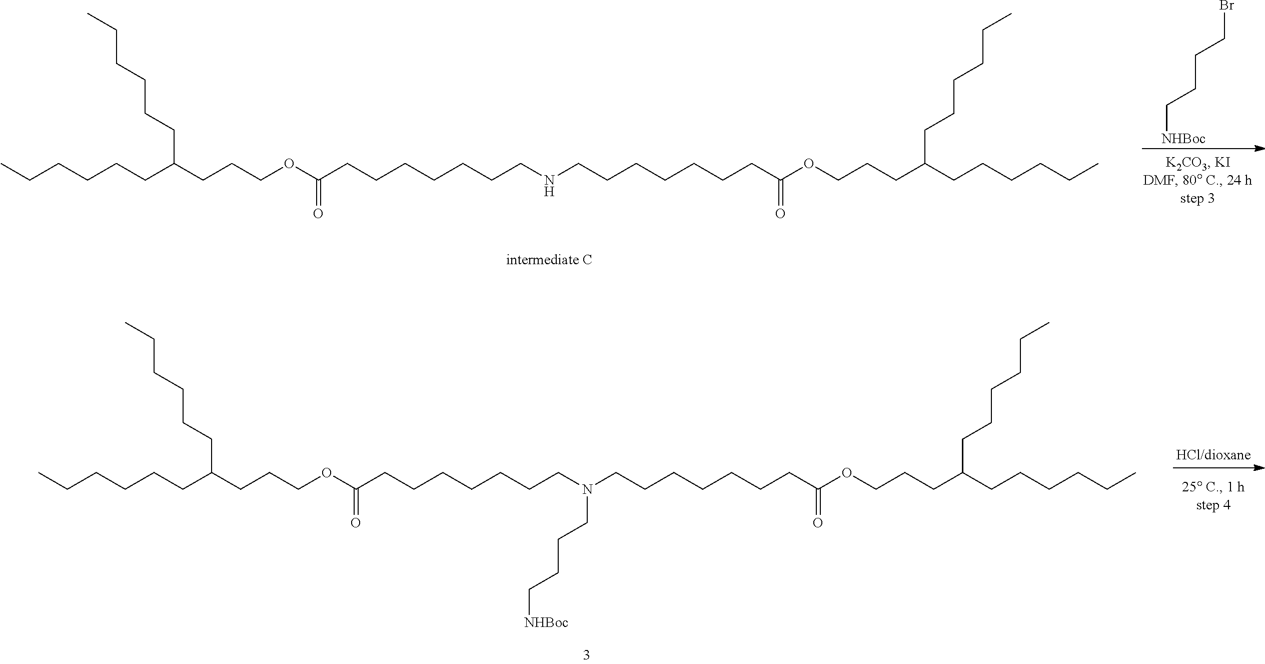

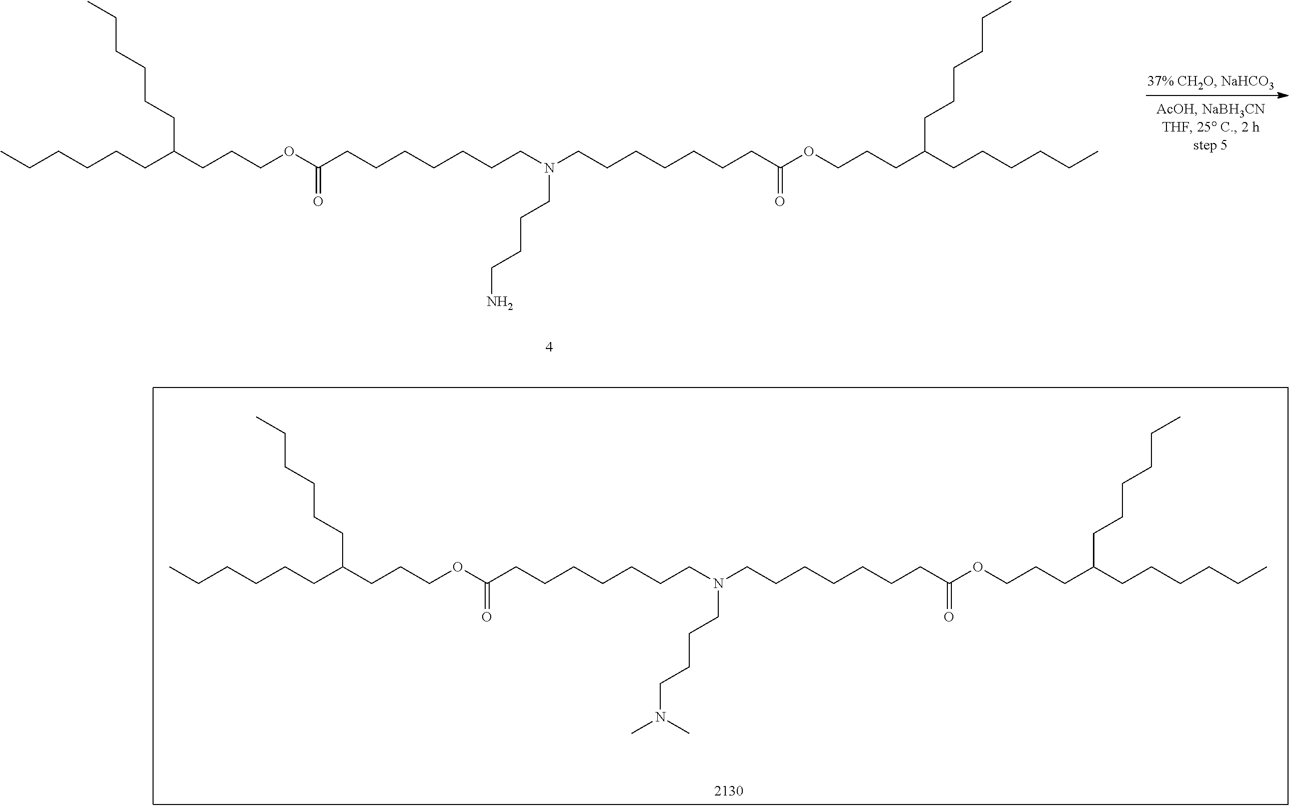

### Step 1:

To a solution of 8-[benzyl(7-carboxyheptyl)amino]octanoic acid (1.7 g, 4.34 mmol, 1 eq) in DCM (20 mL) was added DMAP (265.21 mg, 2.17 mmol, 0.5 eq), 4-hexyldecan-1-ol (2.16 g, 8.90 mmol, 2.05 eq) and EDCI (2.50 g, 13.02 mmol, 3 eq) at 0° C. and stirred at 25° C. for 12 hours under N₂ atmosphere. The reaction mixture was diluted with H₂O (10 mL) and extracted with EtOAc 15 mL (5 mL×3). The combined organic layers were dried over Na₂SO₄, filtered and concentrated under reduced pressure to give a residue. The residue was purified by column chromatography (SiO₂, Petroleum ether/Ethyl acetate=1/0 to 91/9) to give compound 4-hexyldecyl 8-[benzyl-[8-(4-hexyldecoxy)-8-oxo-octyl]amino]octanoate (2.9 g, 3.45 mmol, 79.51% yield) as colorless oil.

### Step 2:

A solution of Pd/C (1 g, 1.78 mmol, 10% purity, 1 eq) in THF (30 mL) was added 4-hexyldecyl 8-[benzyl-[8-(4-hexyldecoxy)-8-oxo-octyl]amino]octanoate (1.5 g, 1.78 mmol, 1 eq) was stirred under H₂ (15 psi) at 25° C. for 12 hours. The mixture is filtered through celite and the filtrate was removed under reduced pressure to get a residue. The residue was purified by column chromatography (SiO₂, Petroleum ether/Ethyl acetate=5/1 to 0/1) to give 4-hexyldecyl 8-[[8-(4-hexyldecoxy)-8-oxo-octyl]amino]octanoate (700 mg, 933.00 μmol, 52.27% yield) as a colorless oil.

### Step 3:

To a solution of 4-hexyldecyl 8-[[8-(4-hexyldecoxy)-8-oxo-octyl]amino]octanoate (150 mg, 199.93 μmol, 1 eq) in DMF (5 mL) was added K₂CO₃ (138.16 mg, 999.64 μmol, 5 eq), KI (66.38 mg, 399.86 μmol, 2 eq) and tert-butyl N-(4-bromobutyl)carbamate (252.06 mg, 999.64 μmol, 204.93 μL, 5 eq) in DMF (2 mL). The mixture was stirred at 80° C. for 24 hours. The reaction mixture was diluted with H₂O 6 mL and extracted with EtOAc 6 mL (2 mL×3). The combined organic layers were washed with Brine 3 mL (1 m×3), dried over Na₂SO₄, filtered and concentrated under reduced pressure to give a residue. The residue was purified by column chromatography (SiO₂, Petroleum ether/Ethyl acetate=6/1 to 0/1) to give 4-hexyldecyl 8-[4-(tert-butoxycarbonylamino)butyl-[8-(4-hexyldecoxy)-8-oxo-octyl]amino]octanoate (140 mg, 151.93 μmol, 75.99% yield) as a white solid.

¹H NMR (400 MHz, CDCl₃), 5.1 (brs, 1H), 4.04 (t, J=6.8 Hz, 4H), 3.10-3.20 (m, 2H), 2.40-2.44 (m, 6H), 2.29 (t, J=7.2 Hz, 4H), 1.53-1.64 (m, 12H), 1.44-1.48 (m, 13H), 1.24-1.32 (m, 58H), 0.89 (t, J=7.2 Hz, 12H).

### Step 4:

A solution of 4-hexyldecyl 8-[4-(tert-butoxycarbonylamino)butyl-[8-(4-hexyldecoxy)-8-oxo-octyl]amino]octanoate (100 mg, 108.52 μmol, 1 eq) in HCl/dioxane (4 M, 4.70 mL, 173.08 eq) was stirred at 25° C. for 1 hour. The reaction mixture was concentrated under reduced pressure to give 4-hexyldecyl 8-[4-aminobutyl-[8-(4-hexyldecoxy)-8-oxo-octyl]amino]octanoate (90 mg, crude) as a colorless oil.

### Step 5:

To a solution of 4-hexyldecyl 8-[4-aminobutyl-[8-(4-hexyldecoxy)-8-oxo-octyl]amino]octanoate (125 mg, 145.71 μmol, 1 eq, HCl) and formaldehyde (509.36 mg, 16.96 mmol, 467.30 μL, 116.42 eq) in MeOH (5 mL) was added NaHCO₃ (36.72 mg, 437.14 μmol, 17.00 μL, 3 eq). The mixture was stirred at 25° C. for 10 minutes, then AcOH (262.51 mg, 4.37 mmol, 250.01 μL, 30 eq) and NaBH₃CN (27.47 mg, 437.14 μmol, 3 eq) was added into the mixture and stirred at 25° C. for 1 hour. The reaction mixture was filtered and then diluted with aq. NaHCO₃ 4 mL and extracted with EtOAc 9 mL (3 mL×3). The combined organic layers were washed with Brine 6 mL (2 mL×3), dried over Na₂SO₄, and concentrated under reduced pressure to give a residue. The residue was purified by column chromatography (SiO2, Petroleum ether/Ethyl acetate=1/1 to 0/1, Ethyl acetate/MeOH=10/1 to 0/1) to give 4-hexyldecyl 8-[4-(dimethylamino)butyl-[8-(4-hexyldecoxy)-8-oxo-octyl]amino]octanoate (24 mg, 28.25 μmol, 19.39% yield, 100% purity) as a colorless oil.

¹H NMR (400 MHz, CDCl₃), 4.04 (t, J=6.8 Hz, 4H), 2.49-2.55 (m, 6H), 2.31-2.37 (m, 2H), 2.28-2.30 (m, 10H), 1.53-1.64 (m, 16H), 1.24-1.32 (m, 58H), 0.89 (t, J=7.2 Hz, 12H).

LCMS: (M+H⁺): 849.6 @ 3.694 min.

4.3. Synthesis of compound 2131

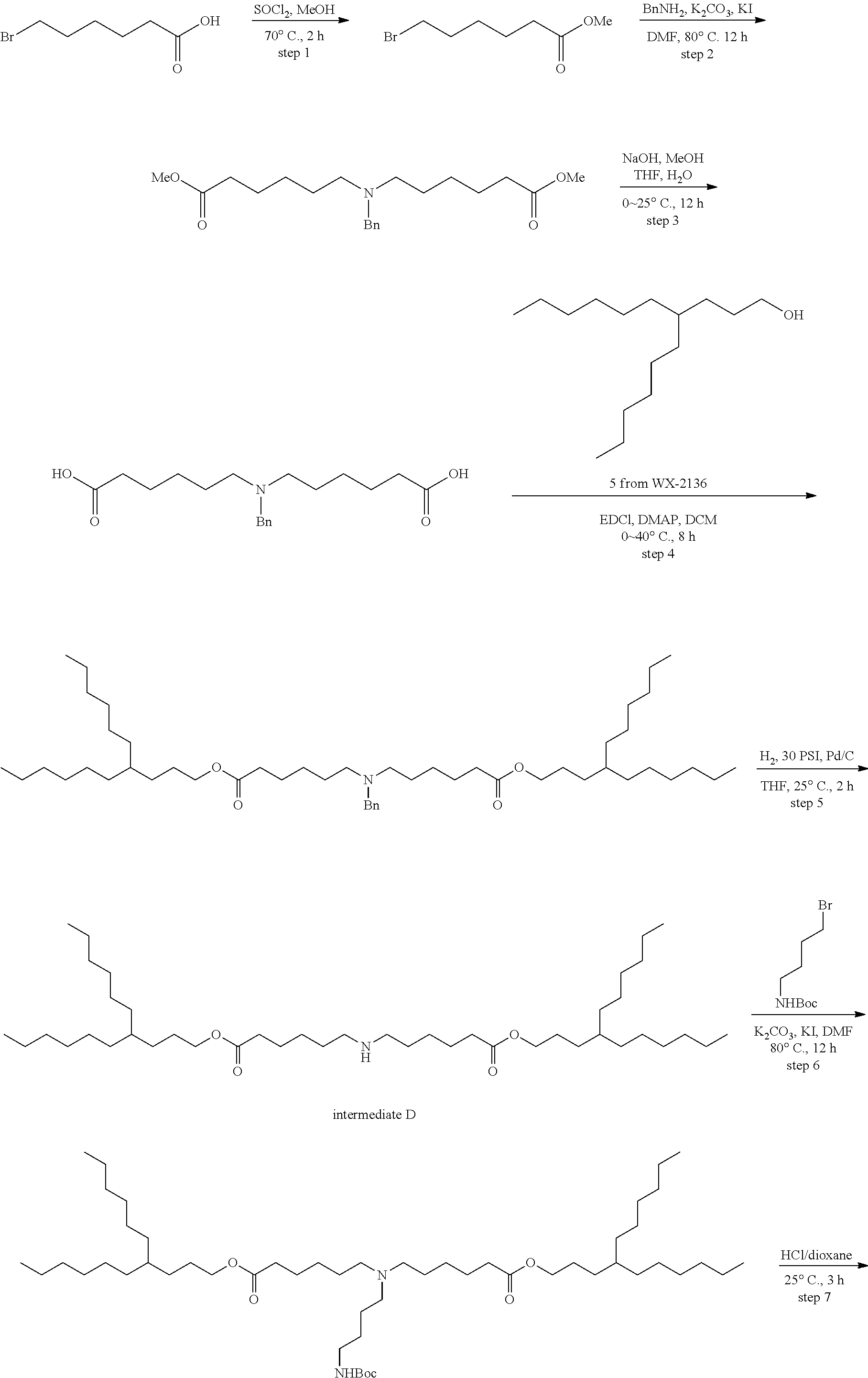

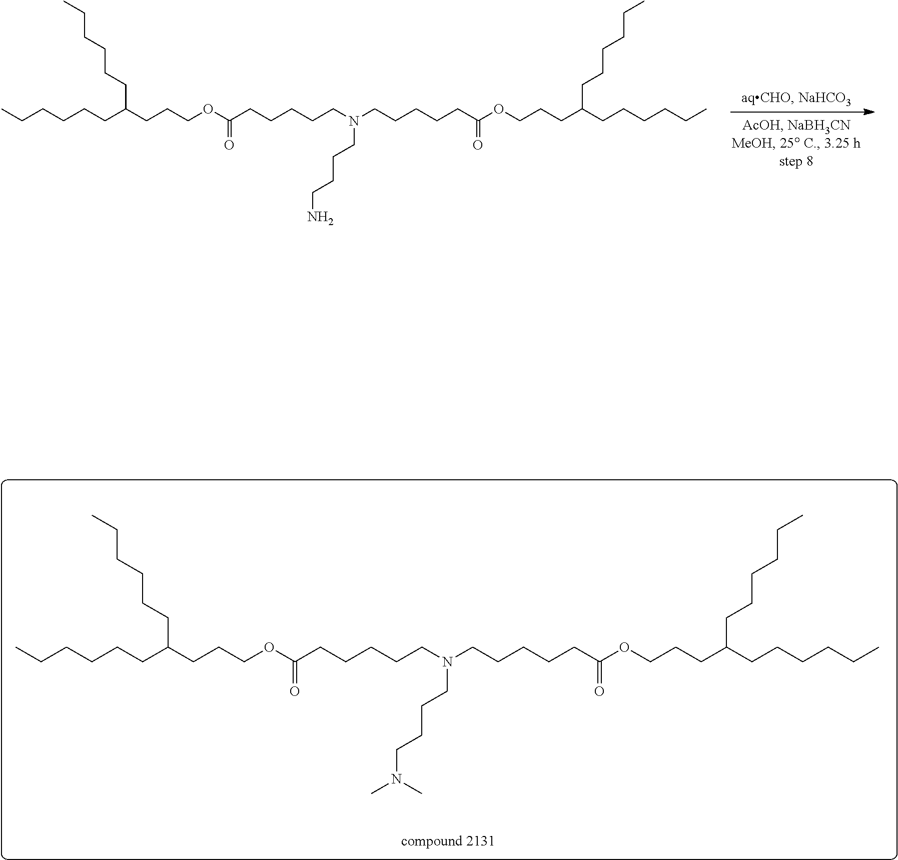

### Step 1:

To a solution of 6-bromohexanoic acid (10 g, 51.27 mmol, 1 eq) in MeOH (200 mL) was added SOCl₂ (12.20 g, 102.54 mmol, 7.44 mL, 2 eq). The mixture was stirred at 70° C. for 2 hours. The reaction mixture was diluted with H₂O 200 mL and washed with PE 600 mL (200 mL×3), extracted with EtOAc 600 mL (200 mL×3). The combined EtOAc layers were dried over Na₂SO₄, filtered and concentrated under reduced pressure to give compound methyl 6-bromohexanoate (10 g, 47.83 mmol, 93.29% yield) as white solid.

### Step 2:

To a solution of BnNH₂ (1.28 g, 11.96 mmol, 1.30 mL, 1 eq) in DMF (50 mL) was added K₂CO₃ (8.26 g, 59.79 mmol, 5 eq) and KI (4.96 g, 29.89 mmol, 2.5 eq), then a solution of methyl 6-bromohexanoate (5 g, 23.91 mmol, 2 eq) in DMF (20 mL) was added to the mixture and stirred at 80° C. for 12 hours. The reaction mixture was filtered and the filtrate was diluted with EtOAc 200 mL and washed with water 600 mL (200 mL×3) and brine 400 mL (200 mL×2). The combined organic layers was dried with anhydrous Na₂SO₄, filtered and concentrated under reduced pressure to give a residue. The residue was purified by column chromatography (SiO₂, Petroleum ether/Ethyl acetate=99/1 to 91/9) to give the compound methyl 6-[benzyl-(6-methoxy-6-oxo-hexyl) amino]hexanoate (8.5 g, 23.38 mmol, 97.78% yield) as white solid.

¹H NMR (400 MHz, CDCl₃), 7.24-7.28 (m, 5H), 3.64 (s, 6H), 3.50 (s, 2H), 2.36 (t, J=7.2 Hz, 4H), 2.26 (t, J=7.6 Hz, 4H), 1.55-1.60 (m, 4H), 1.35-1.48 (m, 4H), 1.24-1.35 (m, 4H).

LCMS: (M+H⁺): 364.1.

### Step 3:

To a solution of methyl 6-[benzyl-(6-methoxy-6-oxo-hexyl)amino]hexanoate (5 g, 13.76 mmol, 1 eq) in MeOH (20 mL), THE (6 mL) was added NaOH (1.88 g, 46.91 mmol, 3.41 eq) in H₂O (10 mL) at 0° C. The mixture was stirred at 25° C. for 12 hours. The reaction mixture was diluted with H₂O 100 mL and extracted with EtOAc 600 mL (200 mL×3). The aqueous phase was freeze-dried after adjusting pH=7 with 1M HCl aqueous. The crude product was triturated with EtOH (100 mL) at 25° C. for 2 hours, then filtered and the filtrate was concentrated under reduced pressure to give the compound 6-[benzyl(5-carboxypentyl)amino]hexanoic acid (4.5 g, 13.42 mmol, 97.53% yield) as white solid.

¹H NMR (400 MHz, DMSO), 7.18-7.28 (m, 5H), 3.46 (s, 2H), 2.29 (t, J=6.8 Hz, 4H), 2.06 (t, J=7.2 Hz, 4H), 1.30-1.50 (m, 8H), 1.15-1.25 (m, 4H).

### Step 4:

A mixture of 6-[benzyl(5-carboxypentyl)amino]hexanoic acid (1 g, 2.98 mmol, 1 eq) in DCM (10 mL) was added DMAP (182.10 mg, 1.49 mmol, 0.5 eq), 4-hexyldecan-1-ol (1.48 g, 6.11 mmol, 2.05 eq), EDCI (1.71 g, 8.94 mmol, 3 eq) at 0° C. and was degassed and purged with N₂ for 3 times. The mixture was stirred at 40° C. for 8 hours under N₂ atmosphere. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=1/0 to 91/9) to give compound 4-hexyldecyl 6-[benzyl-[6-(4-hexyldecoxy)-6-oxo-hexyl]amino] hexanoate (0.96 g, 1.22 mmol, 41.06% yield) as yellow oil.

### Step 5:

A solution of 4-hexyldecyl 6-[benzyl-[6-(4-hexyldecoxy)-6-oxo-hexyl]amino]hexanoate (960 mg, 1.22 mmol, 1 eq) and Pd/C (0.6 g, 1.22 mmol, 10% purity, 1.00 eq) in THE (50 mL) was stirred under H₂ (30 psi) at 25° C. for 2 hours. The mixture is filtered and the solvent is removed under reduced pressure to give a residue. The residue was purified by column chromatography (SiO₂, Petroleum ether/Ethyl acetate=1/0 to 3/1) to give compound 4-hexyldecyl 6-[[6-(4-hexyldecoxy)-6-oxo-hexyl]amino]hexanoate (650 mg, 936.38 μmol, 76.50% yield) as brown oil.

¹H NMR (400 MHz, CDCl₃), 7.27-7.30 (m, 5H), 4.03 (t, J=6.8 Hz, 4H), 3.53 (s, 2H), 2.39 (t, J=7.2 Hz, 4H), 2.27 (t, J=7.6 Hz, 4H), 1.50-1.62 (m, 8H), 1.40-1.48 (m, 4H), 1.24-1.35 (m, 50H), 0.89 (t, J=6.8 Hz, 12H).

### Step 6:

To a solution of 4-hexyldecyl 6-[[6-(4-hexyldecoxy)-6-oxo-hexyl]amino]hexanoate (0.3 g, 432.18 μmol, 1 eq) in DMF (3 mL) was added K₂CO₃ (298.65 mg, 2.16 mmol, 5 eq) and KI (143.48 mg, 864.35 μmol, 2 eq), tert-butyl N-(4-bromobutyl)carbamate (435.89 mg, 1.73 mmol, 354.38 μL, 4 eq). The mixture was stirred at 80° C. for 12 hours. The reaction mixture was diluted with H₂O 20 mL and extracted with EtOAc60 mL (20 mL×3). The combined organic layers were dried over Na₂SO₄, filtered and concentrated under reduced pressure to give a residue. The residue was purified by column chromatography (SiO₂, Petroleum ether/Ethyl acetate=1/1 to Y/1) to give compound 4-hexyldecyl 6-[4-(tert-butoxycarbonylamino)butyl-[6-(4-hexyldecoxy)-6-oxo-hexyl]amino]hexanoate (0.25 g, 288.88 μmol, 66.84% yield) as yellow oil.

¹H NMR (400 MHz, CDCl₃), 5.01 (brs, 1H), 4.04 (t, J=6.8 Hz, 4H), 3.05-3.20 (m, 2H), 2.38-2.50 (m, 6H), 2.30 (t, J=7.6 Hz, 4H), 1.57-1.67 (m, 12H), 1.35-1.50 (m, 13H), 1.24-1.30 (m, 50H), 0.89 (t, J=7.2 Hz, 12H). LCMS: (M+H⁺): 865.8.

### Step 7:

A solution of 4-hexyldecyl 6-[4-(tert-butoxycarbonylamino)butyl-[6-(4-hexyldecoxy)-6-oxo-hexyl]amino]hexanoate (0.2 g, 231.11 μmol, 1 eq) in HCl/dioxane (4 M, 4.00 mL, 69.23 eq) was stirred at 25° C. for 3 hours under N₂ atmosphere. The reaction mixture was concentrated under reduced pressure to get compound 4-hexyldecyl 6-[4-aminobutyl-[6-(4-hexyldecoxy)-6-oxo-hexyl]amino]hexanoate (0.1 g, crude) as yellow oil.

### Step 8:

To a solution of 4-hexyldecyl 6-[4-aminobutyl-[6-(4-hexyldecoxy)-6-oxo-hexyl]amino]hexanoate (0.1 g, 130.67 μmol, 1 eq), formaldehyde (1.09 g, 36.30 mmol, 1.00 mL, 277.81 eq) in MeOH (2 mL) was added NaHCO₃ (32.93 mg, 392.01 μmol, 15.25 μL, 3 eq) at 25° C. and stirred at 25° C. for 15 minutes, then AcOH (235.41 mg, 3.92 mmol, 224.20 μL, 30 eq) and NaBH₃CN (24.63 mg, 392.01 μmol, 3 eq) were added to the mixture at 25° C. The resulting mixture was stirred at 25° C. for 3 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Ethyl acetate:MeOH=1/0 to 10/1) to give compound 4-hexyldecyl 6-[4-(dimethylamino)butyl-[6-(4-hexyldecoxy)-6-oxo-hexyl]amino]hexanoate (38 mg, 47.90 μmol, 36.66% yield, 100% purity) as yellow oil.

¹H NMR (400 MHz, CDCl₃), 4.04 (t, J=6.8 Hz, 4H), 2.35-2.49 (m, 6H), 2.28-2.32 (m, 6H), 2.23 (s, 6H), 1.55-1.65 (m, 8H), 1.43-1.45 (m, 8H), 1.24-1.30 (m, 50H), 0.89 (t, J=6.8 Hz, 12H). LCMS: (M+H⁺): 793.6.

4.4. Synthesis of compound 2132

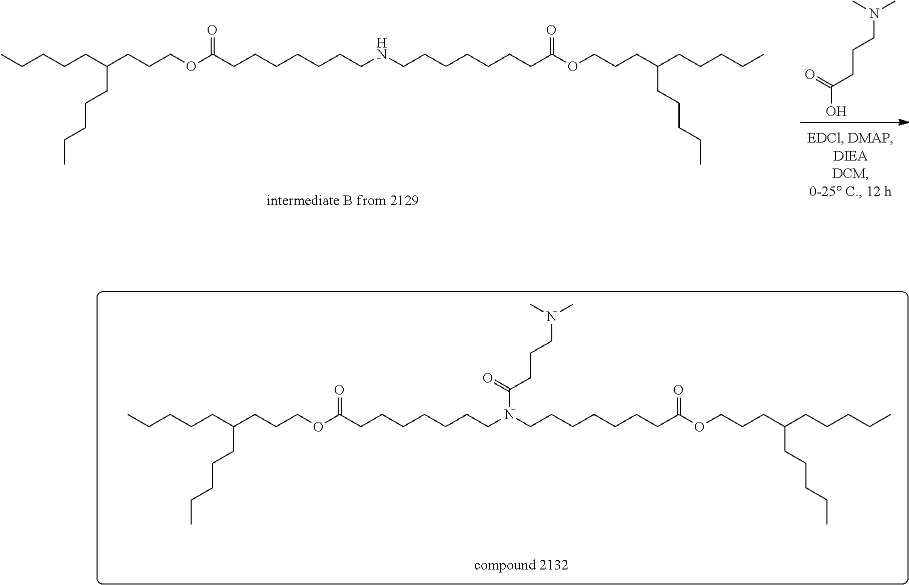

To a solution of 4-pentylnonyl 8-[[8-oxo-8-(4-pentylnonoxy)octyl]amino]octanoate (160 mg, 230.49 μmol, 1 eq) and 4-(dimethylamino)butanoic acid (60.47 mg, 360.72 μmol, 1.56 eq, HCl) in DCM (2 mL) was added DMAP (28.16 mg, 230.49 μmol, 1 eq), DIEA (59.58 mg, 460.99 μmol, 80.30 μL, 2 eq) and EDCI (132.56 mg, 691.48 μmol, 3 eq) at 0° C. and stirred at 25° C. for 12 hours. The mixture was concentrated under reduced pressure. The residue was purified by prep-HPLC (column: Phenomenex Luna C18 100×30 mm×5 μm; mobile phase: [water (HCl)-ACN]; B %: 65%-95%, 10 min) and make it free by sat.NaHCO₃ (5 mL), extracted with EtOAc (5 mL×3), organic layer was washed with brine (5 mL×2), dried over Na₂SO₄, filtered and the filtrate was concentrated under reduced pressure to give the 4-pentylnonyl 8-[4-(dimethylamino) butanoyl-[8-oxo-8-(4-pentylnonoxy)octyl]amino]octanoate (21 mg, 26.01 μmol, 11.29% yield, 100% purity) as a white solid.

¹H NMR (400 MHz, CDCl₃), 4.04 (t, J=6.8 Hz, 4H), 3.27 (t, J=8.0 Hz, 2H), 3.20 (t, J=7.6 Hz, 2H), 2.26-2.37 (m, 14H), 1.81-1.87 (m, 2H), 1.57-1.67 (m, 8H), 1.48-1.53 (m, 4H), 1.24-1.31 (m, 50H), 0.89 (t, J=6.8 Hz, 12H). LCMS: (M+H⁺): 807.6 @ 3.942 min.

### 4.5. Synthesis of Compound 2133

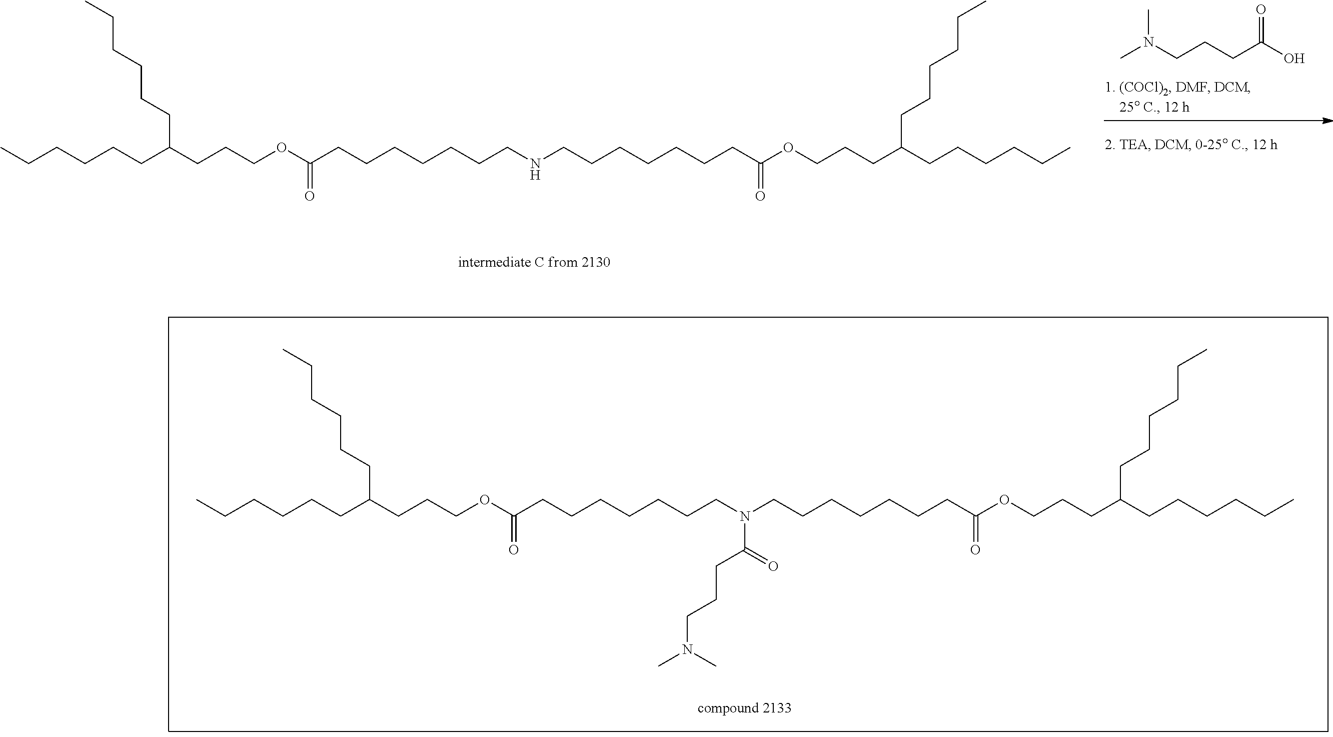

To a solution of 4-(dimethylamino)butanoic acid (100 mg, 596.54 μmol, 1 eq, HCl) in CH₂Cl₂ (5 mL) was added oxalyl dichloride (227.15 mg, 1.79 mmol, 156.65 μL, 3 eq) and DMF (1 mL). The mixture was stirred at 25° C. for 12 hours. The reaction mixture was concentrated under reduced pressure to give 4-(dimethylamino)butanoyl chloride (110 mg, crude, HCl) as a white solid. The 4-(dimethylamino)butanoyl chloride (49.60 mg, 266.57 μmol, 2 eq, HCl) was dropwise added to a solution of 4-hexyldecyl 8-[[8-(4-hexyldecoxy)-8-oxo-octyl]amino]octanoate (100 mg, 133.29 μmol, 1 eq) and TEA (40.46 mg, 399.86 μmol, 55.66 μL, 3 eq) in DCM (2 mL) at 0° C. and stirred at 25° C. for 12 hours. The reaction mixture was diluted with H₂O 6 mL and extracted with EtOAc 9 mL (3 mL×3). The combined organic layers were washed with Brine 6 mL (2 ml×3), dried over Na₂SO₄, filtered and concentrated under reduced pressure to give a residue. The residue was purified by column chromatography (SiO₂, Petroleum ether/Ethyl acetate=5/1 to 0/1, Ethyl acetate/Methanol=10/1 to 8/1) to give 4-hexyldecyl 8-[4-(dimethylamino)butanoyl-[8-(4-hexyldecoxy)-8-oxo-octyl]amino]octanoate (36 mg, 41.11 μmol, 30.84% yield, 98.6% purity) as a white solid.

¹H NMR (400 MHz, CDCl₃), 4.02-4.06 (m, 4H), 3.28 (t, J=7.6 Hz, 2H), 3.20 (t, J=6.8 Hz, 2H), 2.30-2.34 (m, 8H), 2.24 (s, 6H), 1.79-1.86 (m, 2H), 1.61-1.62 (m, 8H), 1.48-1.52 (m, 4H), 1.24-1.31 (m, 58H), 0.89 (t, J=6.8 Hz, 12H). LCMS: (M+H⁺): 863.7 @ 4.085 min.

### 4.6. Synthesis of Compound 2134

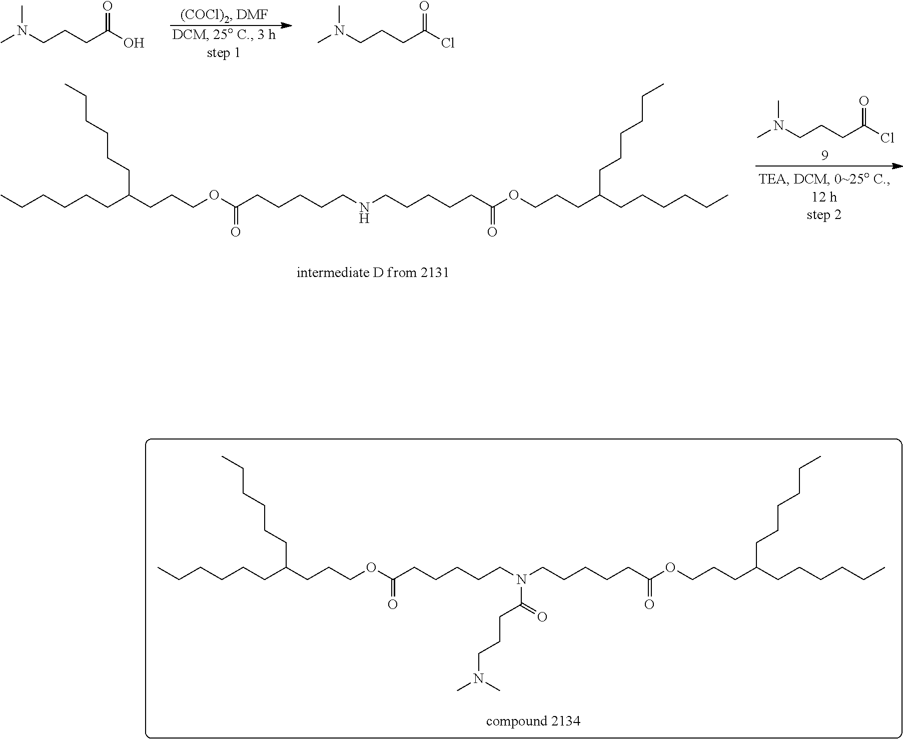

### Step 1:

To a solution of 4-(dimethylamino)butanoic acid; hydrochloride (0.4 g, 2.39 mmol, 1 eq) and oxalyl dichloride (1.77 g, 13.97 mmol, 1.22 mL, 5 eq) in DCM (5 mL) was added two drops of DMF (20.42 mg, 279.36 μmol, 21.49 μL, 0.1 eq), and stirred at 25° C. for 3 hours under N₂ atmosphere. The reaction mixture was concentrated under reduced pressure to give a compound 4-(dimethylamino)butanoyl chloride (0.4 g, crude) as yellow oil.

### Step 2:

To a solution of 4-hexyldecyl 6-[[6-(4-hexyldecoxy)-6-oxo-hexyl]amino]hexanoate (0.4 g, 576.23 μmol, 1 eq), 4-(dimethylamino)butanoyl chloride (344.86 mg, 2.30 mmol, 4 eq) in DCM (3 mL) was added TEA (174.93 mg, 1.73 mmol, 240.61 μL, 3 eq) at 0° C. The mixture was stirred at 25° C. for 12 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Ethyl acetate:MeOH=1/0 to 3/1) to give compound 4-hexyldecyl 6-[4-(dimethylamino) butanoyl-[6-(4-hexyldecoxy)-6-oxo-hexyl]amino]hexanoate (174 mg, 215.53 μmol, 37.40% yield, 100% purity) as yellow oil.

¹H NMR (400 MHz, CDCl₃), 4.01-4.07 (m, 4H), 3.20-3.31 (m, 4H), 2.28-2.35 (m, 8H), 2.24 (S, 6H), 1.80-1.84 (m, 2H), 1.57-1.70 (m, 12H), 1.24-1.34 (m, 50H), 0.89 (t, J=6.8 Hz, 12H).

LCMS: (M+H⁺): 807.6 @ 3.898 min.

### 4.7. Synthesis of Compound 2135

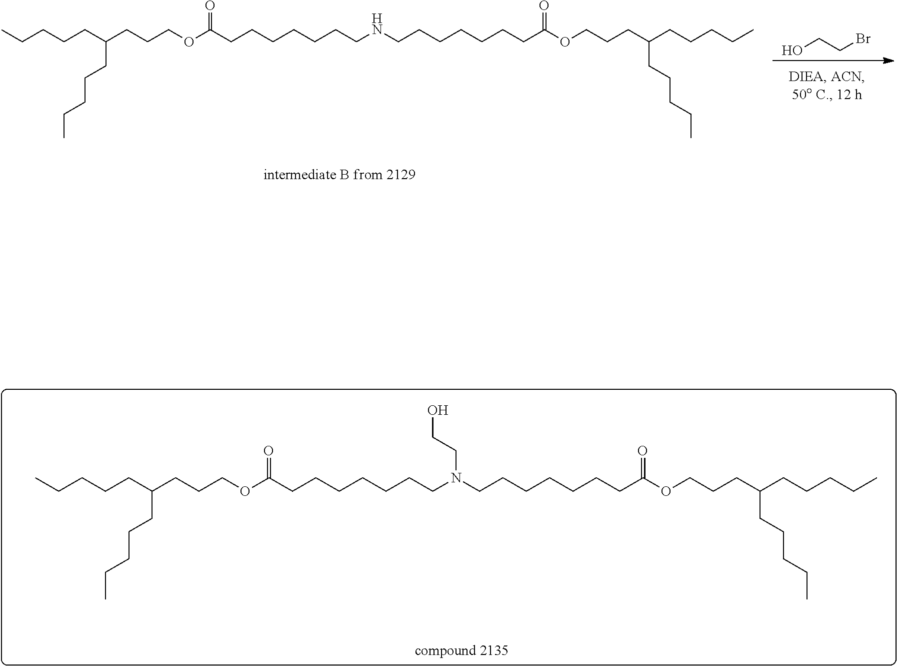

To a solution of 4-pentylnonyl 8-[[8-oxo-8-(4-pentylnonoxy)octyl]amino]octanoate (250 mg, 360.15 μmol, 1 eq) in ACN (5 mL) was added DIEA (93.09 mg, 720.29 μmol, 125.46 μL, 2 eq) and 2-bromoethanol (90.01 mg, 720.29 μmol, 51.14 μL, 2 eq). The mixture was stirred at 50° C. for 12 hours. The reaction mixture was poured in H₂O (10 ml) and extracted with EtOAc 45 mL (15 mL×3). The combined organic layers were washed with brine 50 mL (25 mL×3), dried over Na₂SO₄, filtered and concentrated under reduced pressure to give a residue. The residue was purified by prep-HPLC (column: Phenomenex Luna C18 100×30 mm×5 μm; mobile phase: [water(HCl)-MEOH]; B %: 70%-90%, 10 minutes). The mobile phase was adjusted pH to 7 with NaHCO₃ saturated solution and extracted with EtOAc 15 mL (5 mL×3). The combined organic layers was dried over Na₂SO₄, filtered and concentrated under reduced pressure to give compound 4-pentylnonyl 8-[2-hydroxyethyl-[8-oxo-8-(4-pentylnonoxy)octyl]amino]octanoate (40 mg, 54.18 μmol, 15.00% yield, 100% purity) as a yellow oil.

¹H NMR (400 MHz, CDCl₃), 4.05 (t, J=6.8 Hz 4H), 3.55 (t, J=4.8 Hz, 2H), 2.59 (t, J=5.2 Hz, 2H), 2.46 (t, J=7.2 Hz, 4H), 2.30 (t, J=7.6 Hz, 4H), 1.57-1.64 (m, 8H), 1.40-1.50 (m, 4H), 1.20-1.35 (m, 50H), 0.89 (t, J=6.8 Hz, 12H). LCMS: (M+W): 738.7 @ 3.257 min.

### 4.8. Synthesis of Compound 2136

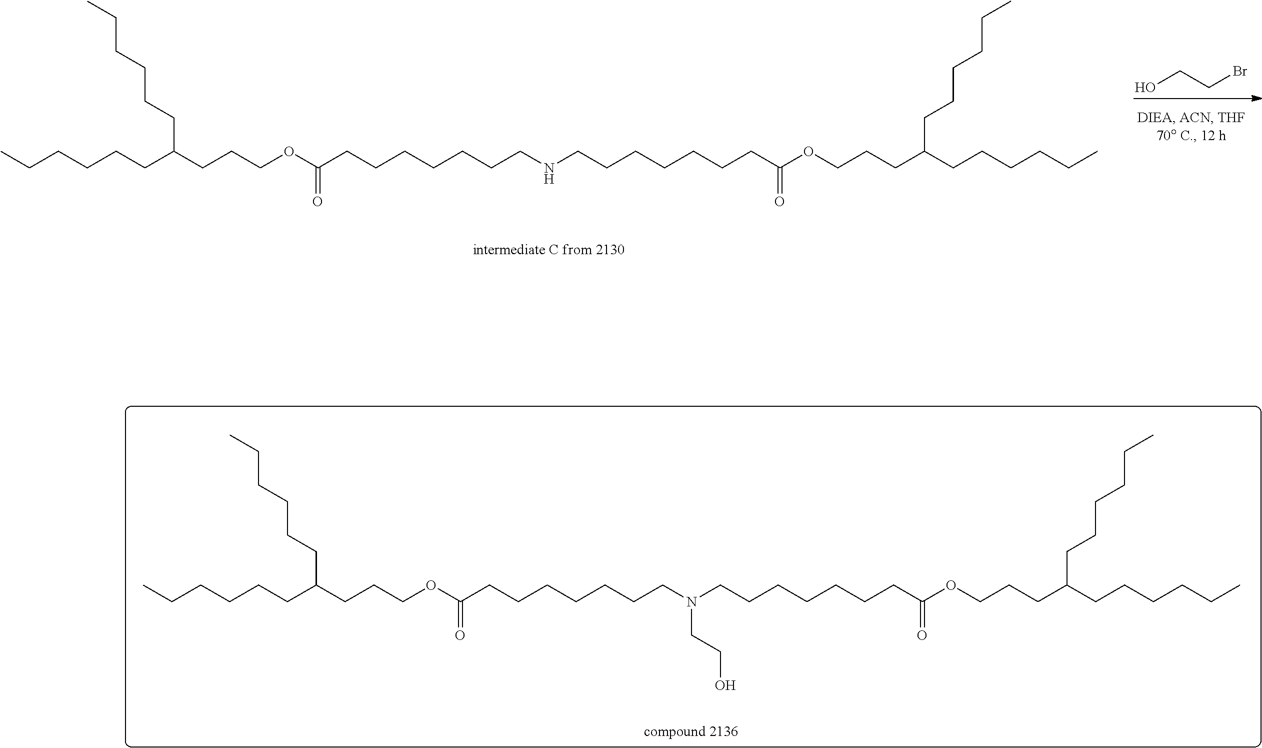

To a solution of 4-hexyldecyl 8-[[8-(4-hexyldecoxy)-8-oxo-octyl]amino]octanoate (150 mg, 199.93 μmol, 1 eq) in ACN (0.5 mL) and THE (1 mL) was added DIEA (51.68 mg, 399.86 μmol, 69.65 μL, 2 eq) and then a solution of 2-bromoethanol (64.55 mg, 399.86 μmol, 36.67 μL, 2 eq, HCl) in THE (0.5 mL) was added into the mixture. The mixture was stirred at 70° C. for 12 hours. The reaction mixture was diluted with H₂O (6 mL) and extracted with PE 6 mL (2 mL×3). The combined organic layers were washed with brine 3 mL (1 mL×3), dried over Na₂SO₄, filtered and concentrated under reduced pressure to give a residue. The residue was purified by column chromatography (SiO₂, Petroleum ether/Ethyl acetate=4/1 to 0/1) to give 4-hexyldecyl 8-[[8-(4-hexyldecoxy)-8-oxo-octyl]-(2-hydroxyethyl)amino]octanoate (60 mg, 75.15 μmol, 37.59% yield, 99.495% purity) as colorless oil.

¹H NMR (400 MHz, CDCl₃), 4.05 (t, J=6.8 Hz 4H), 3.53 (t, J=5.2 Hz, 2H), 2.58 (t, J=4.8 Hz, 2H), 2.45 (t, J=7.2 Hz, 4H), 2.29 (t, J=7.6 Hz, 4H), 1.5-1.62 (m, 8H), 1.42-1.45 (m, 4H), 1.24-1.30 (m, 58H), 0.89 (t, J=6.8 Hz, 12H). LCMS: (M+H⁺): 794.6 @ 4.085 min.

### 4.9. Synthesis of Compound 2137

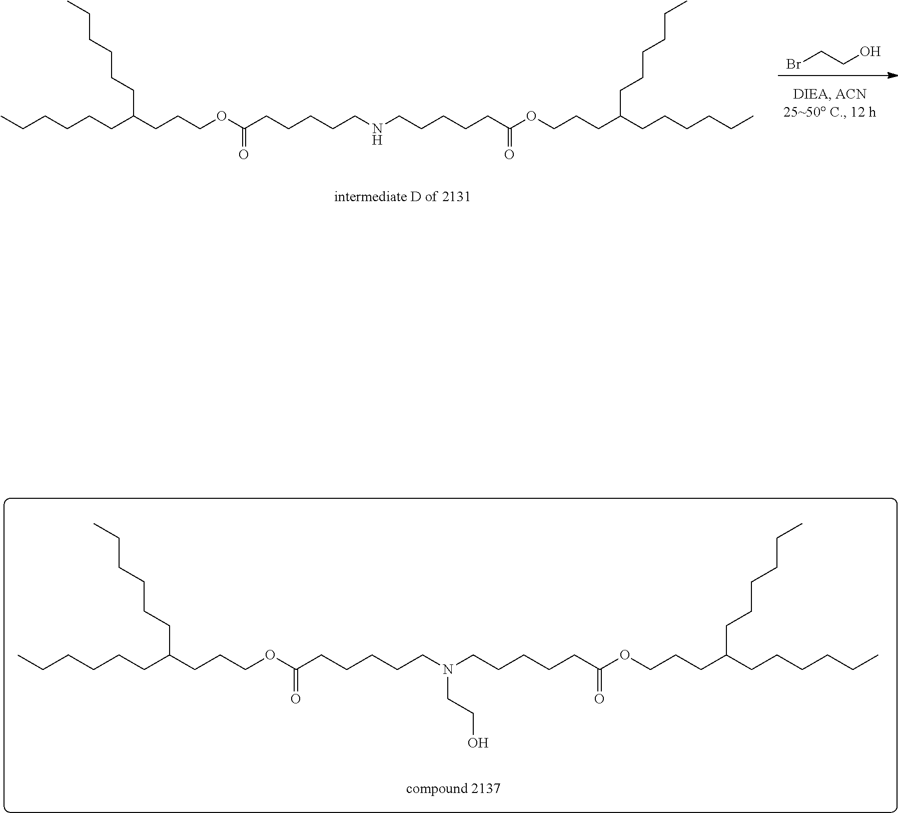

To a solution of 4-hexyldecyl 6-[[6-(4-hexyldecoxy)-6-oxo-hexyl]amino]hexanoate (0.2 g, 288.12 μmol, 1 eq) in ACN (3 mL) was added DIEA (74.47 mg, 576.23 μmol, 100.37 μL, 2 eq), 2-bromoethanol (72.01 mg, 576.23 μmol, 40.91 μL, 2 eq) at 25° C. The mixture was stirred at 50° C. for 12 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Ethyl acetate:MeOH=1/0 to 10/1) to give the compound 4-hexyldecyl6-[[6-(4-hexyldecoxy)-6-oxo-hexyl]-(2-hydroxyethyl)amino]hexanoate (28 mg, 37.93 μmol, 13.16% yield) as yellow oil.

¹H NMR (400 MHz, CDCl₃), 4.04 (t, J=6.8 Hz 4H), 3.54 (t, J=5.2 Hz, 2H), 2.59 (t, J=5.2 Hz, 2H), 2.47 (t, J=7.6 Hz, 4H), 2.3 (t, J=7.6 Hz, 4H), 1.66-1.70 (m, 4H), 1.55-1.57 (m, 4H), 1.43-1.49 (m, 4H), 1.24-1.33 (m, 50H), 0.89 (t, J=6.8 Hz, 12H).

LCMS: (M+H⁺): 738.5 @ 3.419 min.

### 4.10: Synthesis of Compound 2138

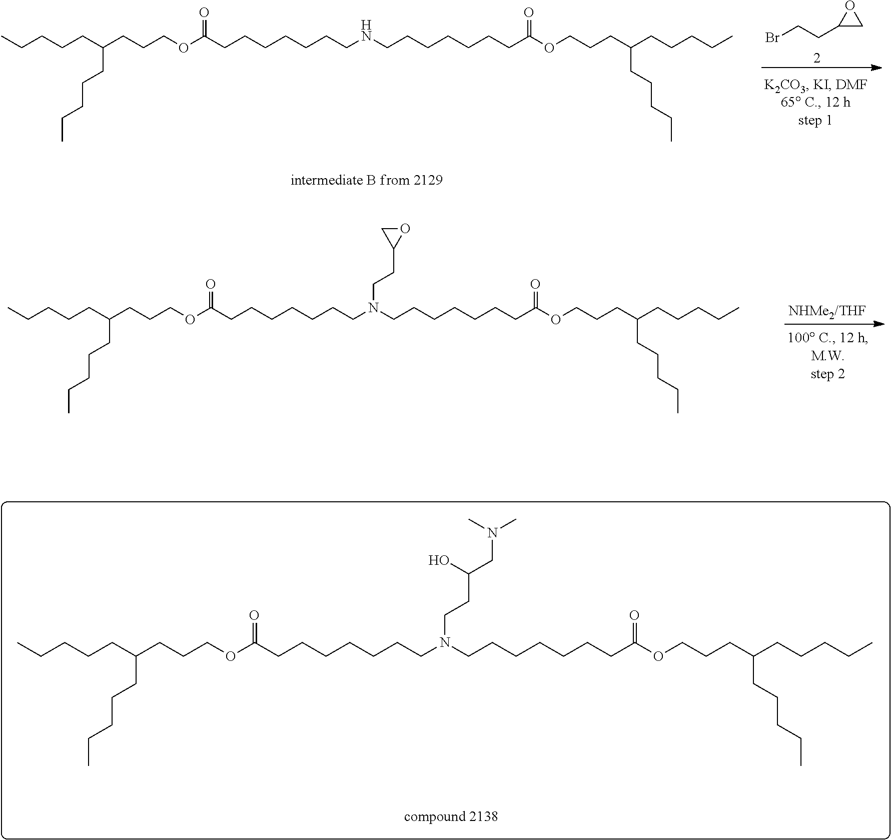

### Step 1:

To a solution of 4-pentylnonyl 8-[[8-oxo-8-(4-pentylnonoxy)octyl]amino]octanoate (500 mg, 720.29 μmol, 1 eq) in DMF (5 mL) was added K₂CO₃ (497.76 mg, 3.60 mmol, 40.10 μL, 5 eq), KI (239.14 mg, 1.44 mmol, 2 eq) and 2-(2-bromoethyl)oxirane (435.06 mg, 2.88 mmol, 58.49 μL, 4 eq). The mixture was stirred at 65° C. for 12 hours. The mixture was filtered and the filtrate was added into H₂O (5 mL), extracted with EtOAc (5 mL×3). The organic layer was washed with brine 10 mL (5 mL×2), dried over Na₂SO₄, filtered and the filtrate was concentrated under reduced pressure. The residue was purified by column chromatography (SiO₂, Petroleum ether/Ethyl acetate=10/1 to 0/1) to give a compound 4-pentylnonyl 8-[2-(oxiran-2-yl)ethyl-[8-oxo-8-(4-pentylnonoxy)octyl]amino]octanoate (400 mg, 523.39 μmol, 72.66% yield, − purity) as colorless oil.

¹H NMR (400 MHz, CDCl₃), 4.05 (t, J=6.4 Hz, 4H), 2.95-2.98 (m, 1H), 2.77 (t, J=4.8 Hz, 1H), 2.55-2.65 (m, 1H), 2.46-2.51 (m, 1H), 2.39-2.40 (m, 3H), 2.30 (t, J=7.6 Hz, 4H), 1.59-1.64 (m, 12H), 1.40-1.44 (m, 4H), 1.24-1.32 (m, 50H), 0.89 (t, J=6.8 Hz, 12H).

(M+H⁺): 764.8.

### Step 2:

A solution of 4-pentylnonyl 8-[2-(oxiran-2-yl)ethyl-[8-oxo-8-(4-pentylnonoxy)octyl]amino]octanoate (200 mg, 261.69 μmol, 1 eq) in Me₂NH (2 M, 20.00 mL) in THE was stirred at 100° C. for 12 hours under microwave. The mixture was purified by prep-HPLC (column: Phenomenex Luna C18 100×30 mm×5 μm; mobile phase: [water(HCl)-ACN]; B %: 50%-80%, 10 minutes) to give a compound 4-pentylnonyl 8-[[4-(dimethylamino)-3-hydroxy-butyl]-[8-oxo-8-(4-pentylnonoxy)octyl]amino]octanoate (52 mg, 64.25 μmol, 26.00% yield) as yellow oil.

¹H NMR (400 MHz, CDCl₃), 4.06 (t, J=6.8 Hz 4H), 3.82-3.88 (m, 1H), 2.50-2.75 (m, 4H), 2.20-2.45 (m, 14H), 1.55-1.68 (m, 10H), 1.43-1.52 (m, 4H), 1.20-1.35 (m, 50H), 0.89 (t, J=6.8 Hz, 12H). LCMS: (M+H⁺): 809.6 @ 3.526 min

### 4.11: Synthesis of Compound 2139

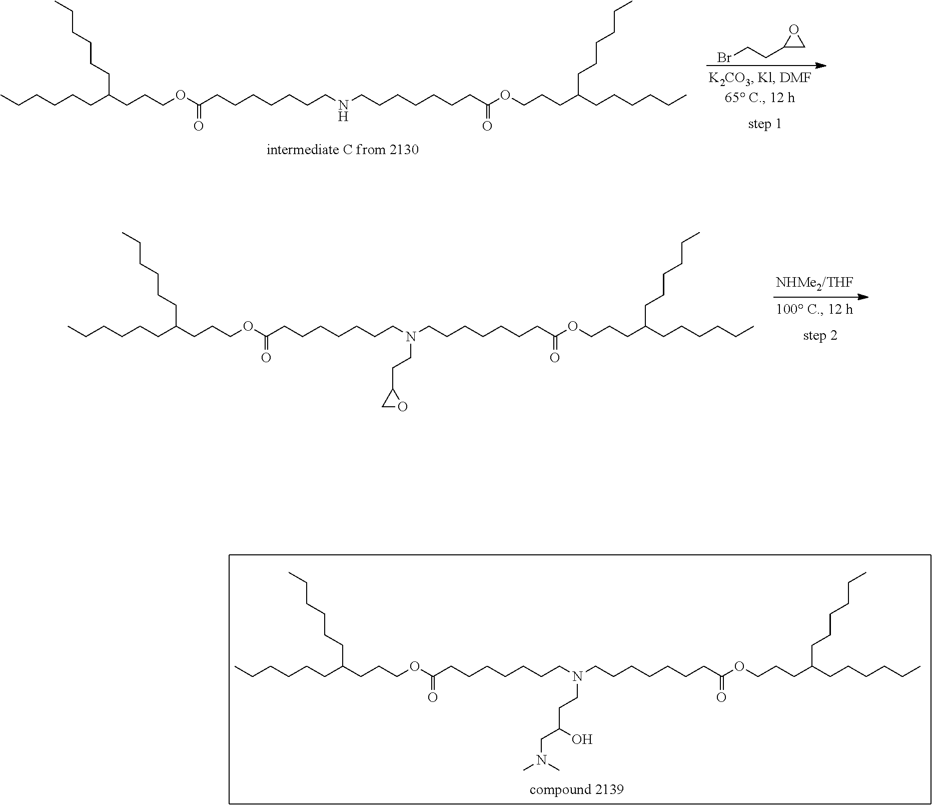

### Step 1:

To a solution of 4-hexyldecyl 8-[[8-(4-hexyldecoxy)-8-oxo-octyl]amino]octanoate (400 mg, 533.14 μmol, 1 eq) in DMF (2 mL) was added K₂CO₃ (368.42 mg, 2.67 mmol, 5 eq) and KI (177.01 mg, 1.07 mmol, 2 eq) then 2-(2-bromoethyl)oxirane (322.02 mg, 2.13 mmol, 4 eq) was added into the mixture. The mixture was stirred at 65° C. for 12 hours. The reaction mixture was filtered and the filtrate was added into H₂O 6 mL and extracted with EtOAc 15 mL (5 mL×3). The combined organic layers were washed with Brine 12 mL (4 mL×3), dried over Na₂SO₄, filtered and concentrated under reduced pressure to give a residue. The residue was purified by column chromatography (SiO₂, Petroleum ether/Ethyl acetate=10/1 to 0/1) to give 4-hexyldecyl 8-[[8-(4-hexyldecoxy)-8-oxo-octyl]-[2-(oxiran-2-yl)ethyl]amino]octanoate (140 mg, 170.66 μmol, 32.01% yield) as a colorless oil.

¹H NMR (400 MHz, CDCl₃), 4.05 (t, J=6.8 Hz, 4H), 2.96-2.98 (m, 1H), 2.77 (t, J=4.8 Hz, 1H), 2.58 (t, J=4.8 Hz, 2H), 2.49 (t, J=4.8 Hz, 1H), 2.37 (t, J=4.8 Hz, 4H), 2.30 (t, J=7.2 Hz, 4H), 1.60-1.62 (m, 10H), 1.38-1.47 (m, 4H), 1.24-1.32 (m, 58H), 0.89 (t, J=6.8 Hz, 12H).

(M+H⁺): 820.8.

### Step 2:

A solution of 4-hexyldecyl 8-[[8-(4-hexyldecoxy)-8-oxo-octyl]-[2-(oxiran-2-yl)ethyl]amino]octanoate (100 mg, 121.90 μmol, 1 eq) in N-methylmethanamine (5.48 g, 121.48 mmol, 6.15 mL, 996.61 eq, THE 2M solution) was stirred at 100° C. for 12 hours. The reaction mixture was diluted with NaHCO₃ 10 mL and extracted with EtOAc 24 mL (8 mL×3). The combined organic layers were washed with Brine 15 mL (5 mL×3), dried over Na₂SO₄, filtered and concentrated under reduced pressure to give a residue. The residue was purified by prep-HPLC (column: Phenomenex Luna C18 100×30 mm×5 μm; mobile phase: [water(HCl)-ACN]; B %: 55%-85%, 10 min) to give 4-hexyldecyl 8-[[4-(dimethylamino)-3-hydroxy-butyl]-[8-(4-hexyldecoxy)-8-oxo-octyl]amino]octanoate (30 mg, 34.66 μmol, 28.44% yield) as yellow oil.

¹H NMR (400 MHz, CDCl₃), 4.05 (t, J=6.4 Hz 4H), 3.82-3.88 (m, 1H), 2.48-2.70 (m, 4H), 2.25-2.45 (m, 14H), 1.52-1.72 (m, 10H), 1.40-1.50 (m, 4H), 1.18-1.35 (m, 58H), 0.89 (t, J=6.8 Hz, 12H). LCMS: (M+H⁺): 865.7 @ 3.699 min.

### 4.12: Synthesis of Compound 2140

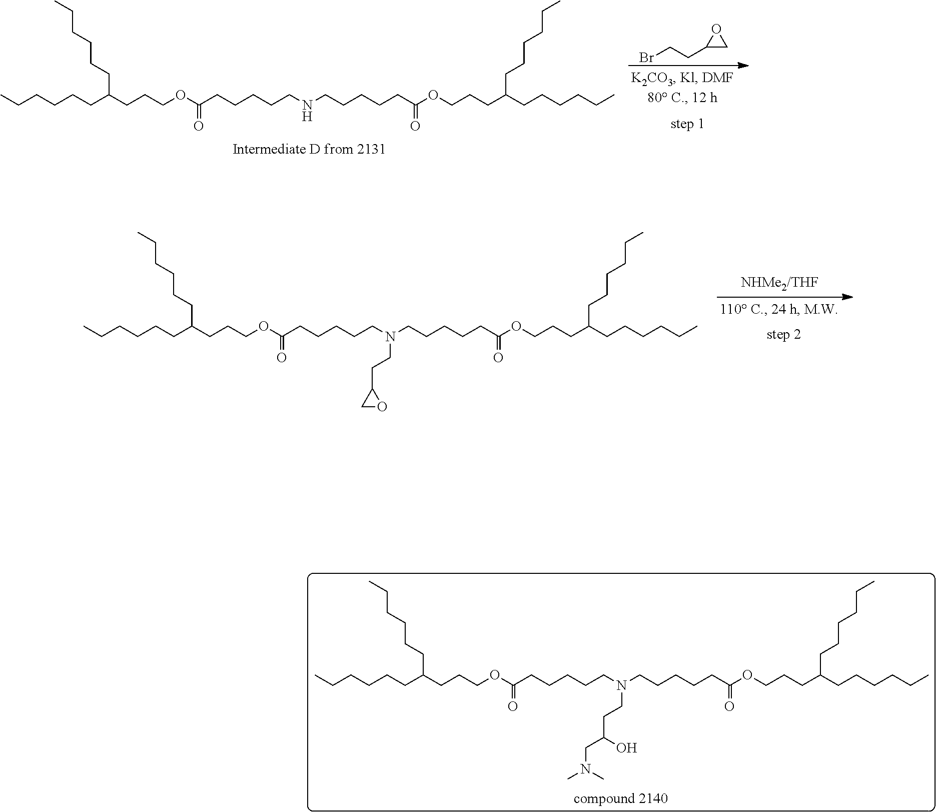

### Step 1:

To a solution of 4-hexyldecyl 6-[[6-(4-hexyldecoxy)-6-oxo-hexyl]amino]hexanoate (1 g, 1.44 mmol, 1 eq) in DMF (10 mL) was added K₂CO₃ (995.52 mg, 7.20 mmol, 5 eq) and KI (478.27 mg, 2.88 mmol, 2 eq), 2-(2-bromoethyl)oxirane (870.12 mg, 5.76 mmol, 354.38 μL, 4 eq). The mixture was stirred at 80° C. for 12 hours. TLC showed 4-hexyldecyl 6-[[6-(4-hexyldecoxy)-6-oxo-hexyl]amino]hexanoate was consumed completely and one new spot formed. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give a compound 4-hexyldecyl 6-[[6-(4-hexyldecoxy)-6-oxo-hexyl]-[2-(oxiran-2-yl)ethyl]amino]hexanoate (0.5 g, 654.23 μmol, 45.41% yield) as yellow oil.

### Step 2:

A mixture of 4-hexyldecyl 6-[[6-(4-hexyldecoxy)-6-oxo-hexyl]-[2-(oxiran-2-yl)ethyl]amino]hexanoate (0.2 g, 261.69 μmol, 1 eq) in Me₂NH (1 M, 261.69 μL, 1 eq) were taken up into a microwave tube. The sealed tube was heated at 110° C. for 24 hours under microwave. TLC showed 4-hexyldecyl 6-[[6-(4-hexyldecoxy)-6-oxo-hexyl]-[2-(oxiran-2-yl)ethyl]amino]hexanoate was remained and one new spot formed. The combined organic phase was diluted with EtOAc 20 mL and washed with water 60 mL (20 mL×3) and brine 40 mL (20 mL×2), dried with anhydrous Na₂SO₄, filtered and concentrated under reduced pressure to give a residue. The residue was purified by prep-HPLC (column: Phenomenex Luna C18 100×30 mm×5 μm; mobile phase: [water(HCl)-ACN]; B %: 45%-75%, 10 min) to give a compound 4-hexyldecyl 6-[[4-(dimethylamino)-3-hydroxy-butyl]-[6-(4-hexyldecoxy)-6-oxo-hexyl]amino]hexanoate (42 mg, 51.89 μmol, 19.83% yield, 100% purity) as yellow oil.

¹H NMR (400 MHz, CDCl₃), 4.05 (t, J=6.8 Hz 4H), 3.65-3.88 (m, 1H), 2.25-2.61 (m, 18H), 1.52-1.70 (m, 10H), 1.42-1.50 (m, 4H), 1.20-1.35 (m, 50H), 0.89 (t, J=6.8 Hz, 12H).

LCMS: (M+H⁺): 809.6 @ 3.602 min.

### 4.13: Synthesis of Compound 2141

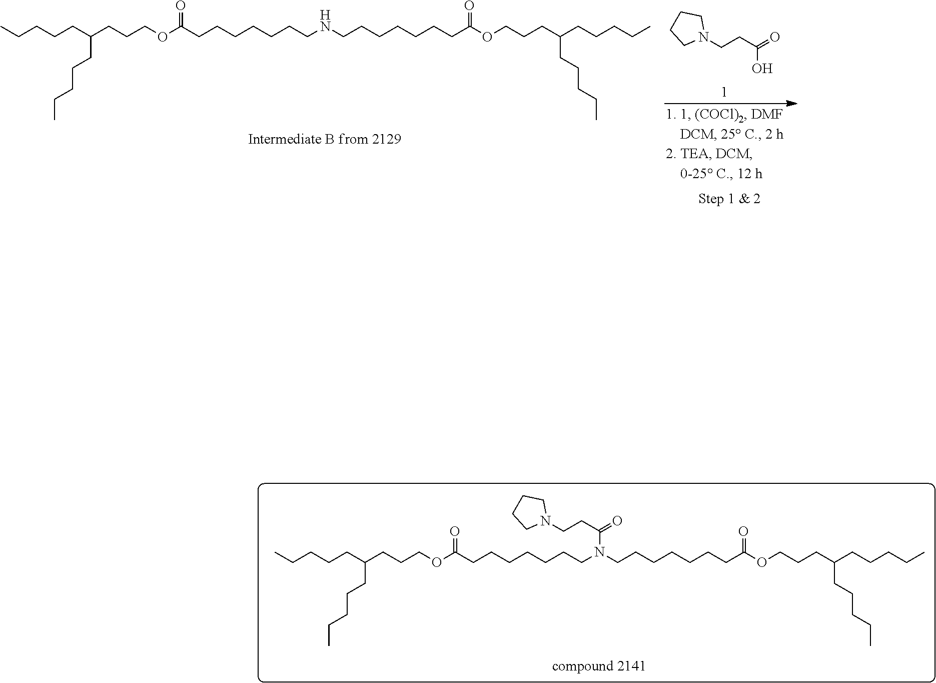

### Step 1:

To a solution of 3-pyrrolidin-1-ylpropanoic acid (100 mg, 698.41 μmol, 1 eq) in DCM (5 mL) was added (COCl)₂ (443.24 mg, 3.49 mmol, 305.69 μL, 5 eq) and DMF (5.10 mg, 69.84 μmol, 5.37 μL, 0.1 eq), stirred at 25° C. for 2 hours. The mixture was concentrated under reduced pressure to give the compound 3-pyrrolidin-1-ylpropanoyl chloride (112 mg, crude) as a yellow solid. The crude was used directly.

### Step 2:

To a solution of 4-pentylnonyl 8-[[8-oxo-8-(4-pentylnonoxy)octyl]amino]octanoate (100 mg, 144.06 μmol, 1 eq) in DCM (2 mL) was added TEA (43.73 mg, 432.18 μmol, 60.15 μL, 3 eq) and 3-pyrrolidin-1-ylpropanoyl chloride (114.15 mg, 576.24 μmol, 4 eq, HCl) at 0° C., stirred at 25° C. for 12 hours. The mixture was concentrated under reduced pressure to give a residue. The residue was purified by column chromatography (SiO₂, Ethyl acetate/MeOH=50/1 to 1/1) to give the compound 4-pentylnonyl 8-[[8-oxo-8-(4-pentylnonoxy)octyl]-(3-pyrrolidin-1-ylpropanoyl)amino]octanoate (87 mg, 101.94 μmol, 70.76% yield) as colorless oil.

¹H NMR (400 MHz, CDCl₃), 4.03-4.07 (m, 4H), 3.15-3.40 (m, 4H), 2.85 (brs, 2H), 2.59 (brs, 6H), 2.27-2.33 (m, 4H), 1.82 (s, 4H), 1.48-1.62 (m, 12H), 1.24-1.32 (m, 50H), 0.89 (t, J=6.8 Hz, 12H). LCMS: (M+H⁺): 819.6 @ 3.842 min.

### 4.14: Synthesis of Compound 2142

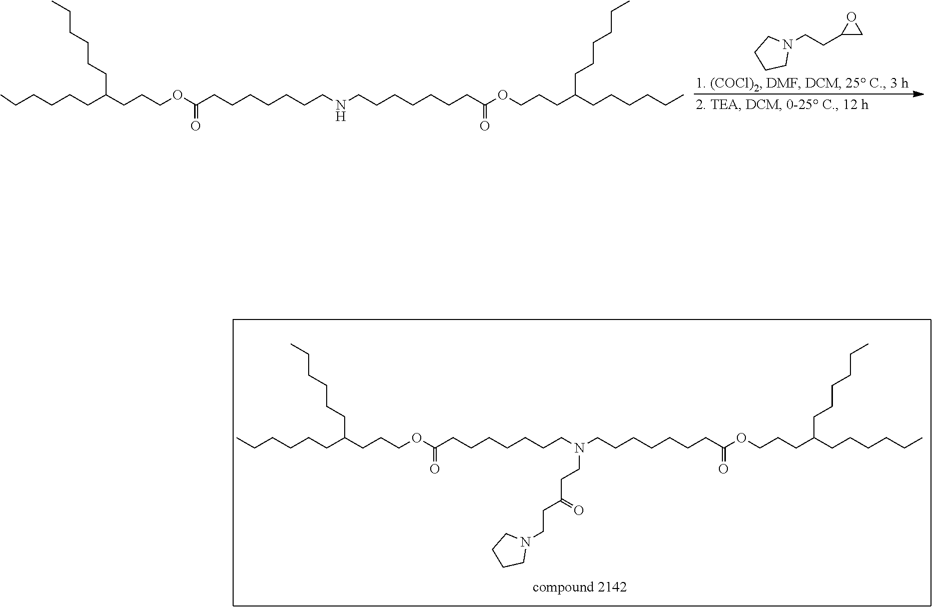

### Step 1:

To a solution of 3-pyrrolidin-1-ylpropanoic acid (450 mg, 3.14 mmol, 1 eq) in CH₂Cl₂ (15 mL) was added (COCl)₂ (1.20 g, 9.43 mmol, 825.32 μL, 3 eq) and DMF (3 mL). The mixture was stirred at 25° C. for 3 hours. The reaction mixture was concentrated under reduced pressure to give 3-pyrrolidin-1-ylpropanoyl chloride (600 mg, crude, HCl) as a yellow solid. The 3-pyrrolidin-1-ylpropanoyl chloride (396.04 mg, 2.00 mmol, 5 eq, HCl) in DCM (4 mL) was dropwise added to a solution of 4-hexyldecyl 8-[[8-(4-hexyldecoxy)-8-oxo-octyl]amino]octanoate (300 mg, 399.86 μmol, 1 eq) and TEA (121.38 mg, 1.20 mmol, 166.96 μL, 3 eq) in DCM (3 mL) added at 0° C. The mixture was stirred at 25° C. for 12 hours. The reaction mixture was diluted with H₂O 5 mL and extracted with EtOAc 12 mL (4 mL×3). The combined organic layers were washed with Brine 9 mL (3 mL×3), dried over Na₂SO₄, filtered and concentrated under reduced pressure to give a residue. The residue was purified by prep-HPLC (column: Phenomenex Luna C18 100×30 mm×5 μm; mobile phase: [water(HCl)-ACN]; B %: 70%-95%, 10 min), then concentrated under reduced pressure to remove ACN, then adjusted pH=8 with aq. NaHCO₃ 20 ml and extracted with EtOAc 30 mL (10 mL×3). The combined organic layers were washed with Brine 24 mL (8 mL×3), dried over Na₂SO₄, and concentrated under reduced pressure to give a residue. The residue was purified by column chromatography (SiO₂, Petroleum ether/Ethyl acetate=1/1 to 0/1, Ethyl acetate/MeOH=10/1 to 0/1) to give 4-hexyldecyl 8-[[8-(4-hexyldecoxy)-8-oxo-octyl]-(3-pyrrolidin-1-ylpropanoyl)amino]octanoate (116 mg, 128.53 μmol, 32.14% yield, 97% purity) as colourless oil.

¹H NMR (400 MHz, CDCl₃), 4.02-4.06 (m, 4H), 3.28 (t, J=7.6 Hz, 2H), 3.21 (t, J=7.6 Hz, 2H), 2.86 (t, J=7.6 Hz, 2H), 2.48-2.68 (m, 6H), 2.27-2.32 (m, 4H), 1.82 (brs, 4H), 1.57-1.65 (m, 8H), 1.45-1.54 (m, 4H), 1.23-1.32 (m, 58H), 0.89 (t, J=6.4 Hz, 12H).

LCMS: (M+H⁺): 875.7 @ 3.678 min.

### 4.15: Synthesis of Compound 2143

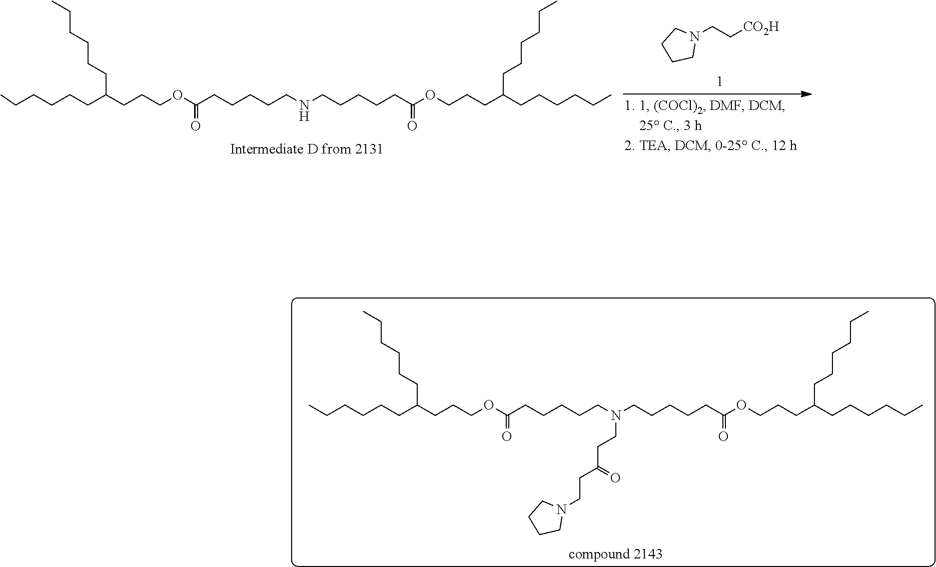

To a solution of 3-pyrrolidin-1-ylpropanoic acid (0.4 g, 2.79 mmol, 1 eq) and oxalyl dichloride (1.77 g, 13.97 mmol, 1.22 mL, 5 eq) in DCM (5 mL) was added two drops of DMF (20.42 mg, 279.36 μmol, 21.49 μL, 0.1 eq). The mixture was stirred at 25° C. for 3 hours under N₂ atmosphere. The reaction mixture was concentrated under reduced pressure to give compound 3-pyrrolidin-1-ylpropanoyl chloride (0.5 g, crude, HCl) as yellow oil. The crude was used directly.

To a solution of 4-hexyldecyl 6-[[6-(4-hexyldecoxy)-6-oxo-hexyl]amino]hexanoate (0.4 g, 576.23 μmol, 1 eq), 3-pyrrolidin-1-ylpropanoyl chloride (372.54 mg, 2.30 mmol, 4 eq) in DCM (3 mL) was added TEA (174.93 mg, 1.73 mmol, 240.61 μL, 3 eq) at 0° C. The mixture was stirred at 25° C. for 12 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Ethyl acetate:MeOH=1/0 to 3/1) to give compound 4-hexyldecyl 6-[[6-(4-hexyldecoxy)-6-oxo-hexyl]-(3-pyrrolidin-1-ylpropanoyl)amino]hexanoate (190 mg, 231.90 μmol, 40.24% yield, 100% purity) as yellow oil.

¹H NMR (400 MHz, CDCl₃), 3.97-4.08 (m, 4H), 3.21-331 (m, 4H), 2.89 (t, J=7.6 Hz, 2H), 2.52-2.65 (m, 6H), 2.25-2.35 (m, 4H), 1.93 (brs, 4H), 1.52-1.67 (m, 12H), 1.15-1.35 (m, 50H), 0.89 (t, J=6.4 Hz, 12H). LCMS: (M+H⁺): 819.6 @ 3.905 min.

### 4.16: Synthesis of Compound 2144

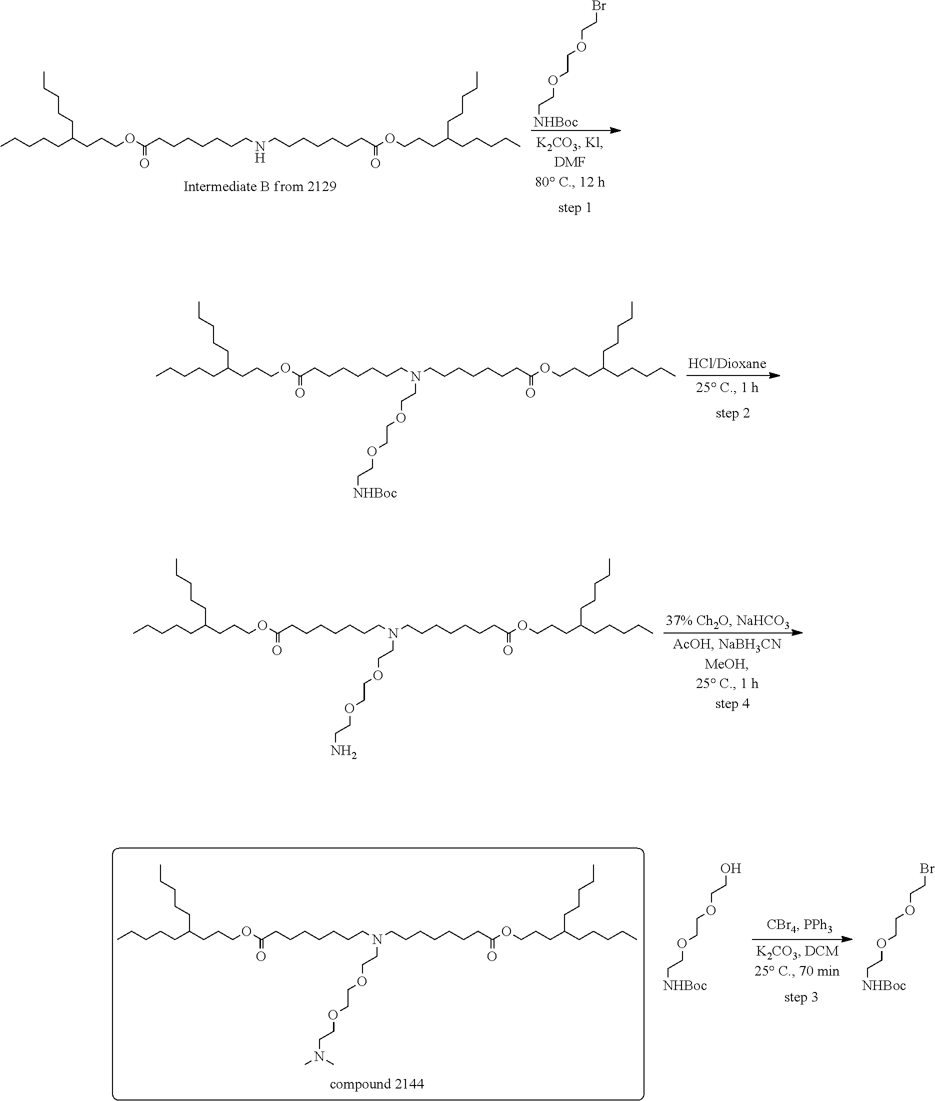

### Step 1:

To a solution of 4-pentylnonyl 8-[[8-oxo-8-(4-pentylnonoxy)octyl]amino]octanoate (300 mg, 432.18 μmol, 1 eq) in DMF (10 mL) was added K₂CO₃ (298.65 mg, 2.16 mmol, 40.10 μL, 5 eq), KI (143.48 mg, 864.35 μmol, 2 eq) and tert-butyl N-[2-[2-(2-bromoethoxy)ethoxy]ethyl]carbamate (674.63 mg, 2.16 mmol, 5 eq), stirred at 80° C. for 12 hours. The reaction mixture was filtered and the filtrate was quenched with water (10 mL) and extracted with dichloromethane (3×10 mL). The combined organic layer was washed with brine (2×5 mL), dried over anhydrous sodium sulfate, filtered and concentrated in vacuo to get a residue. The residue was purified by column chromatography (SiO₂, Petroleum ether/Ethyl acetate=1/1 to 0/1) to give 4-pentylnonyl 8-[2-[2-[2-(tert-butoxycarbonylamino)ethoxy]ethoxy]ethyl-[8-oxo-8-(4-pentylnonoxy)octyl]amino]octanoate (350 mg, 378.19 μmol, 87.51% yield) as yellow oil.

### Step 2:

A solution of 4-pentylnonyl 8-[2-[2-[2-(tert-butoxycarbonylamino)ethoxy]ethoxy]ethyl-[8-oxo-8-(4-pentylnonoxy)octyl]amino]octanoate (300 mg, 324.17 μmol, 1 eq) in HCl/dioxane (4 M, 6.00 mL, 74.04 eq) was stirred at 25° C. for 1 hour. The reaction mixture was concentrated in vacuo to give 4-pentylnonyl 8-[2-[2-(2-aminoethoxy)ethoxy]ethyl-[8-oxo-8-(4-pentylnonoxy)octyl]amino] octanoate (300 mg, crude, HCl) as a yellow solid.

¹H NMR (400 MHz, CDCl₃), 5.10 (brs, 1H), 4.05 (t, J=6.8 Hz, 4H), 3.64 (s, 4H), 3.55-3.56 (m, 4H), 3.32-3.33 (m, 2H), 2.66 (t, J=5.2 Hz, 2H), 2.44 (t, J=5.2 Hz, 4H), 2.30 (t, J=7.6 Hz, 4H), 1.55-1.65 (m, 8H), 1.44-1.45 (m, 13H), 1.24-1.31 (m, 50H), 0.89 (t, J=7.2 Hz, 12H). LCMS: (M+H⁺): 825.8 @ 0.980 min.

### Step 3:

To a solution of tert-butyl N-[2-[2-(2-hydroxyethoxy)ethoxy]ethyl]carbamate (2 g, 8.02 mmol, 1 eq) in DCM (20 mL) was added CBr₄ (3.46 g, 10.43 mmol, 1.3 eq) and K₂CO₃ (1.44 g, 10.43 mmol, 1.3 eq), then a solution of PPh₃ (3.37 g, 12.84 mmol, 1.6 eq) in DCM (40 mL) was stirred at 25° C. for 1 hour. The reaction mixture was filtered and concentrated in vacuo. The residue was purified by column chromatography (SiO₂, Petroleum ether/Ethyl acetate=30/1 to 5/1) to give tert-butyl N-[2-[2-(2-bromoethoxy)ethoxy]ethyl]carbamate (1.2 g, 3.84 mmol, 47.91% yield) as colorless oil.

### Step 4:

To a solution of 4-pentylnonyl 8-[2-[2-(2-aminoethoxy)ethoxy]ethyl-[8-oxo-8-(4-pentylnonoxy) octyl]amino]octanoate (300 mg, 348.11 μmol, 1 eq, HCl) and formaldehyde (2.81 g, 34.62 mmol, 2.58 mL, 37% purity, 99.44 eq) in MeOH (5 mL) was added NaHCO₃ (87.73 mg, 1.04 mmol, 40.62 μL, 3 eq), and then stirred at 25° C. for 10 minutes. AcOH (627.14 mg, 10.44 mmol, 597.28 μL, 30 eq) and NaBH₃CN (65.63 mg, 1.04 mmol, 3 eq) were added into the mixture and stirred at 25° C. for 1 hour. The reaction mixture was quenched with sat.NaHCO₃ (10 mL) and extracted with EtOAc (3×10 mL). The combined organic layer was washed with brine (2×5 mL), dried over anhydrous sodium sulfate, filtered and concentrated in vacuo to get a residue. The residue was purified by column chromatography (SiO₂, Ethyl acetate:MeOH=20/1 to 1/1) to give 4-pentylnonyl8-[2-[2-[2-(dimethylamino)ethoxy]ethoxy]ethyl-[8-oxo-8-(4-pentylnonoxy)octyl]amino]octanoate (104 mg, 121.87 μmol, 35.01% yield, 100% purity) as colorless oil.

¹H NMR (400 MHz, CDCl₃), 4.05 (t, J=6.8 Hz, 4H), 3.50-3.61 (m, 8H), 2.67 (t, J=3.2 Hz, 2H), 2.53 (t, J=5.6 Hz, 2H), 2.44-2.48 (m, 4H), 2.27-2.32 (m, 10H), 1.55-1.64 (m, 8H), 1.40-1.47 (m, 4H), 1.20-1.35 (m, 50H), 0.89 (t, J=6.8 Hz, 12H).

LCMS: (M+H⁺): 853.6 @ 3.493 min.

### 4.17: Synthesis of Compound 2145

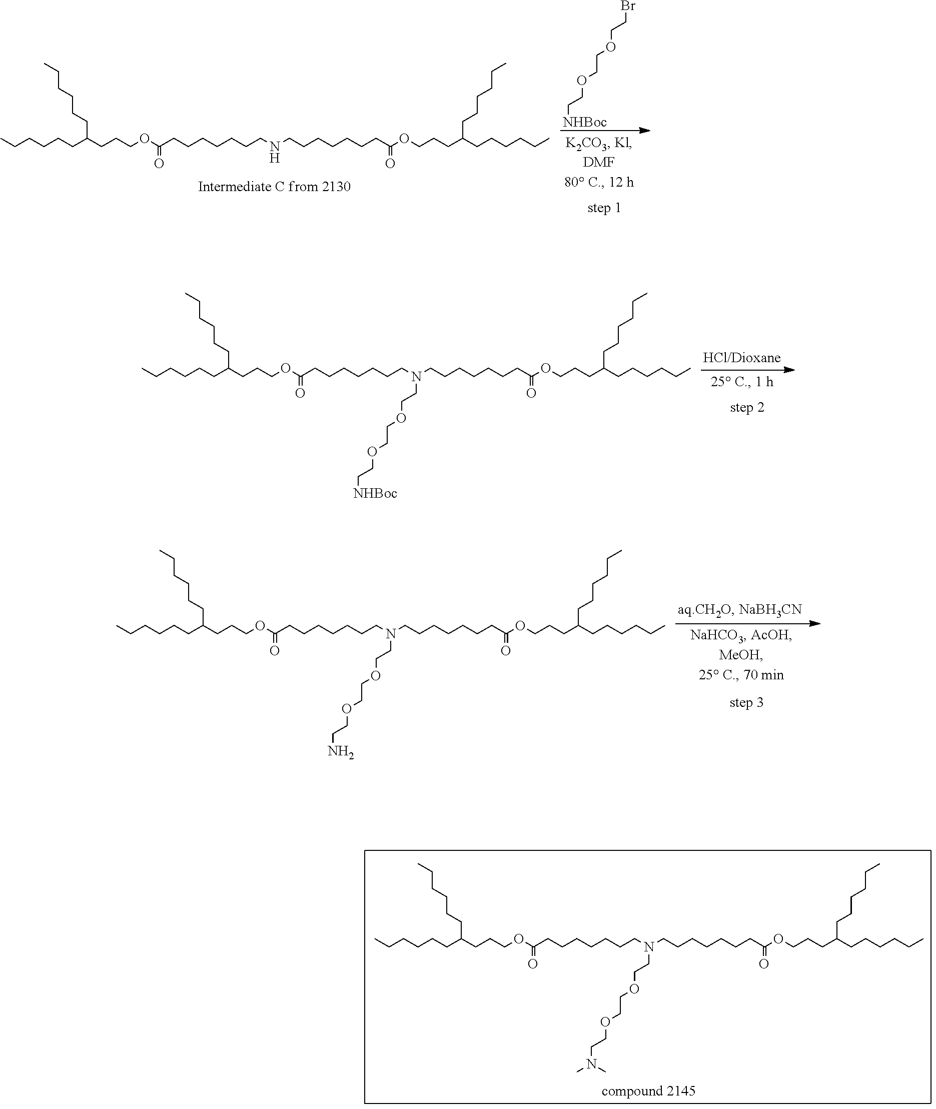 [SMILES: CCCCCCC(CCCCCC)CCCOC(=O)CCCCCCCNCCCCCCCC(=O)OCCCC(CCCCCC)CCCCCC ; CC(C)(C)OC(=O)NCCOCCOCCBr ; CCCCCCC(CCCCCC)CCCOC(=O)CCCCCCCN(CCCCCCCC(=O)OCCCC(CCCCCC)CCCCCC)CCOCCOCCNC(=O)OC(C)(C)C ; CCCCCCC(CCCCCC)CCCOC(=O)CCCCCCCN(CCCCCCCC(=O)OCCCC(CCCCCC)CCCCCC)CCOCCOCCN ; CCCCCCC(CCCCCC)CCCOC(=O)CCCCCCCN(CCCCCCCC(=O)OCCCC(CCCCCC)CCCCCC)CCOCCOCCN(C)C]

### Step 1:

To a solution of 4-hexyldecyl 8-[[8-(4-hexyldecoxy)-8-oxo-octyl]amino]octanoate (200 mg, 266.57 μmol, 1 eq) in DMF (5 mL) was added K₂CO₃ (184.21 mg, 1.33 mmol, 5 eq), KI (88.50 mg, 533.14 μmol, 2 eq) and tert-butyl N-[2-[2-(2-bromoethoxy)ethoxy]ethyl]carbamate (416.12 mg, 1.33 mmol, 5 eq) in DMF (2 mL), then the mixture was stirred at 80° C. for 12 hours. The reaction mixture was diluted with H₂O 9 mL and extracted with EtOAc 12 mL (4 mL×3). The combined organic layers were washed with Brine 9 mL (3 mL×3), dried over Na₂SO₄, filtered and concentrated under reduced pressure to give a residue. The residue was purified by column chromatography (SiO₂, Petroleum ether/Ethyl acetate=10/1 to 0/1, Ethyl acetate/MeOH=10/1 to 0/1) to give 4-hexyldecyl 8-[2-[2-[2-(tert-butoxycarbonylamino)ethoxy]ethoxy]ethyl-[8-(4-hexyldecoxy)-8-oxo-octyl]amino]octanoate (150 mg, 152.82 μmol, 57.33% yield) as a yellow oil.

### Step 2:

A solution of 4-hexyldecyl 8-[2-[2-[2-(tert-butoxycarbonylamino)ethoxy]ethoxy]ethyl-[8-(4-hexyldecoxy)-8-oxo-octyl]amino]octanoate (140 mg, 142.63 μmol, 1 eq) in HCl/dioxane (4 M, 6.17 mL, 173.08 eq) was stirred at 25° C. for 1 hour. The reaction mixture was concentrated under reduced pressure to give 4-hexyldecyl 8-[2-[2-(2-aminoethoxy)ethoxy]ethyl-[8-(4-hexyldecoxy)-8-oxo-octyl]amino]octanoate (125 mg, crude) as a yellow oil.

### Step 3:

To a solution of 4-hexyldecyl 8-[2-[2-(2-aminoethoxy)ethoxy]ethyl-[8-(4-hexyldecoxy)-8-oxo-octyl]amino]octanoate (125 mg, 141.81 μmol, 1 eq) and formaldehyde (495.72 mg, 16.51 mmol, 454.79 μL, 116.42 eq) in MeOH (10 mL) was added NaHCO₃ (35.74 mg, 425.44 μmol, 16.55 μL, 3 eq) and stirred at 25° C. for 10 minutes. Then AcOH (255.49 mg, 4.25 mmol, 243.32 μL, 30 eq) and NaBH₃CN (26.74 mg, 425.44 μmol, 3 eq) were added to the mixture at 25° C. for 1 hour. The reaction mixture was filtered and the filtrate was diluted with aq. NaHCO₃ 20 mL and extracted with EtOAc 30 mL (10 mL×3). The combined organic layers were washed with brine 24 mL (8 mL×3), dried over Na₂SO₄, and concentrated under reduced pressure to give a residue. The residue was purified by prep-HPLC (column: Phenomenex Luna C18 100×30 mm×5 μm; mobile phase: [water(HCl)-ACN]; B %: 55%-85%, 10 minutes), concentrated under reduced pressure to remove ACN, then diluted with aqueous NaHCO₃ 20 ml and extracted with EtOAc 30 mL (10 mL×3). The combined organic layers were washed with Brine 24 mL (8 mL×3), dried over Na₂SO₄, and concentrated under reduced pressure to give a residue. The residue was purified by column chromatography (SiO₂, Petroleum ether/Ethyl acetate=1/1 to 0/1, Ethyl acetate:Methanol=10/1 to 0/1) to give 4-hexyldecyl 8-[2-[2-[2-(dimethylamino)ethoxy]ethoxy]ethyl-[8-(4-hexyldecoxy)-8-oxo-octyl]amino]octanoate (25 mg, 27.21 μmol, 19.19% yield, 99% purity) as a colorless oil.

¹H NMR (400 MHz, CDCl₃), 4.04 (t, J=7.2 Hz, 4H), 3.50-3.61 (m, 8H), 2.67 (t, J=6.4 Hz, 2H), 2.55 (t, J=6.0 Hz, 2H), 2.40-2.50 (m, 4H), 2.25-2.35 (m, 10H), 1.55-1.65 (m, 8H), 1.40-1.48 (m, 4H), 1.20-1.35 (m, 58H), 0.89 (t, J=6.8 Hz, 12H).

LCMS: (M+H⁺): 909.7 @ 3.641 min.

### 4.18: Synthesis of Compound 2146

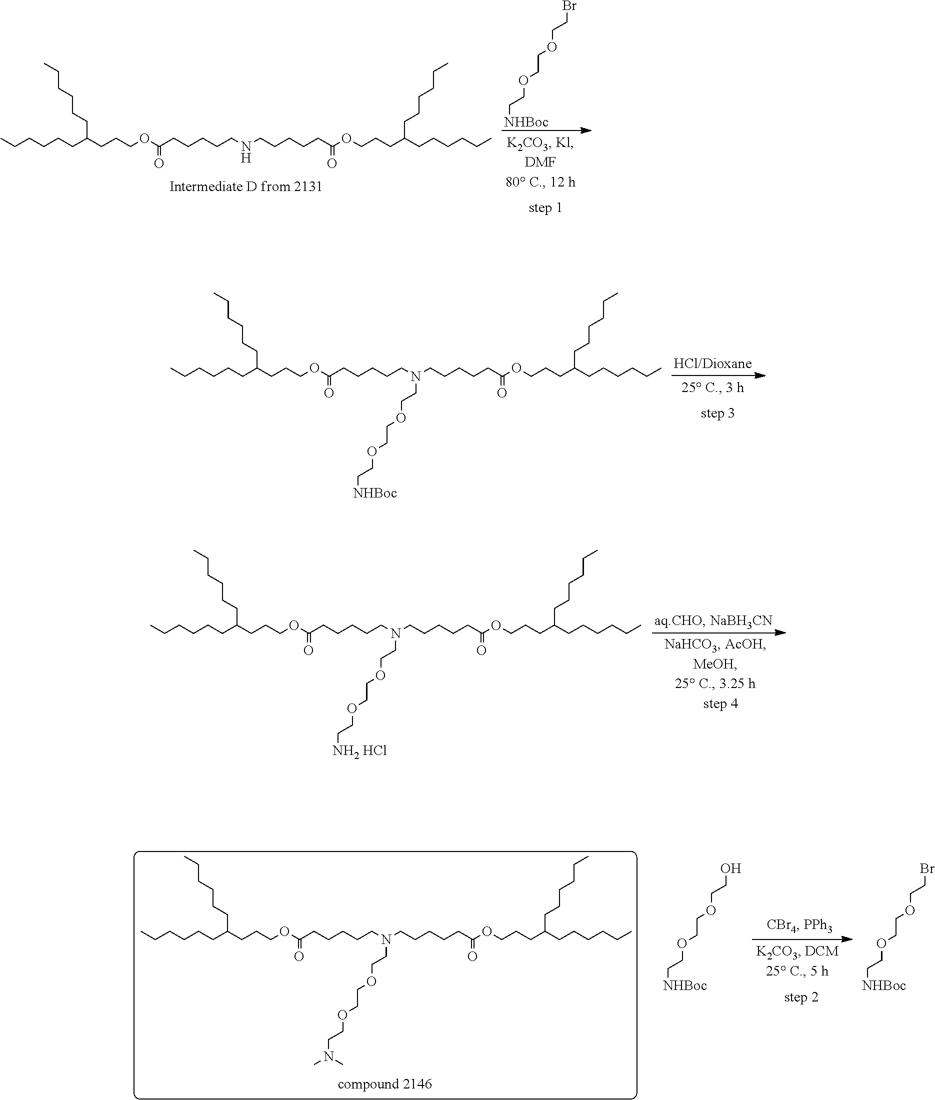

### Step 1:

To a solution of 4-hexyldecyl 6-[[6-(4-hexyldecoxy)-6-oxo-hexyl]amino]hexanoate (1 g, 1.44 mmol, 1 eq) in DMF (3 mL) was added K₂CO₃ (995.52 mg, 7.20 mmol, 5 eq), KI (478.28 mg, 2.88 mmol, 2 eq), and tert-butyl N-[2-[2-(2-bromoethoxy)ethoxy]ethyl]carbamate (1.80 g, 5.76 mmol, 354.38 μL, 4 eq). The mixture was stirred at 80° C. for 12 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=20/1 to 10/1) to compound 4-hexyldecyl 6-[2-[2-[2-(tert-butoxycarbonylamino)ethoxy]ethoxy]ethyl-[6-(4-hexyldecoxy)-6-oxo-hexyl]amino]hexanoate (0.5 g, 540.28 μmol, 37.50% yield) as yellow oil.

### Step 2:

To a solution of tert-butyl N-[2-[2-(2-hydroxyethoxy)ethoxy]ethyl]carbamate (1 g, 4.01 mmol, 1 eq) in DCM (50 mL) was added carbon tetrabromide (1.73 g, 5.21 mmol, 1.3 eq), K₂CO₃ (720.68 mg, 5.21 mmol, 1.3 eq) and PPh₃ (1.68 g, 6.42 mmol, 1.6 eq) in DCM (10 mL). The mixture was stirred at 25° C. for 5 hours. The reaction mixture was filtered and concentrated under reduced pressure to give a residue. The residue was purified by column chromatography (SiO₂, Petroleum ether/Ethyl acetate=20/1 to 0/1) to give compound tert-butyl N-[2-[2-(2-bromoethoxy)ethoxy] ethyl] carbamate (2.6 g, 8.33 mmol, 41.52% yield) as white solid.

### Step 3:

A solution of 4-hexyldecyl 6-[2-[2-[2-(tert-butoxycarbonylamino)ethoxy]ethoxy]ethyl-[6-(4-hexyldecoxy)-6-oxo-hexyl]amino]hexanoate (0.5 g, 540.28 μmol, 1 eq) in HCl/dioxane (4 M, 9.35 mL, 69.23 eq) was stirred at 25° C. for 3 hours under N₂ atmosphere. The reaction mixture was concentrated under reduced pressure to give compound 4-hexyldecyl 6-[2-[2-(2-aminoethoxy) ethoxy]ethyl-[6-(4-hexyldecoxy)-6-oxo-hexyl]amino]hexanoate (0.55 g, crude, HCl) as yellow oil.

### Step 4:

To a solution of 4-hexyldecyl 6-[2-[2-(2-aminoethoxy)ethoxy]ethyl-[6-(4-hexyldecoxy)-6-oxo-hexyl]amino]hexanoate (0.55 g, 638.20 μmol, 1 eq, HCl), formaldehyde (5.45 g, 67.16 mmol, 5 mL, 37% purity, 105.23 eq) in MeOH (10 mL) added NaHCO₃ (160.85 mg, 1.91 mmol, 74.47 μL, 3 eq) at 25° C. and stirred at 25° C. for 15 minutes. Then AcOH (1.16 g, 19.23 mmol, 1.10 mL, 30.14 eq) and NaBH₃CN (120.31 mg, 1.91 mmol, 3 eq) were added to the mixture. The resulting mixture was stirred at 25° C. for 3 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Ethyl acetate:MeOH=1/0 to 10/1) to give compound 4-hexyldecyl 6-[2-[2-[2-(dimethylamino)ethoxy]ethoxy]ethyl-[6-(4-hexyldecoxy)-6-oxo-hexyl]amino]hexanoate (227 mg, 266.00 μmol, 41.68% yield, 100% purity) as yellow oil.

¹H NMR (400 MHz, CDCl₃), 4.04 (t, J=7.2 Hz, 4H), 3.61 (s, 4H), 3.58 (t, J=6.4 Hz, 2H), 3.53 (t, J=6.4 Hz, 2H), 2.65 (t, J=6.4 Hz, 2H), 2.52 (t, J=5.6 Hz, 2H), 2.45 (t, J=7.2 Hz, 4H), 2.30 (t, J=7.6 Hz, 4H), 2.27 (s, 6H), 1.55-1.69 (m, 8H), 1.40-1.49 (m, 4H), 1.20-1.35 (m, 50H), 0.89 (t, J=6.4 Hz, 12H). LCMS: (M+H⁺): 853.7 @ 2.979 min.

### 4.18: Synthesis of Compound 2215

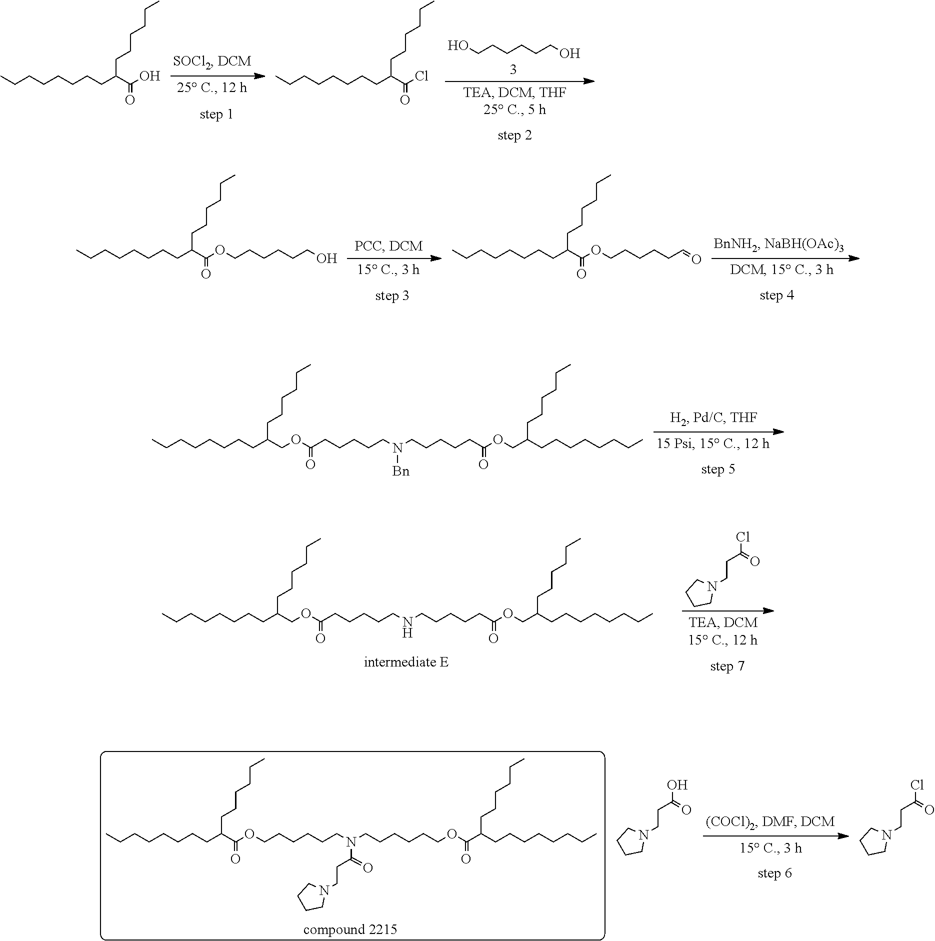

### Step 1:

A mixture of 2-hexyldecanoic acid (30 g, 116.99 mmol, 1 eq) in DCM (200 mL) was added SOCl₂ (13.92 g, 116.99 mmol, 8.49 mL, 1.5 eq) was stirred at 25° C. for 12 hours under N₂ atmosphere. The reaction mixture was concentrated under reduced pressure to give compound 2-hexyldecanoyl chloride (32 g, crude) as a yellow oil. The crude product was used to next step directly for next step.

### Step 2:

A mixture of hexane-1,6-diol (13.76 g, 116.42 mmol, 13.90 mL, 1 eq) in DCM (20 mL) and THF (20 mL) was added TEA (11.78 g, 116.42 mmol, 16.20 mL, 1 eq) and 2-hexyldecanoyl chloride (32 g, 116.42 mmol, 1 eq), then the mixture was stirred at 25° C. for 5 hours under N₂ atmosphere. The reaction mixture was filtered and the filtrate was concentrated under reduced pressure to give a residue. The residue was purified by column chromatography (SiO₂, Petroleum ether/Ethyl acetate=10/1 to 1/1) to give compound 6-hydroxyhexyl 2-hexyldecanoate (16 g, 44.87 mmol, 38.54% yield) as yellow oil.

¹H NMR (400 MHz, CDCl₃), 4.07 (t, J=6.4 Hz, 2H), 3.65 (t, J=6.4 Hz, 2H), 2.31-2.36 (m, 1H), 1.57-1.66 (m, 6H), 1.35-1.45 (m, 6H), 1.20-1.30 (m, 20H), 0.88 (t, J=6.8 Hz, 6H).

### Step 3:

To a solution of 6-hydroxyhexyl 2-hexyldecanoate (12 g, 33.65 mmol, 1 eq) in DCM (150 mL) was added PCC (8.70 g, 40.38 mmol, 1.2 eq) at 15° C., then stirred at 15° C. for 3 hours under N₂ atmosphere. The reaction mixture was filtered and the filtrate was concentrated under reduced pressure to give a residue. The residue was purified by column chromatography (SiO₂, Petroleum ether/Ethyl acetate=1/0 to 50/1) to give compound 6-oxohexyl 2-hexyldecanoate (8.1 g, 22.84 mmol, 67.88% yield) as colorless oil.

¹H NMR (400 MHz, CDCl₃), 9.76 (s, 1H), 4.07 (t, J=6.4 Hz, 2H), 2.42-2.46 (m, 2H), 2.26-2.34 (m, 1H), 1.55-1.70 (m, 6H), 1.36-1.46 (m, 4H), 1.24-1.28 (m, 20H), 0.87 (t, J=6.8 Hz, 6H).

### Step 4:

A mixture of 6-oxohexyl 2-hexyldecanoate (7.7 g, 21.72 mmol, 2.5 eq), phenylmethanamine (930.80 mg, 8.69 mmol, 946.90 μL, 1 eq) and sodium; triacetoxyboranuide (5.52 g, 26.06 mmol, 3 eq) in DCM (150 mL) was stirred at 15° C. for 3 hours under N₂ atmosphere. The reaction mixture was extracted with EtOAc 90 mL (30 mL×3). The combined organic layers were dried over Na₂SO₄, filtered and the filtrate was concentrated under reduced pressure to give a residue. The residue was purified by column chromatography (SiO₂, Petroleum ether/Ethyl acetate=20/1 to 8/1) to give compound 6-[benzyl-[6-(2-hexyldecanoyloxy)hexyl]amino]hexyl 2-hexyldecanoate (3 g, 3.83 mmol, 44.03% yield) as colorless oil.

¹H NMR (400 MHz, CDCl₃), 7.23-7.31 (m, 5H), 4.05 (t, J=6.8 Hz, 4H), 3.54 (s, 2H), 2.40 (m, 4H), 2.28-2.35 (m, 2H), 1.59-1.62 (m, 8H), 1.40-1.47 (m, 8H), 1.26-1.32 (m, 48H), 0.88 (t, J=7.2 Hz, 12H). LCMS. (M+H⁺): 784.7.

### Step 5:

To a suspension of Pd/C (1 g, 10% purity) in THE (50 mL) was added 6-[benzyl-[6-(2-hexyldecanoyloxy)hexyl]amino]hexyl 2-hexyldecanoate (3 g, 3.83 mmol, 1 eq) in THE (10 mL). The mixture was stirred at 15° C. for 12 hours under H₂ (15 Psi) atmosphere. The reaction mixture was filtered and the filtrate was concentrated under reduced pressure to give a residue. The residue was purified by column chromatography (SiO₂, Petroleum ether/Ethyl acetate=10/1 to 5/1) to give compound 6-[6-(2-hexyldecanoyloxy)hexylamino]hexyl 2-hexyldecanoate (2.1 g, 3.03 mmol, 79.09% yield) as colorless oil.

¹H NMR (400 MHz, CDCl₃), 4.06 (t, J=6.8 Hz, 4H), 2.56 (t, J=7.2 Hz, 4H), 2.67-2.34 (m, 2H), 1.25-1.66 (m, 65H), 0.87 (t, J=6.8 Hz, 12H).

### Step 6:

To a solution of 3-pyrrolidin-1-ylpropanoic acid (200 mg, 1.40 mmol, 1 eq) in DCM (8 mL) was added DMF (19.00 mg, 259.94 μmol, 0.02 mL, 1.86e-1 eq) and oxalyl dichloride (886.46 mg, 6.98 mmol, 611.35 μL, 5 eq) at 15° C., then stirred at 15° C. for 3 hours under N₂ atmosphere. The reaction mixture was concentrated under reduced pressure to give crude product 3-pyrrolidin-1-ylpropanoyl chloride (200 mg, crude) as yellow oil. The crude product was used to next step directly for next step.

### Step 7:

To a solution of 3-pyrrolidin-1-ylpropanoyl chloride (186.27 mg, 1.15 mmol, 4 eq) in DCM (8 mL) was added 6-[6-(2-hexyldecanoyloxy)hexylamino]hexyl 2-hexyldecanoate (200 mg, 288.12 μmol, 1 eq) and TEA (291.54 mg, 2.88 mmol, 401.02 μL, 10 eq) in DCM (8 mL) at 15° C., then stirred at 15° C. for 12 hours under N₂ atmosphere. The reaction mixture was extracted with EtOAc 150 mL (50 mL×3). The combined organic layers were dried over Na₂SO₄, filtered and concentrated under reduced pressure to give a residue. The residue was purified by column chromatography (SiO₂, Petroleum ether/Ethyl acetate=10/1 to 1/1, 10% NH₃·H₂O) to give compound 6-[6-(2-hexyldecanoyloxy)hexyl-(3-pyrrolidin-1-ylpropanoyl)amino]hexyl 2-hexyldecanoate (64 mg, 78.11 μmol, 27.11% yield, 100% purity) as yellow oil.

¹H NMR (400 MHz, CDCl₃), 4.06 (q, J=6.4 Hz, 4H), 3.20-3.31 (m, 4H), 2.81 (t, J=8.4 Hz, 2H), 2.52-2.56 (m, 6H), 2.27-2.35 (m, 2H), 1.78-1.81 (m, 4H), 1.50-1.66 (m, 14H), 1.26-1.45 (m, 50H), 0.88 (t, J=6.8 Hz, 12H). LCMS: (M+H⁺): 819.6 @ 3.495 min.

### 4.19: Synthesis of Compound 2216

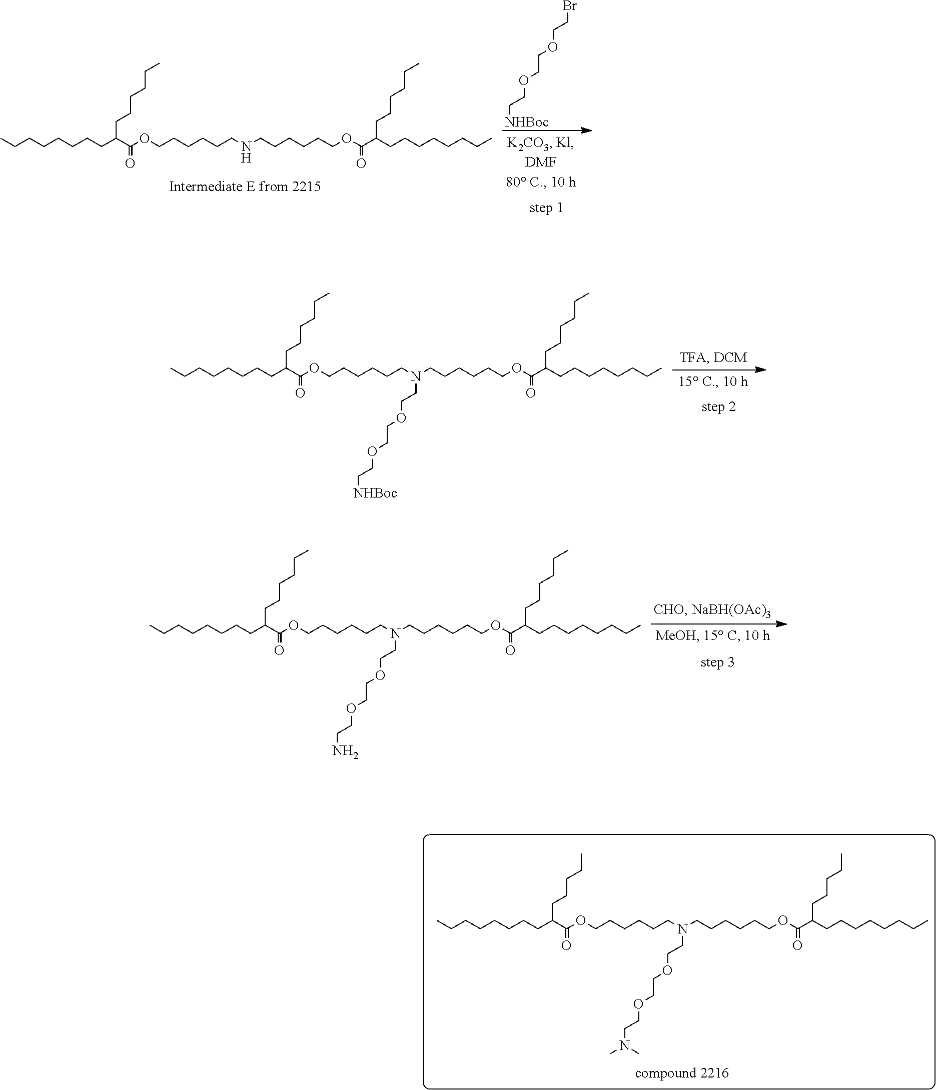

### Step 1:

To a solution of 6-[6-(2-hexyldecanoyloxy)hexylamino]hexyl 2-hexyldecanoate (500 mg, 720.29 μmol, 1 eq) in DMF (10 mL) was added tert-butyl N-[2-[2-(2-bromoethoxy)ethoxy]ethyl] carbamate (899.50 mg, 2.88 mmol, 4 eq), K₂CO₃ (497.74 mg, 3.60 mmol, 5 eq) and KI (239.14 mg, 1.44 mmol, 2 eq) at 15° C. The mixture was degassed and purged with N₂ for 3 times, and then stirred at 80° C. for 10 hours under N₂ atmosphere. The reaction mixture was diluted with 50 ml H₂O and extracted with EtOAc 30 mL (15 mL×2). The combined organic layers were dried over Na₂SO₄, filtered and concentrated under reduced pressure to give a residue. The residue was purified by column chromatography (SiO₂, Petroleum ether/Ethyl acetate=10/1 to 5/1, 5% NH₃·H₂O) to give compound 6-[2-[2-[2-(tert-butoxycarbonylamino)ethoxy]ethoxy]ethyl-[6-(2-hexyldecanoyloxy) hexyl]amino]hexyl 2-hexyldecanoate (430 mg, 464.64 μmol, 64.51% yield) as colorless oil.

¹H NMR (400 MHz, CDCl₃), 5.04 (brs, 1H), 4.06 (t, J=6.8 Hz, 4H), 3.60 (s, 4H), 3.52-3.56 (m, 4H), 3.32 (d, J=4.8 Hz, 2H), 2.65 (t, J=6.4 Hz, 2H), 2.44 (t, J=7.2 Hz, 4H), 2.28-2.35 (m, 2H), 1.59-1.64 (m, 8H), 1.26-1.45 (m, 66H), 0.88 (t, J=6.8 Hz, 12H).

LCMS: (M+H⁺): 925.8.

### Step 2:

To a solution of 6-[2-[2-[2-(tert-butoxycarbonylamino)ethoxy]ethoxy]ethyl-[6-(2-hexyldecanoyloxy)hexyl]amino]hexyl 2-hexyldecanoate (430 mg, 464.64 μmol, 1 eq) in DCM (8 mL) was added TFA (3.08 g, 27.01 mmol, 2 mL, 58.14 eq) at 15° C. The mixture was degassed and purged with N₂ for 3 times, and then stirred at 15° C. for 10 hours under N₂ atmosphere. The reaction mixture was adjusted to pH=7.0 with aqueous saturated NaHCO₃ and extracted with EtOAc 50 mL (25 mL×2). The combined organic layers were dried over Na₂SO₄, filtered and concentrated under reduced pressure to give compound 6-[2-[2-(2-aminoethoxy)ethoxy]ethyl-[6-(2-hexyldecanoyloxy)hexyl]amino]hexyl 2-hexyldecanoate (350 mg, crude) as colorless oil. The crude product was used to next step.

### Step 3:

To a solution of 6-[2-[2-(2-aminoethoxy)ethoxy]ethyl-[6-(2-hexyldecanoyloxy)hexyl]amino]hexyl 2-hexyldecanoate (250 mg, 302.91 μmol, 1 eq) in MeOH (5 mL) was added formaldehyde (5.45 g, 67.16 mmol, 5.00 mL, 37% purity, 221.71 eq) and NaBH(OAc)₃ (192.60 mg, 908.72 μmol, 3 eq) at 15° C. The mixture was degassed and purged with N₂ for 3 times, and then stirred at 15° C. for 10 hours under N₂ atmosphere. The reaction mixture was adjusted to pH=7.0 with aqueous saturated NaHCO₃ and extracted with EtOAc 100 mL (50 mL×2). The combined organic layers were dried over Na₂SO₄, filtered and concentrated under reduced pressure to give a residue. The residue was purified by column chromatography (SiO₂, Petroleum ether/Ethyl acetate=5/1 to 2/1, 5% NH₃·H₂O) to give compound 6-[2-[2-[2-(dimethylamino)ethoxy]ethoxy]ethyl-[6-(2-hexyldecanoyloxy)hexyl]amino] hexyl 2-hexyldecanoate (66 mg, 76.18 μmol, 25.15% yield, 98.5% purity) as colorless oil.

¹H NMR (400 MHz, CDCl₃), 4.06 (t, J=6.4 Hz, 4H), 3.52-3.61 (m, 8H), 2.66 (t, J=6.8 Hz, 2H), 2.52 (t, J=6.0 Hz, 2H), 2.45 (t, J=7.2 Hz, 4H), 2.27-2.2 (m, 8H), 1.56-1.64 (m, 8H), 1.26-1.44 (m, 56H), 0.88 (t, J=7.2 Hz, 12H). LCMS: (M+H⁺): 853.7 @ 2.960 min.

### 4.20: Synthesis of Compound 2225

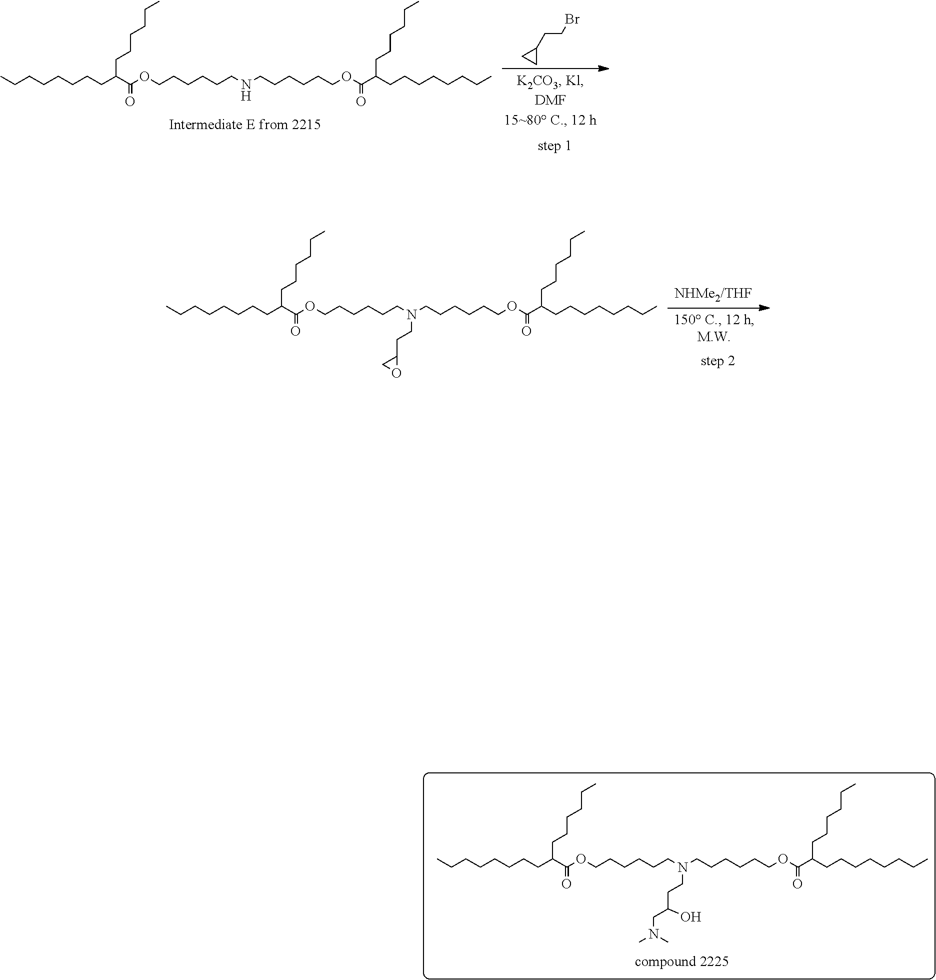

### Step 1:

To a solution of 6-[6-(2-hexyldecanoyloxy)hexylamino]hexyl 2-hexyldecanoate (500 mg, 720.29 μmol, 1 eq) in DMF (15 mL) was added 2-(2-bromoethyl)oxirane (435.06 mg, 2.88 mmol, 4 eq), K₂CO₃ (497.74 mg, 3.60 mmol, 5 eq) and KI (239.14 mg, 1.44 mmol, 2 eq) at 15° C. and then stirred at 80° C. for 12 h under N₂ atmosphere. The reaction mixture was diluted with H₂O 20 mL and extracted with EtOAc 80 mL (40 mL×2). The combined organic layers were dried over Na₂SO₄, filtered and concentrated under reduced pressure to give a residue. The residue was purified by column chromatography (SiO₂, Petroleum ether/Ethyl acetate=5/1 to 0/1, 5% NH₃·H₂O) to give compound 6-[6-(2-hexyldecanoyloxy)hexyl-[2-(oxiran-2-yl)ethyl]amino]hexyl 2-hexyldecanoate (200 mg, crude) as colorless oil.

LCMS: (M+H⁺): 764.7 @ 1.072 min.

### Step 2:

A mixture of 6-[6-(2-hexyldecanoyloxy)hexyl-[2-(oxiran-2-yl)ethyl]amino]hexyl 2-hexyldecanoate (150 mg, 196.27 μmol, 1 eq) and Me₂NH/THF (2 M, 8 mL, 81.52 eq) was taken up into a microwave tube. The sealed tube was heated at 150° C. for 12 h under microwave. The reaction mixture was concentrated under reduced pressure to give a residue. The residue was purified by prep-HPLC (column: Phenomenex Luna C18 100×30 mm×5 μm; mobile phase: [water(HCl)-MEOH]; B %: 60%-90%, 10 min), then adjusted the pH=8 with aqueous saturated NaHCO₃ and extracted with EtOAc (30 mL×3). The combined organic layers were dried over Na₂SO₄, filtered and concentrated under reduced pressure to give compound 6-[[4-(dimethylamino)-3-hydroxy-butyl]-[6-(2-hexyldecanoyloxy)hexyl]amino]hexyl 2-hexyldecanoate (30 mg, 37.07 μmol, 18.89% yield, 100% purity) as yellow oil.

¹H NMR (400 MHz, CDCl₃), 4.06 (t, J=6.8 Hz, 4H), 3.68-3.90 (m, 1H), 2.30-2.73 (m, 16H), 1.56-1.65 (m, 10H), 1.26-1.46 (m, 56H), 0.88 (t, J=6.8 Hz, 12H).

LCMS: (M+H⁺): 809.6 @ 3.202 min.

### 4.21: Synthesis of Compound 2229

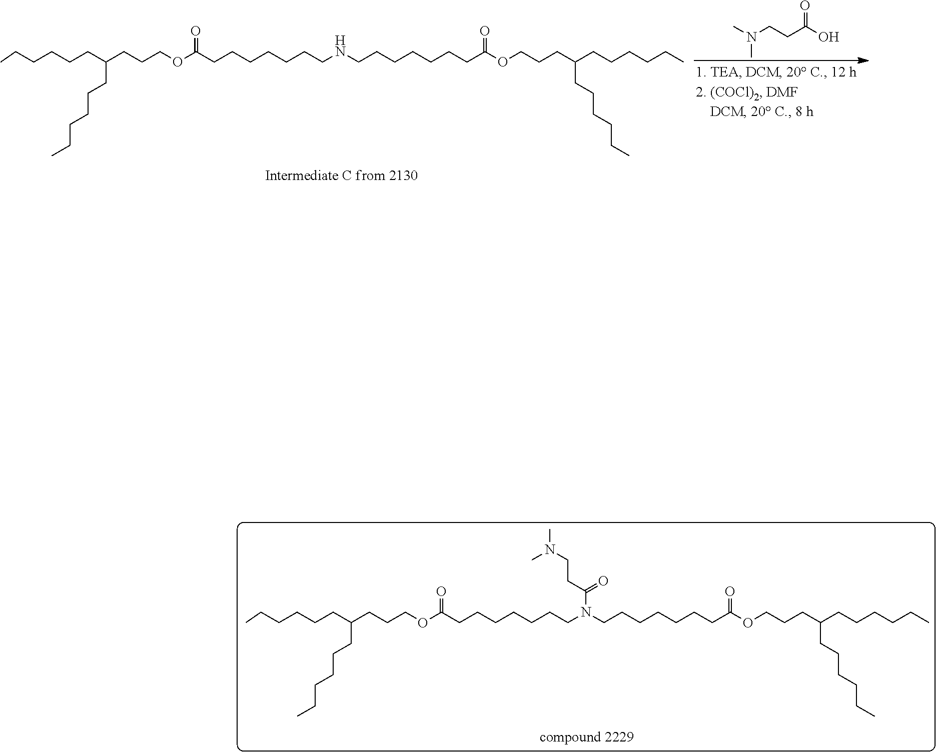

To a solution of 3-(dimethylamino)propanoic acid (170 mg, 1.11 mmol, 1 eq, HCl) in DCM (5 mL) was added (COCl)₂ (702.38 mg, 5.53 mmol, 484.40 μL, 5 eq) and DMF (8.09 mg, 110.67 μmol, 8.51 μL, 0.1 eq), stirred at 20° C. for 12 hours. The mixture was concentrated under reduced pressure to give 3-(dimethylamino)propanoyl chloride (191 mg, crude, HCl) as a yellow solid.

To a solution of 4-hexyldecyl 8-[[8-(4-hexyldecoxy)-8-oxo-octyl]amino]octanoate (150 mg, 199.93 μmol, 1 eq) in DCM (2 mL) was added TEA (141.62 mg, 1.40 mmol, 194.79 μL, 7 eq) and 3-(dimethylamino)propanoyl chloride (190 mg, 1.10 mmol, 5.52 eq, HCl) at 0° C., stirred at 20° C. for 8 hours. The mixture was concentrated under reduced pressure to give a residue. The residue was purified by column chromatography (SiO₂, Ethyl acetate/MeOH=50/1 to 1/1) to give 4-hexyldecyl 8-[3-(dimethylamino)propanoyl-[8-(4-hexyldecoxy)-8-oxo-octyl]amino]octanoate (39 mg, 45.91 μmol, 22.97% yield) as colorless oil.

¹H NMR (400 MHz, CDCl₃), 4.02-4.07 (m, 4H), 3.28 (t, J=8.0 Hz, 2H), 3.21 (t, J=7.2 Hz, 2H), 2.68 (brs, 2H), 2.52 (brs, 2H), 2.25-2.32 (m, 10H), 1.55-1.66 (m, 8H), 1.48-1.55 (m, 4H), 1.24-1.35 (m, 58H), 0.89 (t, J=6.8 Hz, 12H). LCMS: (M+H⁺): 849.7 @ 3.383 min.

### 4.22: Synthesis of Compound 2233

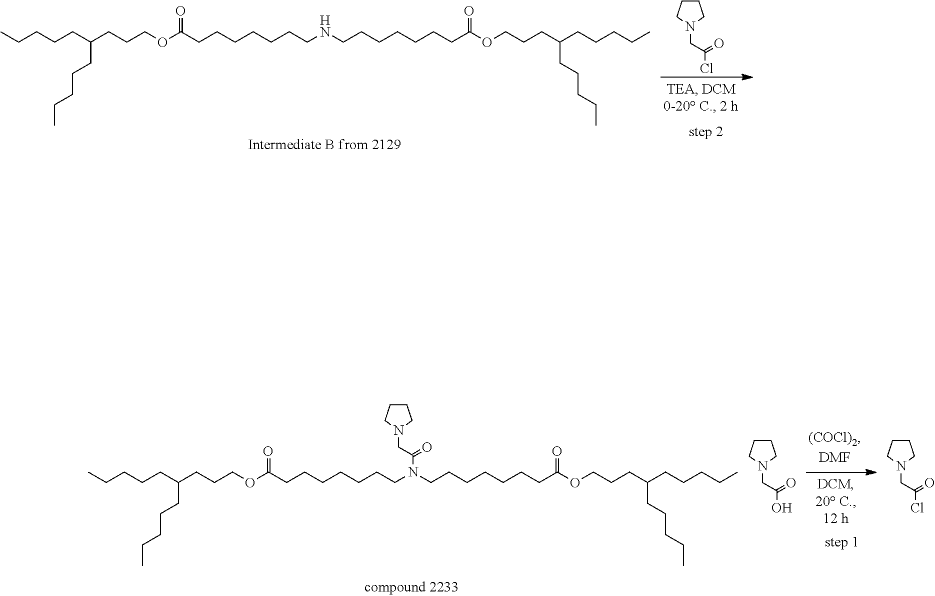

### Step 1:

To a solution of 2-pyrrolidin-1-ylacetic acid (100 mg, 774.25 μmol, 1 eq) in DCM (5 mL) was added (COCl)₂ (491.38 mg, 3.87 mmol, 338.88 μL, 5 eq) and DMF (5.66 mg, 77.43 μmol, 5.96 μL, 0.1 eq), stirred at 20° C. for 12 hours. The mixture was concentrated under reduced pressure. 2-pyrrolidin-1-ylacetyl chloride (114 mg, crude) was obtained as a yellow solid. The crude product was used for the next step without purification.

### Step 2:

To a solution of 4-pentylnonyl 8-[[8-oxo-8-(4-pentylnonoxy)octyl]amino]octanoate (100 mg, 144.06 μmol, 1 eq) in DCM (2 mL) was added TEA (58.31 mg, 576.24 μmol, 80.20 μL, 4 eq) and 2-pyrrolidin-1-ylacetyl chloride (106.06 mg, 576.24 μmol, 4 eq, HCl) at 0° C., stirred at 20° C. for 2 hours. LCMS showed the starting reactant consumed. The mixture was concentrated under reduced pressure get a residue. The residue was purified by column chromatography (SiO₂, Ethyl acetate/MeOH=50/1 to 1/1) to give 4-pentylnonyl 8-[[8-oxo-8-(4-pentylnonoxy)octyl]-(2-pyrrolidin-1-ylacetyl)amino] octanoate (58 mg, 72.02 μmol, 49.99% yield) as yellow oil.

¹H NMR (400 MHz, (CD₃)₂CO), 4.03 (t, J=6.8 Hz, 4H), 3.41 (t, J=7.6 Hz, 2H), 3.28 (t, J=7.2 Hz, 2H), 3.24 (s, 2H), 2.49-2.52 (m, 4H), 2.26-2.30 (m, 4H), 1.71-1.75 (m, 4H), 1.50-1.63 (m, 12H), 1.28-1.35 (m, 50H), 0.89 (t, J=6.8 Hz, 12H). LCMS: (M+H⁺): 805.7 @ 1.079 min.

### 4.23: Synthesis of Compound 2234

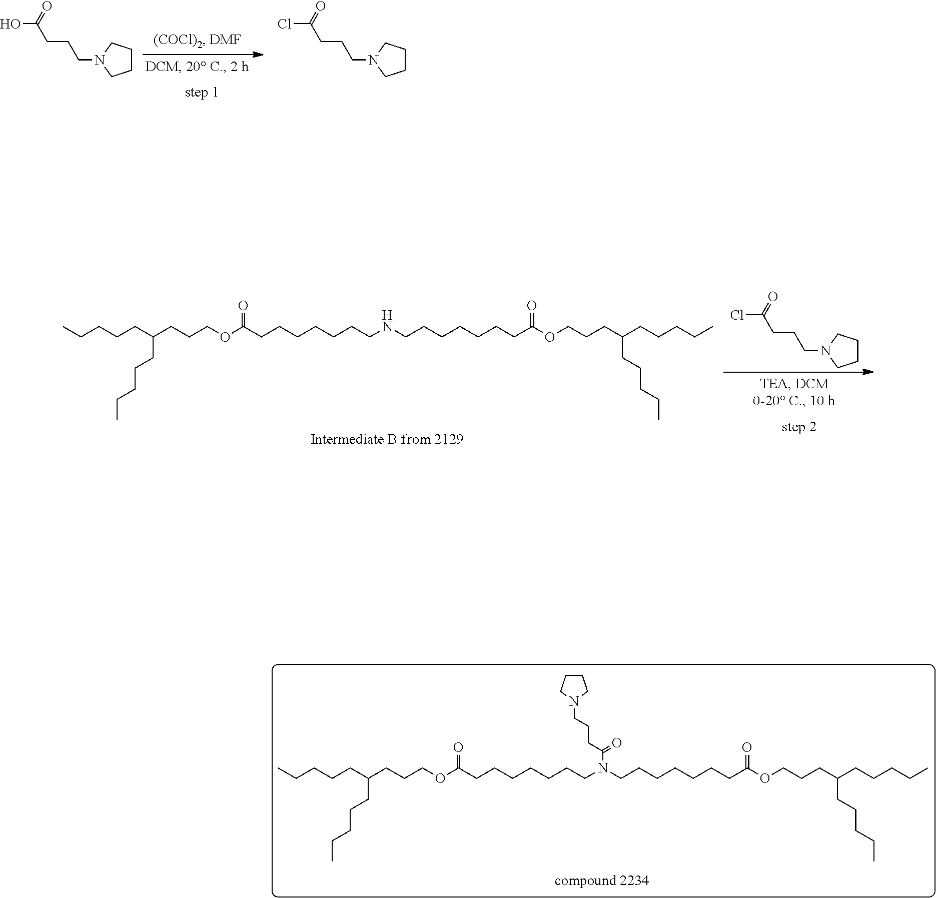

### Step 1:

To a solution of 4-pyrrolidin-1-ylbutanoic acid (160 mg, 1.02 mmol, 1 eq) in DCM (5 mL) was added (COCl)₂ (645.91 mg, 5.09 mmol, 445.46 μL, 5 eq) and DMF (7.44 mg, 101.77 μmol, 7.83 μL, 0.1 eq), and the reaction mixture was stirred at 20° C. for 2 hours. TLC showed the starting reactant consumed (quenched with MeOH). The mixture was concentrated under reduced pressure. 4-pyrrolidin-1-ylbutanoyl chloride (216 mg, crude, HCl) was obtained as a yellow solid. The crude was used for next step directly.

### Step 2:

To a solution of 4-pentylnonyl 8-[[8-oxo-8-(4-pentylnonoxy)octyl]amino]octanoate (170 mg, 244.90 μmol, 1 eq) in DCM (2 mL) was added TEA (99.13 mg, 979.60 μmol, 136.35 μL, 4 eq) and 4-pyrrolidin-1-ylbutanoyl chloride (207.79 mg, 979.60 μmol, 4 eq, HCl) at 0° C., and stirred at 20° C. for 10 hours. The mixture was concentrated under reduced pressure to give a residue. The residue was purified by column chromatography (SiO₂, ethyl acetate/MeOH=50/1 to 1/1) to give 4-pentylnonyl 8-[[8-oxo-8-(4-pentylnonoxy)octyl]-(4-pyrrolidin-1-ylbutanoyl)amino]octanoate (101 mg, 119.98 μmol, 48.99% yield, 99% purity) as colorless oil.

¹H NMR (400 MHz, CDCl₃), 4.02-4.07 (m, 4H), 3.28 (t, J=8.0 Hz, 2H), 3.21 (t, J=8.0 Hz, 2H), 2.45-2.52 (m, 6H), 2.27-2.36 (m, 6H), 1.86 (t, J=7.2 Hz, 2H), 1.77 (brs, 4H), 1.58-1.66 (m, 8H), 1.48-1.55 (m, 4H), 1.24-1.35 (m, 50H), 0.89 (t, J=7.2 Hz, 12H).

LCMS: (M+H⁺): 833.7 @ 3.207 min.

### 4.24: Synthesis of Compound 2235

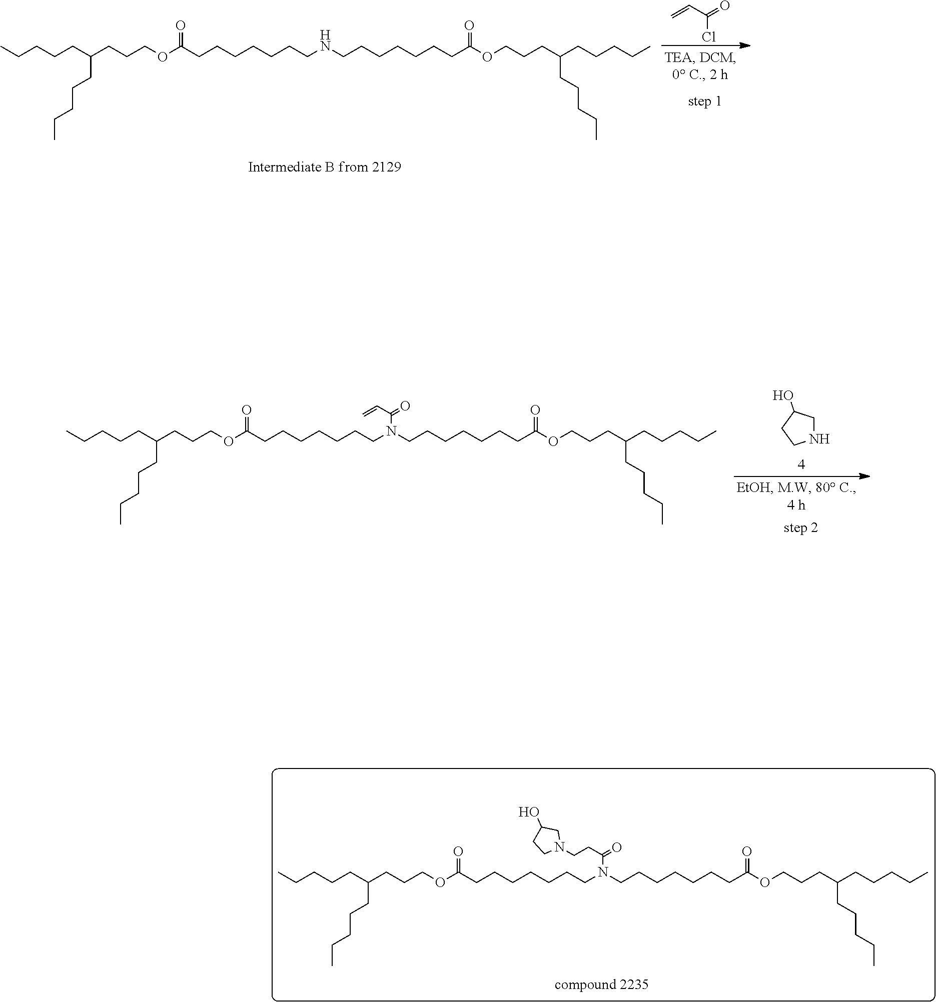

### Step 1:

To a solution of 4-pentylnonyl 8-[[8-oxo-8-(4-pentylnonoxy)octyl]amino]octanoate (500 mg, 720.29 μmol, 1 eq) in DCM (2 mL) was added TEA (364.43 mg, 3.60 mmol, 501.28 μL, 5 eq) and prop-2-enoyl chloride (260.77 mg, 2.88 mmol, 234.93 μL, 4 eq) at 0° C. Then the reaction mixture was stirred at 0° C. for 2 hours. The mixture was concentrated under reduced pressure to give the residue. The residue was purified by column chromatography (SiO₂, Petroleum ether/Ethyl acetate=20/1 to 1/1) to get 4-pentylnonyl 8-[[8-oxo-8-(4-pentylnonoxy)octyl]-prop-2-enoyl-amino]octanoate (400 mg, crude) as colorless oil.

### Step 2:

To a solution of 4-pentylnonyl 8-[[8-oxo-8-(4-pentylnonoxy)octyl]-prop-2-enoyl-amino]octanoate (200 mg, 267.30 μmol, 1 eq) in EtOH (2 mL) was added pyrrolidin-3-ol (69.86 mg, 801.91 μmol, 64.69 μL, 3 eq). Then the reaction mixture was stirred at 80° C. for 4 hours under M.W. condition. The mixture was concentrated under reduced pressure to give a residue. The residue was purified by column chromatography (SiO₂, Ethyl acetate/MeOH=50/1 to 1/1) to give 4-pentylnonyl 8-[3-(3-hydroxypyrrolidin-1-yl)propanoyl-[8-oxo-8-(4-pentylnonoxy)octyl]amino]octanoate (88 mg, 104.29 μmol, 39.02% yield, 99% purity) as colorless oil.

¹H NMR (400 MHz, CDCl₃), 4.35-4.38 (m, 1H), 4.02-4.07 (m, 4H), 3.19-3.31 (m, 4H), 2.75-2.99 (m, 4H), 2.50-2.59 (m, 2H), 2.35-2.40 (m, 1H), 2.25-2.30 (m, 4H), 2.15-2.25 (m, 1H), 1.70-1.85 (m, 1H), 1.45-1.66 (m, 13H), 1.24-1.35 (m, 50H), 0.89 (t, J=7.2 Hz, 12H).

LCMS: (M+H⁺): 835.8 @ 2.227 min.

### 4.25. Synthesis of Compound 2237

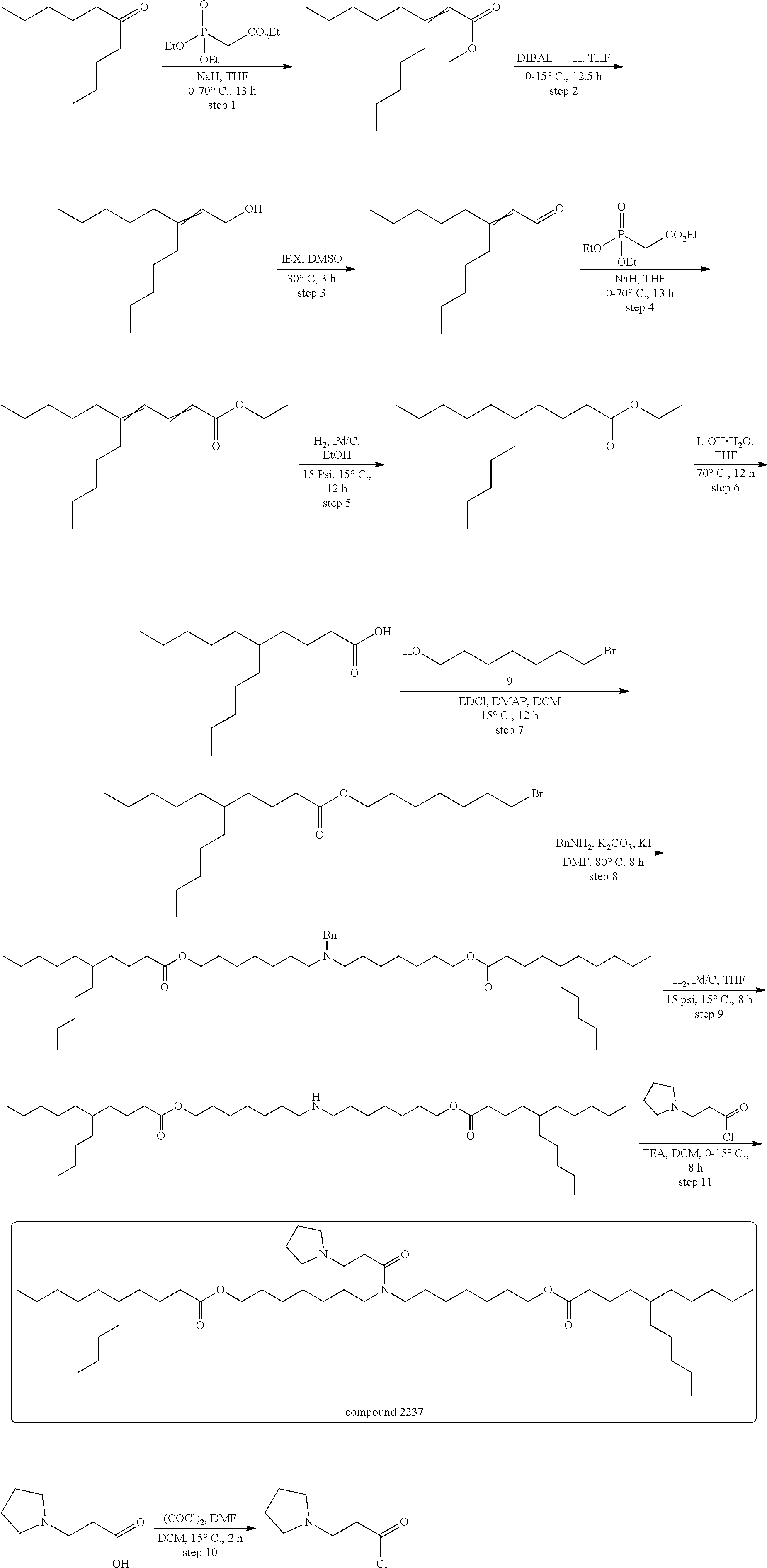

### Step 1:

To a solution of NaH (1.17 g, 29.36 mmol, 60% purity, 1 eq) in THE (40 mL) was added dropwise ethyl 2-diethoxyphosphorylacetate (9.87 g, 44.04 mmol, 8.74 mL, 1.5 eq) at 15° C., then stirred at 15° C. for 30 minutes, and then cooled to 0° C. Undecan-6-one (5 g, 29.36 mmol, 1 eq) was added dropwise to the mixture at 0° C. The mixture was stirred for 30 minutes at 15° C. and at 70° C. for 12 hours. The reaction mixture was quenched by addition aqueous saturated NaHCO₃ 50 mL at 15° C., then extracted with EtOAc 150 mL (50 mL×3). The combined organic layers were washed with brine 100 mL (50 mL×2), dried over Na₂SO₄, filtered and concentrated under reduced pressure to give a residue. The residue was purified by column chromatography (SiO₂, Petroleum ether/Ethyl acetate=1/0 to 10/1) to give compound ethyl 3-pentyloct-2-enoate (16.58 g, 68.97 mmol, 58.73% yield, 4 batches) as a colorless oil.

¹H NMR (400 MHz, CDCl₃), 5.62 (s, 1H), 4.10-4.17 (m, 2H), 2.58-2.61 (m, 2H), 2.10-2.15 (m, 2H), 1.43-1.48 (m, 4H), 1.20-1.45 (m, 12H), 0.89 (t, J=6.8 Hz, 6H).

### Step 2:

To a solution of ethyl 3-pentyloct-2-enoate (11.5 g, 47.84 mmol, 1 eq) in THE (100 mL) was added DIBAL-H (1 M, 143.55 mL, 3.00 eq) at 0° C. The mixture was stirred at 0° C. for 0.5 hour. The mixture was stirred at 15° C. for 12 hours. The reaction mixture was quenched by addition Na₂SO₄·10H₂O (20 g) at 0° C., then added 30 ml H₂O and Na₂SO₄. After that filtered and the filtrate was concentrated under reduced pressure to give a residue. The residue was purified by column chromatography (SiO₂, Petroleum ether/Ethyl acetate=1/0 to 10/1) to give compound 3-pentyloct-2-en-1-ol (7.8 g, 39.33 mmol, 82.20% yield) as a colorless oil.

¹H NMR (400 MHz, CDCl₃), 5.38 (t, J=6.8 Hz, 1H), 4.10-4.17 (m, 2H), 2.00-2.06 (m, 5H), 1.20-1.45 (m, 14H), 0.89 (t, J=6.8 Hz, 6H).

### Step 3:

To a solution of 3-pentyloct-2-en-1-ol (5.8 g, 29.24 mmol, 1 eq) in DMSO (60 mL) was added IBX (12.28 g, 43.86 mmol, 1.5 eq). The mixture was stirred at 30° C. for 3 hours. The reaction mixture was quenched by addition H₂O 60 mL at 15° C., and then filtered to give filtrate and extracted with EtOAc 180 mL (60 mL×3). The combined organic layers were washed with brine 120 mL (60 mL×2), dried over Na₂SO₄, filtered and concentrated under reduced pressure to give a residue. The residue was purified by column chromatography (SiO₂, Petroleum ether/Ethyl acetate=1/0 to 80/1) to give compound 3-pentyloct-2-enal (2.7 g, 13.75 mmol, 47.03% yield) as a colorless oil.

¹H NMR (400 MHz, CDCl₃), 9.99 (d, J=8.0 Hz, 1H), 5.86 (d, J=8.4 Hz, 1H), 2.55 (t, J=8.0 Hz, 2H), 2.21 (t, J=6.8 Hz, 2H), 1.31-1.53 (m, 12H), 0.89 (t, J=6.8 Hz, 6H).

### Step 4:

To a solution of NaH (825.07 mg, 20.63 mmol, 60% purity, 1.5 eq) in THF (30 mL) was added dropwise ethyl 2-diethoxyphosphorylacetate (6.17 g, 27.50 mmol, 5.46 mL, 2 eq) at 15° C. and stirred at 15° C. for 30 minutes, then cooled to 0° C. 3-pentyloct-2-enal (2.7 g, 13.75 mmol, 1 eq) was added dropwise to the mixture. The mixture was stirred at 15° C. for 30 minutes and at 70° C. for 12 hours. The reaction mixture was quenched by addition NaHCO₃ 50 mL at 15° C., and then extracted with EtOAc 150 mL (50 mL×3). The combined organic layers were washed with brine 100 mL (50 mL×2), dried over Na₂SO₄, filtered and concentrated under reduced pressure to give a residue. The residue was purified by column chromatography (SiO₂, Petroleum ether/Ethyl acetate=1/0 to 40/1) to give compound ethyl 5-pentyldeca-2,4-dienoate (3.5 g, 13.14 mmol, 95.53% yield) as a colorless oil.

¹H NMR (400 MHz, CDCl₃), 7.55-7.62 (m, 1H), 5.97 (d, J=11.6 Hz, 1H), 5.78 (d, J=15.2 Hz, 1H), 4.17-4.23 (m, 2H), 2.27 (t, J=7.6 Hz, 2H), 2.14 (t, J=7.2 Hz, 2H), 1.26-1.50 (m, 16H), 0.89 (t, J=6.8 Hz, 6H).

### Step 5:

To a solution of Pd/C (500 mg, 13.14 mmol, 10% purity, 1 eq) in EtOH (35 mL) was added ethyl 5-pentyldeca-2,4-dienoate (3.5 g, 13.14 mmol, 1 eq) under N₂ atmosphere. The suspension was degassed and purged with H₂ for 3 times. The mixture was stirred under H₂ (15 Psi) at 15° C. for 12 hours. The reaction mixture was filtered and concentrated under reduced pressure to give compound ethyl 5-pentyldecanoate (2.5 g, 9.24 mmol, 70.36% yield) as a colorless oil.

¹H NMR (400 MHz, CDCl₃), 4.10-4.16 (m, 2H), 2.27 (t, J=7.2 Hz, 2H), 1.57-1.62 (m, 2H), 1.23-1.32 (m, 22H), 0.89 (t, J=6.8 Hz, 6H).

### Step 6:

To a solution of ethyl 5-pentyldecanoate (2.5 g, 9.24 mmol, 1 eq) in THE (50 mL) and H₂O (10 mL) added LiOH·H₂O (581.86 mg, 13.87 mmol, 1.5 eq). The mixture was stirred at 70° C. for 12 hours. The reaction mixture was concentrated under reduced pressure to get a residue. The residue was extracted with PE 150 mL (50 mL×3). The aqueous phase was dropwise added 1M HCl until the pH was 6-7 and extracted with EtOAc 150 mL (50 mL×3). The combined organic layers were washed with brine 100 mL (50 mL×2), dried over Na₂SO₄, filtered and concentrated under reduced pressure to give compound 5-pentyldecanoic acid (1.95 g, 8.02 mmol, 86.80% yield) as a colorless oil.

¹H NMR (400 MHz, CDCl₃), 2.34 (t, J=7.6 Hz, 2H), 1.57-1.62 (m, 2H), 1.23-1.32 (m, 20H), 0.89 (t, J=6.8 Hz, 6H).

### Step 7:

To a solution of 5-pentyldecanoic acid (1.9 g, 7.84 mmol, 1 eq) and 7-bromoheptan-1-ol (1.84 g, 9.41 mmol, 1.2 eq) in DCM (30 mL) was added EDCI (2.25 g, 11.76 mmol, 1.5 eq) and DMAP (478.80 mg, 3.92 mmol, 0.5 eq). The mixture was stirred at 15° C. for 12 hours. The reaction mixture was filtered and concentrated under reduced pressure to give a residue. The residue was purified by column chromatography (SiO₂, Petroleum ether/Ethyl acetate=1/0 to 80/1) to give compound 7-bromoheptyl 5-pentyldecanoate (3 g, 7.15 mmol, 91.24% yield) as a colorless oil.

¹H NMR (400 MHz, CDCl₃), 4.05-4.15 (m, 2H), 3.41 (t, J=6.8 Hz, 2H), 2.28 (t, J=7.2 Hz, 2H), 1.86-1.88 (m, 2H), 1.57-1.80 (m, 4H), 1.23-1.32 (m, 26H), 0.89 (t, J=6.8 Hz, 6H).

### Step 8:

To a solution of phenylmethanamine (377.50 mg, 3.52 mmol, 384.03 μL, 1 eq) in DMF (75 mL) was added K₂CO₃ (2.43 g, 17.62 mmol, 5 eq) and KI (1.46 g, 8.81 mmol, 2.5 eq), then a solution of 7-bromoheptyl 5-pentyldecanoate (3 g, 7.15 mmol, 2.03 eq) in DMF (30 mL) was added to the mixture. The mixture was stirred at 80° C. for 8 hours. The reaction mixture was quenched by addition H₂O 100 mL at 15° C., and then extracted with EtOAc 150 mL (50 mL×3). The combined organic layers were washed with brine 100 mL (50 mL×2), dried over Na₂SO₄, filtered and concentrated under reduced pressure to give a residue. The residue was purified by column chromatography (SiO₂, Petroleum ether/Ethyl acetate=10/1 to 3/1) to give compound 7-[benzyl-[7-(5-pentyl decanoyloxy)heptyl]amino] heptyl 5-pentyldecanoate (1.5 g, 1.91 mmol, 54.29% yield) as a colorless oil.

### Step 9:

A solution of 7-[benzyl-[7-(5-pentyldecanoyloxy)heptyl]amino]heptyl 5-pentyldecanoate (1.5 g, 1.91 mmol, 1 eq) and Pd/C (150 mg, 10% purity) in THF (10 mL) was stirred under H₂ (15 Psi) at 15° C. for 8 hours. The reaction mixture was filtered and concentrated under reduced pressure to give a residue. The residue was purified by column chromatography (SiO₂, Petroleum ether/Ethyl acetate=10/1 to 0/1) to give compound 7-[7-(5-pentyldecanoyloxy) heptylamino]heptyl 5-pentyldecanoate (391 mg, 563.27 μmol, 29.45% yield) as colorless oil.

¹H NMR (400 MHz, CDCl₃), 4.06 (t, J=6.8 Hz, 2H), 2.59 (t, J=7.2 Hz, 2H), 2.28 (t, J=7.2 Hz, 4H), 1.58-1.66 (m, 7H), 1.48-1.55 (m, 5H), 1.22-1.35 (m, 44H), 0.89 (t, J=6.8 Hz, 12H).

### Step 10:

To a solution of 3-pyrrolidin-1-ylpropanoic acid (170 mg, 1.19 mmol, 1 eq) in DCM (5 mL) was added (COCl)₂ (753.49 mg, 5.94 mmol, 519.65 μL, 5 eq) and DMF (8.68 mg, 118.73 μmol, 9.14 μL, 0.1 eq). The mixture was stirred at 15° C. for 2 hours. The reaction mixture was concentrated under reduced pressure to give a compound 3-pyrrolidin-1-ylpropanoyl chloride (235.19 mg, 1.19 mmol, crude, HCl salt) as a yellow solid.

### Step 11:

To a solution of 7-[7-(5-pentyldecanoyloxy)heptylamino]heptyl 5-pentyldecanoate (150 mg, 216.09 μmol, 1 eq) in DCM (5 mL) was added TEA (153.06 mg, 1.51 mmol, 210.54 μL, 7 eq) and 3-pyrrolidin-1-ylpropanoyl chloride (235.19 mg, 1.19 mmol, 5.49 eq, HCl) at 0° C. The mixture was stirred at 15° C. for 8 hours. The reaction mixture was filtered and concentrated under reduced pressure to give a residue. The residue was purified by column chromatography (SiO₂, Petroleum ether/Ethyl acetate=10/1 to 0/1) to give compound 7-[7-(5-pentyldecanoyloxy)heptyl-(3-pyrrolidin-1-ylpropanoyl)amino]heptyl 5-pentyldecanoate (29 mg, 35.39 μmol, 16.38% yield) as a yellow oil.

¹H NMR (400 MHz, CDCl₃), 4.03-4.09 (m, 4H), 3.29 (t, J=8.0 Hz, 2H), 3.23 (t, J=7.2 Hz, 2H), 2.41-3.10 (m, 8H), 2.25-2.30 (m, 4H), 1.70-2.05 (brs, 4H), 1.58-1.66 (m, 8H), 1.48-1.55 (m, 4H), 1.17-1.35 (m, 50H), 0.89 (t, J=6.8 Hz, 12H. LCMS: (M+H⁺): 819.6 @ 3.180 min.

### 4.26. Synthesis of Compound 2238

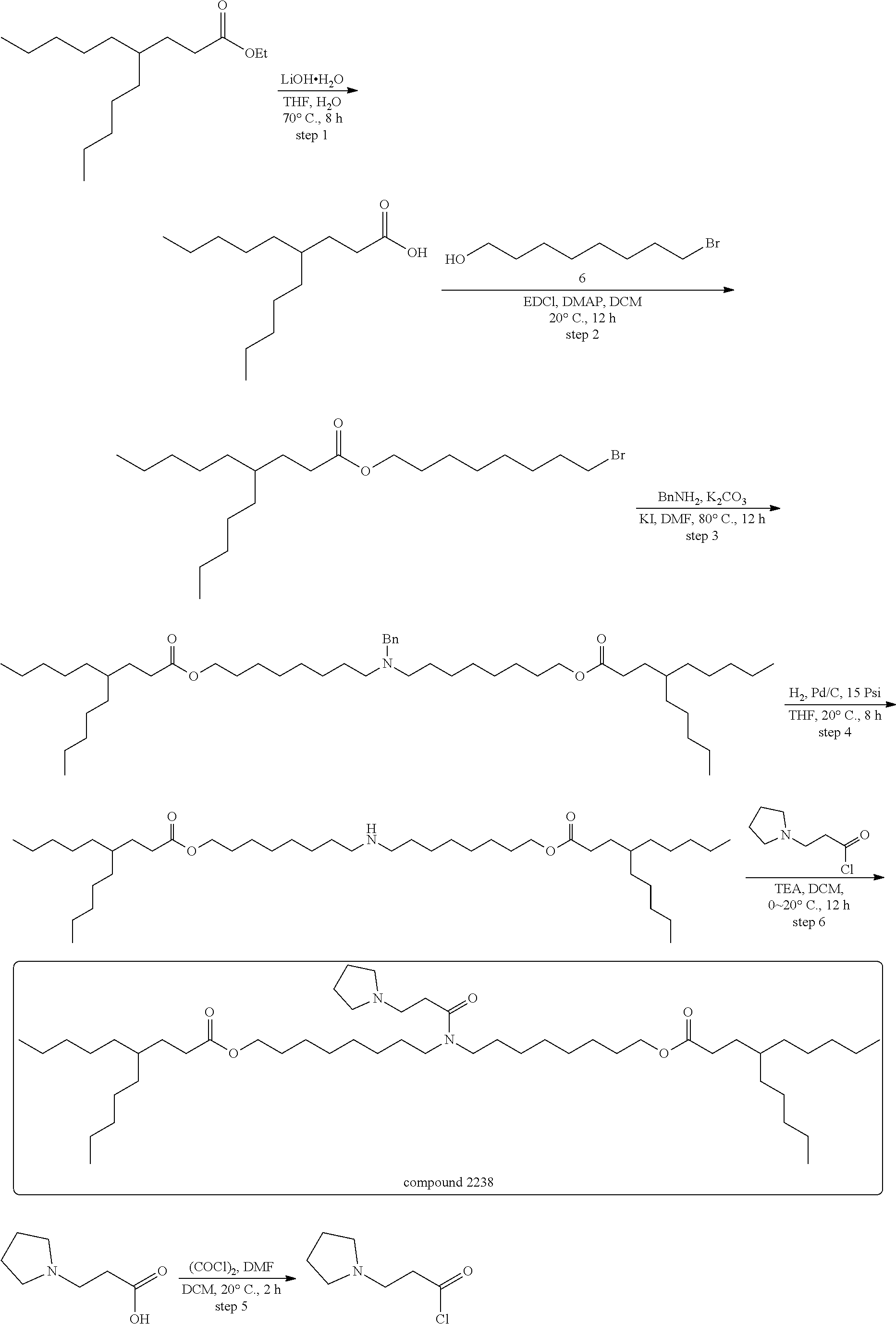

### Step 1:

To a solution of ethyl 4-pentylnonanoate (1.2 g, 4.68 mmol, 1 eq) in THF (10 mL) was added a solution of LiOH·H₂O (294.57 mg, 7.02 mmol, 7.05 mL, 1.5 eq) in H₂O (2 mL), the mixture was stirred at 70° C. for 8 hours. The mixture was added H₂O (20 mL) at 0° C., then concentrated under reduced pressure to removed THF. The water phase was extracted with petroleum ether (10 mL×3), then adjust the pH=˜3 with 1N aq.HCl and extracted with EtOAc (20 mL×2). The combined organic layer was washed with brine (50 mL×2), dried over Na₂SO₄, filtered and the filtrate was concentrated under reduced pressure to give the 4-pentylnonanoic acid (1 g, crude) as colorless oil.

### Step 2:

To a solution of 4-pentylnonanoic acid (1 g, 4.38 mmol, 1 eq) and 8-bromooctan-1-ol (1.01 g, 4.82 mmol, 825.65 μL, 1.1 eq) in DCM (5 mL) was added DMAP (267.48 mg, 2.19 mmol, 0.5 eq) and EDCI (1.26 g, 6.57 mmol, 1.5 eq), and stirred at 20° C. for 12 hours. The mixture was added into H₂O (5 mL) and extracted with EtOAc (5 mL×3). The organic layer was washed with brine (5 mL×2), dried over Na₂SO₄, filtered and the filtrate was concentrated under reduced pressure. The residue was purified by column chromatography (SiO₂, Petroleum ether/Ethyl acetate=5/1 to 0/1) to give 8-bromooctyl 4-pentylnonanoate (1.7 g, 4.05 mmol, 92.55% yield) as colorless oil.

### Step 3:

To a solution of BnNH₂ (210 mg, 1.96 mmol, 213.63 μL, 1 eq) in DMF (5 mL) was added K₂CO₃ (1.35 g, 9.80 mmol, 5 eq) and KI (813.33 mg, 4.90 mmol, 2.5 eq). Then a solution of 8-bromooctyl 4-pentylnonanoate (1.67 g, 3.97 mmol, 2.03 eq) in DMF (5 mL) was added to the mixture and stirred at 80° C. for 12 hours. The mixture was filtered and the filtrate was poured into H₂O (50 mL) and extracted with EtOAc (10 mL×3). The combined organic layer was washed with brine (10 mL×2), dried over Na₂SO₄, filtered and the filtrate was concentrated under reduced pressure. The residue was purified by column chromatography (SiO₂, Petroleum ether/Ethyl acetate=100/1 to 20/1) to give 8-[benzyl-[8-(4-pentylnonanoyloxy)octyl]amino]octyl 4-pentylnonanoate (700 mg, 892.53 μmol, 45.54% yield) as yellow oil.

### Step 4:

To a solution of Pd/C (100 mg, 892.53 μmol, 10% purity, 1 eq) in THE (50 mL) was added 8-[benzyl-[8-(4-pentylnonanoyloxy)octyl]amino]octyl 4-pentylnonanoate (700 mg, 892.53 μmol, 1 eq), and was stirred at 20° C. for 8 hours under H₂ under 15 Psi. The mixture was filtered and the filtrate was concentrated under reduced pressure to give 8-[8-(4-pentylnonanoyloxy)octylamino]octyl 4-pentylnonanoate (300 mg, 432.18 μmol, 48.42% yield) as brown oil.

¹H NMR (400 MHz, CDCl₃), 4.06 (t, J=6.4 Hz, 4H), 2.60 (t, J=7.6 Hz, 4H), 2.25-2.30 (m, 4H), 1.45-1.66 (m, 16H), 1.20-1.31 (m, 46H), 0.89 (t, J=6.8 Hz, 12H).

### Step 5:

To a solution of 3-pyrrolidin-1-ylpropanoic acid (320.00 mg, 2.23 mmol, 1 eq) in DCM (5 mL) was added (COCl)₂ (1.42 g, 11.17 mmol, 978.19 μL, 5 eq) and DMF (16.33 mg, 223.49 μmol, 17.19 μL, 0.1 eq), and was stirred at 20° C. for 2 hours. The mixture was concentrated under reduced pressure to give 3-pyrrolidin-1-ylpropanoyl chloride (443 mg, crude) as a yellow solid.

### Step 6:

To a solution of 8-[8-(4-pentylnonanoyloxy)octylamino]octyl 4-pentylnonanoate (300 mg, 432.18 μmol, 1 eq) in DCM (2 mL) was added TEA (306.12 mg, 3.03 mmol, 421.08 μL, 7 eq) and 3-pyrrolidin-1-ylpropanoyl chloride (428.05 mg, 2.16 mmol, 5 eq, HCl) at 0° C., and was stirred at 20° C. for 12 hours. The mixture was concentrated under reduced pressure to get a residue. The residue was purified by column chromatography (SiO₂, Ethyl acetate/MeOH=50/1 to 1/1) to give 8-[8-(4-pentylnonanoyloxy)octyl-(3-pyrrolidin-1-ylpropanoyl)amino]octyl 4-pentylnonanoate (34 mg, 40.67 μmol, 9.41% yield) as yellow oil.

¹H NMR (400 MHz, CDCl₃), 4.02-4.07 (m, 4H), 3.28 (t, J=7.6 Hz, 2H), 3.22 (t, J=7.6 Hz, 2H), 2.78-2.83 (m, 2H), 2.56 (brs, 6H), 2.25-2.30 (m, 4H), 1.81 (brs, 4H), 1.58-1.66 (m, 8H), 1.48-1.55 (m, 4H), 1.24-1.35 (m, 50H), 0.89 (t, J=6.8 Hz, 12H).

LCMS: (M+H⁺): 819.8 @ 2.258 min.

### 4.26. Synthesis of Compound 2239

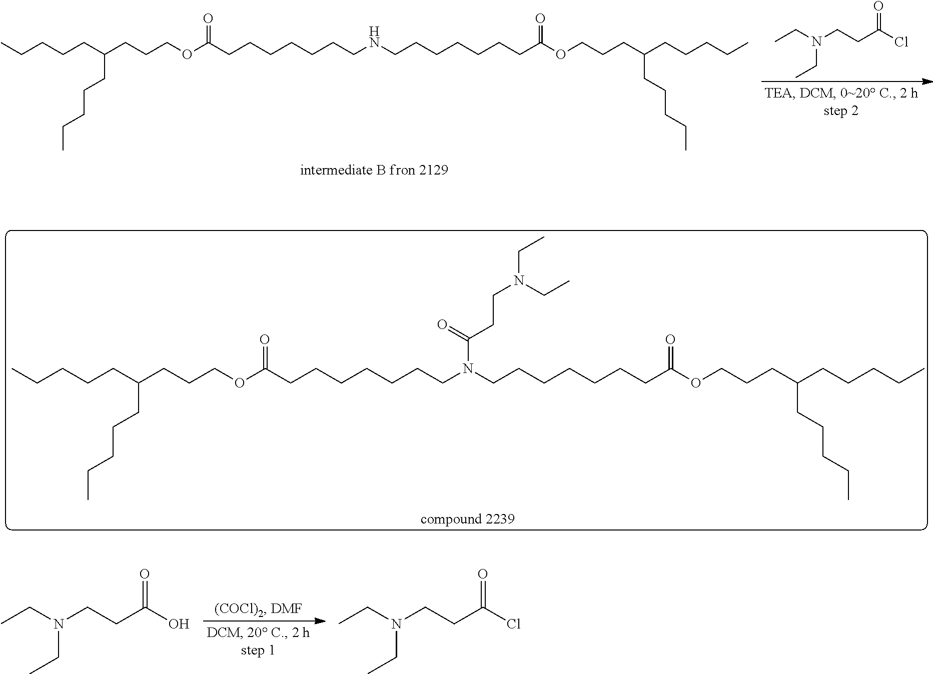

### Step 1:

To a solution of 3-(diethylamino)propanoic acid (130 mg, 715.62 μmol, 1 eq, HCl) in DCM (5 mL) was added (COCl)₂ (363.33 mg, 2.86 mmol, 250.57 μL, 4 eq) and DMF (5.23 mg, 71.56 μmol, 5.51 μL, 0.1 eq), then the mixture was stirred at 20° C. for 2 hours. The mixture was concentrated under reduced pressure. 3-(diethylamino)propanoyl chloride (150 mg, crude, HCl) was obtained as a yellow solid.

### Step 2:

To a solution of 4-pentylnonyl 8-[[8-oxo-8-(4-pentylnonoxy)octyl]amino]octanoate (130 mg, 187.28 μmol, 1 eq) in DCM (2 mL) was added TEA (94.75 mg, 936.38 μmol, 130.33 μL, 5 eq) and 3-(diethylamino)propanoyl chloride (149.90 mg, 749.10 μmol, 4.00 eq, HCl) at 0° C., then the mixture was stirred at 20° C. for 12 hours. The mixture was concentrated under reduced pressure. The residue was purified by column chromatography (SiO₂, Petroleum ether:Ethyl acetate=10/1 to 0/1). 4-pentylnonyl 8-[3-(diethylamino)propanoyl-[8-oxo-8-(4-pentylnonoxy)octyl]amino]octanoate (70 mg, 83.52 μmol, 44.60% yield, 98% purity) was obtained as colorless oil.

¹H NMR (400 MHz, CDCl₃), 4.02-4.07 (m, 4H), 3.28 (t, J=7.6 Hz, 2H), 3.22 (t, J=8.0 Hz, 2H), 2.78-1.83 (m, 2H), 2.52-2.57 (m, 4H), 2.35-2.48 (m, 2H), 2.27-2.30 (m, 4H), 1.58-1.66 (m, 8H), 1.48-1.55 (m, 4H), 1.24-1.35 (m, 50H), 1.01-1.07 (m, 6H), 0.89 (t, J=7.2 Hz, 12H).

LCMS: (M+H⁺): 821.9 @ 2.562 min.

### 4.27: Synthesis of Compound 2241

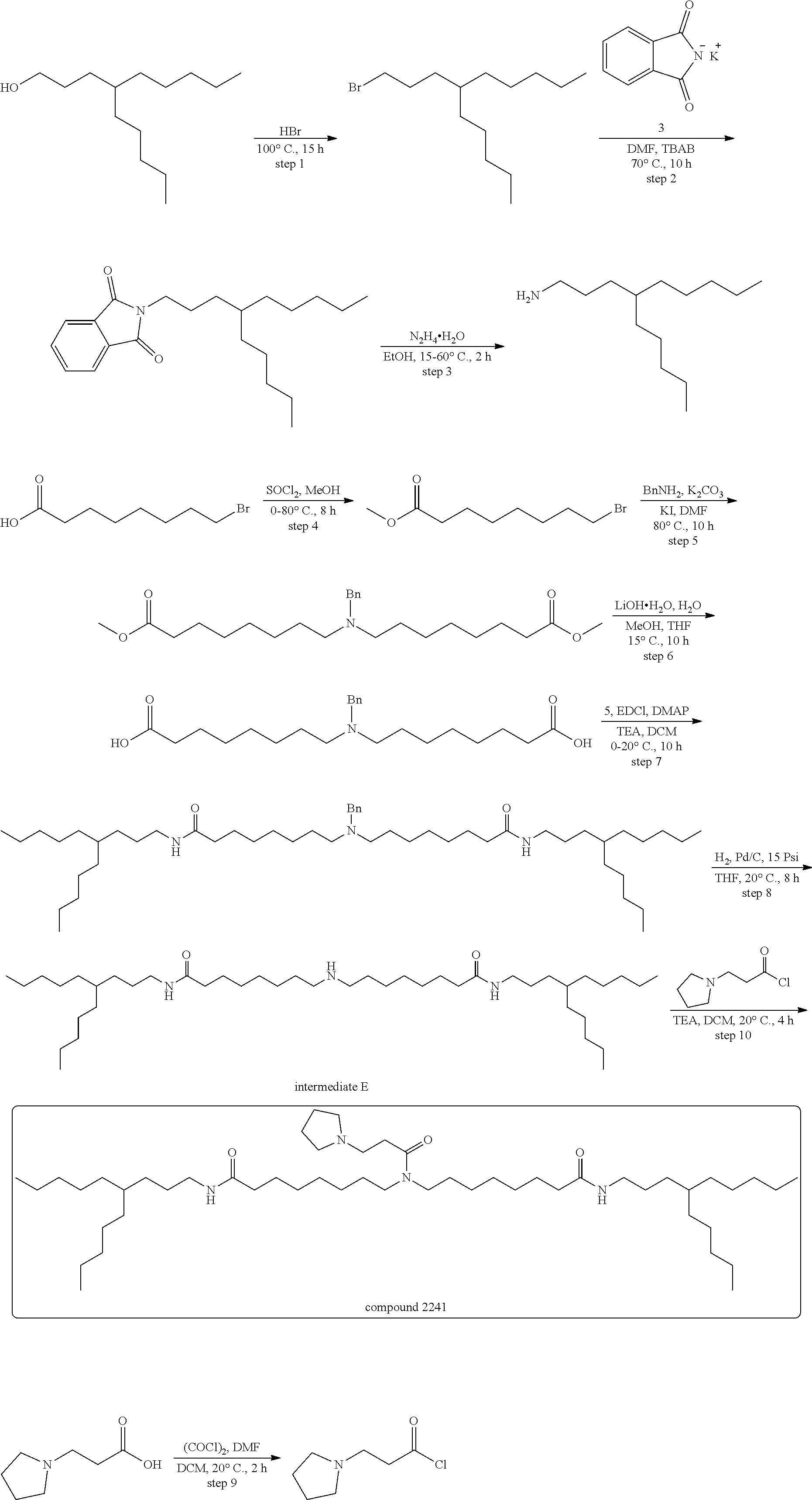

### Step 1:

A mixture of 4-pentylnonan-1-ol (10 g, 46.64 mmol, 1 eq and HBr (74.50 g, 368.30 mmol, 50 mL, 40% purity, 7.90 eq was stirred at 100° C. for 15 hours under N₂ atmosphere. The reaction mixture was diluted with H₂O 50 mL extracted with EtOAc 400 mL (200 mL×2). The combined organic layers were dried over Na₂SO₄, filtered and concentrated under reduced pressure to give a residue. The residue was purified by column chromatography (SiO₂, Petroleum ether/Ethyl acetate=1/0 to 8/1) to give compound 6-(3-bromopropyl)undecane (9.68 g, 34.91 mmol, 74.84% yield) as colorless oil.

¹H NMR (400 MHz, CDCl₃), 3.40 (t, J=6.8 Hz, 2H), 1.80-1.86 (m, 2H), 1.25-1.39 (m, 19H), 0.88 (t, J=6.8 Hz, 6H).

### Step 2:

A mixture of 6-(3-bromopropyl)undecane (9.68 g, 34.91 mmol, 1 eq), tetrabutylammonium; bromide (2.25 g, 6.98 mmol, 0.2 eq) and (1,3-dioxoisoindolin-2-yl)potassium (9.70 g, 52.37 mmol, 1.5 eq) in DMF (150 mL) was stirred at 70° C. for 10 hours under N₂ atmosphere. The reaction mixture was filtered and diluted with aqueous saturated NaCl 150 mL extracted with EtOAc 500 mL (250 mL×2). The combined organic layers were washed with brine 750 mL (250 mL×3) and dried over Na₂SO₄, filtered and concentrated under reduced pressure to give a residue. The residue was purified by column chromatography (SiO₂, Petroleum ether/Ethyl acetate=8/1 to 5/1) to give compound 2-(4-pentylnonyl)isoindoline-1,3-dione (10.4 g, 30.28 mmol, 86.73% yield) as colorless oil.

¹H NMR (400 MHz, CDCl₃), 7.84-7.86 (m, 2H), 7.27-7.72 (m, 2H), 3.66 (t, J=6.8 Hz, 2H), 1.64-1.68 (m, 2H), 1.22-1.39 (m, 19H), 0.87 (t, J=6.8 Hz, 6H).

### Step 3:

To a solution of 2-(4-pentylnonyl)isoindoline-1,3-dione (10.4 g, 30.28 mmol, 1 eq) in EtOH (120 mL) was added dropwise hydrazine; hydrate (20.52 g, 327.87 mmol, 19.92 mL, 80% purity, 10.83 eq) at 15° C. The mixture was degassed and purged with N₂ for 3 times, and then stirred at 60° C. for 2 hours under N₂ atmosphere. The mixture was filtered and concentrated under reduced pressure to give a residue. The residue was purified by column chromatography (SiO₂, Petroleum ether/Ethyl acetate=3/1 to 0/1, 5% NH₃·H₂O) to give compound 4-pentylnonan-1-amine (3.76 g, 17.62 mmol, 58.20% yield) as colorless oil.

¹H NMR (400 MHz, CDCl₃), 2.66 (t, J=6.8 Hz, 2H), 1.38-1.40 (m, 2H), 1.23-1.30 (m, 19H), 1.16 (s, 2H), 0.88 (t, J=6.8 Hz, 6H).

### Step 4:

To a solution of 8-bromooctanoic acid (10 g, 44.82 mmol, 1 eq) in MeOH (150 mL) was added dropwise SOCl₂ (16.00 g, 134.46 mmol, 9.75 mL, 3 eq) at 0° C. The mixture was degassed and purged with N₂ for 3 times, and then stirred at 80° C. for 8 hours under N₂ atmosphere. The reaction mixture was concentrated under reduced pressure to give crude product methyl 8-bromooctanoate (8 g, crude) as colorless oil and used into the next step without further purification.

### Step 5:

A mixture of methyl 8-bromooctanoate (8 g, 33.74 mmol, 2 eq), phenylmethanamine (1.81 g, 16.87 mmol, 1.84 mL, 1 eq), K₂CO₃ (11.66 g, 84.34 mmol, 5 eq) and KI (7.00 g, 42.17 mmol, 2.5 eq) in DMF (250 mL) was stirred at 80° C. for 10 hours under N₂ atmosphere. The reaction mixture was filtered and diluted with H₂O 300 mL then extracted with EtOAc 1000 mL (250 mL×4). The combined organic layers were dried over Na₂SO₄, filtered and concentrated under reduced pressure to give a residue. The residue was purified by column chromatography (SiO₂, Petroleum ether/Ethyl acetate=0/1 to 1/1) to give compound methyl 8-[benzyl-(8-methoxy-8-oxo-octyl)amino]octanoate (4 g, 9.53 mmol, 56.51% yield) was obtained as colorless oil.

¹H NMR (400 MHz, CDCl₃), 7.22-7.33 (m, 5H), 3.67 (s, 6H), 3.53 (s, 2H), 2.38 (t, J=6.8 Hz, 4H), 2.30 (t, J=6.8 Hz, 4H), 1.57-1.62 (m, 4H), 1.43-1.46 (m, 4H), 1.27-1.38 (m, 12H).

### Step 6:

To a solution of methyl 8-[benzyl-(8-methoxy-8-oxo-octyl)amino]octanoate (4 g, 9.53 mmol, 1 eq) in THF (10 mL) and MeOH (30 mL) was added dropwise NaOH (1.30 g, 32.51 mmol, 3.41 eq) in H₂O (10 mL) at 15° C. The mixture was degassed and purged with N₂ for 3 times, and then stirred at 15° C. for 10 hours under N₂ atmosphere. The reaction mixture was adjusted to pH=3.0 with 1N HCl 23 mL and extracted with EtOAc/MeOH=10/1 200 mL (100 mL×2). The combined organic layers were concentrated under reduced pressure to give compound 8-[benzyl(7-carboxyheptyl)amino]octanoic acid (3.2 g, 8.17 mmol, 85.73% yield) as colorless oil.

### Step 7:

To a solution of 8-[benzyl(7-carboxyheptyl)amino]octanoic acid (3 g, 7.66 mmol, 1 eq) and 4-pentylnonan-1-amine (3.60 g, 16.86 mmol, 2.2 eq) in DCM (150 mL) was added EDCI (4.41 g, 22.99 mmol, 3 eq), DMAP (468.03 mg, 3.83 mmol, 0.5 eq) and TEA (2.33 g, 22.99 mmol, 3.20 mL, 3 eq) at 0° C. The mixture was degassed and purged with N₂ for 3 times, and then stirred at 20° C. for 10 hours under N₂ atmosphere. The reaction mixture was diluted with H₂O 100 mL and extracted with EtOAc 800 mL (400 mL×2). The combined organic layers were dried over Na₂SO₄, filtered and concentrated under reduced pressure to give a residue. The residue was purified by column chromatography (SiO₂, Petroleum ether/Ethyl acetate=10/1 to 5/1, 5% NH₃·H₂O) to give compound 8-[benzyl-[8-oxo-8-(4-pentylnonylamino)octyl]amino]-N-(4-pentylnonyl)octanamide (2.87 g, 3.67 mmol, 47.88% yield) as colorless oil.

¹H NMR (400 MHz, CDCl₃), 7.23-7.31 (m, 5H), 5.50 (brs, 2H), 3.53 (s, 2H), 3.21 (q, J=8 Hz, 4H), 2.38 (t, J=6.8 Hz, 4H), 2.14 (t, J=6.8 Hz, 4H), 1.57-1.62 (m, 4H), 1.42-1.48 (m, 8H), 1.22-1.32 (m, 50H), 0.88 (t, J=6.8 Hz, 12H).

### Step 8:

To a suspension of Pd/C (1 g, 3.58 mmol, 10% purity) in THF (20 mL) was added 8-[benzyl-[8-oxo-8-(4-pentylnonylamino)octyl]amino]-N-(4-pentylnonyl)octanamide (2.8 g, 3.58 mmol, 1 eq) in THF (30 mL). The mixture was stirred at 20° C. for 8 hours under H₂ (15 Psi) atmosphere. The reaction mixture was filtered and concentrated under reduced pressure to give a residue. The residue was purified by column chromatography (SiO₂, Petroleum ether/Ethyl acetate=10/1 to 1/1, 5% NH₃·H₂O) to give compound 8-[[8-oxo-8-(4-pentylnonylamino)octyl]amino]-N-(4-pentylnonyl)octanamide (2 g, 2.89 mmol, 80.73% yield) as a white solid.

¹H NMR (400 MHz, CDCl₃), 5.45 (brs, 2H), 3.22 (q, J=8 Hz, 4H), 2.55 (t, J=6.8 Hz, 4H), 2.14 (t, J=6.8 Hz, 4H), 1.50-1.63 (m, 4H), 1.44-1.48 (m, 8H), 1.22-1.32 (m, 50H), 0.89 (t, J=6.8 Hz, 12H).

### Step 9:

To a solution of 3-pyrrolidin-1-ylpropanoic acid (70 mg, 488.88 μmol, 1 eq) in DCM (9 mL) was added DMF (19.00 mg, 259.94 μmol, 0.02 mL, 5.32e-1 eq) and oxalyl dichloride (310.26 mg, 2.44 mmol, 213.97 μL, 5 eq) at 20° C. The mixture was degassed and purged with N₂ for 3 times, and then stirred at 20° C. for 2 hours under N₂ atmosphere. The reaction mixture was concentrated under reduced pressure to give crude product 3-pyrrolidin-1-ylpropanoyl chloride (80 mg, crude, HCl) as yellow solid and used into the next step without further purification.

### Step 10:

To a solution of 8-[[8-oxo-8-(4-pentylnonylamino)octyl]amino]-N-(4-pentylnonyl)octanamide (100 mg, 144.47 μmol, 1 eq) and TEA (43.86 mg, 433.41 μmol, 60.32 μL, 3 eq) in DCM (8 mL) was added dropwise 3-pyrrolidin-1-ylpropanoyl chloride (70.05 mg, 433.41 μmol, 3 eq, HCl) in DCM (2 mL) at 20° C. The mixture was degassed and purged with N₂ for 3 times, and then stirred at 20° C. for 4 hours under N₂ atmosphere. The reaction mixture was diluted with H₂O 10 mL extracted with EtOAc 40 mL (20 mL×2). The combined organic layers were dried over Na₂SO₄, filtered and concentrated under reduced pressure to give a residue. The residue was purified by column chromatography (SiO₂, Petroleum ether/Ethyl acetate=4/1 to 1/1, 5% NH₃·H₂O) to give crude product. Then the crude product was purified by prep-TLC (SiO₂, Petroleum ether/Ethyl acetate=0/1, 5% NH₃·H₂O) to give compound 8-[[8-oxo-8-(4-pentylnonylamino)octyl]-(3-pyrrolidin-1-ylpropanoyl)amino]-N-(4-pentylnonyl)octanamide (23 mg, 28.14 μmol, 19.48% yield, 100% purity) as colorless oil.

¹H NMR (400 MHz, CDCl₃), 5.58-5.65 (m, 2H), 3.18-3.30 (m, 8H), 2.82 (t, J=8.0 Hz, 2H), 2.50-2.57 (m, 6H), 2.12-2.17 (m, 4H), 1.80 (s, 4H), 1.59-1.64 (m, 4H), 1.44-1.52 (m, 8H), 1.22-1.32 (m, 50H), 0.88 (t, J=6.8 Hz, 12H). LCMS: (M+H⁺): 817.7 @ 3.041 min.

### 4.28. Synthesis of Compound 2242

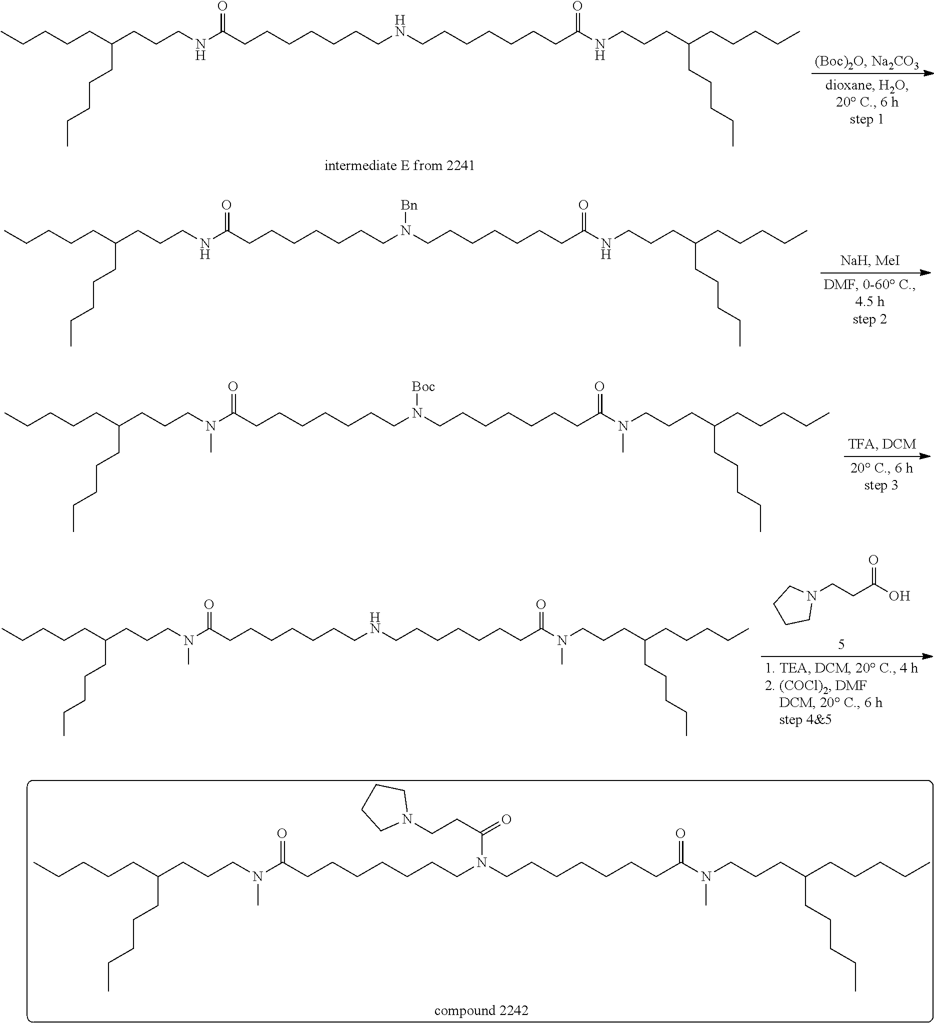

### Step 1:

To a solution of 8-[[8-oxo-8-(4-pentylnonylamino)octyl]amino]-N-(4-pentylnonyl)octanamide (500 mg, 722.34 μmol, 1 eq) in H₂O (15 mL) and dioxane (15 mL) was added (Boc)₂O (236.47 mg, 1.08 mmol, 248.92 μL, 1.5 eq) and Na₂CO₃ (153.12 mg, 1.44 mmol, 2 eq) at 20° C. The mixture was degassed and purged with N₂ for 3 times, and then stirred at 20° C. for 6 hours under N₂ atmosphere. The reaction mixture was diluted with H₂O 15 mL extracted with EtOAc 100 mL (50 mL×2). The combined organic layers were dried over Na₂SO₄, filtered and concentrated under reduced pressure to give a residue. The residue was purified by column chromatography (SiO₂, Petroleum ether/Ethyl acetate=8/1 to 3/1, 5% NH₃·H₂O) to give compound tert-butyl N,N-bis[8-oxo-8-(4-pentylnonylamino)octyl]carbamate (425 mg, 536.41 μmol, 74.26% yield) as colorless oil.

### Step 2:

To a solution of tert-butyl N,N-bis[8-oxo-8-(4-pentylnonylamino)octyl]carbamate (325 mg, 410.19 μmol, 1 eq) in DMF (15 mL) was added NaH (82.03 mg, 2.05 mmol, 60% purity, 5 eq) at 0° C. The mixture was degassed and purged with N₂ for 3 times, and then stirred at 0° C. for 0.5 hour under N₂ atmosphere. Then to the mixture was added dropwise MeI (1.16 g, 8.20 mmol, 510.72 μL, 20 eq) in DMF (5 mL) at 20° C. The mixture was degassed and purged with N₂ for 3 times, and then stirred at 60° C. for 4 hours under N₂ atmosphere. The reaction mixture was quenched by addition H₂O 20 mL at 0° C., and extracted with EtOAc 200 mL (100 mL×2). The combined organic layers were washed with brine 100 mL, dried over Na₂SO₄, filtered and concentrated under reduced pressure to give a residue. The residue was purified by column chromatography (SiO₂, Petroleum ether/Ethyl acetate=10/1 to 5/1, 5% NH₃·H₂O) to give compound tert-butyl N,N-bis[8-[methyl(4-pentylnonyl)amino]-8-oxo-octyl]carbamate (245 mg, 298.65 μmol, 72.81% yield) as colorless oil.

¹H NMR (400 MHz, CDCl₃), 3.34 (t, J=8 Hz, 2H), 3.23 (t, J=8 Hz, 2H), 3.13 (s, 4H), 2.97 (s, 3H), 2.91 (s, 3H), 2.29 (q, J=4.8 Hz, 4H), 1.60-1.68 (m, 4H), 1.46-1.50 (m, 17H), 1.22-1.33 (m, 50H), 0.89 (t, J=6.8 Hz, 12H).

### Step 3:

To a solution of tert-butyl N,N-bis[8-[methyl(4-pentylnonyl)amino]-8-oxo-octyl]carbamate (105 mg, 127.99 μmol, 1 eq) in DCM (9 mL) was added dropwise TFA (1.54 g, 13.51 mmol, 1.00 mL, 105.52 eq) at 20° C. The mixture was degassed and purged with N₂ for 3 times, and then stirred at 20° C. for 6 hours under N₂ atmosphere. The reaction mixture was adjusted to pH=7.0 with sat. NaHCO₃ aq. and extracted with EtOAc 150 mL (50 mL×3). The combined organic layer was dried over Na₂SO₄, filtered and concentrated under reduced pressure to give a residue. The residue was purified by column chromatography (SiO₂, Petroleum ether/Ethyl acetate=3/1 to 0/1, 5% NH₃·H₂O) to give compound N-methyl-8-[[8-[methyl(4-pentylnonyl)amino]-8-oxo-octyl]amino]-N-(4-pentylnonyl)octanamide (150 mg, 208.26 μmol, 81.36% yield) as colorless oil.

¹H NMR (400 MHz, CDCl₃), 3.34 (t, J=8 Hz, 2H), 3.23 (t, J=8 Hz, 2H), 2.97 (s, 3H), 2.91 (s, 3H), 2.60 (t, J=8 Hz, 4H), 2.29 (q, J=4.8 Hz, 4H), 1.46-1.55 (m, 12H), 1.22-1.33 (m, 50H), 0.89 (t, J=6.8 Hz, 12H).

### Step 4:

To a solution of 3-pyrrolidin-1-ylpropanoic acid (50 mg, 349.20 μmol, 1 eq) in DCM (8 mL) was added DMF (7.92 mg, 108.31 μmol, 8.33 μL, 0.31 eq) and oxalyl dichloride (221.61 mg, 1.75 mmol, 152.84 μL, 5 eq) at 20° C. The mixture was stirred at 20° C. for 6 hours under N₂ atmosphere. The reaction mixture was concentrated under reduced pressure to give crude product 3-pyrrolidin-1-ylpropanoyl chloride (70 mg, crude, HCl) as yellow solid and used into the next step without further purification.

### Step 5:

To a solution of N-methyl-8-[[8-[methyl(4-pentylnonyl)amino]-8-oxo-octyl]amino]-N-(4-pentylnonyl)octanamide (100 mg, 138.84 μmol, 1 eq) and TEA (56.20 mg, 555.37 μmol, 77.30 μL, 4 eq) in DCM (8 mL) was added dropwise 3-pyrrolidin-1-ylpropanoyl chloride (68.76 mg, 347.10 μmol, 2.5 eq, HCl) in DCM (4 mL) at 20° C. The mixture was degassed and purged with N₂ for 3 times, and then stirred at 20° C. for 4 hours under N₂ atmosphere.

The reaction mixture was quenched by addition H₂O 15 mL at 0° C., and extracted with EtOAc 80 mL (40 mL×2). The combined organic layers were washed with brine 15 mL, dried over Na₂SO₄, filtered and concentrated under reduced pressure to give a residue. The residue was purified by column chromatography (SiO₂, Petroleum ether/Ethyl acetate=5/1 to 1/1, 5% NH₃·H₂O). Then the crude product was purified by prep-TLC (SiO₂, Petroleum ether/Ethyl acetate=0/1, 5% NH₃·H₂O) to give compound N-methyl-8-[[8-[methyl(4-pentylnonyl)amino]-8-oxo-octyl]-(3-pyrrolidin-1-ylpropanoyl)amino]-N-(4-pentylnonyl) octanamide (22 mg, 25.76 μmol, 18.56% yield, 99% purity) as yellow oil.

¹H NMR (400 MHz, CDCl₃), 3.21-3.28 (m, 8H), 2.97 (d, J=3.6 Hz, 3H), 2.91 (d, J=2.8 Hz, 3H), 2.75-2.83 (m, 2H), 2.48-2.60 (m, 6H), 2.18-2.30 (m, 4H), 1.80 (s, 4H), 1.55-1.69 (m, 4H), 1.46-1.50 (m, 8H), 1.22-1.33 (m, 50H), 0.88 (t, J=6.8 Hz, 12H).

LCMS: (M+H⁺): 845.7 @ 2.836 min.

### 4.29. Synthesis of Compound 2244

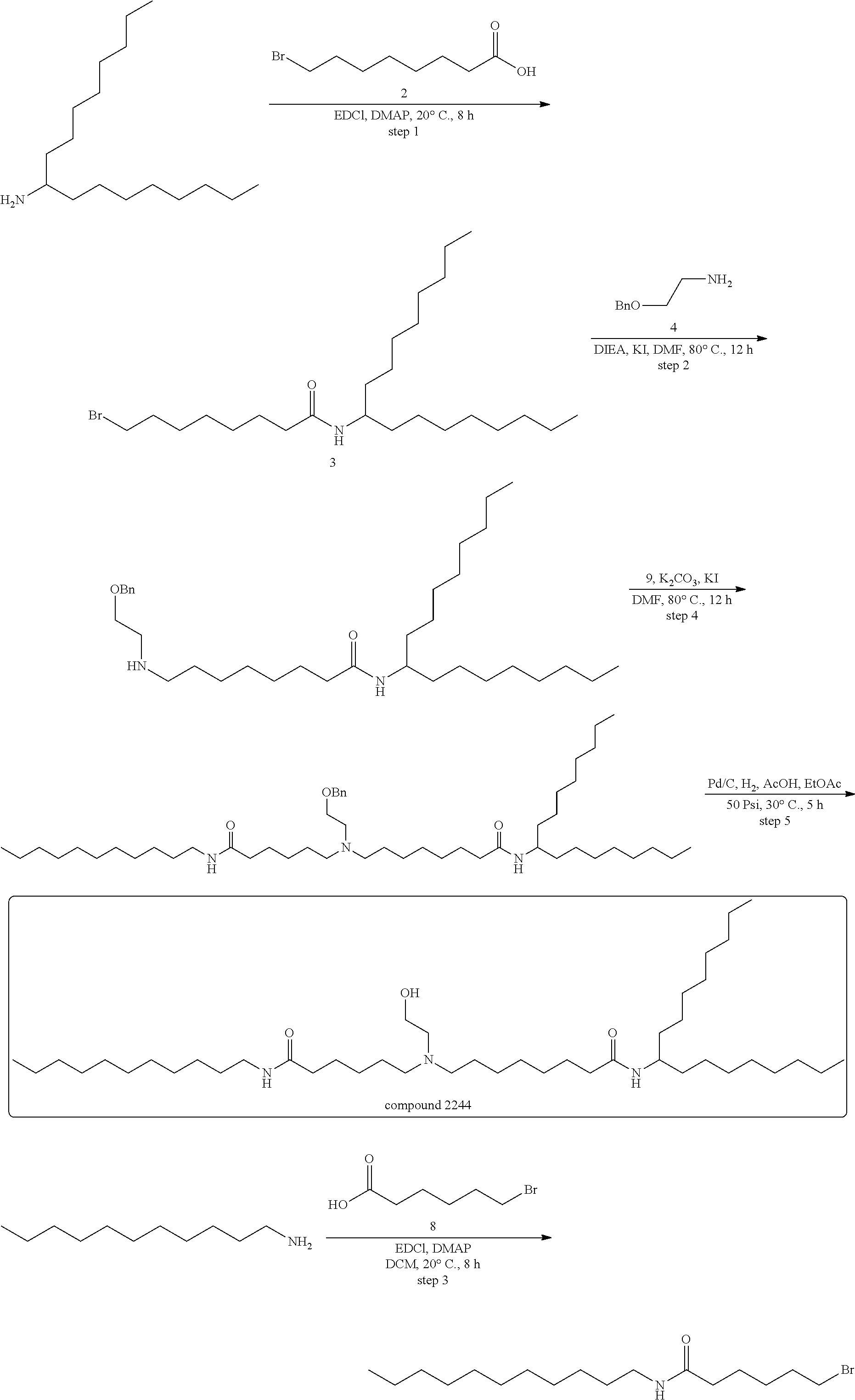

### Step 1:

To a solution of heptadecan-9-amine (2 g, 7.83 mmol, 1 eq) and 8-bromooctanoic acid (1.75 g, 7.83 mmol, 1 eq) in DCM (20 mL) was added DMAP (478.20 mg, 3.91 mmol, 0.5 eq) and EDCI (1.80 g, 9.39 mmol, 1.2 eq), stirred at 20° C. for 8 hours. The mixture was added into H₂O (20 mL) and extracted with EtOAc (20 mL×3). The organic layer was washed with brine (20 mL×2), dried over Na₂SO₄, filtered and the filtrate was concentrated under reduced pressure. The residue was purified by column chromatography (SiO₂, Petroleum ether/Ethyl acetate=1/0 to 5/1) to give 8-bromo-N-(1-octylnonyl)octanamide (2.5 g, 5.43 mmol, 69.34% yield) as a white solid.

### Step 2:

To a solution of 2-benzyloxyethanamine (620 mg, 4.10 mmol, 1 eq) in DMF (20 mL) was added KI (748.73 mg, 4.51 mmol, 1.1 eq) and DIEA (1.06 g, 8.20 mmol, 1.43 mL, 2 eq), then a solution of 8-bromo-N-(1-octylnonyl)octanamide (1.98 g, 4.31 mmol, 1.05 eq) in DMF (10 mL) was added to the mixture and stirred at 80° C. for 12 hours. The mixture was filtered and the filtrate was added into H₂O (50 mL) and extracted with EtOAc (10 mL×3). The combined organic layer was washed with brine (10 mL×2), dried over Na₂SO₄, filtered and the filtrate was concentrated under reduced pressure. The residue was purified by column chromatography (SiO₂, Petroleum ether/Ethyl acetate=100/1 to 20/1) to give 8-(2-benzyloxyethylamino)-N-(1-octylnonyl)octanamide (1 g, 1.88 mmol, 45.94% yield) as yellow oil.

¹H NMR (400 MHz, CDCl₃), 7.29-7.36 (m, 5H), 5.05 (d, J=8.8 Hz, 1H), 4.54 (s, 2H), 3.90-3.92 (m, 1H), 3.62 (t, J=5.2 Hz, 2H), 2.83 (t, J=5.2 Hz, 2H), 2.61 (t, J=7.2 Hz, 2H), 2.15 (t, J=7.2 Hz, 2H), 1.55-1.65 (m, 2H), 1.47-1.55 (m, 4H), 1.25-1.33 (m, 34H), 0.89 (t, J=6.8 Hz, 6H). LCMS: (M+H⁺): 531.3 @ 0.888 min.

### Step 3:

To a solution of undecan-1-amine (1 g, 5.84 mmol, 1 eq) and 6-bromohexanoic acid (1.14 g, 5.84 mmol, 1 eq) in DCM (10 mL) was added DMAP (356.55 mg, 2.92 mmol, 0.5 eq) and EDCI (1.34 g, 7.00 mmol, 1.2 eq), stirred at 20° C. for 8 hours. The mixture was added into H₂O (20 mL) and extracted with EtOAc (20 mL×3). The organic layer was washed with brine (20 mL×2), dried over Na₂SO₄, filtered and the filtrate was concentrated under reduced pressure. The residue was purified by column chromatography (SiO₂, Petroleum ether/Ethyl acetate=1/0 to 5/1) to give 6-bromo-N-undecyl-hexanamide (1.7 g, 4.88 mmol, 83.61% yield) as a white solid.

### Step 4:

To a solution of 8-(2-benzyloxyethylamino)-N-(1-octylnonyl)octanamide (500 mg, 941.86 μmol, 1 eq) in DMF (10 mL) was added KI (187.62 mg, 1.13 mmol, 1.2 eq) and K₂CO₃ (325.42 mg, 2.35 mmol, 32.81 μL, 2.5 eq), then a solution of 6-bromo-N-undecyl-hexanamide (360.92 mg, 1.04 mmol, 1.1 eq) in DMF (5 mL) was added to the mixture, then stirred at 80° C. for 12 hours. The mixture was filtered and the filtrate was added into H₂O (50 mL) and extracted with EtOAc (10 mL×3). The combined organic layer was washed with brine (10 mL×2), dried over Na₂SO₄, filtered and the filtrate was concentrated under reduced pressure. The residue was purified by column chromatography (SiO₂, Petroleum ether/Ethyl acetate=10/1 to 0/1) to give 8-[2-benzyloxyethyl-[6-oxo-6-(undecylamino)hexyl]amino]-N-(1-octylnonyl)octanamide (600 mg, 751.58 μmol, 39.90% yield) as yellow oil.

¹H NMR (400 MHz, CDCl₃), 7.29-7.36 (m, 5H), 5.60 (s, 1H), 5.18 (d, J=8.8 Hz, 1H), 4.52 (s, 2H), 3.89 (s, 1H), 3.55 (t, J=6.4 Hz, 2H), 3.20-3.25 (m, 2H), 2.53-2.70 (m, 2H), 2.35-2.46 (m, 4H), 2.14 (t, J=7.8 Hz, 4H), 1.51-1.63 (m, 4H), 1.40-1.50 (m, 8H), 1.25-1.33 (m, 50H), 0.89 (t, J=6.8 Hz, 9H). LCMS: (M+H): 798.5 @ 1.004 min.

### Step 5:

To a solution of Pd(OH)/C (20 mg, 10% purity) in EtOAc (10 mL) was added 8-[2-benzyloxyethyl-[6-oxo-6-(undecylamino)hexyl]amino]-N-(1-octylnonyl)octanamide (150 mg, 187.90 μmol, 1 eq) and AcOH (112.83 μg, 1.88 μmol, 1.07e-1 μL, 0.01 eq), then stirred at 30° C. for 5 hours under H₂ under 50 psi. The mixture was filtered and the filtrate was concentrated under reduced pressure. The residue was purified by column chromatography (SiO₂, Petroleum ether/Ethyl acetate=10/1 to 0/1) and by prep-TLC (SiO₂, Ethyl acetate:MeOH=3:1) to give 8-[2-hydroxyethyl-[6-oxo-6-(undecyl amino)hexyl]amino]-N-(1-octylnonyl)octanamide (30 mg, 42.36 μmol, 22.55% yield) as a white solid.

¹H NMR (400 MHz, CDCl₃), 5.61 (s, 1H), 5.21 (d, J=9.2 Hz, 1H), 3.91 (s, 1H), 3.56 (s, 2H), 3.20-3.25 (m, 2H), 2.53 (s, 2H), 2.48 (s, 4H), 2.13-2.18 (m, 4H), 1.51-1.63 (m, 4H), 1.43-1.50 (m, 8H), 1.25-1.33 (m, 51H), 0.89 (t, J=6.8 Hz, 9H). LCMS: (M+H⁺): 708.4 @ 3.194 min.

### 4.30. Synthesis of Compound 2249

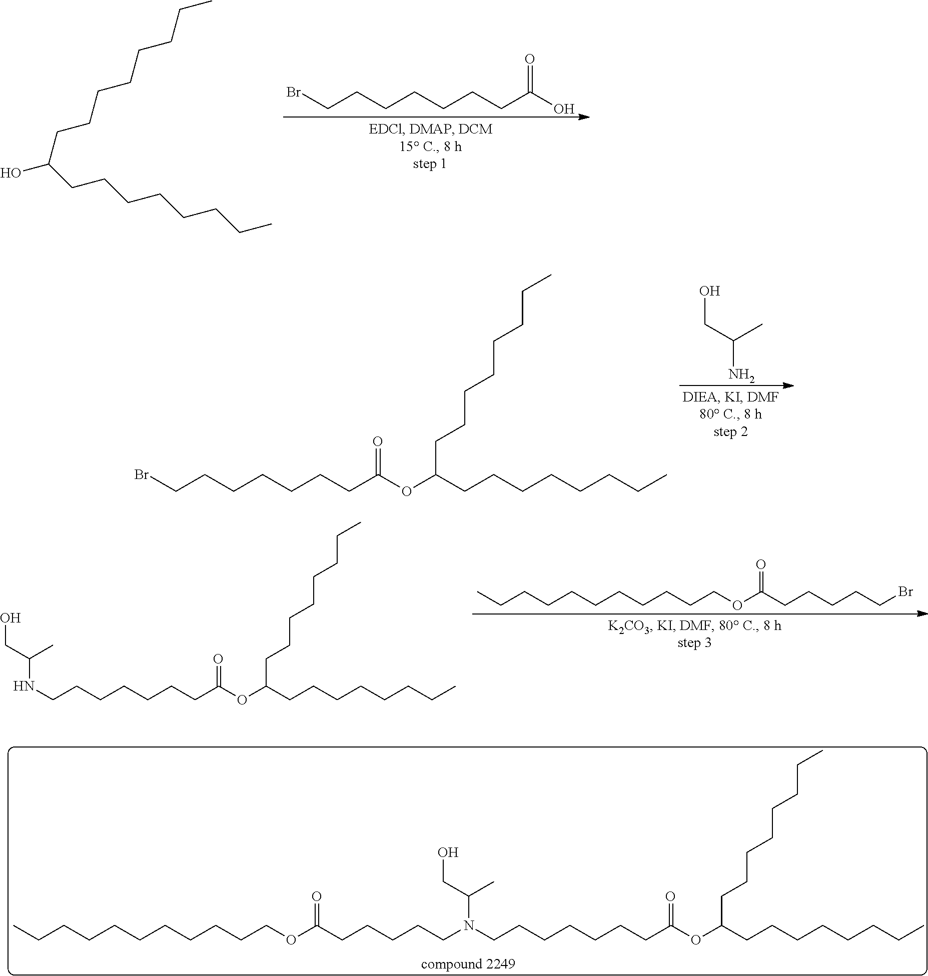

### Step 1:

To a solution of heptadecan-9-ol (5 g, 19.50 mmol, 1 eq) and 8-bromooctanoic acid (4.78 g, 21.45 mmol, 1.1 eq) in DCM (100 mL) was added EDCI (4.48 g, 23.39 mmol, 1.2 eq) and DMAP (1.19 g, 9.75 mmol, 0.5 eq). The mixture was stirred at 15° C. for 8 hours. The reaction mixture was quenched by addition H₂O 100 mL at 15° C., and then extracted with EtOAc 300 mL (100 mL×3). The combined organic layers were washed with brine 200 mL (100 mL×2), dried over Na₂SO₄, filtered and concentrated under reduced pressure to give a residue. The residue was purified by column chromatography (SiO₂, Petroleum ether/Ethyl acetate=20/1 to 2/1) to give compound 1-octylnonyl 8-bromooctanoate (8.5 g, 18.42 mmol, 94.46% yield) as colorless oil.

¹H NMR (400 MHz, CDCl₃), 4.86-4.90 (m, 1H), 3.41 (t, J=6.8 Hz, 2H), 2.29 (t, J=7.6 Hz, 2H), 1.80-1.90 (m, 2H), 1.60-1.70 (m, 2H), 1.40-1.55 (m, 6H), 1.20-1.40 (m, 28H), 0.89 (t, J=6.8 Hz, 6H).

### Step 2:

To a solution of 1-octylnonyl 8-bromooctanoate (3.23 g, 6.99 mmol, 1.05 eq) and 2-aminopropan-1-ol (500 mg, 6.66 mmol, 530.22 μL, 1 eq) in DMF (10 mL) was added DIEA (1.72 g, 13.31 mmol, 2.32 mL, 2 eq) and KI (1.22 g, 7.32 mmol, 1.1 eq). The mixture was stirred at 80° C. for 8 hours. The reaction mixture was quenched by addition H₂O 30 mL at 15° C., and then extracted with EtOAc 90 mL (30 mL×3). The combined organic layers were washed with brine 60 mL (30 mL×2), dried over Na₂SO₄, filtered and concentrated under reduced pressure to give a residue. The residue was purified by column chromatography (SiO₂, Petroleum ether/Ethyl acetate=5/1 to 1/4) to give compound 1-octylnonyl 8-[(2-hydroxy-1-methyl-ethyl)amino]octanoate (1.5 g, 3.29 mmol, 49.44% yield) as a white solid.

¹H NMR (400 MHz, CDCl₃), 4.86-4.90 (m, 1H), 3.59-3.64 (m, 1H), 3.28-3.33 (m, 1H), 2.70-2.90 (m, 2H), 2.50-2.60 (m, 2H), 2.28 (t, J=7.6 Hz, 2H), 1.52-1.70 (m, 2H), 1.40-1.55 (m, 6H), 1.20-1.40 (m, 32H), 1.09 (d, J=6.4 Hz, 3H), 0.88 (t, J=6.8 Hz, 6H).

### Step 3:

To a solution of 1-octylnonyl 8-[(2-hydroxy-1-methyl-ethyl)amino]octanoate (250 mg, 548.54 μmol, 1 eq) and undecyl 6-bromohexanoate (210.79 mg, 603.39 μmol, 1.1 eq) in DMF (10 mL) was added KI (109.27 mg, 658.25 μmol, 1.2 eq) and K₂CO₃ (189.53 mg, 1.37 mmol, 2.5 eq). The mixture was stirred at 80° C. for 8 hours. The reaction mixture was quenched by addition H₂O 30 mL at 15° C., and then extracted with EtOAc 90 mL (30 mL×3). The combined organic layers were washed with brine 60 mL (30 mL×2), dried over Na₂SO₄, filtered and concentrated under reduced pressure to give a residue. The residue was purified by column chromatography (SiO₂, Petroleum ether/Ethyl acetate=10/1 to 0/1) to give compound 1-octylnonyl 8-[(2-hydroxy-1-methyl-ethyl)-(6-oxo-6-undecoxy-hexyl)amino]octanoate (96 mg, 132.56 μmol, 12.08% yield, 100% purity) as colorless oil.

¹H NMR (400 MHz, CDCl₃), 4.76-4.83 (m, 1H), 3.98 (t, J=6.8 Hz, 2H), 3.25-3.30 (m, 1H), 3.12-3.18 (m, 1H), 2.70-2.90 (m, 1H), 2.30-2.45 (m, 2H), 2.15-2.25 (m, 6H), 1.51-1.58 (m, 6H), 1.30-1.45 (m, 8H), 1.10-1.30 (m, 48H), 0.76-0.83 (m, 12H).

LCMS: (M+H⁺): 724.4 @ 13.085 min.

### 4.31. Synthesis of Compound 2250

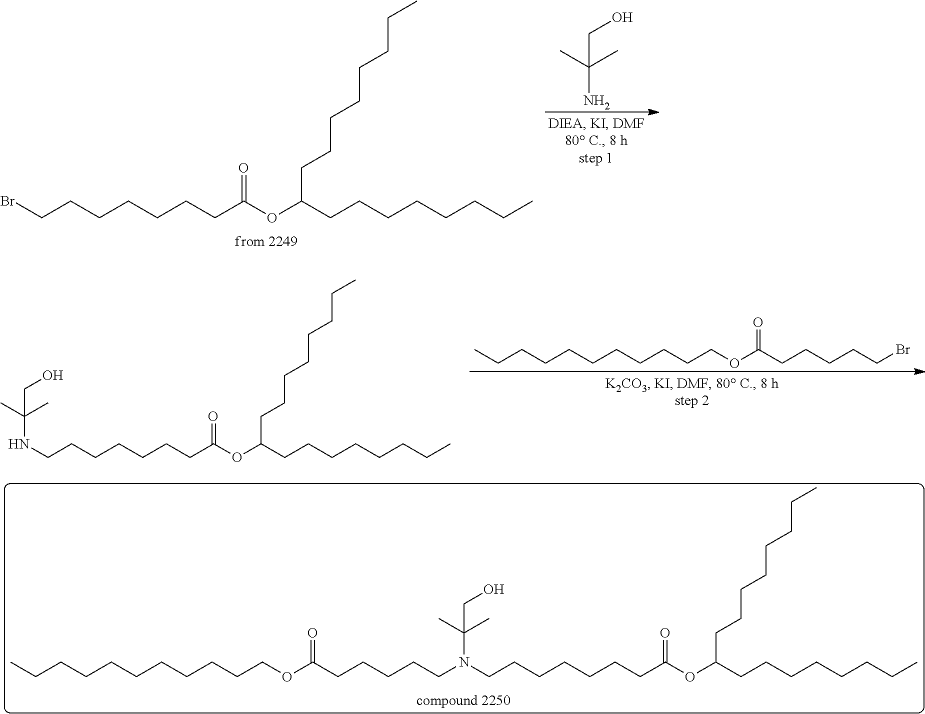

### Step 1:

To a solution of 1-octylnonyl 8-bromooctanoate (3 g, 6.50 mmol, 1.05 eq) and 2-amino-2-methyl-propan-1-ol (551.77 mg, 6.19 mmol, 590.76 μL, 1 eq) in DMF (30 mL) was added DIEA (1.60 g, 12.38 mmol, 2.16 mL, 2 eq) and KI (1.13 g, 6.81 mmol, 1.1 eq). The mixture was stirred at 80° C. for 8 hours. The reaction mixture was quenched by addition H₂O 30 mL at 15° C., and then extracted with EtOAc 90 mL (30 mL×3). The combined organic layers were washed with brine 60 mL (30 mL×2), dried over Na₂SO₄, filtered and concentrated under reduced pressure to give a residue. The residue was purified by column chromatography (SiO₂, Petroleum ether/Ethyl acetate=5/1 to 1/4) to give compound 1-octylnonyl 8-[(2-hydroxy-1,1-dimethyl-ethyl)amino]octanoate (2 g, 4.26 mmol, 68.77% yield) as colorless oil.

¹H NMR (400 MHz, CDCl₃), 4.85-4.90 (m, 1H), 3.35 (s, 2H), 2.55 (t, J=7.2 Hz, 2H), 2.29 (t, J=7.6 Hz, 2H), 1.55-1.70 (m, 2H), 1.40-1.55 (m, 6H), 1.20-1.40 (m, 30H), 1.12 (s, 6H), 0.89 (t, J=6.8 Hz, 6H).

### Step 2:

To a solution of undecyl 6-bromohexanoate (297.45 mg, 851.46 μmol, 1.6 eq) and 1-octylnonyl 8-[(2-hydroxy-1,1-dimethyl-ethyl)amino]octanoate (250 mg, 532.16 μmol, 1 eq) in DMF (10 mL) was added KI (106.01 mg, 638.60 μmol, 1.2 eq) and K₂CO₃ (183.87 mg, 1.33 mmol, 2.5 eq). The mixture was stirred at 80° C. for 8 hours. The reaction mixture was quenched by addition H₂O 30 mL at 15° C., and then extracted with EtOAc 90 mL (30 mL×3). The combined organic layers were washed with brine 60 mL (30 mL×2), dried over Na₂SO₄, filtered and concentrated under reduced pressure to give a residue. The residue was purified by prep-TLC (SiO₂, PE:EA=0:1) to give compound 1-octylnonyl 8-[(2-hydroxy-1,1-dimethyl-ethyl)-(6-oxo-6-undecoxy-hexyl)amino]octanoate (44 mg, 59.25 μmol, 5.57% yield, 99.4% purity) as colorless oil.

¹H NMR (400 MHz, CDCl₃), 4.85-4.90 (m, 1H), 4.07 (t, J=7.2 Hz, 2H), 3.23 (s, 2H), 2.40-2.50 (m, 4H), 2.26-2.31 (m, 4H), 1.60-1.70 (m, 6H), 1.45-1.55 (m, 4H), 1.35-1.45 (m, 4H), 1.20-1.35 (m, 48H), 1.04 (s, 6H), 0.87-0.91 (m, 9H). LCMS: (M+H⁺): 738.4 @ 13.185 min.

4.32: Synthesis of compound 2276

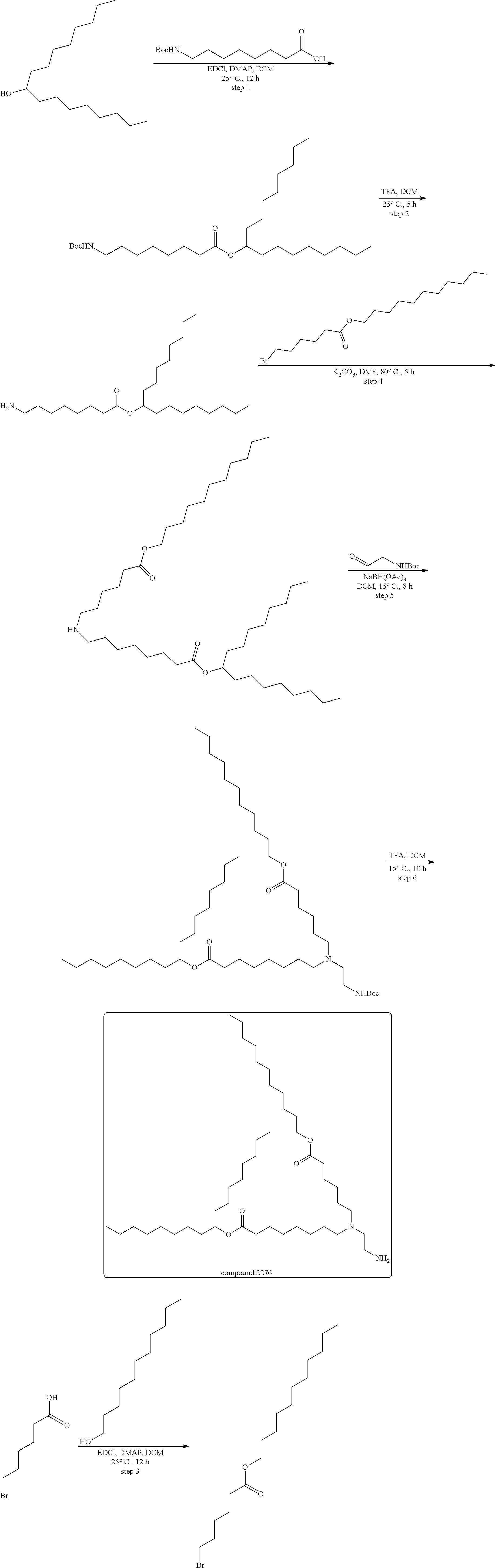

### Step 1:

A mixture of 8-(tert-butoxycarbonylamino)octanoic acid (25 g, 96.40 mmol, 1.2 eq) in DCM (1000 mL) was added DMAP (4.91 g, 40.17 mmol, 0.5 eq), heptadecan-9-ol (20.60 g, 80.33 mmol, 1 eq), EDCI (46.20 g, 241.00 mmol, 3 eq). The mixture was stirred at 25° C. for 12 hours under N₂ atmosphere. LCMS showed 48% of desired product. The reaction mixture was diluted with EtOAc (200 mL×3) and washed with H₂O 200 mL. The combined organic layers were dried over Na₂SO₄, filtered and concentrated under reduced pressure to give a residue. The residue was purified by column chromatography (SiO₂, Petroleum ether/Ethyl acetate=1/0 to 1/0) to give compound 1-octylnonyl 8-(tert-butoxycarbonylamino)octanoate (24 g, crude) as yellow oil.

### Step 2:

To a solution of 1-octylnonyl 8-(tert-butoxycarbonylamino)octanoate (12 g, 24.11 mmol, 1 eq) in DCM (100 mL) was added TFA (46.20 g, 405.18 mmol, 30 mL, 16.81 eq). The mixture was stirred at 25° C. for 5 hours. The reaction mixture was adjusted pH=7 with saturated NaHCO₃ aqueous and extracted with EtOAc (200 mL×3), dried over Na₂SO₄, filtered and the filtrate was concentrated under reduced pressure to give a residue. The residue was purified by column chromatography (SiO₂, Petroleum ether/Ethyl acetate=0/1 to Ethyl acetate/MeOH=3/1) to give compound 1-octylnonyl 8-aminooctanoate (15 g, 37.72 mmol, 78.23% yield) as yellow oil.

¹H NMR (400 MHz, CDCl₃), 5.64 (brs, 2H), 4.84-4.88 (m, 1H), 2.84 (t, J=7.6 Hz, 2H), 2.28 (t, J=7.6 Hz, 2H), 1.50-1.61 (m, 8H), 1.26-1.33 (m, 30H), 0.88 (t, J=6.8 Hz, 6H).

### Step 3:

To a mixture of 6-bromohexanoic acid (22.64 g, 116.07 mmol, 1 eq) in DCM (1 mL) was added DMAP (2.84 g, 23.21 mmol, 0.2 eq), undecan-1-ol (20 g, 116.07 mmol, 1 eq), EDCI (22.25 g, 116.07 mmol, 1 eq). The mixture was stirred at 25° C. for 12 hours under N₂ atmosphere. The reaction mixture was diluted with H₂O 200 mL and extracted with EtOAc (200 mL×3). The combined organic layers were dried over Na₂SO₄, filtered and the filtrate concentrated under reduced pressure to give a residue. The residue was purified by column chromatography (SiO₂, Petroleum ether/Ethyl acetate=1/0 to 40/1) to give compound undecyl 6-bromohexanoate (36 g, 103.05 mmol, 88.78% yield) as yellow oil.

¹H NMR (400 MHz, CDCl₃), 4.07 (t, J=6.8 Hz, 2H), 3.41 (t, J=6.8 Hz, 2H), 2.33 (t, J=7.2 Hz, 2H), 1.87-1.91 (m, 2H), 1.63-1.68 (m, 4H), 1.48-1.50 (m, 2H), 1.27-1.32 (m, 16H), 0.89 (t, J=6.4 Hz, 3H).

### Step 4:

To a solution of 1-octylnonyl 8-aminooctanoate (1 g, 2.51 mmol, 1 eq), undecyl 6-bromohexanoate (878.47 mg, 2.51 mmol, 1 eq) in DMF (20 mL) was added K₂CO₃ (1.04 g, 7.54 mmol, 3 eq). The mixture was stirred at 80° C. for 5 hours. LCMS showed 56% of desired product. The reaction mixture was diluted with H₂O 20 mL and extracted with EtOAc (20 mL×3). The combined organic layers were dried over Na₂SO₄, filtered and concentrated under reduced pressure to give a residue. The residue was purified by column chromatography (SiO₂, Ethyl acetate:MeOH=1/0 to 10/1) to give compound 1-octylnonyl 8-[(6-oxo-6-undecoxy-hexyl)amino] octanoate (0.5 g, 750.63 μmol, 29.85% yield) as yellow oil.

¹H NMR (400 MHz, CDCl₃), 4.86-4.89 (m, 1H), 4.06 (t, J=6.8 Hz, 2H), 2.59-2.60 (m, 4H), 2.28-2.31 (m, 4H), 1.60-1.65 (m, 6H), 1.50-1.52 (m, 8H), 1.27-1.36 (m, 48H), 0.89 (t, J=6.4 Hz, 9H). LCMS: (M+H⁺): 666.8 @ 1.168 min.

### Step 5:

To a solution of 1-octylnonyl 8-[(6-oxo-6-undecoxy-hexyl)amino]octanoate (0.5 g, 0.75 mmol, 1 eq) and tert-butyl N-(2-oxoethyl)carbamate (0.24 g, 1.50 mmol, 2 eq) in DCM (10 mL) was added sodium; triacetoxyboranuide (0.48 g, 2.25 mmol, 3 eq) at 15° C. The mixture was degassed and purged with N₂ for 3 times, and then stirred at 15° C. for 8 hours under N₂ atmosphere. The reaction mixture was filtered and concentrated under reduced pressure to give a residue. The residue was diluted with H₂O 20 mL extracted with EtOAc (30 mL×2).

The combined organic layers were dried over Na₂SO₄, filtered and concentrated under reduced pressure to give a residue. The residue was purified by column chromatography (SiO₂, Petroleum ether/Ethyl acetate=10/1 to 3/1, 5% NH₃·H₂O) to give compound 1-octylnonyl 8-[2-(tert-butoxycarbonylamino)ethyl-(6-oxo-6-undecoxy-hexyl)amino]octanoate (0.36 g, 0.44 mmol, 58.79% yield) as colorless oil.

LCMS: (M+H⁺): 809.7 @ 1.083 min.

### Step 6:

To a solution of 1-octylnonyl 8-[2-(tert-butoxycarbonylamino)ethyl-(6-oxo-6-undecoxy-hexyl)amino]octanoate (0.36 g, 0.44 mmol, 1 eq) in DCM (5 mL) was added dropwise TFA (1.18 g, 10.34 mmol, 0.76 mL, 1.67 eq) at 15° C. The mixture was stirred at 15° C. for 10 hours under N₂ atmosphere. The reaction mixture was adjusted to pH=7.0 with sat. NaHCO₃ aq. 15 ml and extracted with EtOAc 45 mL (15 mL×3). The combined organic layers were dried over Na₂SO₄, filtered and concentrated under reduced pressure to give a residue. The residue was purified by column chromatography (SiO₂, Petroleum ether/Ethyl acetate=3/1 to 0/1, 5% NH₃·H₂O) to give compound 1-octylnonyl 8-[2-aminoethyl-(6-oxo-6-undecoxy-hexyl)amino]octanoate (0.22 g, 0.31 mmol, 99% purity, 70.75% yield) as colorless oil.

¹H NMR (400 MHz, CDCl₃), 4.84-4.90 (m, 1H), 4.06 (t, J=6.8 Hz, 2H), 2.71 (t, J=6.4 Hz, 2H), 2.37-2.46 (m, 6H), 2.29 (q, J=7.6 Hz, 4H), 1.59-1.68 (m, 6H), 1.38-1.52 (m, 8H), 1.27-1.31 (m, 48H), 0.88 (t, J=6.8 Hz, 9H). LCMS: (M+H⁺): 709.4 @ 12.315 min.

### 4.33. Synthesis of Compound 2277

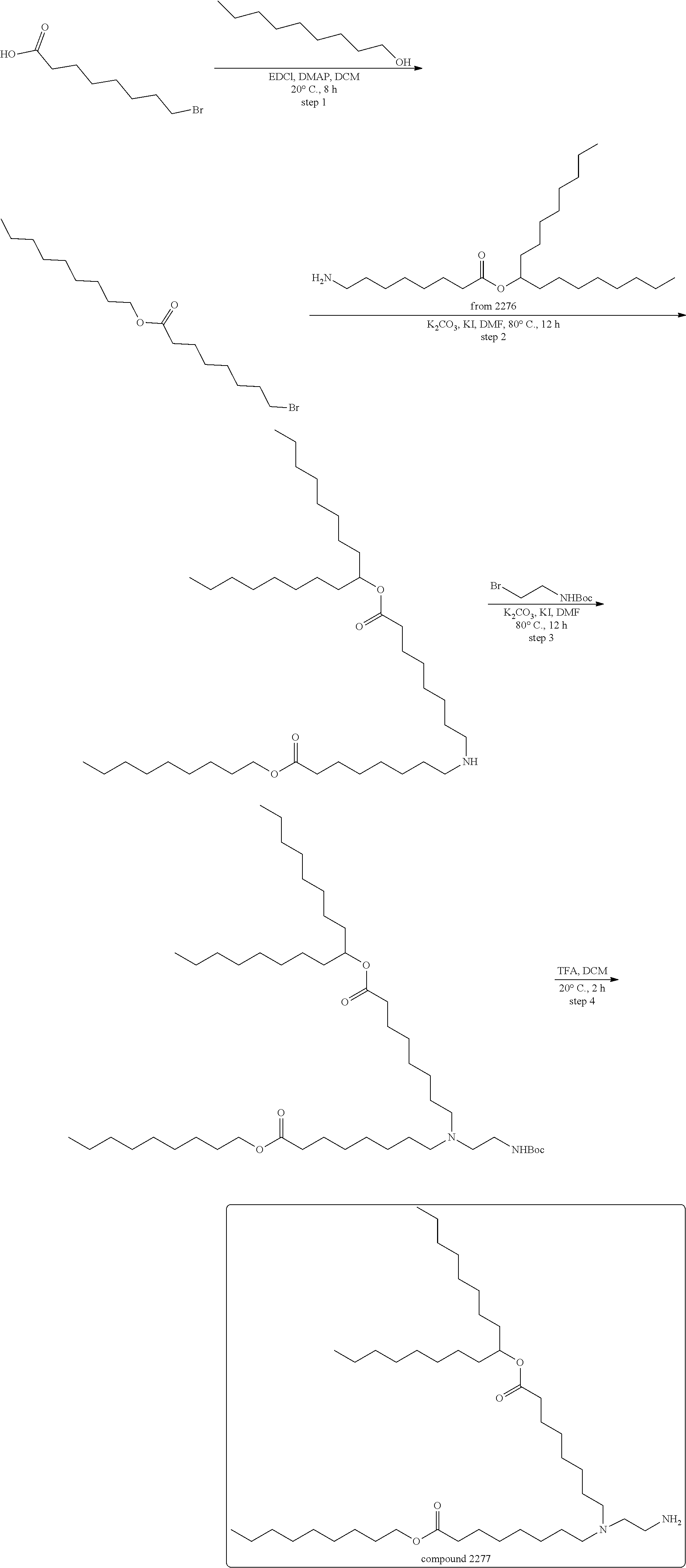

### Step 1:

To a solution of 8-bromooctanoic acid (10.21 g, 45.75 mmol, 1.1 eq) and nonan-1-ol (6 g, 41.59 mmol, 1 eq) in DCM (100 mL) was added DMAP (1.02 g, 8.32 mmol, 0.2 eq) and EDCI (9.57 g, 49.91 mmol, 1.2 eq), stirred at 20° C. for 8 hours. The mixture was added into H₂O (50 mL), extracted with EtOAc (50 mL×3), organic layer was washed with brine (50 mL×2), dried over Na₂SO₄, filtered and the filtrate was concentrated under reduced pressure. The residue was purified by column chromatography (SiO₂, Petroleum ether/Ethyl acetate=50/1 to 10/1) to give nonyl 8-bromooctanoate (10 g, 27.19 mmol, 65.38% yield) as colorless oil.

### Step 2:

To a solution of 1-octylnonyl 8-aminooctanoate (5 g, 12.57 mmol, 1.1 eq) in DMF (100 mL) was added KI (2.28 g, 13.74 mmol, 1.2 eq) and K₂CO₃ (4.75 g, 34.35 mmol, 3 eq), then a solution of nonyl 8-bromooctanoate (4 g, 11.45 mmol, 1 eq) in DMF (20 mL) was added to the mixture, then stirred at 80° C. for 12 hours. The mixture was filtered and the filtrate was added into H₂O (50 mL), extracted with EtOAc (30 mL×3), combined organic layer was washed with brine (30 mL×2), dried over Na₂SO₄, filtered and the filtrate was concentrated under reduced pressure. The residue was purified by column chromatography (SiO₂, Petroleum ether/Ethyl acetate=10/1 to 0/1) to give compound nonyl 8-[[8-(1-octylnonoxy)-8-oxo-octyl]amino]octanoate (4 g, 5.40 mmol, 47.20% yield) as yellow oil.

¹H NMR (400 MHz, CDCl₃), 4.85-4.90 (m, 1H), 4.06 (t, J=7.2 Hz, 2H), 2.59 (t, J=6.8 Hz, 3H), 2.26-2.31 (m, 4H), 1.60-1.70 (m, 6H), 1.40-1.55 (m, 8H), 1.20-1.35 (m, 49H), 0.86-0.91 (m, 9H).

### Step 3:

To a solution of nonyl 8-[[8-(1-octylnonoxy)-8-oxo-octyl]amino]octanoate (250 mg, 375.31 μmol, 1 eq) in DMF (5 mL) was added K₂CO₃ (259.36 mg, 1.88 mmol, 5 eq) and KI (62.30 mg, 375.31 μmol, 1 eq), and then tert-butyl N-(2-bromoethyl)carbamate (336.42 mg, 1.50 mmol, 4 eq) was added into the mixture. The mixture was stirred at 80° C. for 12 hours. The mixture was filtered and the filtrate was added into H₂O (5 mL), extracted with EtOAc (5 mL×3), organic layer was washed with brine (5 mL×2), dried over Na₂SO₄, filtered and the filtrate was concentrated under reduced pressure. The residue was purified by column chromatography (SiO2, Petroleum ether/Ethyl acetate=10/1 to 0/1) to give compound nonyl 8-[2-(tert-butoxycarbonylamino)ethyl-[8-(1-octylnonoxy)-8-oxo-octyl]amino]octanoate (200 mg, 247.13 μmol, 65.85% yield) as colorless oil.

### Step 4:

A solution of nonyl 8-[2-(tert-butoxycarbonylamino)ethyl-[8-(1-octylnonoxy)-8-oxo-octyl]amino]octanoate (160 mg, 197.70 μmol, 1 eq) in TFA (3.08 g, 27.01 mmol, 2 mL, 136.63 eq) and DCM (4 mL) was stirred at 20° C. for 2 hours. The mixture was filtered and the filtrate was concentrated under reduced pressure. The residue was purified by column chromatography (SiO₂, Ethyl acetate:Methanol=1/0 to 5/1) to give compound nonyl 8-[2-aminoethyl-[8-(1-octylnonoxy)-8-oxo-octyl]amino]octanoate (45 mg, 63.45 μmol, 32.10% yield) as colorless oil.

¹H NMR (400 MHz, CDCl₃), 4.85-4.90 (m, 1H), 4.06 (t, J=6.4 Hz, 2H), 2.93 (t, J=5.6 Hz, 2H), 2.69 (t, J=6.0 Hz, 2H), 2.59 (t, J=7.6 Hz, 4H), 2.26-2.32 (m, 4H), 1.60-1.70 (m, 6H), 1.40-1.55 (m, 8H), 1.20-1.35 (m, 48H), 0.89 (t, J=6.4 Hz, 9H). (M+H⁺): 709.4.

LCMS: (M+H⁺): 709.4 @ 10.079 min.

### 4.34. Synthesis of Compound 2300

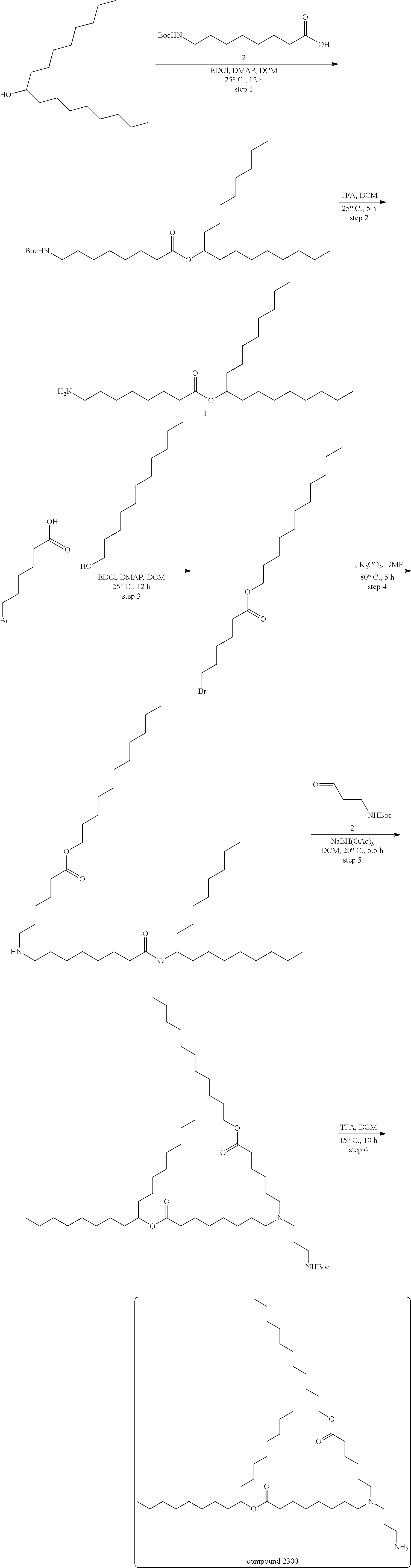

### Step 1:

A mixture of 8-(tert-butoxycarbonylamino)octanoic acid (25 g, 96.40 mmol, 1.2 eq) in DCM (1000 mL) was added DMAP (4.91 g, 40.17 mmol, 0.5 eq), heptadecan-9-ol (20.60 g, 80.33 mmol, 1 eq), EDCI (46.20 g, 241.00 mmol, 3 eq). The mixture was stirred at 25° C. for 12 hours under N₂ atmosphere. LCMS showed 48% of desired product. The reaction mixture was diluted with EtOAc 600 mL (200 mL×3) and washed with H₂O 200 mL. The combined organic layers were dried over Na₂SO₄, filtered and concentrated under reduced pressure to give a residue. The residue was purified by column chromatography (SiO₂, Petroleum ether/Ethyl acetate=1/0 to 1/0) to give compound 1-octylnonyl 8-(tert-butoxycarbonylamino)octanoate (24 g, crude) as yellow oil. The crude product was used for next step without detection by ¹H NMR.

### Step 2:

To a solution of 1-octylnonyl 8-(tert-butoxycarbonylamino)octanoate (12 g, 24.11 mmol, 1 eq) in DCM (100 mL) was added TFA (46.20 g, 405.18 mmol, 30 mL, 16.81 eq). The mixture was stirred at 25° C. for 5 hours. The reaction mixture was adjusted pH=7 with saturated NaHCO₃ aqueous and extracted with EtOAc 600 mL (200 mL×3), dried over Na₂SO₄, filtered and the filtrate was concentrated under reduced pressure to give a residue. The residue was purified by column chromatography (SiO₂, Petroleum ether/Ethyl acetate=0/1 to Ethyl acetate/MeOH=3/1) to give compound 1-octylnonyl 8-aminooctanoate (15 g, 37.72 mmol, 78.23% yield) as yellow oil.

¹H NMR (400 MHz, CDCl₃), 5.64 (brs, 2H), 4.84-4.88 (m, 1H), 2.84 (t, J=7.6 Hz, 2H), 2.28 (t, J=7.6 Hz, 2H), 1.50-1.61 (m, 8H), 1.26-1.33 (m, 30H), 0.88 (t, J=6.8 Hz, 6H).

LCMS: (M+H⁺): 398.6 @ 1.010 min.

### Step 3:

To a mixture of 6-bromohexanoic acid (22.64 g, 116.07 mmol, 1 eq) in DCM (1 mL) was added DMAP (2.84 g, 23.21 mmol, 0.2 eq), undecan-1-ol (20 g, 116.07 mmol, 1 eq), EDCI (22.25 g, 116.07 mmol, 1 eq). The mixture was stirred at 25° C. for 12 hours under N₂ atmosphere. The reaction mixture was diluted with H₂O 200 mL and extracted with EtOAc 600 mL (200 mL×3). The combined organic layers were dried over Na₂SO₄, filtered and the filtrate concentrated under reduced pressure to give a residue. The residue was purified by column chromatography (SiO₂, Petroleum ether/Ethyl acetate=1/0 to 40/1) to give compound undecyl 6-bromohexanoate (36 g, 103.05 mmol, 88.78% yield) as yellow oil.

¹H NMR (400 MHz, CDCl₃), 4.07 (t, J=6.8 Hz, 2H), 3.41 (t, J=6.8 Hz, 2H), 2.33 (t, J=7.2 Hz, 2H), 1.87-1.91 (m, 2H), 1.63-1.68 (m, 4H), 1.48-1.50 (m, 2H), 1.27-1.32 (m, 16H), 0.89 (t, J=6.4 Hz, 3H).

### Step 4:

To a solution of 1-octylnonyl 8-aminooctanoate (1 g, 2.51 mmol, 1 eq), undecyl 6-bromohexanoate (878.47 mg, 2.51 mmol, 1 eq) in DMF (20 mL) was added K₂CO₃ (1.04 g, 7.54 mmol, 3 eq). The mixture was stirred at 80° C. for 5 hours. LCMS showed 56% of desired product. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Ethyl acetate:MeOH=1/0 to 10/1) to give compound 1-octylnonyl 8-[(6-oxo-6-undecoxy-hexyl)amino] octanoate (0.5 g, 750.63 μmol, 29.85% yield) as yellow oil.

¹H NMR (400 MHz, CDCl₃), 4.86-4.89 (m, 1H), 4.06 (t, J=6.8 Hz, 2H), 2.59-2.60 (m, 4H), 2.28-2.31 (m, 4H), 1.60-1.65 (m, 6H), 1.50-1.52 (m, 8H), 1.27-1.36 (m, 48H), 0.89 (t, J=6.4 Hz, 9H). LCMS: (M+H⁺): 666.8 @ 1.168 min.

### Step 5:

To a solution of 1-octylnonyl 8-[(6-oxo-6-undecoxy-hexyl)amino]octanoate (1.5 g, 2.25 mmol, 1 eq) in DCM (15 mL) was added tert-butyl N-(3-oxopropyl)carbamate (585.07 mg, 3.38 mmol, 1.5 eq) at 20° C. The mixture was degassed and purged with N₂ for 3 times, then stirred at 20° C. for 0.5 hour under N₂ atmosphere. To the mixture was added sodium; triacetoxyboranuide (954.53 mg, 4.50 mmol, 2 eq) and then stirred at 20° C. for 5 hours under N₂ atmosphere. The reaction mixture was diluted with H₂O 20 mL extracted with EtOAc 300 mL (150 mL×2). The combined organic layers were dried over Na₂SO₄, filtered and concentrated under reduced pressure to give a residue. The residue was purified by column chromatography (SiO₂, Petroleum ether/Ethyl acetate=8/1 to 0/1) to give compound 1-octylnonyl 8-[3-(tert-butoxycarbonylamino)propyl-(6-oxo-6-undecoxy-hexyl)amino]octanoate (1.6 g, 1.94 mmol, 86.30% yield) as colorless oil.

### Step 6:

To a solution of 1-octylnonyl 8-[2-(tert-butoxycarbonylamino)ethyl-(6-oxo-6-undecoxy-hexyl)amino]octanoate (5 g, 6.18 mmol, 1 eq) in DCM (45 mL) was added dropwise TFA (16.50 g, 144.71 mmol, 10.71 mL, 23.42 eq) at 15° C. The mixture was degassed and purged with N₂ for 3 times, and then stirred at 15° C. for 10 hours under N₂ atmosphere. The reaction mixture was adjusted to pH=7.0 with sat. NaHCO₃ aq. 80 ml and extracted with EtOAc 450 mL (150 mL×3). The combined organic layers were dried over Na₂SO₄, filtered and concentrated under reduced pressure to give a residue. The residue was purified by column chromatography (SiO₂, Petroleum ether/Ethyl acetate=3/1 to 0/1, 5% NH₃·H₂O) to give compound 1-octylnonyl 8-[2-aminoethyl-(6-oxo-6-undecoxy-hexyl)amino]octanoate (3.1 g, 4.37 mmol, 70.75% yield) as colorless oil.

¹H NMR (400 MHz, CDCl₃), 4.84-4.90 (m, 1H), 4.06 (t, J=6.8 Hz, 2H), 2.71 (t, J=6.4 Hz, 2H), 2.48 (t, J=6.8 Hz, 2H), 2.37-2.46 (m, 4H), 2.29 (q, J=7.6 Hz, 4H), 1.58-1.68 (m, 8H), 1.44-1.48 (m, 4H), 1.50-1.52 (m, 4H), 1.26-1.31 (m, 48H), 0.88 (t, J=6.8 Hz, 9H).

LCMS: (M/2+H⁺): 723.4 @ 9.826 min.

### 4.35. Synthesis of Compound 2301

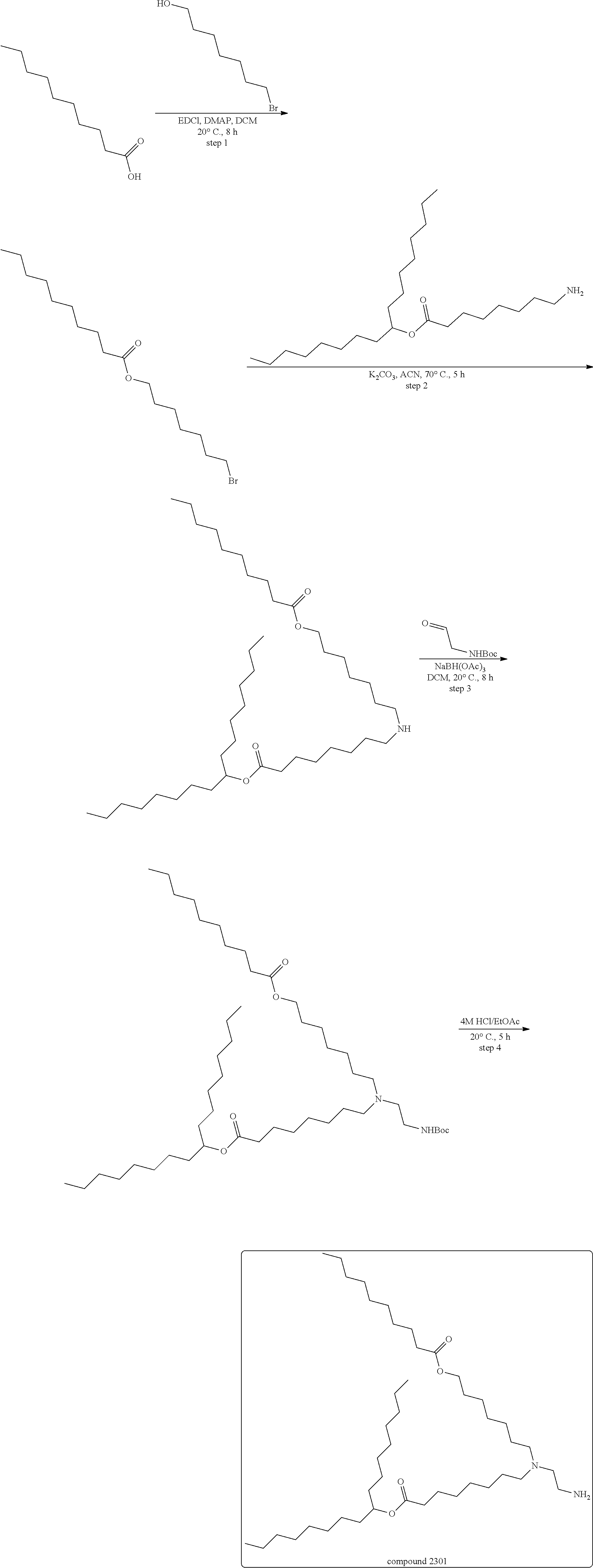

### Step 1:

To a mixture of decanoic acid (17.66 g, 102.51 mmol, 19.78 mL, 1 eq) in DCM (500 mL) was added DMAP (2.50 g, 20.50 mmol, 0.2 eq), 7-bromoheptan-1-ol (20 g, 102.51 mmol, 1 eq), EDCI (19.65 g, 102.51 mmol, 1 eq). The mixture was stirred at 20° C. for 8 hours under N₂ atmosphere. The reaction mixture was diluted with H₂O 200 mL and extracted with EtOAc 600 mL (200 mL×3). The combined organic layers were dried over Na₂SO₄, filtered and concentrated under reduced pressure to give a residue. The residue was purified by column chromatography (SiO₂, Petroleum ether/Ethyl acetate=1/0 to 40/1) to give compound 7-bromoheptyl decanoate (30 g, 85.87 mmol, 83.77% yield) as yellow oil.

¹H NMR (400 MHz, CDCl₃), 4.07 (t, J=6.8 Hz, 2H), 3.41 (t, J=6.8 Hz, 2H), 2.31 (t, J=7.2 Hz, 2H), 1.85-1.87 (m, 2H), 1.62-1.63 (m, 4H), 1.45-1.48 (m, 2H), 1.37-1.42 (m, 4H), 1.27-1.31 (m, 12H), 0.89 (t, J=6.8 Hz, 3H).

### Step 2:

To a solution of 1-octylnonyl 8-aminooctanoate (6 g, 15.09 mmol, 1 eq), 7-bromoheptyl decanoate (5.27 g, 15.09 mmol, 1 eq) in ACN (50 mL) was added K₂CO₃ (8.34 g, 60.35 mmol, 4 eq). The mixture was stirred at 70° C. for 5 hours. The reaction mixture was diluted with H₂O 200 mL and extracted with EtOAc 300 mL (100 mL×3). The combined organic layers were dried over Na₂SO₄, filtered and concentrated under reduced pressure to give a residue. The residue was purified by column chromatography (SiO₂, Ethyl acetate:MeOH=1/0 to 10/1) to give compound 7-[[8-(1-octylnonoxy)-8-oxo-octyl]amino]heptyl decanoate (3 g, 4.50 mmol, 29.85% yield) as yellow oil.

### Step 3:

To a solution of 7-[[8-(1-octylnonoxy)-8-oxo-octyl]amino]heptyl decanoate (3 g, 4.50 mmol, 1 eq), tert-butyl N-(2-oxoethyl)carbamate (1.43 g, 9.01 mmol, 2 eq) in DCM (50 mL) was added NaBH(OAc)₃ (1.91 g, 9.01 mmol, 2 eq). The mixture was stirred at 20° C. for 8 hours. The mixture was diluted with EtOAc 60 mL and washed with water 180 mL (60 mL×3) and brine 40 mL (20 mL×2), dried with anhydrous Na₂SO₄, filtered and concentrated under reduced pressure to give a residue. The residue was purified by column chromatography (SiO₂, Petroleum ether/Ethyl acetate=10/1 to 5/1) to give compound 7-[2-(tert-butoxycarbonylamino)ethyl-[8-(1-octylnonoxy)-8-oxo-octyl]amino]heptyl decanoate (2 g, 2.47 mmol, 54.84% yield) as yellow oil.

¹H NMR (400 MHz, CDCl₃), 5.06 (brs, 1H), 4.86-4.89 (m, 1H), 4.06 (t, J=6.8 Hz, 2H), 3.17 (brs, 2H), 2.28-2.66 (m, 10H), 1.63-1.66 (m, 6H), 1.48-1.62 (m, 15H), 1.26-1.42 (m, 50H), 0.89 (t, J=6.0 Hz, 9H).

### Step 4:

A solution of 7-[2-(tert-butoxycarbonylamino)ethyl-[8-(1-octylnonoxy)-8-oxo-octyl]amino]heptyl decanoate (2 g, 2.47 mmol, 1 eq) in HCl/EtOAc (4 M, 617.82 μL, 1 eq) was stirred at 20° C. for 5 hours. The crude reaction mixture was adjusted pH=7 with saturated Sat.NaHCO₃ and extracted with EtOAc 120 mL (40 mL×3). The combined organic layers were dried over Na₂SO₄, filtered and concentrated under reduced pressure to give a residue. The residue was purified by column chromatography (SiO₂, Petroleum ether/Ethyl acetate/NH₃·H₂O=5/1/0 to 2/1/0.1) and p-TLC (Petroleum ether/Ethyl acetate/NH₃·H₂O=2/1/0.1) to give compound 7-[2-aminoethyl-[8-(1-octylnonoxy)-8-oxo-octyl]amino]heptyl decanoate (1 g, 1.41 mmol, 57.06% yield) as yellow oil.

¹H NMR (400 MHz, CDCl₃), 4.84-4.90 (m, 1H), 4.06 (t, J=6.8 Hz, 2H), 2.83 (t, J=6.4 Hz, 2H), 2.59 (t, J=6.4 Hz, 2H), 2.52 (t, J=5.2 Hz, 4H), 2.27-2.32 (m, 4H), 1.61-1.64 (m, 6H), 1.48-1.52 (m, 6H), 1.27-1.32 (m, 50H), 0.89 (t, J=6.4 Hz, 9H).

LCMS: (M+H⁺): 709.4 @ 10.026 min.

### Step 5:

To a solution of 7-[2-aminoethyl-[8-(1-octylnonoxy)-8-oxo-octyl]amino]heptyl decanoate (0.2 g, 282.02 μmol, 1 eq), TEA (28.54 mg, 282.02 μmol, 39.25 μL, 1 eq), DMAP (6.89 mg, 56.40 μmol, 0.2 eq) in DCM (5 mL) was added butanedioyl dichloride (21.85 mg, 141.01 μmol, 15.50 μL, 0.5 eq) at 0° C. The mixture was stirred at 20° C. for 3 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate/NH₃·H₂O=5/1/0.1 to 2/1/0.1) and prep-TLC (SiO₂, Petroleum ether/Ethyl acetate/NH₃·H₂O=3/1/0.1) to give compound 7-[2-[[4-[2-[7-decanoyloxyheptyl-[8-(1-octylnonoxy)-8-oxo-octyl]amino]ethylamino]-4-oxo-butanoyl]amino]ethyl-[8-(1-octylnonoxy)-8-oxo-octyl]amino]heptyl decanoate (0.15 g, 99.97 μmol, 35.45% yield) as yellow oil.

¹H NMR (400 MHz, CDCl₃), 6.28 (brs, 1H), 4.84-4.90 (m, 2H), 4.06 (t, J=6.4 Hz, 4H), 3.28 (brs, 4H), 2.27-2.51 (m, 24H), 1.62-1.65 (m, 10H), 1.52-1.61 (m, 8H), 1.50-1.52 (m, 8H), 1.27-1.32 (m, 98H), 0.89 (t, J=6.4 Hz, 18H). LCMS: (½M+H): 750.5 @ 12.157 min.

### 4.36. Synthesis of Compound 2312

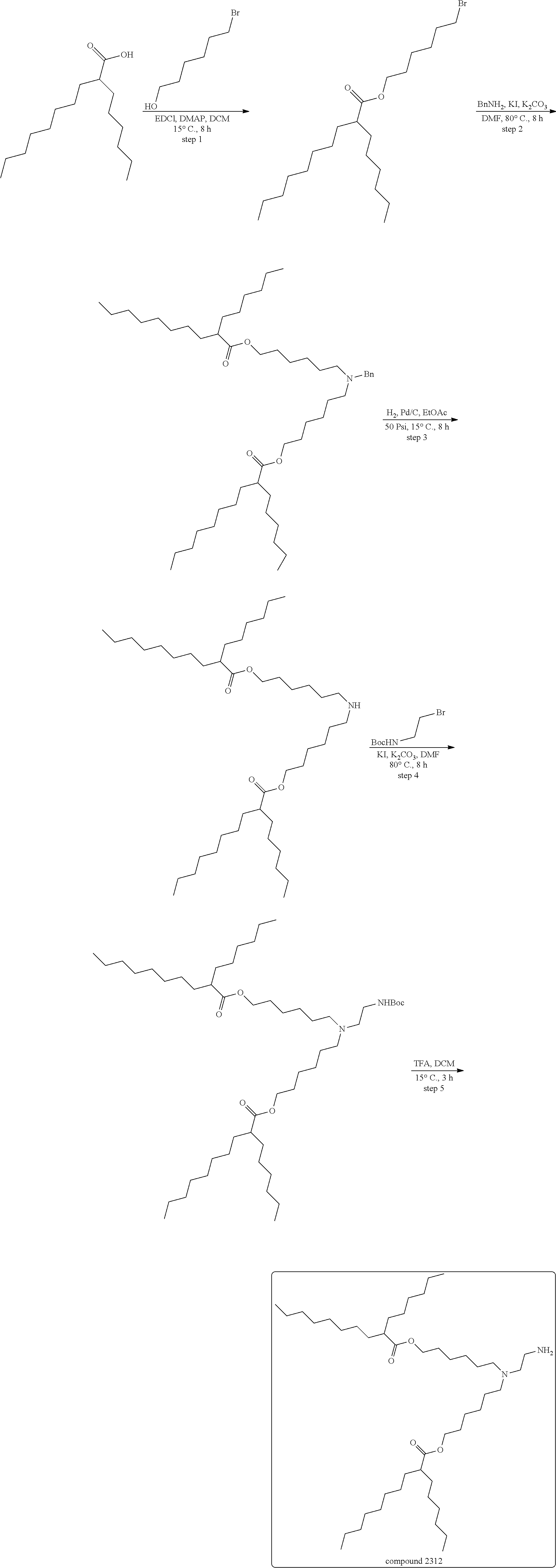

### Step 1:

To a solution of 2-hexyldecanoic acid (2.5 g, 9.75 mmol, 1 eq) and 6-bromohexan-1-ol (1.77 g, 9.75 mmol, 1.28 mL, 1 eq) in DCM (50 mL) was added EDCI (2.24 g, 11.70 mmol, 1.2 eq) and DMAP (595.55 mg, 4.87 mmol, 0.5 eq). The mixture was stirred at 15° C. for 8 hours. The reaction mixture was quenched by addition H₂O 200 mL at 15° C., and then extracted with EtOAc 600 mL (200 mL×3). The combined organic layers were washed with brine 400 mL (200 mL×2), dried over Na₂SO₄, filtered and concentrated under reduced pressure to give a residue. The residue was purified by column chromatography (SiO₂, Petroleum ether/Ethyl acetate=1/0 to 20/1) to give compound 6-bromohexyl 2-hexyldecanoate (14 g, 33.37 mmol, 85.58% yield 4 batches) as colorless oil.

¹H NMR (400 MHz, CDCl₃), 4.08 (t, J=6.8 Hz, 2H), 3.42 (t, J=6.8 Hz, 2H), 2.28-2.35 (m, 1H), 1.84-1.91 (m, 2H), 1.57-1.69 (m, 4H), 1.38-1.48 (m, 6H), 1.26-1.29 (m, 20H), 0.88 (t, J=6.8 Hz, 6H).

### Step 2:

To a solution of phenylmethanamine (851.48 mg, 7.95 mmol, 866.20 μL, 1 eq) in DMF (75 mL) was added K₂CO₃ (5.49 g, 39.73 mmol, 5 eq) and KI (3.30 g, 19.87 mmol, 2.5 eq), then a solution of 6-bromohexyl 2-hexyldecanoate (7 g, 16.69 mmol, 2.1 eq) in DMF (25 mL) was added to the mixture. The mixture was stirred at 80° C. for 8 hours. The reaction mixture was quenched by addition H₂O 200 mL at 15° C., extracted with EtOAc 300 mL (100 mL×3). The combined organic layers were washed with brine 200 mL (100 mL×2), dried over Na₂SO₄, filtered and concentrated under reduced pressure to give a residue. The residue was purified by column chromatography (SiO₂, Petroleum ether/Ethyl acetate=10/1 to 3/1) to give compound 6-[benzyl-[6-(2-hexyldecanoyloxy) hexyl]amino]hexyl 2-hexyldecanoate (9 g, 11.48 mmol, 72.21% yield) as a colorless oil.

¹H NMR (400 MHz, CDCl₃), 7.27-7.33 (m, 4H), 7.20-7.25 (m, 1H), 4.05 (t, J=6.8 Hz, 4H), 2.27-2.41 (m, 4H), 1.56-1.62 (m, 10H), 1.40-1.48 (m, 8H), 1.26-1.32 (m, 50H), 0.88 (t, J=7.2 Hz, 12H).

### Step 3:

A solution of Pd/C (1 g, 10% purity) and 6-[benzyl-[6-(2-hexyldecanoyloxy)hexyl]amino]hexyl 2-hexyldecanoate (4.5 g, 5.74 mmol, 1 eq) in EtOAc (500 mL) was stirred under H₂ under 50 Psi at 15° C. for 8 hours. The reaction mixture was filtered and concentrated under reduced pressure to give a residue. The residue was purified by column chromatography (SiO₂, Petroleum ether/Ethyl acetate=5/1 to 1/0) to give compound 6-[6-(2-hexyldecanoyloxy)hexylamino]hexyl 2-hexyldecanoate (1.8 g, 2.59 mmol, 45.19% yield) as colorless oil.

¹H NMR (400 MHz, CDCl₃), 4.07 (t, J=6.4 Hz, 4H), 2.63 (t, J=7.6 Hz, 4H), 2.28-2.35 (m, 2H), 1.52-1.66 (m, 12H), 1.26-1.45 (m, 52H), 0.88 (t, J=7.2 Hz, 12H).

### Step 4:

To a solution of 6-[6-(2-hexyldecanoyloxy)hexylamino]hexyl 2-hexyldecanoate (800 mg, 1.15 mmol, 1 eq) in DMF (10 mL) was added K₂CO₃ (796.39 mg, 5.76 mmol, 5 eq) and KI (191.31 mg, 1.15 mmol, 1 eq) and then added tert-butyl N-(2-bromoethyl)carbamate (1.16 g, 5.19 mmol, 4.5 eq) in DMF (5 mL). The mixture was stirred at 80° C. for 8 hours. The reaction mixture was quenched by addition H₂O 20 mL at 15° C., extracted with EtOAc 30 mL (10 mL×3). The combined organic layers were washed with brine 20 mL (10 mL×2), dried over Na₂SO₄, filtered and concentrated under reduced pressure to give a residue. The residue was purified by column chromatography (SiO₂, Petroleum ether/Ethyl acetate=20/1 to 3/1) to give compound 6-[2-(tert-butoxycarbonylamino) ethyl-[6-(2-hexyldecanoyloxy)hexyl]amino]hexyl 2-hexyldecanoate (560 mg, 668.78 μmol) as a colorless oil.

### Step 5:

A solution of 6-[2-(tert-butoxycarbonylamino)ethyl-[6-(2-hexyldecanoyloxy)hexyl]amino]hexyl 2-hexyldecanoate (560 mg, 668.78 μmol, 1 eq) in DCM (4 mL) and TFA (3.59 g, 31.51 mmol, 2.33 mL, 47.12 eq) was stirred at 15° C. for 3 hours. The reaction mixture was concentrated under reduced pressure to give a residue. The residue was purified by prep-TLC (SiO₂, EA:MeOH=10:1) to give compound 6-[2-aminoethyl-[6-(2-hexyldecanoyloxy)hexyl]amino]hexyl 2-hexyldecanoate (420 mg, 552.61 μmol, 82.63% yield, 97% purity) as a yellow oil.

¹H NMR (400 MHz, CDCl₃), 4.07 (t, J=6.8 Hz, 4H), 2.79 (t, J=6.0 Hz, 2H), 2.53 (t, J=6.0 Hz, 2H), 2.45 (t, J=7.2 Hz, 4H), 2.29-2.34 (m, 2H), 2.21 (brs, 2H), 1.59-1.65 (m, 8H), 1.43-1.45 (m, 8H), 1.26-1.42 (m, 48H), 0.89 (t, J=7.2 Hz, 12H). LCMS. (M+H⁺): 737.5 @ 11.219 min.

### 4.37. Synthesis of Compound 2313

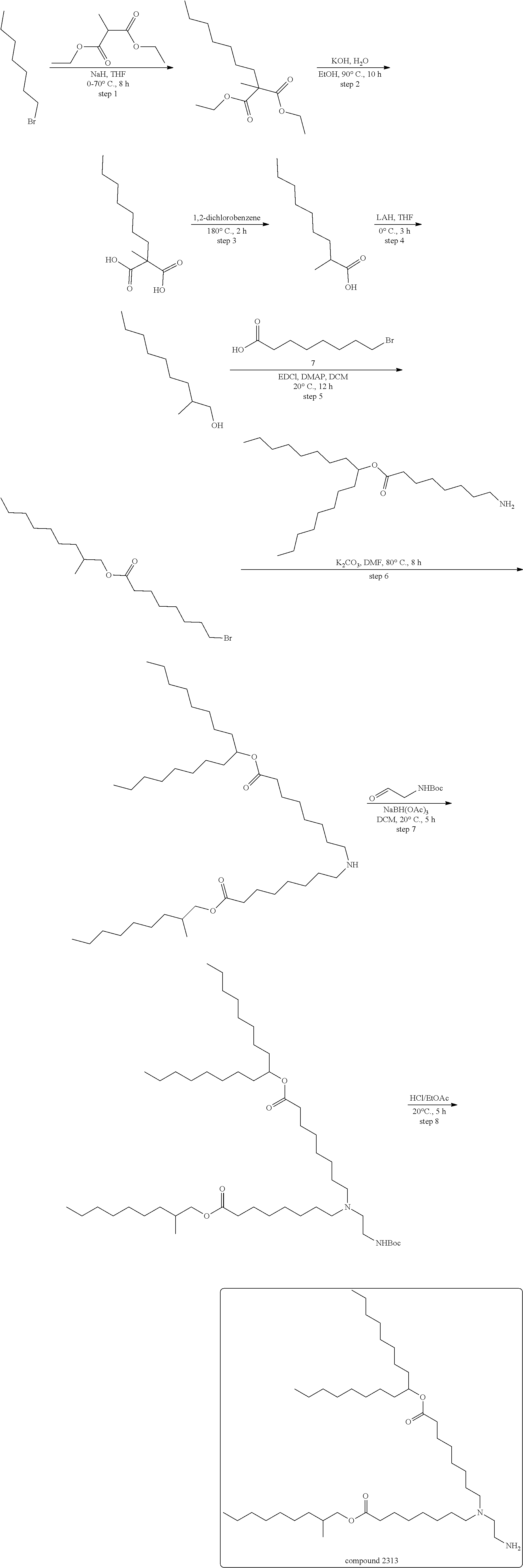

### Step 1:

To a solution of diethyl 2-methylpropanedioate (10 g, 57.41 mmol, 9.80 mL, 1 eq) in THE (1000 mL) in three-necked flask was added NaH (2.30 g, 57.41 mmol, 60% purity, 1 eq) slowly at 0° C. and stirred at 0° C. for 1 hour. 1-bromoheptane (10.28 g, 57.41 mmol, 9.02 mL, 1 eq) was added and stirred at 20° C. for 0.5 hour and stirred at 70° C. for 6.5 hours. The reaction mixture was quenched by addition H₂O 2000 mL at 0° C. The mixture was extracted with EtOAc 3000 mL (1000 mL×3) and the combined organic layers were dried over Na₂SO₄, filtered and concentrated under reduced pressure to give a residue. The residue was purified by column chromatography (SiO₂, Petroleum ether/Ethyl acetate=1/0 to 50/1) to give compound diethyl 2-heptyl-2-methyl-propanedioate (45 g, 165.21 mmol, 71.95% yield, 3 batches) as colorless oil.

¹H NMR (400 MHz, CDCl₃), 4.15-4.21 (m, 4H), 1.85-1.87 (m, 2H), 1.40 (s, 3H), 1.23-1.28 (m, 16H), 0.88 (t, J=6.8 Hz, 3H).

### Step 2:

To a solution of diethyl 2-heptyl-2-methyl-propanedioate (10 g, 36.71 mmol, 1 eq) in EtOH (100 mL) and H₂O (100 mL) was added KOH (6.18 g, 110.14 mmol, 3 eq). The mixture was stirred at 90° C. for 10 hours. The reaction mixture was concentrated under reduced pressure to remove most of EtOH and washed with EtOAc 120 mL (40 mL×3). Then the aqueous phase was adjusted pH=2 with 1M HCl aqueous and extracted with EtOAc 120 mL (40 mL×3). The combined organic layers were dried over Na₂SO₄, filtered and concentrated under reduced pressure to give a compound 2-heptyl-2-methyl-propanedioic acid (45 g, 208.07 mmol, 94.46% yield) as a white solid without purification.

### Step 3:

The solution of 2-heptyl-2-methyl-propanedioic acid (10 g, 46.24 mmol, 1 eq) in 1,2-dichlorobenzene (104.80 g, 712.92 mmol, 80.00 mL, 15.42 eq) was stirred at 180° C. for 2 hours. The reaction mixture was diluted with H₂O 200 mL and extracted with EtOAc 600 mL (200 mL×3). The combined organic layers were dried over Na₂SO₄, filtered and concentrated under reduced pressure to give a residue. The residue was purified by column chromatography (SiO₂, Petroleum ether/Ethyl acetate=1/0 to 3/1) to give compound 2-methylnonanoic acid (35 g, 203.18 mmol, 87.88% yield) as a white solid.

### Step 4:

To a solution of LiAlH₄ (3.08 g, 81.27 mmol, 2 eq) in THE (500 mL) was added 2-methylnonanoic acid (7 g, 40.64 mmol, 1 eq). The mixture was stirred at 0° C. for 3 hours. The reaction mixture was quenched by addition H₂O 200 mL at 0° C. The mixture was extracted with EtOAc 300 mL (100 mL×3) and the combined organic layers were dried over Na₂SO₄, filtered and concentrated under reduced pressure to give a residue. The residue was purified by column chromatography (SiO₂, Petroleum ether/Ethyl acetate=20/1 to 10/1) to give compound 2-methylnonan-1-ol (15 g, 94.77 mmol, 46.64% yield) as colorless oil.

¹H NMR (400 MHz, CDCl₃), 3.50-3.54 (m, 1H), 3.42-3.45 (m, 1H), 1.61-1.64 (m, 1H), 1.20-1.39 (m, 11H), 1.05-1.15 (m, 1H), 0.87-0.93 (m, 6H).

### Step 5:

To a mixture of 8-bromooctanoic acid (9.87 g, 44.23 mmol, 1 eq) in DCM (1000 mL) was added DMAP (1.08 g, 8.85 mmol, 0.2 eq), 2-methylnonan-1-ol (7 g, 44.23 mmol, 1 eq), EDCI (8.48 g, 44.23 mmol, 1 eq) at 20° C. The mixture was stirred at 20° C. for 12 hours under N₂ atmosphere. The reaction mixture was diluted with EtOAc 600 mL (200 mL×3) and washed with H₂O 200 mL, 10% aq. citric acid 200 mL (100 mL×2). The combined organic layers were dried over Na₂SO₄, filtered and concentrated under reduced pressure to give a residue. The residue was purified by column chromatography (SiO₂, Petroleum ether/Ethyl acetate=1/0 to 1/0) to give compound 2-methylnonyl 8-bromooctanoate (12 g, 33.02 mmol, 74.67% yield) as yellow oil.

### Step 6:

To a solution of 1-octylnonyl 8-aminooctanoate (4.38 g, 11.01 mmol, 1 eq), 2-methylnonyl 8-bromooctanoate (4 g, 11.01 mmol, 1 eq) in ACN (100 mL) was added K₂CO₃ (1.52 g, 11.01 mmol, 1 eq). The mixture was stirred at 80° C. for 8 hours. The reaction mixture was diluted with H₂O 200 mL and extracted with EtOAc 600 mL (200 mL×3). The combined organic layers were dried over Na₂SO₄, filtered and concentrated under reduced pressure to give a residue. The residue was purified by column chromatography (SiO₂, Petroleum ether/Ethyl acetate/NH₃·H₂O=10/1/1 to 1/1/0.5) to give a compound 2-methylnonyl 8-[[8-(1-octylnonoxy)-8-oxo-octyl]amino]octanoate (3.5 g, 5.15 mmol, 23.37% yield) as yellow oil.

¹H NMR (400 MHz, CDCl₃), 4.85-4.89 (m, 1H), 3.93-3.98 (m, 1H), 3.83-3.88 (m, 1H), 2.59 (t, J=7.2 Hz, 4H), 2.26-2.33 (m, 4H), 1.60-1.80 (m, 1H), 1.40-1.60 (m, 10H), 1.20-1.40 (m, 50H), 0.86-0.93 (m, 12H).

### Step 7:

To a solution of 2-methylnonyl 8-[[8-(1-octylnonoxy)-8-oxo-octyl]amino]octanoate (3 g, 4.41 mmol, 1 eq), tert-butyl N-(2-oxoethyl)carbamate (1.05 g, 6.62 mmol, 1.5 eq) in DCM (50 mL) was added NaBH(OAc)₃ (1.87 g, 8.82 mmol, 2 eq). The mixture was stirred at 20° C. for 5 hours. The combined organic phase was diluted with EtOAc 20 mL and washed with water 60 mL (20 mL×3) and brine 40 mL (20 mL×2), dried over Na₂SO₄, filtered and concentrated under reduced pressure to give a residue. The residue was purified by column chromatography (SiO₂, Petroleum ether/Ethyl acetate=10/1 to 5/1) to give compound 2-methylnonyl 8-[2-(tert-butoxycarbonylamino) ethyl-[8-(1-octylnonoxy)-8-oxo-octyl]amino] octanoate (2 g, 2.43 mmol, 55.07% yield) as a white solid.

¹H NMR (400 MHz, CDCl₃), 4.85-4.89 (m, 1H), 3.93-3.98 (m, 1H), 3.83-3.87 (m, 1H), 3.14 (brs, 2H), 2.28-2.40 (m, 10H), 1.60-1.80 (m, 1H), 1.26-1.55 (m, 69H), 0.86-0.93 (m, 12H).

### Step 8:

A solution of 2-methylnonyl 8-[2-(tert-butoxycarbonylamino)ethyl-[8-(1-octylnonoxy)-8-oxo-octyl]amino] octanoate (2 g, 2.43 mmol, 1 eq) in HCl/EtOAc (4 M, 9.37 mL, 15.42 eq) was stirred at 20° C. for 5 hours. The reaction mixture was adjusted pH=7 with saturated NaHCO₃ aqueous and extracted with EtOAc 150 mL (50 mL×3). The combined organic layers were dried over Na₂SO₄, filtered and concentrated under reduced pressure to give a residue. The residue was purified by column chromatography (SiO₂, Petroleum ether/Ethyl acetate=5/1 to 0/1) to give compound 2-methylnonyl 8-[2-aminoethyl-[8-(1-octylnonoxy)-8-oxo-octyl]amino]octanoate (1.5 g, 2.07 mmol, 85.38% yield, 100% purity) as a white solid without purification.

¹H NMR (400 MHz, CDCl₃), 4.84-4.90 (m, 1H), 3.94-3.98 (m, 1H), 3.83-3.87 (m, 1H), 2.73 (t, J=6.0 Hz, 2H), 2.46 (t, J=6.0 Hz, 2H), 2.40 (t, J=7.2 Hz, 4H), 2.27-2.36 (m, 4H), 1.72-1.82 (m, 1H), 1.60-1.65 (m, 4H), 1.50-1.54 (m, 4H), 1.39-1.45 (m, 4H), 1.23-1.34 (m, 46H), 1.12-1.27 (m, 2H), 0.87-0.93 (m, 12H). LCMS: (M+H⁺): 723.4 @ 10.618 min.

### 4.38. Synthesis of Compound 2314

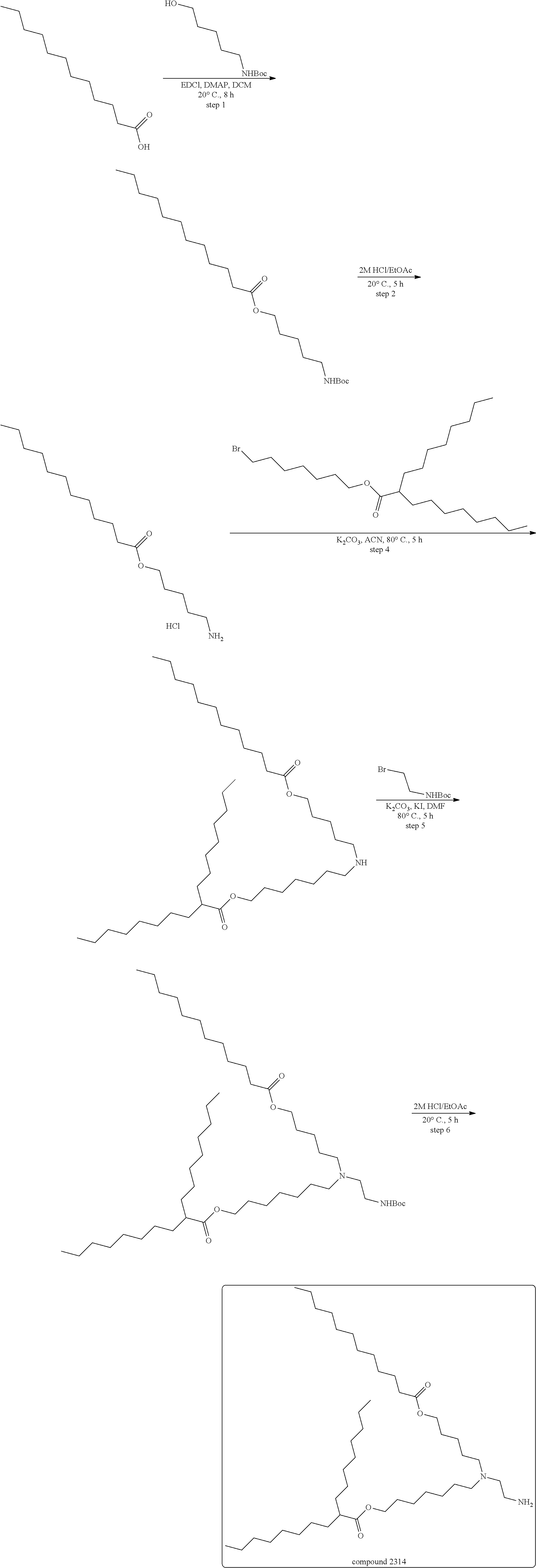

### Step 1:

A mixture of dodecanoic acid (4.93 g, 24.60 mmol, 1 eq) in DCM (1000 mL) was added DMAP (1.50 g, 12.30 mmol, 0.5 eq), tert-butyl N-(5-hydroxypentyl)carbamate (5 g, 24.60 mmol, 5.00 mL, 1 eq), EDCI (9.43 g, 49.19 mmol, 2 eq) and was degassed and purged with N₂ for 3 times. The mixture was stirred at 20° C. for 8 hours under N₂ atmosphere. The reaction mixture was diluted with EtOAc 200 mL and washed with H₂O 200 mL. The combined organic layers were dried over Na₂SO₄, filtered and concentrated under reduced pressure to give a residue. The residue was purified by column chromatography (SiO₂, Petroleum ether/Ethyl acetate=1/0 to 1/0) to give compound 5-(tert-butoxycarbonylamino) pentyl dodecanoate (6 g, 15.56 mmol, 63.26% yield) as yellow oil.

¹H NMR (400 MHz, CDCl₃), 4.52 (brs, 1H), 4.06 (t, J=6.4 Hz, 2H), 3.10-3.13 (m, 2H), 2.29 (t, J=7.6 Hz, 2H), 1.64-1.66 (m, 4H), 1.51-1.61 (m, 2H), 1.49 (s, 9H), 1.44-1.45 (m, 2H), 1.26-1.38 (m, 16H), 0.88 (t, J=6.4 Hz, 3H).

### Step 2:

5-(tert-butoxycarbonylamino)pentyl dodecanoate (6 g, 15.56 mmol, 1 eq) in HCl/EtOAc (2 M, 60.00 mL, 15.42 eq) was stirred at 20° C. for 5 hours. The mixture was filtered and the filter cake was concentrated under reduced pressure to give compound 5-aminopentyl dodecanoate (4 g, 12.43 mmol, 79.85% yield, HCl) as a white solid without purification.

LCMS: (M+H⁺): 386.3 @ 0.887 min.

### Step 3:

A mixture of 7-bromoheptan-1-ol (3.43 g, 17.58 mmol, 1 eq) in DCM (1000 mL) was added DMAP (429.46 mg, 3.52 mmol, 0.2 eq), 2-octyldecanoic acid (5 g, 17.58 mmol, 1 eq), EDCI (3.37 g, 17.58 mmol, 1 eq) and was degassed and purged with N₂ for 3 times. The mixture was stirred at 20° C. for 8 hours under N₂ atmosphere. The reaction mixture was diluted with EtOAc 600 mL (200 mL×3) and washed with H₂O 200 mL. The combined organic layers were dried over Na₂SO₄, filtered and concentrated under reduced pressure to give a residue. The residue was purified by column chromatography (SiO₂, Petroleum ether/Ethyl acetate=1/0 to 1/0) to give a compound 7-bromoheptyl 2-octyldecanoate (5 g, 10.83 mmol, 61.63% yield) as yellow oil.

### Step 4:

To a solution of 5-aminopentyl dodecanoate (2 g, 6.21 mmol, 1 eq, HCl), 7-bromoheptyl 2-octyldecanoate (2.87 g, 6.21 mmol, 1 eq) in ACN (100 mL) was added K₂CO₃ (2.58 g, 18.64 mmol, 3 eq). The mixture was stirred at 80° C. for 5 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate/NH₃·H₂O=10/1/1 to 1/1/0.5) to give compound 7-(5-dodecanoyloxypentylamino) heptyl 2-octyldecanoate (1.5 g, 2.25 mmol, 36.25% yield) as yellow oil.

¹H NMR (400 MHz, CDCl₃), 4.05-4.09 (m, 4H), 2.59-2.63 (m, 4H), 2.27-2.31 (m, 3H), 1.60-1.70 (m, 8H), 1.45-1.60 (m, 4H), 1.35-1.45 (m, 8H), 1.20-1.35 (m, 44H), 0.88 (t, J=6.8 Hz, 9H).

### Step 5:

To a solution of 7-(5-dodecanoyloxypentylamino)heptyl 2-octyldecanoate (1.5 g, 2.25 mmol, 1 eq), tert-butyl N-(2-bromoethyl)carbamate (2.52 g, 11.26 mmol, 30.22 μL, 5 eq) in DMF (10 mL) was added K₂CO₃ (1.56 g, 11.26 mmol, 5 eq), KI (373.81 mg, 2.25 mmol, 1 eq) and stirred at 80° C. for 5 hours. The reaction mixture was diluted with H₂O 20 mL and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=10/1 to 1/1) to give compound 7-[2-(tert-butoxycarbonylamino) ethyl-(5-dodecanoyloxypentyl)amino]heptyl 2-octyldecanoate (1 g, 1.24 mmol, 54.87% yield) as yellow oil.

### Step 6:

7-[2-(tert-butoxycarbonylamino)ethyl-(5-dodecanoyloxypentyl)amino]heptyl 2-octyldecanoate (1 g, 1.24 mmol, 1 eq) in HCl/EtOAc (2 M, 4.76 mL, 15.42 eq) was stirred at 20° C. for 5 hours. The crude reaction mixture was adjusted pH=7 with saturated NaHCO₃ aqueous and extracted with EtOAc 150 mL (50 mL×3). The combined organic layers were dried over Na₂S₄, filtered and concentrated under reduced pressure to give a residue. The residue was purified by column chromatography (SiO₂, Petroleum ether/Ethyl acetate=5/1 to 0/1) to give compound 7-[2-aminoethyl(5-dodecanoyloxypentyl)amino]heptyl 2-octyldecanoate (0.7 g, 977.19 μmol, 79.08% yield, 99% purity) as yellow oil.

¹H NMR (400 MHz, CDCl₃), 4.07 (t, J=6.8 Hz, 4H), 2.73 (t, J=6.0 Hz, 2H), 2.47 (t, J=6.0 Hz, 2H), 2.39-2.44 (m, 4H), 2.30 (t, J=7.2 Hz, 3H), 1.61-1.66 (m, 6H), 1.44-1.47 (m, 6H), 1.20-1.36 (m, 50H), 0.89 (t, J=6.8 Hz, 9H). LCMS: (M+H⁺): 709.3 @ 10.360 m.

### Example 5. Preparation of Lipid Nanoparticle Compositions

Exemplary lipid nanoparticle compositions were prepared to result in an ionizable lipid:structural lipid:sterol:PEG-lipid at a molar ratio shown in the below charts.

Molar ratios of the lipid components of each lipid nanoparticle composition are summarized below.

| Ionizable | | Molar ratio | | | |
|---|---|---|---|---|---|
| Lipid | | Ionizable | Structural | Plant | DMPE- |
| No. | mRNA | component | DSPC | Cholesterol | PEG2k |
| 2129 | CRE/FLUC 1:1 | 50 | 10 | 46.5 | 1.5 |
| 2130 | FLUC/EPO 1:1 | 50 | 10 | 46.5 | 1.5 |
| 2131 | CRE/FLUC 1:1 | 50 | 10 | 46.5 | 1.5 |
| 2132 | EGFP | 50 | 10 | 46.5 | 1.5 |
| 2132 | CRE/FLUC 1:1 | 50 | 10 | 46.5 | 1.5 |
| 2133 | FLUC/EPO 1:1 | 50 | 10 | 46.5 | 1.5 |
| 2134 | EGFP | 50 | 10 | 46.5 | 1.5 |
| 2134 | CRE/FLUC 1:1 | 50 | 10 | 46.5 | 1.5 |
| 2135 | EGFP | 50 | 10 | 46.5 | 1.5 |
| 2136 | EGFP | 50 | 10 | 46.5 | 1.5 |
| 2138 | CRE/FLUC 1:1 | 50 | 10 | 46.5 | 1.5 |
| 2139 | FLUC/EPO 1:1 | 50 | 10 | 46.5 | 1.5 |
| 2140 | CRE/FLUC 1:1 | 50 | 10 | 46.5 | 1.5 |
| 2141 | EGFP | 50 | 10 | 46.5 | 1.5 |
| 2141 | CRE/FLUC 1:1 | 50 | 10 | 46.5 | 1.5 |
| 2141 | FLUC/EPO 1:1 | 50 | 10 | 38.5 | 1.5 |
| 2142 | FLUC/EPO 1:1 | 50 | 10 | 46.5 | 1.5 |
| 2143 | EGFP | 50 | 10 | 46.5 | 1.5 |
| 2143 | CRE/FLUC 1:1 | 50 | 10 | 46.5 | 1.5 |
| 2144 | EGFP | 50 | 10 | 46.5 | 1.5 |
| 2144 | CRE/FLUC 1:1 | 50 | 10 | 46.5 | 1.5 |
| 2145 | FLUC/EPO 1:1 | 50 | 10 | 46.5 | 1.5 |
| 2146 | CRE/FLUC 1:1 | 50 | 10 | 46.5 | 1.5 |
| 2215 | FLUC/EPO 1:1 | 50 | 10 | 38.5 | 1.5 |
| 2216 | FLUC/EPO 1:1 | 50 | 10 | 38.5 | 1.5 |
| 2225 | FLUC/EPO 1:1 | 50 | 10 | 38.5 | 1.5 |
| 2233 | FLUC/EPO 1:1 | 50 | 10 | 38.5 | 1.5 |
| 2235 | FLUC/EPO 1:1 | 50 | 10 | 38.5 | 1.5 |
| 2241 | FLUC/EPO 1:1 | 50 | 10 | 38.5 | 1.5 |
| 2242 | FLUC/EPO 1:1 | 50 | 10 | 38.5 | 1.5 |
| 2243 | FLUC/EPO 1:1 | 50 | 10 | 38.5 | 1.5 |
| 2244 | FLUC/EPO 1:1 | 50 | 10 | 38.5 | 1.5 |
| 2249 | FLUC/EPO 1:1 | 50 | 10 | 38.5 | 1.5 |
| 2250 | FLUC/EPO 1:1 | 50 | 10 | 38.5 | 1.5 |
| 2335 | FLUC/EPO 1:1 | 50 | 10 | 38.5 | 1.5 |

To prepare the exemplary lipid nanoparticle compositions, the lipid components according to the above chart were solubilized in ethanol, mixed at the above-indicated molar ratios, and diluted in ethanol (organic phase) to obtain total lipid concentration of 5.5 mM.

An mRNA solution (aqueous phase, fluc:EPO mRNA, cre:fluc mRNA, or EGFP mRNA), according to the above chart for each LNP composition, was prepared with RNAse-free water and 100 mM citrate buffer pH 3 for a final concentration of 50 mM citrate buffer and 0.167 mg/mL mRNA concentration (1:1 Fluc:EPO, 1:1 cre:Fluc, or EGFP). The formulations were maintained at an ionizable lipid to mRNA at an ionizable lipid nitrogen:mRNA phosphate (N:P) ratio of 6:1.

For each LNP composition, the lipid mix and mRNA solution were mixed at a 1:3 ratio by volume, respectively, on a NanoAssemblr Ignite (Precision Nanosystems) at a total flow rate of 9 mL/min. The resulting compositions were then loaded into Slide-A-Lyzer G2 dialysis cassettes (10 k MWCO) and dialyzed in 200 times sample volume of 1×PBS for 2 hours at room temperature with gentle stirring. The PBS was refreshed, and the compositions were further dialyzed for at least 14 hours at 4° C. with gentle stirring. The dialyzed compositions were then collected and concentrated by centrifugation at 3000×g using Amicon Ultra centrifugation filters (100 k MWCO). The concentrated particles were characterized for size, polydispersity, and particle concentration using Zetasizer Ultra (Malvern Panalytical) and for mRNA encapsulation efficiency using Quant-iT RiboGreen RNA Assay Kit (ThermoFisher Scientific).

For pKa measurement, a TNA assay was conducted according to those described in Sabnis et al., Molecular Therapy, 26(6):1509-19), which is incorporated herein by reference in its entirety. Briefly, 20 buffers (10 mM sodium phosphate, 10 mM sodium borate, 10 mM sodium citrate, and 150 mM sodium chloride, in distilled Water) of unique pH values ranging from 3.0-12.0 were prepared using 1M sodium hydroxide and 1M hydrochloric acid. 3.25 μL of a LNP composition (0.04 mg/mL mRNA, in PBS) was incubated with 2 μL of TNS reagent (0.3 mM, in DMSO) and 90 μL of buffer for each pH value (described above) in a 96-well black-walled plate. Each pH condition was performed in triplicate wells. The TNS fluorescence was measured using a Biotek Cytation Plate reader at excitation/emission wavelengths of 321/445 nm. The fluorescence values were then plotted and fit using a 4-parameter sigmoid curve. From the fit, the pH value yielding the half-maximal fluorescence was calculated and reported as the apparent LNP pKa value.

The particle characterization data for each exemplary lipid nanoparticle compositions are shown in the table below.

| Ionizable | | | | | |
|---|---|---|---|---|---|
| Lipid No. | mRNA | Size (nm) | PDI | % EE | pKa (TNS) |
| 2129 | CRE/FLUC 1:1 | 81.1 | 0.07 | 97.0 | — |
| 2130 | FLUC/EPO 1:1 | 68.7 | 0.13 | 98.5 | — |
| 2131 | CRE/FLUC 1:1 | 71.2 | 0.13 | 97.1 | — |
| 2132 | EGFP | 90.4 | 0.20 | 97.2 | — |
| 2132 | CRE/FLUC 1:1 | 76.3 | 0.13 | 97.7 | — |
| 2133 | FLUC/EPO 1:1 | 87.7 | 0.08 | 98.7 | 7.96 |
| 2134 | EGFP | 107.1 | 0.20 | 98.3 | — |
| 2134 | CRE/FLUC 1:1 | 92.9 | 0.11 | 98.3 | — |
| 2135 | EGFP | 81.8 | 0.04 | 96.2 | — |
| 2136 | EGFP | 77.5 | 0.04 | 95.5 | — |
| 2138 | CRE/FLUC 1:1 | 112.7 | 0.35 | 97 | — |
| 2139 | FLUC/EPO 1:1 | 88.6 | 0.18 | 98.3 | — |
| 2140 | CRE/FLUC 1:1 | 86.9 | 0.16 | 96.9 | — |
| 2141 | EGFP | 109.3 | 0.21 | 95.4 | — |
| 2141 | CRE/FLUC 1:1 | 79.4 | 0.11 | 95.6 | — |
| 2141 | FLUC/EPO 1:1 | 73.83 | 0.06 | 95.9 | 7.57 |
| 2142 | FLUC/EPO 1:1 | 69.2 | 0.06 | 96.7 | — |
| 2143 | EGFP | 83.4 | 0.08 | 97.3 | — |
| 2143 | CRE/FLUC 1:1 | 78.6 | 0.11 | 97.1 | — |
| 2144 | EGFP | 73.5 | 0.29 | 98.3 | — |
| 2144 | CRE/FLUC 1:1 | 78.0 | 0.26 | 96.8 | — |
| 2145 | FLUC/EPO 1:1 | 95.4 | 0.21 | 97.6 | — |
| 2146 | CRE/FLUC 1:1 | 81.0 | 0.26 | 96.5 | — |
| 2215 | FLUC/EPO 1:1 | 80.18 | 0.1 | 96.3 | 7.12 |
| 2216 | FLUC/EPO 1:1 | 88.81 | 0.11 | 96.8 | 7 |
| 2225 | FLUC/EPO 1:1 | 81.88 | 0.07 | 97.9 | 7.25 |
| 2233 | FLUC/EPO 1:1 | 85.5 | 0.04 | 91.2 | 6.83 |
| 2235 | FLUC/EPO 1:1 | 84.84 | 0.067 | 97.5 | 7.76 |
| 2241 | FLUC/EPO 1:1 | 100.89 | 0.15 | 94.7 | — |
| 2242 | FLUC/EPO 1:1 | 154.2 | 0.079 | 89.5 | — |
| 2243 | FLUC/EPO 1:1 | 87.77 | 0.04 | 94.9 | 7.1 |
| 2249 | FLUC/EPO 1:1 | 86.05 | 0.08 | 93.3 | 5.84 |
| 2250 | FLUC/EPO 1:1 | 73.75 | 0.068 | 92.2 | 6.31 |
| 2335 | FLUC/EPO 1:1 | 87.74 | 0.02 | 93.3 | 6.4 |

### Example 6. In-Vivo Bioluminescent Imaging

The exemplary lipid nanoparticle compositions prepared according to Example 5, with encapsulating an mRNA according to the table shown above in Example 5, were used in this example.

8-9 week old female Balb/c mice were utilized for bioluminescence-based ionizable lipid screening efforts. Mice were obtained from Jackson Laboratories (JAX Stock: 000651) and allowed to acclimate for one week prior to manipulations. Animals were placed under a heat lamp for a few minutes before introducing them to a restraining chamber. The tail was wiped with alcohol pads (Fisher Scientific) and, for each LNP composition described above, 100 uL of a lipid nanoparticle composition described above containing 10 g total mRNA (5 g Fluc+5 μg EPO, 5 μg Fluc+5 μg Cre, or 5 μg EGFP) was injected intravenously using a 29G insulin syringe (Covidien).

4-6 hours post-dose, animals were injected with 200 μL of 15 mg/mL D-Luciferin (GoldBio), and placed in set nose cones inside the IVIS Lumina LT imager (PerkinElmer). LivingImage software was utilized for imaging. Whole body bio-luminescence was captured at auto-exposure after which animals are removed from the IVIS and placed into a CO₂ chamber for euthanasia. Cardiac puncture was performed on each animal after placing it in dorsal recumbency, and blood collection was performed using a 25G insulin syringe (BD). Once all blood samples were collected, tubes are spun at 2000G for 10 minutes using a tabletop centrifuge and plasma was aliquoted into individual Eppendorf tubes (Fisher Scientific) and stored at −80° C. for subsequent EPO quantification. EPO levels in plasma were determined using EPO MSD kit (Meso Scale Diagnostics).

The EPO levels determined by the in-vivo bioluminescent imaging for each lipid nanoparticle compositions are shown in the table below.

| | | Bioluminescence (IV) | | | | | |
|---|---|---|---|---|---|---|---|
| | | | | | | | Spleen: |
| Ionizable | mRNA | Whole | | | | | Liver |
| Lipid No. | dose | Body | Liver | Spleen | Lung | hEPO | Ratio |
| 2129 | 5 μg FLUC | 1.9E + 04 | 1.4E + 05 | 9.2E + 03 | 2.1E + 03 | | 0.066 |
| 2130 | 5 μg FLUC + | 1.2E + 04 | 7.0E + 04 | 3.0E + 03 | 1.4E + 05 | 1.5E + 02 | 0.045 |
| | 5 μg EPO | | | | | | |
| 2131 | 5 μg FLUC | 1.2E + 04 | 1.3E + 05 | 9.5E + 03 | 2.4E + 03 | | 0.076 |
| 2132 | 5 μg FLUC | 7.0E + 03 | 1.4E + 05 | 2.5E + 04 | 1.7E + 03 | | 0.212 |
| 2133 | 5 μg FLUC + | 3.8E + 04 | 4.4E + 04 | 3.9E + 04 | 5.8E + 04 | 2.5E + 02 | 1.574 |
| | 5 μg EPO | | | | | | |
| 2134 | 5 μg FLUC | 5.4E + 03 | 7.5E + 04 | 4.3E + 03 | 1.4E + 03 | | 0.063 |
| 2138 | 5 μg FLUC | 3.7E + 03 | 1.7E + 05 | 2.8E + 04 | 1.2E + 03 | | 0.236 |
| 2139 | 5 μg FLUC + | 4.1E + 04 | 1.7E + 05 | 1.0E + 05 | 2.2E + 05 | 2.0E + 02 | 0.583 |
| | 5 μg EPO | | | | | | |
| 2140 | 5 μg FLUC | 4.3E + 04 | 1.6E + 03 | 1.2E + 05 | 5.3E + 03 | | 76.18 |
| 2141 | 5 μg FLUC | 5.5E + 06 | 5.6E + 05 | 2.3E + 05 | 7.1E + 03 | | 0.527 |
| 2141 | 5 μg FLUC + | 1.9E + 05 | 3.1E + 04 | 1.3E + 05 | 6.8E + 04 | 2.1E + 03 | 6.131 |
| | 5 μg EPO | | | | | | |
| 2142 | 5 μg FLUC + | 7.8E + 05 | 2.4E + 05 | 5.6E + 05 | 2.0E + 05 | 7.3E + 03 | 2.758 |
| | 5 μg EPO | | | | | | |
| 2143 | 5 μg FLUC | 1.7E + 05 | 1.3E + 05 | 6.5E + 05 | 6.3E + 03 | | 5.050 |
| 2144 | 5 μg FLUC | 7.1E + 03 | 7.4E + 04 | 3.0E + 04 | 1.2E + 03 | | 0.401 |
| 2145 | 5 μg FLUC + | 4.0E + 03 | 1.1E + 05 | 1.2E + 03 | 7.2E + 04 | 8.4E + 02 | 0.012 |
| | 5 μg EPO | | | | | | |
| 2146 | 5 μg FLUC | 7.4E + 03 | 9.7E + 04 | 4.7E + 04 | 1.2E + 03 | | 0.505 |
| 2215 | 5 μg FLUC + | 3.2E + 07 | 2.2E + 06 | 6.6E + 05 | 6.5E + 04 | | 0.449 |
| | 5 μg EPO | | | | | | |
| 2216 | 5 μg FLUC + | 1.5E + 06 | 3.2E + 05 | 3.8E + 05 | 9.0E + 03 | | 1.539 |
| | 5 μg EPO | | | | | | |
| 2225 | 5 μg FLUC + | 3.5E + 05 | 1.8E + 05 | 2.2E + 05 | 1.0E + 04 | | 1.261 |
| | 5 μg EPO | | | | | | |
| 2233 | 5 μg FLUC + | 5.4E + 08 | 6.1E + 07 | 4.7E + 06 | 4.1E + 04 | 2.8E + 06 | 0.077 |
| | 5 μg EPO | | | | | | |
| 2235 | 5 μg FLUC + | 9.8E + 03 | 2.0E + 03 | 2.9E + 04 | 7.6E + 03 | 2.2E + 02 | 14.407 |
| | 5 μg EPO | | | | | | |
| 2241 | 5 μg FLUC + | 3.1E + 04 | 3.5E + 03 | 7.6E + 04 | 5.4E + 04 | 1.6E + 03 | 22.241 |
| | 5 μg EPO | | | | | | |
| 2243 | 5 μg FLUC + | 1.6E + 08 | 3.0E + 07 | 4.2E + 06 | 1.0E + 05 | 2.7E + 06 | 0.138 |
| | 5 μg EPO | | | | | | |
| 2249 | 5 μg FLUC + | 7.2E + 07 | 1.2E + 07 | 3.2E + 06 | 7.9E + 03 | 7.6E + 05 | 0.258 |
| | 5 μg EPO | | | | | | |
| 2250 | 5 μg FLUC + | 1.5E + 08 | 2.7E + 07 | 2.7E + 06 | 7.9E + 03 | 9.5E + 05 | 0.099 |
| | 5 μg EPO | | | | | | |
| 2335 | 5 μg FLUC + | 3.00E + 08 | 4.30E + 07 | 4.80E + 06 | 8.80E + 04 | 2.60E + 06 | 0.1147 |
| | 5 μg EPO | | | | | | |

As can be seen, the lipid nanoparticle compositions containing the novel ionizable lipid compounds demonstrate selective delivery of the therapeutic cargos outside the liver and, due to the lower lipid levels in the liver, lower liver toxicity is expected.

While this disclosure has been described in relation to some embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications, and equivalents. In particular, this disclosure includes any combination of the features, terms, or elements of the various illustrative components and examples.

1. A compound of formula (I) or (IA): 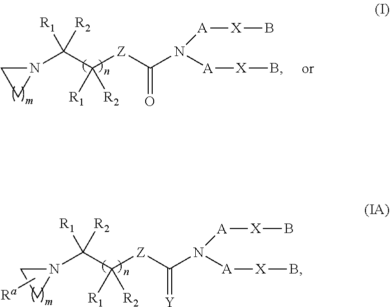
a pharmaceutically acceptable salt thereof, or a stereoisomer of any of the foregoing,
wherein
R^{a} is H, C₁-C₃ branched or unbranched alkyl, C₂-C₃ branched or unbranched alkenyl, halogen, OH, or SH;
each R₁ and each R₂ is independently H, C₁-C₃ branched or unbranched alkyl, C₂-C₃ branched or unbranched alkenyl, OH, halogen, SH, or NR₁₀R₁₁ or R₁ and R₂ are taken together to form a cyclic ring; each R₁₀ and R₁₁ is independently H, C₁-C₃ alkyl, or R₁₀ and R₁₁ are taken together to form a heterocyclic ring;
m is 1, 2, 3, 4, 5, 6, 7 or 8;
n is 0, 1, 2, 3 or 4;
Y is O or S;
Z is absent, O, S, or NR₁₂, wherein R₁₂ is H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH;
each A is each independently C₁-C₁₆ branched or unbranched alkyl, or C₂-C₁₆ branched or unbranched alkenyl, optionally substituted with heteroatom or optionally substituted with OH, SH, or halogen;
each B is each independently C₁-C₁₆ branched or unbranched alkyl, or C₂-C₁₆ branched or unbranched alkenyl, optionally substituted with heteroatom or optionally substituted with OH, SH, or halogen; and
each X is independently a biodegradable moiety.

2. The compound of claim 1, wherein X is —OCO—, —COO—, —NHCO—, —CONH—, —C(O—R₁₃)—O—, —COO(CH₂)ₛ—, —CONH(CH₂)ₛ—, —C(O—R₁₃)—O—(CH₂)ₛ, wherein R₁₃ is C₃-C₁₀ alkyl and s is 1, 2, 3, 4, or 5.

3. The compound of claim 1, wherein:
X is —OCO— or —COO—;
each R₁ and R₂ are independently H or OH;
B is a C₃-C₂₀ alkyl;
m is 1, 3 or 4;
n is 0, 1, or 2; or
combinations thereof.

4. The compound of claim 1, wherein Z is absent, O, S, or NH.

5-11. (canceled)

12. A compound of formula (V), (VA), or (VB): 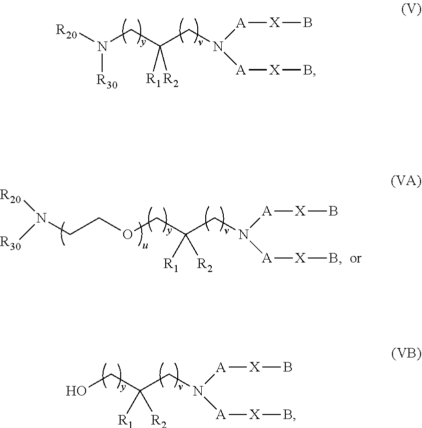
a pharmaceutically acceptable salt thereof, or a stereoisomer of any of the foregoing,
wherein
R₁ is H, C₁-C₃ branched or unbranched alkyl, C₂-C₃ branched or unbranched alkenyl, OH, halogen, SH, or NR₁₀R₁₁; and R₂ is OH, halogen, SH, or NR₁₀R₁₁, wherein
R₁₀ and R₁₁ are each independently H or C₁-C₃ alkyl or R₁₀ and R₁₁ are taken together to form a heterocyclic ring, or
R₁ and R₂ are taken together to form a cyclic ring;
R₂₀ and R₃₀ are each independently H or C₁-C₅ alkyl, or R₂₀ and R₃₀ are taken together to form a cyclic ring;
u is 0, 1, 2, 3, 4, 5, 6, 7, or 8;
v is 1, 2, 3, or 4;
y is 1, 2, 3, or 4;
each A is independently C₁-C₁₆ branched or unbranched alkyl, or C₂-C₁₆ branched or unbranched alkenyl, optionally substituted with heteroatom or substituted with OH, SH, or halogen;
each B is each independently C₁-C₁₆ branched or unbranched alkyl, or C₂-C₁₆ branched or unbranched alkenyl, optionally substituted with heteroatom or substituted with OH, SH, or halogen; and
X is a biodegradable moiety.

13. The compound of claim 12, wherein X is —OCO—, —COO—, —NHCO—, —CONH—, —C(O—R₁₃)—O—, —COO(CH₂)ₛ—, —CONH(CH₂)ₛ—, —C(O—R₁₃)—O—(CH₂)ₛ—, wherein R₁₃ is C₃-C₁₀ alkyl and s is 1, 2, 3, 4, or 5.

14. The compound of claim 12, wherein:
X is —OCO— or —COO—;
each R₁ and R₂ are independently H or OH;
B is a C₃-C₂₀ alkyl;
m is 1, 3 or 4;
n is 0, 1, or 2; or
combinations thereof.

15. The compound of claim 1, wherein Z is absent, O, S, or NH.

16-21. (canceled)

22. The compound of claim 1, wherein the pKa of the protonated form of the compound is from about 5.1 to about 8.0.

23-26. (canceled)

27. A combination of the compound of claim 1 and a lipid component.

28. The combination of claim 27, wherein the combination comprises about a 1:1 ratio of the compound and the lipid component.

29. The combination of claim 27, wherein the combination is a LNP composition.

30. The combination of claim 27, wherein the lipid component comprises a helper lipid and a PEG lipid, and optionally a neutral lipid.

31-33. (canceled)

34. The combination of claim 27, further comprising a nucleic acid component.

35. The combination of claim 34, wherein the nucleic acid component is an RNA or DNA component.

36. The combination of claim 34, having an N/P ratio of about 3-10.

37-39. (canceled)

40. The combination of claim 35, wherein the nucleic acid component is a RNA component, and wherein the RNA component comprises a mRNA.

41. A method for delivering a therapeutic cargo to a target organ of a subject in need thereof comprising administering to said subject a composition comprising one or more compounds according to claim 1.

42. The method of claim 41, wherein the target organ is the pancreas or the lung, and wherein:
less than 50%, 30%, or 10% of the therapeutic cargo is delivered to the liver of the subject; and/or
more than 50%, 70%, or 90% of the therapeutic cargo is delivered to the pancreas and/or lung of the subject.

43. (canceled)

44. A compound having one of the following formulas:

| Lipid | |
|---|---|
| No. | Structure |
| 7677 (2140) | 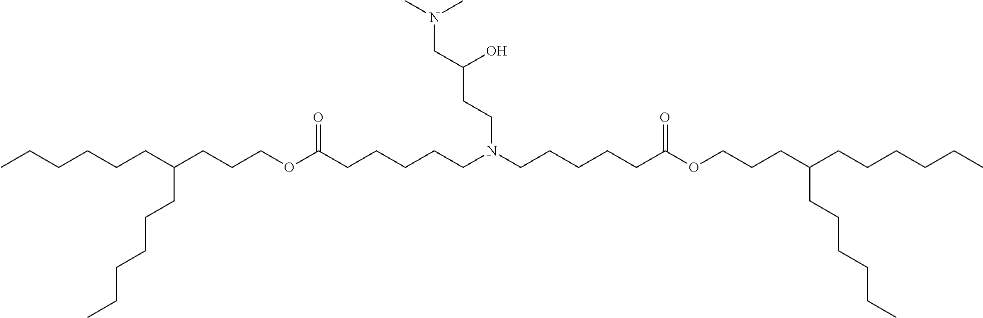 [SMILES: CCCCCCC(CCCCCC)CCCOC(=O)CCCCCN(CCCCCC(=O)OCCCC(CCCCCC)CCCCCC)CCC(O)CN(C)C] |
| 7676 (2139) | 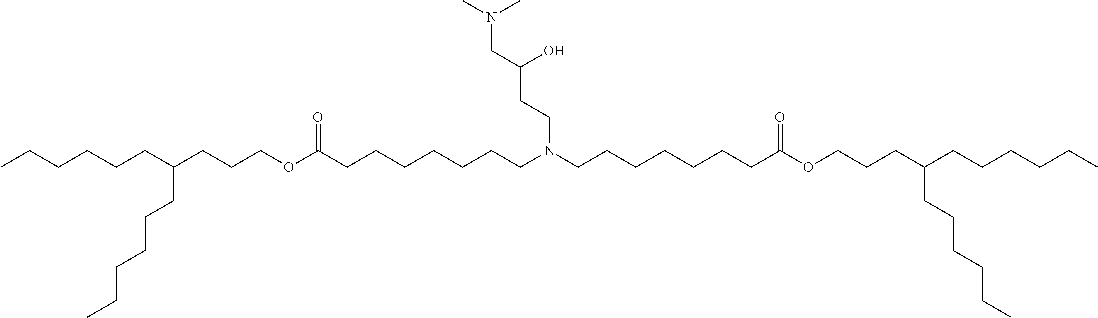 [SMILES: CCCCCCC(CCCCCC)CCCOC(=O)CCCCCCCN(CCCCCCCC(=O)OCCCC(CCCCCC)CCCCCC)CCC(O)CN(C)C] |
| 7675 (2138) | 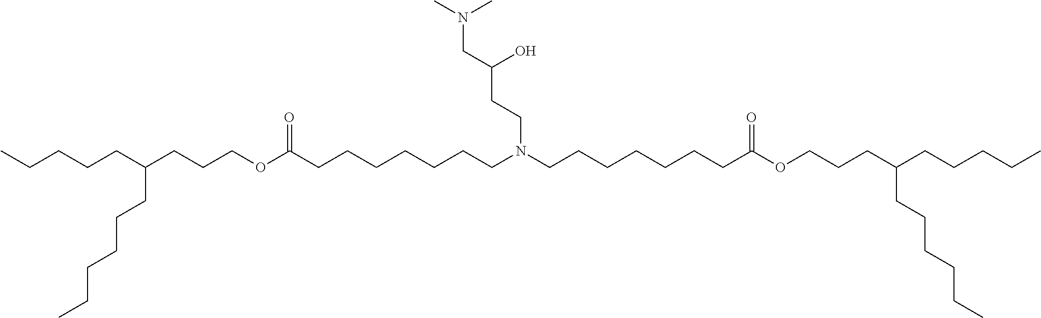 [SMILES: CCCCCCC(CCCCC)CCCOC(=O)CCCCCCCN(CCCCCCCC(=O)OCCCC(CCCCC)CCCCCC)CCC(O)CN(C)C] |
| 7671 (2142) | 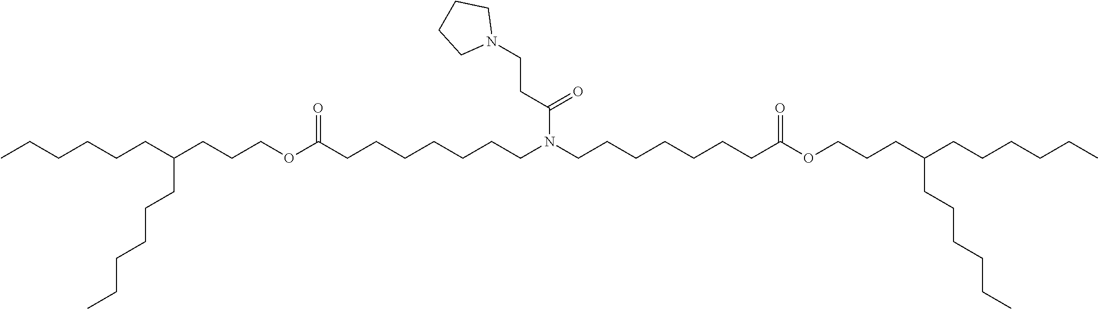 [SMILES: CCCCCCC(CCCCCC)CCCOC(=O)CCCCCCCN(CCCCCCCC(=O)OCCCC(CCCCCC)CCCCCC)C(=O)CCN1CCCC1] |
| 7670 (2146) | 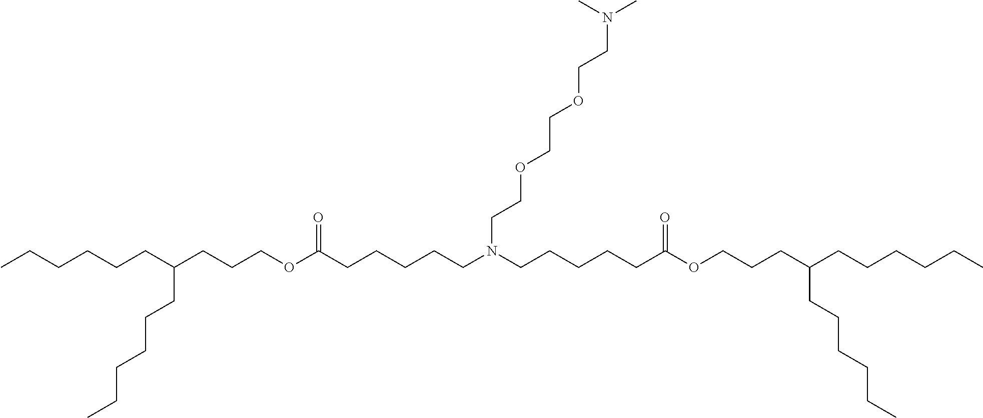 [SMILES: CCCCCCC(CCCCCC)CCCOC(=O)CCCCCN(CCCCCC(=O)OCCCC(CCCCCC)CCCCCC)CCOCCOCCN(C)C] |
| 7669 (2145) | 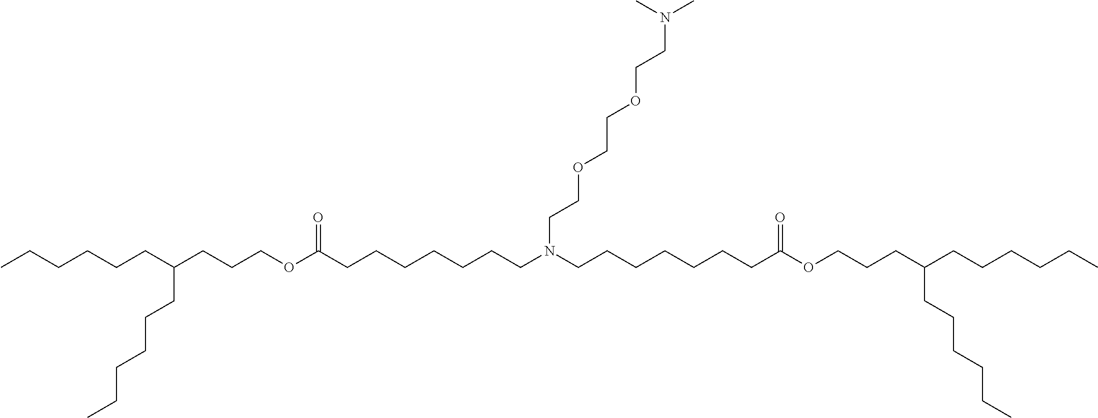 [SMILES: CCCCCCC(CCCCCC)CCCOC(=O)CCCCCCCN(CCCCCCCC(=O)OCCCC(CCCCCC)CCCCCC)CCOCCOCCN(C)C] |
| 7668 (2133) | 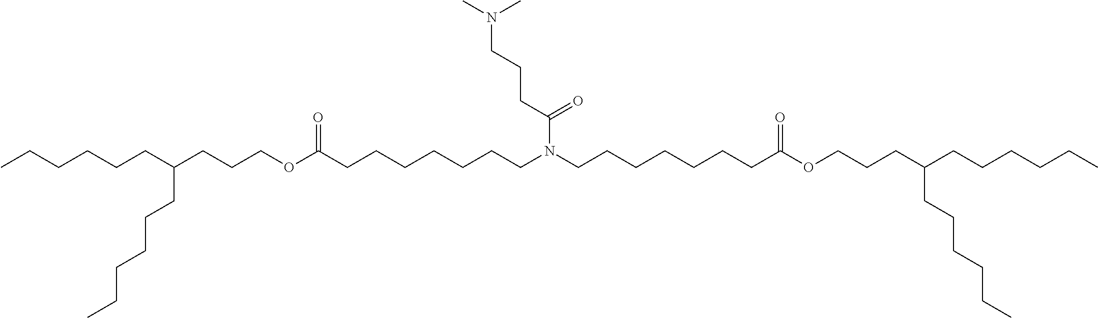 [SMILES: CCCCCCC(CCCCCC)CCCOC(=O)CCCCCCCN(CCCCCCCC(=O)OCCCC(CCCCCC)CCCCCC)C(=O)CCCN(C)C] |
| 7667 (2137) | 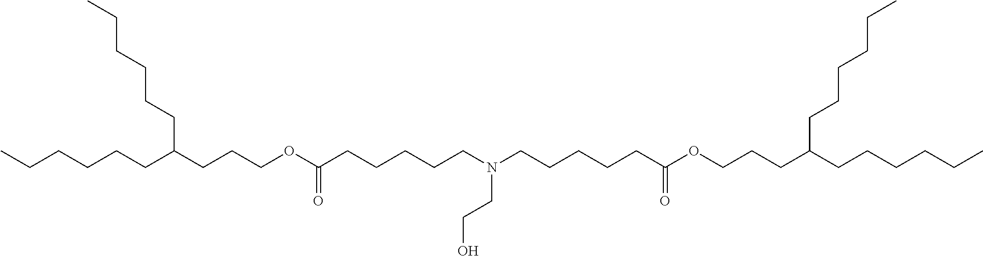 [SMILES: CCCCCCC(CCCCCC)CCCOC(=O)CCCCCN(CCO)CCCCCC(=O)OCCCC(CCCCCC)CCCCCC] |
| 7651 (2131) | 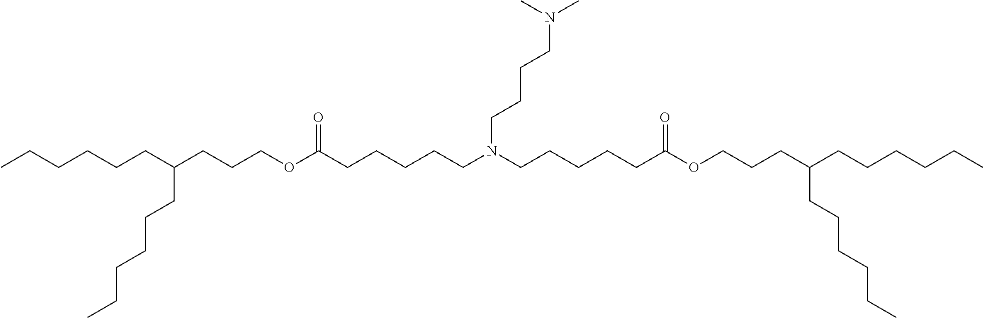 [SMILES: CCCCCCC(CCCCCC)CCCOC(=O)CCCCCN(CCCCCC(=O)OCCCC(CCCCCC)CCCCCC)CCCCN(C)C] |
| 7650 (2130) | 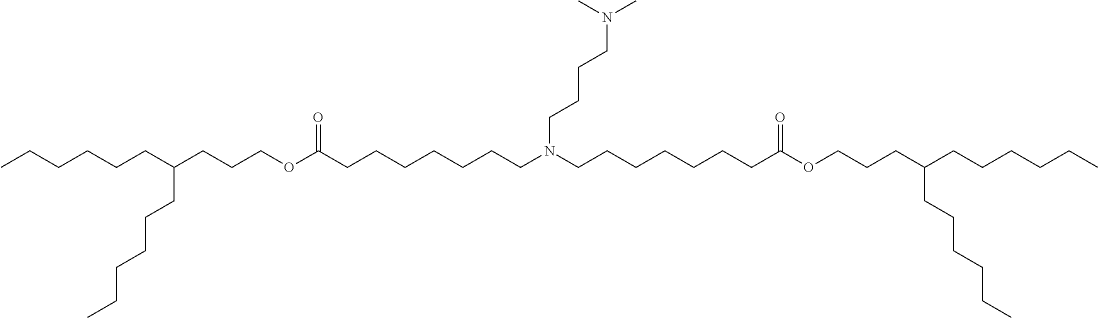 [SMILES: CCCCCCC(CCCCCC)CCCOC(=O)CCCCCCCN(CCCCCCCC(=O)OCCCC(CCCCCC)CCCCCC)CCCCN(C)C] |
| 7649 (2129) | 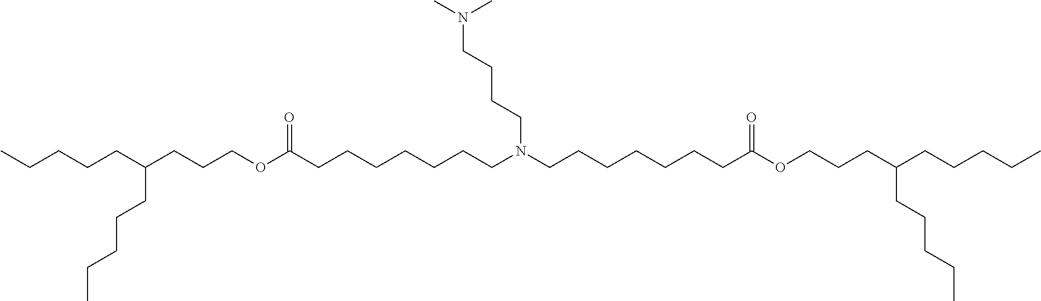 [SMILES: CCCCCC(CCCCC)CCCOC(=O)CCCCCCCN(CCCCCCCC(=O)OCCCC(CCCCC)CCCCC)CCCCN(C)C] |
| 7633 (2144) | 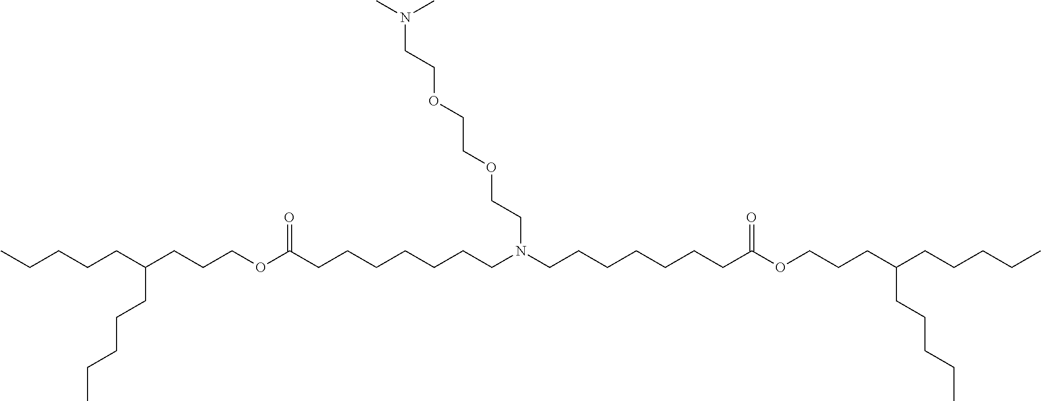 [SMILES: CCCCCC(CCCCC)CCCOC(=O)CCCCCCCN(CCCCCCCC(=O)OCCCC(CCCCC)CCCCC)CCOCCOCCN(C)C] |
| 7632 (2143) | 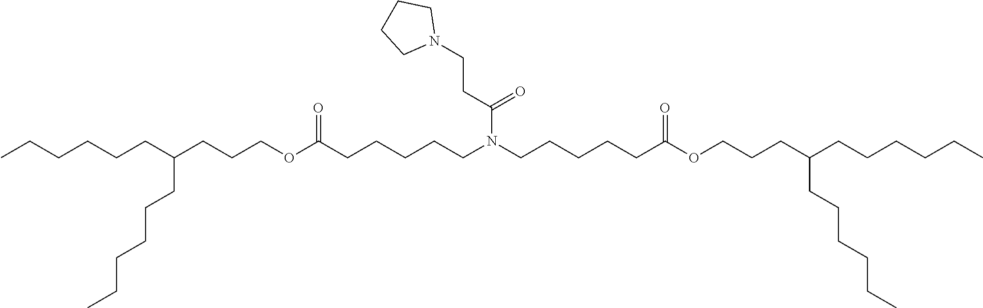 [SMILES: CCCCCCC(CCCCCC)CCCOC(=O)CCCCCN(CCCCCC(=O)OCCCC(CCCCCC)CCCCCC)C(=O)CCN1CCCC1] |
| 7631 (2134) | 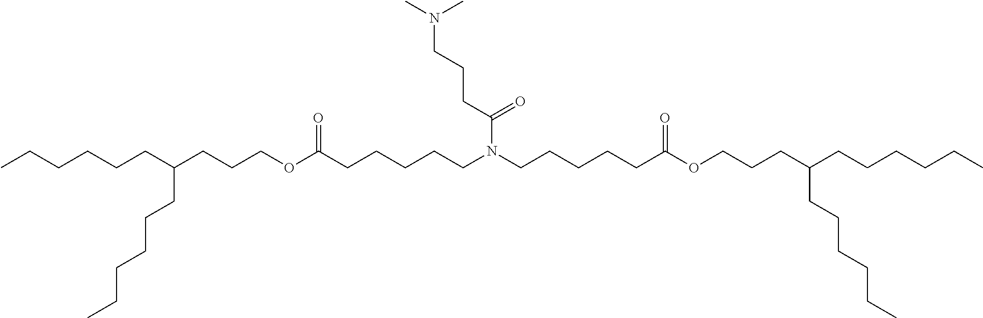 [SMILES: CCCCCCC(CCCCCC)CCCOC(=O)CCCCCN(CCCCCC(=O)OCCCC(CCCCCC)CCCCCC)C(=O)CCCN(C)C] |
| 7608 (2135) | 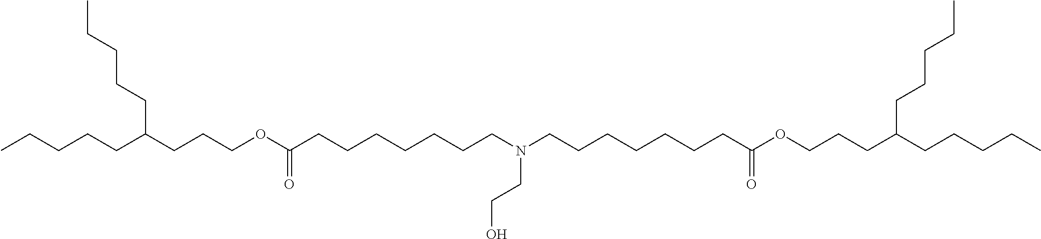 [SMILES: CCCCCC(CCCCC)CCCOC(=O)CCCCCCCN(CCO)CCCCCCCC(=O)OCCCC(CCCCC)CCCCC] |
| 7607 (2136) | 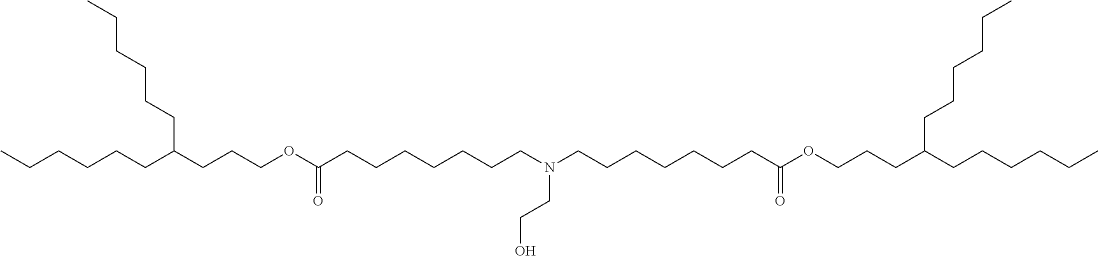 [SMILES: CCCCCCC(CCCCCC)CCCOC(=O)CCCCCCCN(CCO)CCCCCCCC(=O)OCCCC(CCCCCC)CCCCCC] |
| 7596 (2141) | 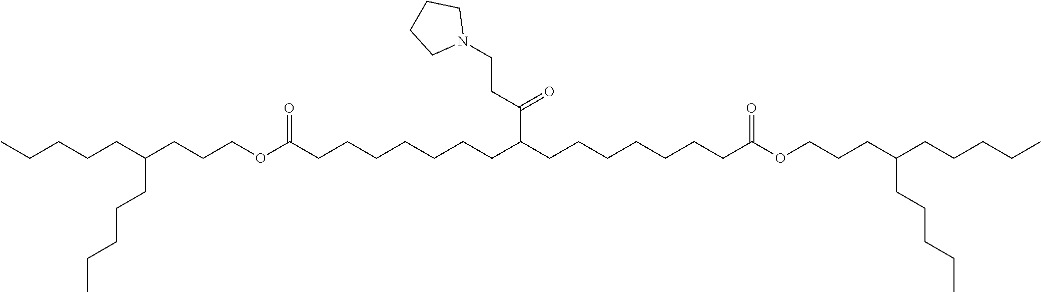 [SMILES: CCCCCC(CCCCC)CCCOC(=O)CCCCCCCC(CCCCCCCC(=O)OCCCC(CCCCC)CCCCC)C(=O)CCN1CCCC1] |
| 7593 (2132) | 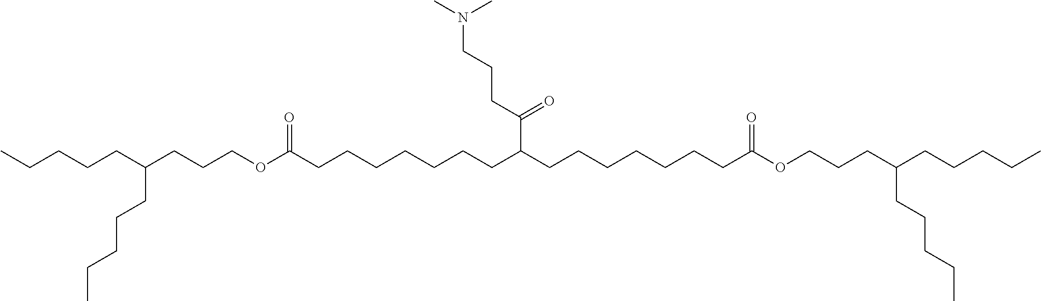 [SMILES: CCCCCC(CCCCC)CCCOC(=O)CCCCCCCC(CCCCCCCC(=O)OCCCC(CCCCC)CCCCC)C(=O)CCCN(C)C] |
| 2229 | 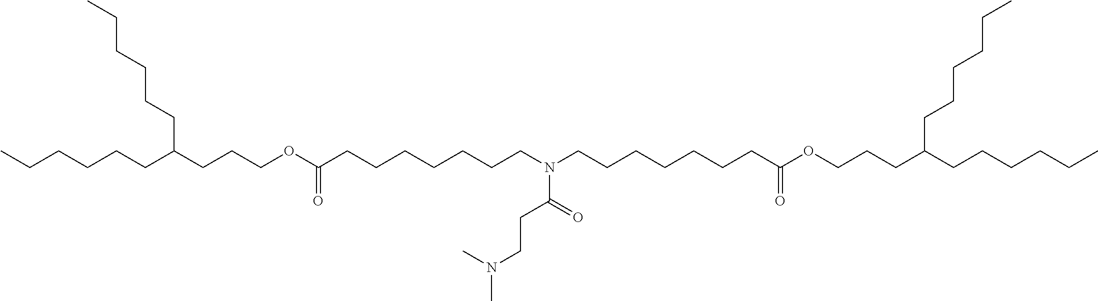 [SMILES: CCCCCCC(CCCCCC)CCCOC(=O)CCCCCCCN(CCCCCCCC(=O)OCCCC(CCCCCC)CCCCCC)C(=O)CCN(C)C] |
| 2228 | 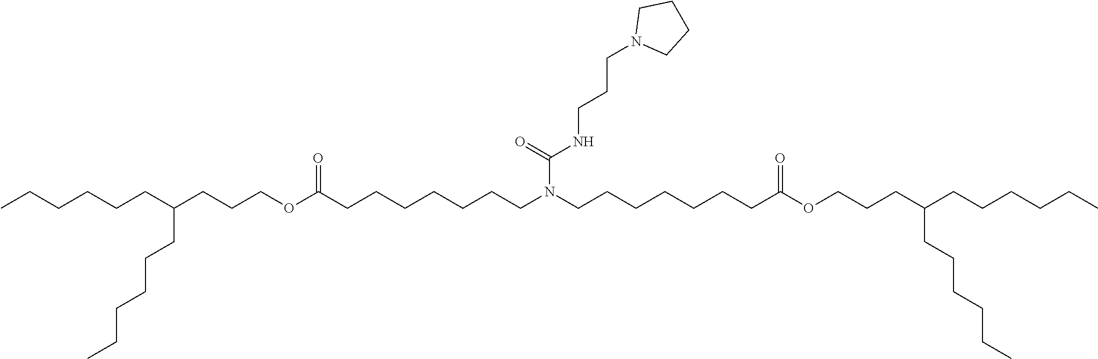 [SMILES: CCCCCCC(CCCCCC)CCCOC(=O)CCCCCCCN(CCCCCCCC(=O)OCCCC(CCCCCC)CCCCCC)C(=O)NCCCN1CCCC1] |
| 2227 | 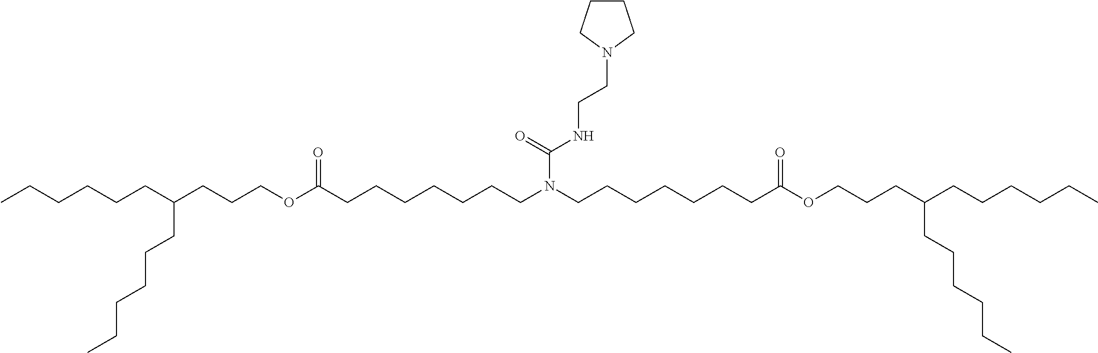 [SMILES: CCCCCCC(CCCCCC)CCCOC(=O)CCCCCCCN(CCCCCCCC(=O)OCCCC(CCCCCC)CCCCCC)C(=O)NCCN1CCCC1] |
| 2226 | 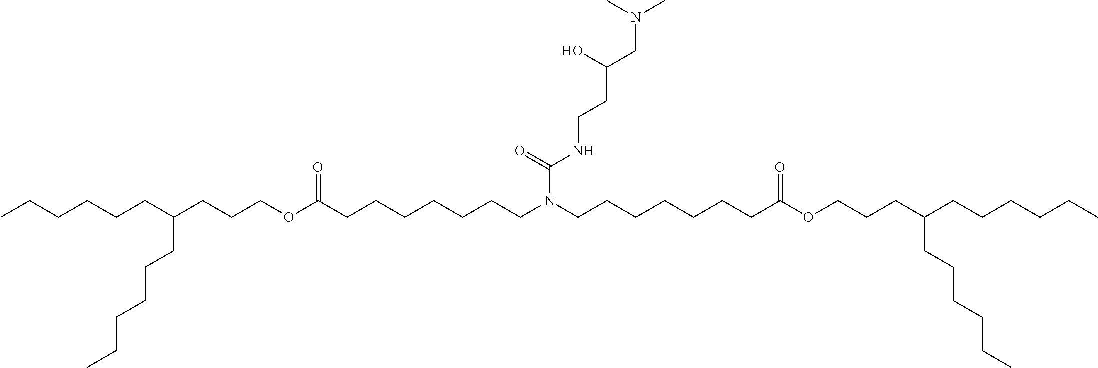 [SMILES: CCCCCCC(CCCCCC)CCCOC(=O)CCCCCCCN(CCCCCCCC(=O)OCCCC(CCCCCC)CCCCCC)C(=O)NCCC(O)CN(C)C] |
| 2225 | 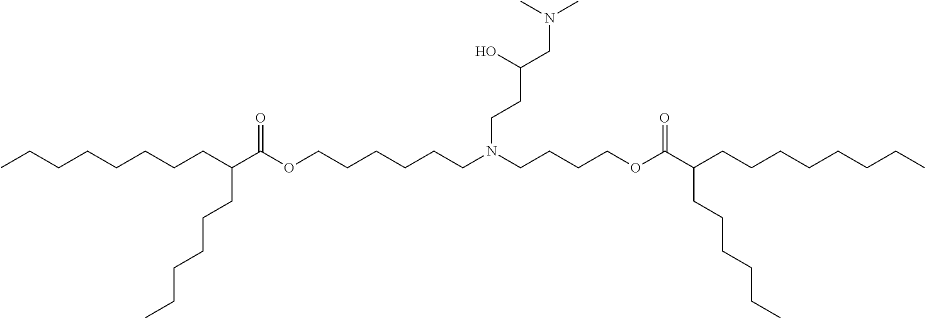 [SMILES: CCCCCCCCC(CCCCCC)C(=O)OCCCCCCN(CCCCOC(=O)C(CCCCCC)CCCCCCCC)CCC(O)CN(C)C] |
| 2216 | 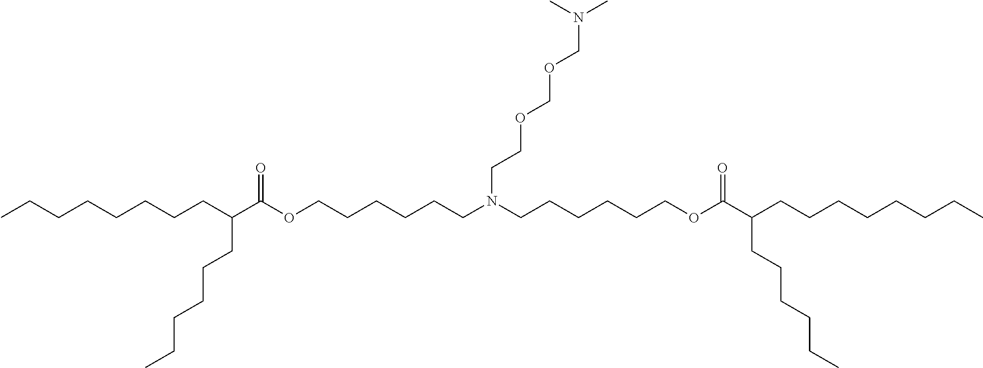 [SMILES: CCCCCCCCC(CCCCCC)C(=O)OCCCCCCN(CCCCCCOC(=O)C(CCCCCC)CCCCCCCC)CCOCOCN(C)C] |
| 2215 | 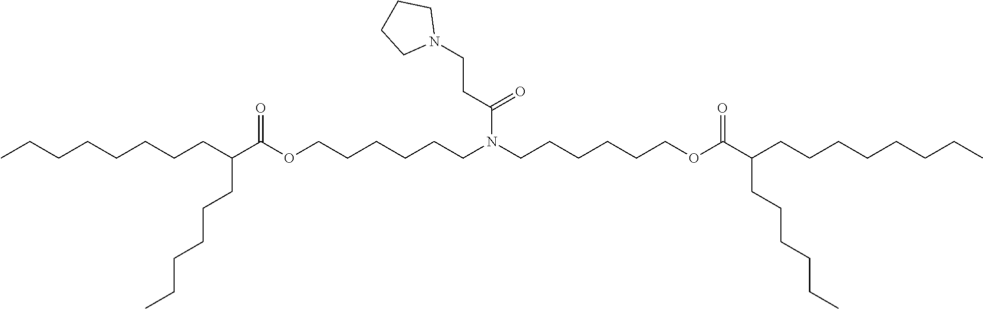 [SMILES: CCCCCCCCC(CCCCCC)C(=O)OCCCCCCN(CCCCCCOC(=O)C(CCCCCC)CCCCCCCC)C(=O)CCN1CCCC1] |
| | 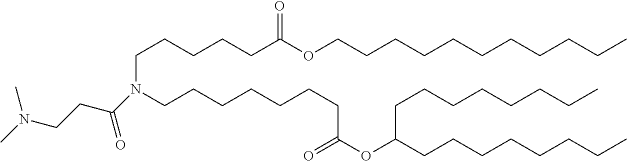 [SMILES: CCCCCCCCCCCOC(=O)CCCCCN(CCCCCCCC(=O)OC(CCCCCCCC)CCCCCCCC)C(=O)CCN(C)C] |
| | 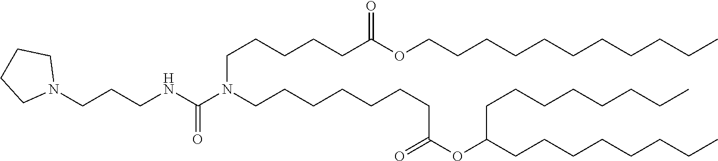 [SMILES: CCCCCCCCCCCOC(=O)CCCCCN(CCCCCCCC(=O)OC(CCCCCCCC)CCCCCCCC)C(=O)NCCCN1CCCC1] |
| | 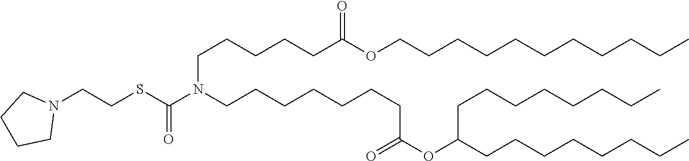 [SMILES: CCCCCCCCCCCOC(=O)CCCCCN(CCCCCCCC(=O)OC(CCCCCCCC)CCCCCCCC)C(=O)SCCN1CCCC1] |
| | 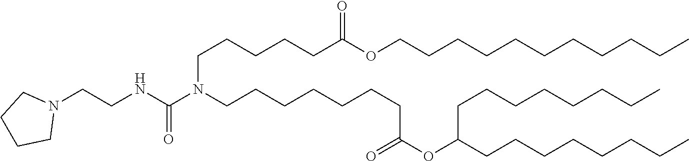 [SMILES: CCCCCCCCCCCOC(=O)CCCCCN(CCCCCCCC(=O)OC(CCCCCCCC)CCCCCCCC)C(=O)NCCN1CCCC1] |
| | 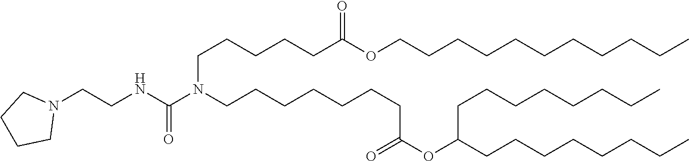 [SMILES: CCCCCCCCCCCOC(=O)CCCCCN(CCCCCCCC(=O)OC(CCCCCCCC)CCCCCCCC)C(=O)NCCN1CCCC1] |
| | 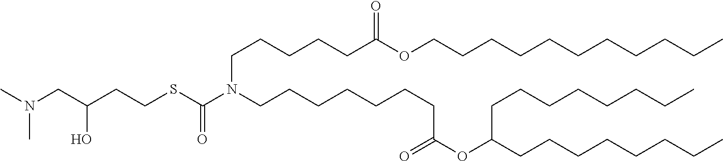 [SMILES: CCCCCCCCCCCOC(=O)CCCCCN(CCCCCCCC(=O)OC(CCCCCCCC)CCCCCCCC)C(=O)SCCC(O)CN(C)C] |
| | 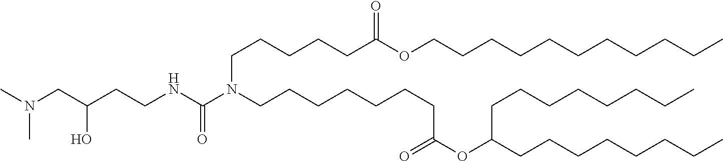 [SMILES: CCCCCCCCCCCOC(=O)CCCCCN(CCCCCCCC(=O)OC(CCCCCCCC)CCCCCCCC)C(=O)NCCC(O)CN(C)C] |
| | 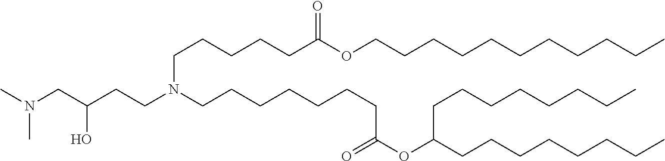 [SMILES: CCCCCCCCCCCOC(=O)CCCCCN(CCCCCCCC(=O)OC(CCCCCCCC)CCCCCCCC)CCC(O)CN(C)C] |
| | 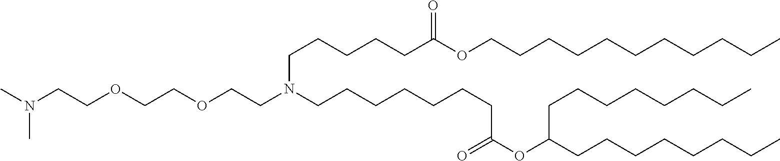 [SMILES: CCCCCCCCCCCOC(=O)CCCCCN(CCCCCCCC(=O)OC(CCCCCCCC)CCCCCCCC)CCOCCOCCN(C)C] |
| | 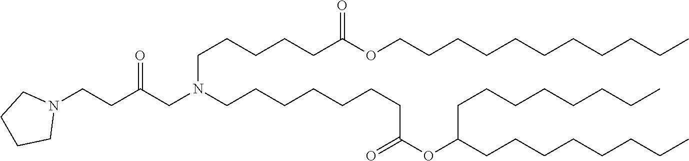 [SMILES: CCCCCCCCCCCOC(=O)CCCCCN(CCCCCCCC(=O)OC(CCCCCCCC)CCCCCCCC)CC(=O)CCN1CCCC1] |
| | 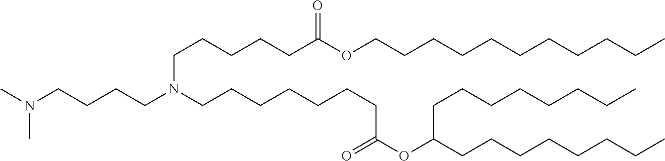 [SMILES: CCCCCCCCCCCOC(=O)CCCCCN(CCCCCCCC(=O)OC(CCCCCCCC)CCCCCCCC)CCCCN(C)C] |
| | 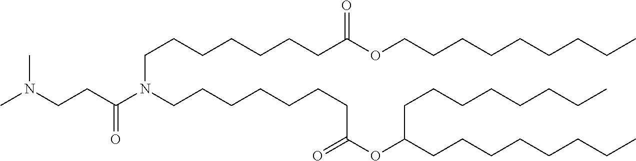 [SMILES: CCCCCCCCCOC(=O)CCCCCCCN(CCCCCCCC(=O)OC(CCCCCCCC)CCCCCCCC)C(=O)CCN(C)C] |
| | 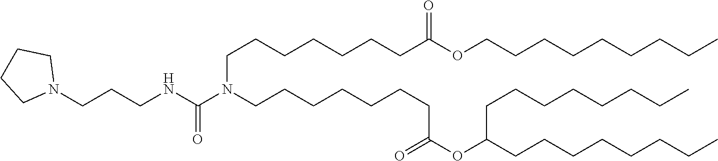 [SMILES: CCCCCCCCCOC(=O)CCCCCCCN(CCCCCCCC(=O)OC(CCCCCCCC)CCCCCCCC)C(=O)NCCCN1CCCC1] |
| | 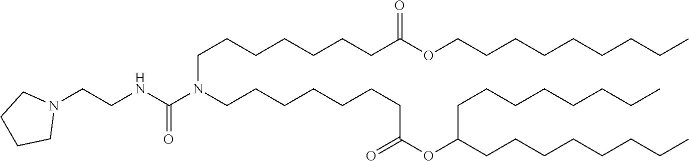 [SMILES: CCCCCCCCCOC(=O)CCCCCCCN(CCCCCCCC(=O)OC(CCCCCCCC)CCCCCCCC)C(=O)NCCN1CCCC1] |
| | 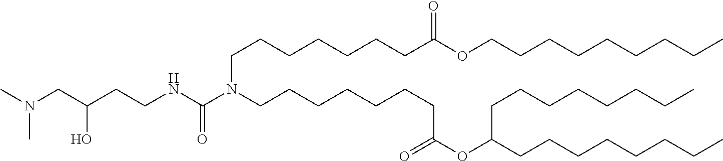 [SMILES: CCCCCCCCCOC(=O)CCCCCCCN(CCCCCCCC(=O)OC(CCCCCCCC)CCCCCCCC)C(=O)NCCC(O)CN(C)C] |
| | 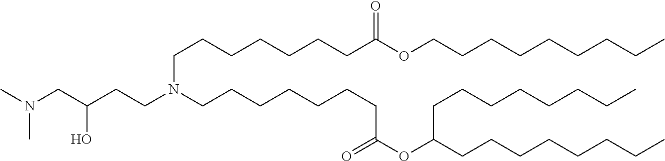 [SMILES: CCCCCCCCCOC(=O)CCCCCCCN(CCCCCCCC(=O)OC(CCCCCCCC)CCCCCCCC)CCC(O)CN(C)C] |
| | 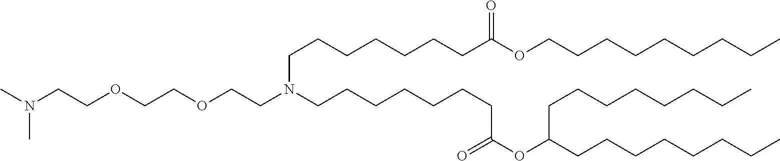 [SMILES: CCCCCCCCCOC(=O)CCCCCCCN(CCCCCCCC(=O)OC(CCCCCCCC)CCCCCCCC)CCOCCOCCN(C)C] |
| | 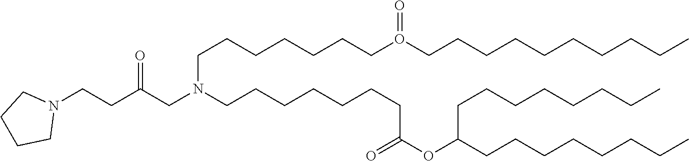 |
| | 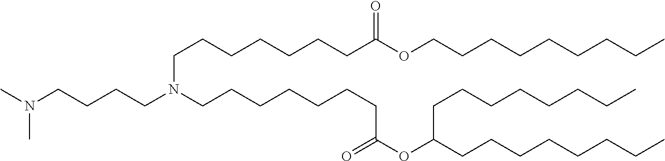 [SMILES: CCCCCCCCCOC(=O)CCCCCCCN(CCCCCCCC(=O)OC(CCCCCCCC)CCCCCCCC)CCCCN(C)C] |
| | 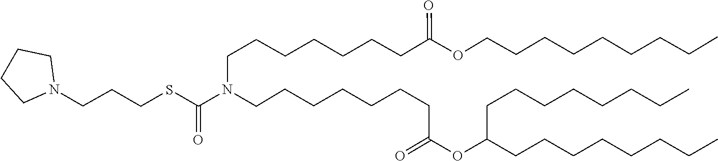 [SMILES: CCCCCCCCCOC(=O)CCCCCCCN(CCCCCCCC(=O)OC(CCCCCCCC)CCCCCCCC)C(=O)SCCCN1CCCC1] |
| | 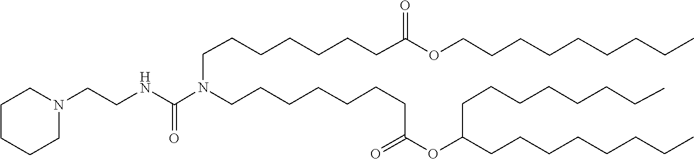 [SMILES: CCCCCCCCCOC(=O)CCCCCCCN(CCCCCCCC(=O)OC(CCCCCCCC)CCCCCCCC)C(=O)NCCN1CCCCC1] |
| | 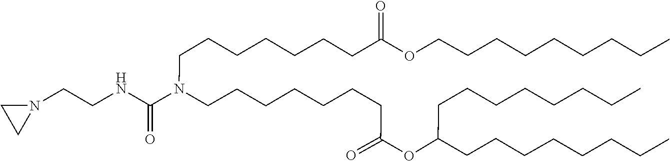 [SMILES: CCCCCCCCCOC(=O)CCCCCCCN(CCCCCCCC(=O)OC(CCCCCCCC)CCCCCCCC)C(=O)NCCN1CC1] |
| | 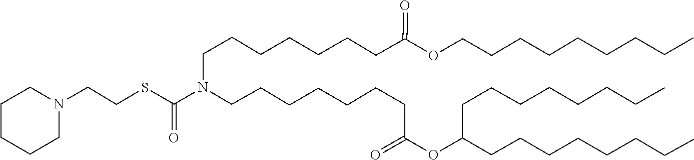 [SMILES: CCCCCCCCCOC(=O)CCCCCCCN(CCCCCCCC(=O)OC(CCCCCCCC)CCCCCCCC)C(=O)SCCN1CCCCC1] |
| | 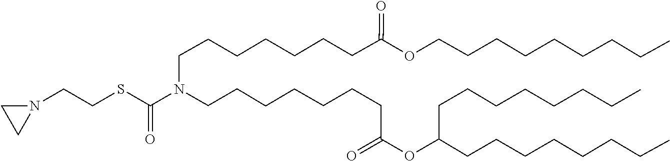 [SMILES: CCCCCCCCCOC(=O)CCCCCCCN(CCCCCCCC(=O)OC(CCCCCCCC)CCCCCCCC)C(=O)SCCN1CC1] |
| | 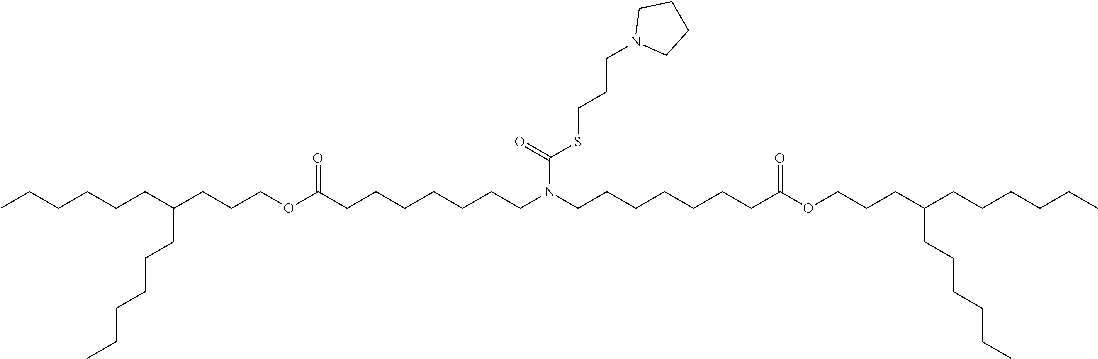 [SMILES: CCCCCCC(CCCCCC)CCCOC(=O)CCCCCCCN(CCCCCCCC(=O)OCCCC(CCCCCC)CCCCCC)C(=O)SCCCN1CCCC1] |
| | 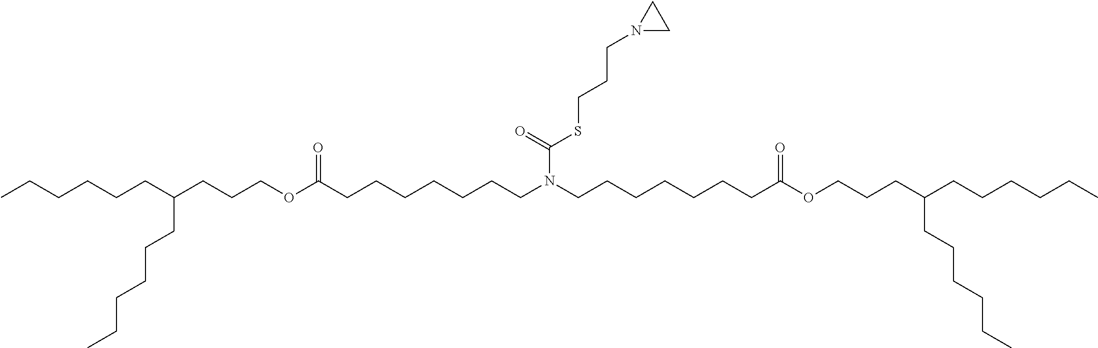 [SMILES: CCCCCCC(CCCCCC)CCCOC(=O)CCCCCCCN(CCCCCCCC(=O)OCCCC(CCCCCC)CCCCCC)C(=O)SCCCN1CC1] |
| | 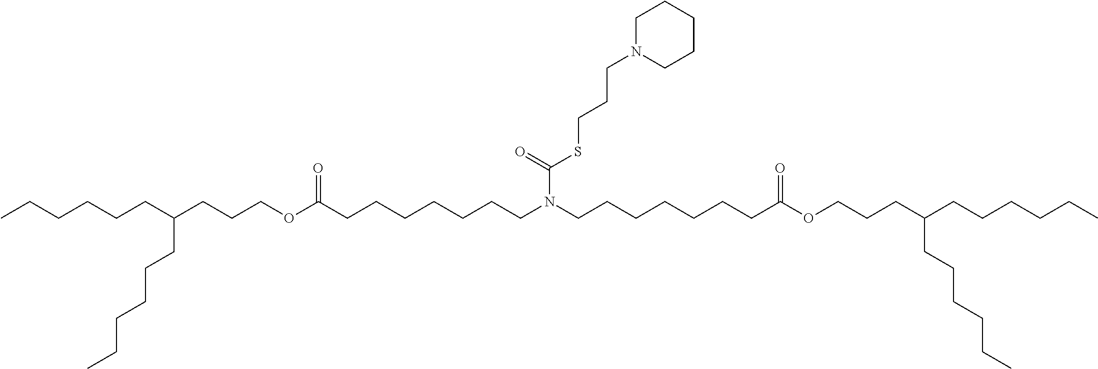 [SMILES: CCCCCCC(CCCCCC)CCCOC(=O)CCCCCCCN(CCCCCCCC(=O)OCCCC(CCCCCC)CCCCCC)C(=O)SCCCN1CCCCC1] |
| | 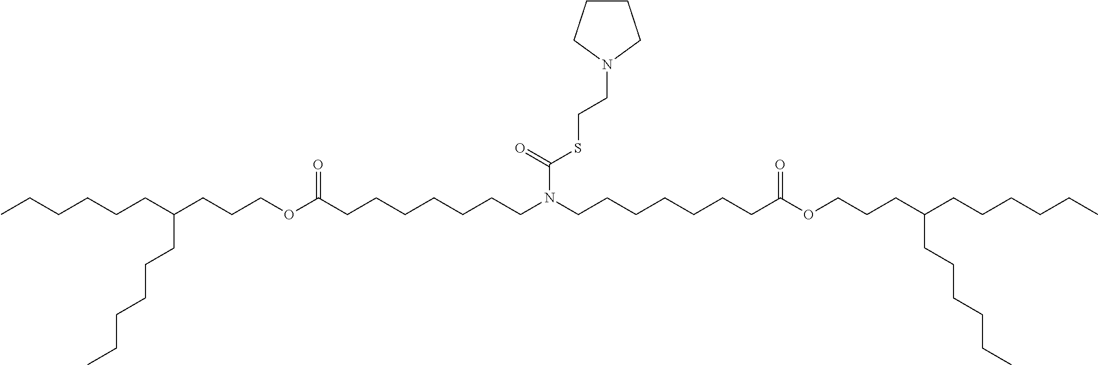 [SMILES: CCCCCCC(CCCCCC)CCCOC(=O)CCCCCCCN(CCCCCCCC(=O)OCCCC(CCCCCC)CCCCCC)C(=O)SCCN1CCCC1] |
| | 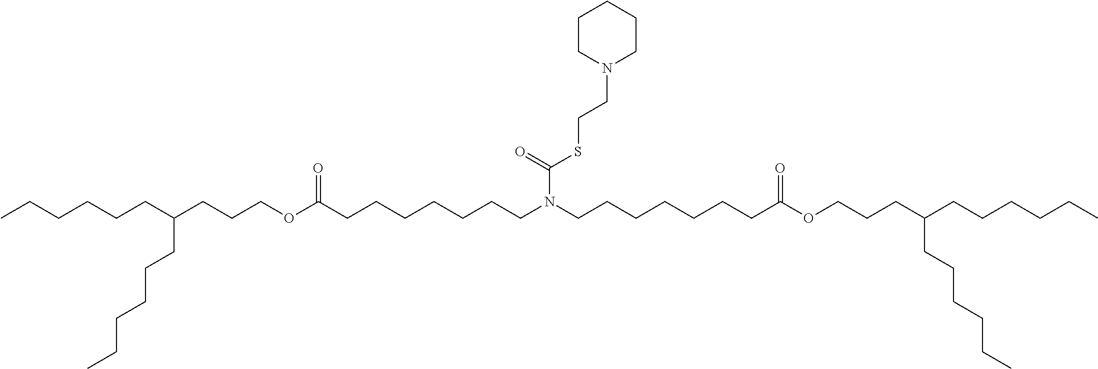 [SMILES: CCCCCCC(CCCCCC)CCCOC(=O)CCCCCCCN(CCCCCCCC(=O)OCCCC(CCCCCC)CCCCCC)C(=O)SCCN1CCCCC1] |
| | 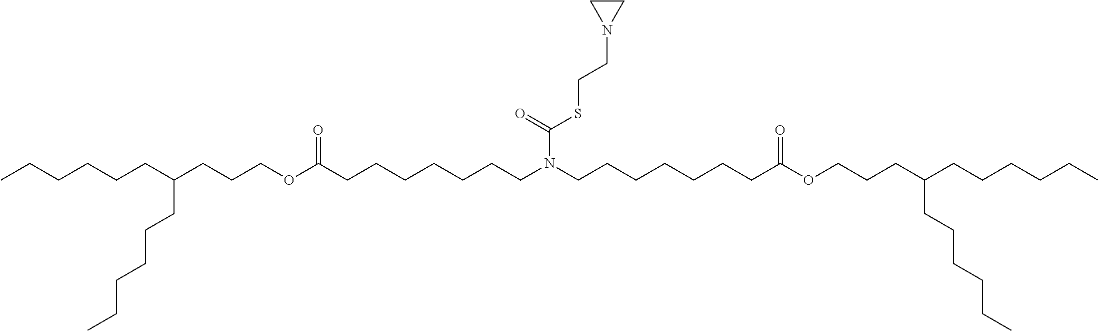 [SMILES: CCCCCCC(CCCCCC)CCCOC(=O)CCCCCCCN(CCCCCCCC(=O)OCCCC(CCCCCC)CCCCCC)C(=O)SCCN1CC1] |
| 2233 | 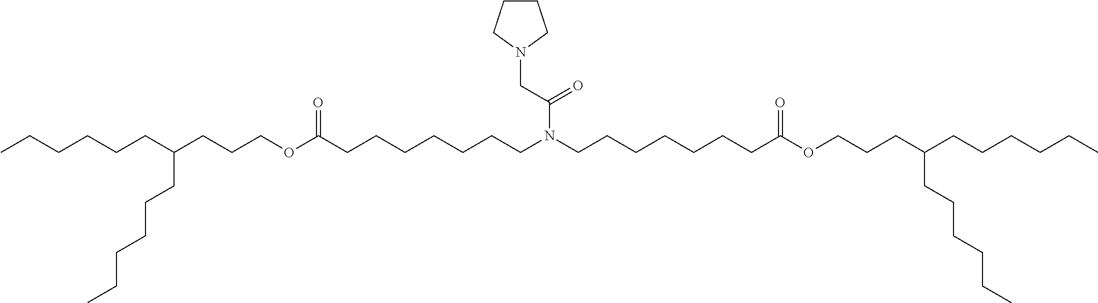 [SMILES: CCCCCCC(CCCCCC)CCCOC(=O)CCCCCCCN(CCCCCCCC(=O)OCCCC(CCCCCC)CCCCCC)C(=O)CN1CCCC1] |
| 2234 | 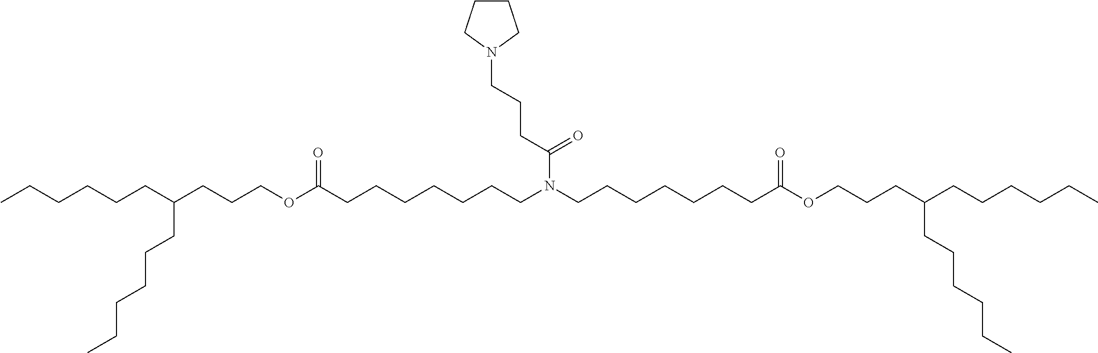 [SMILES: CCCCCCC(CCCCCC)CCCOC(=O)CCCCCCCN(CCCCCCCC(=O)OCCCC(CCCCCC)CCCCCC)C(=O)CCCN1CCCC1] |
| 2235 | 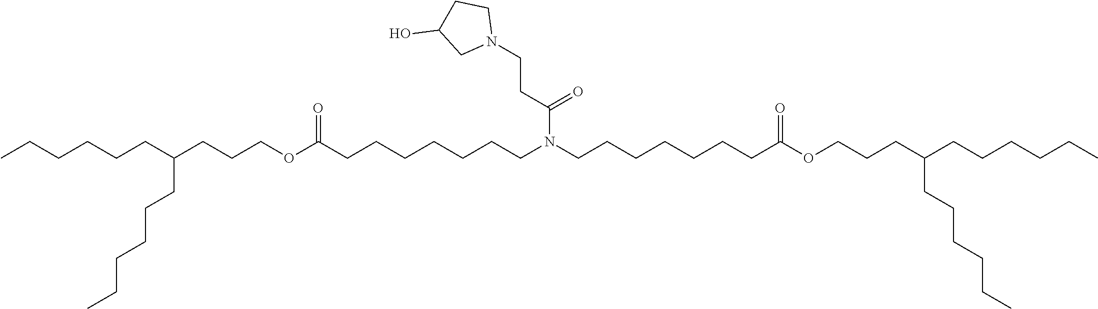 [SMILES: CCCCCCC(CCCCCC)CCCOC(=O)CCCCCCCN(CCCCCCCC(=O)OCCCC(CCCCCC)CCCCCC)C(=O)CCN1CCC(O)C1] |
| 2236 | 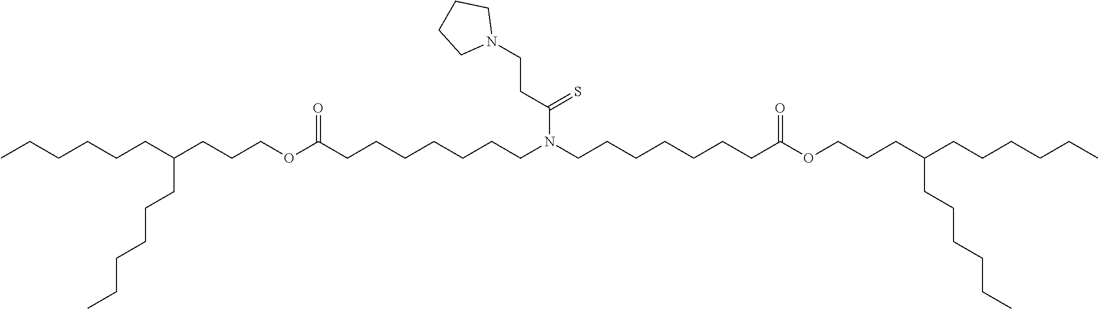 [SMILES: CCCCCCC(CCCCCC)CCCOC(=O)CCCCCCCN(CCCCCCCC(=O)OCCCC(CCCCCC)CCCCCC)C(=S)CCN1CCCC1] |
| 2237 | 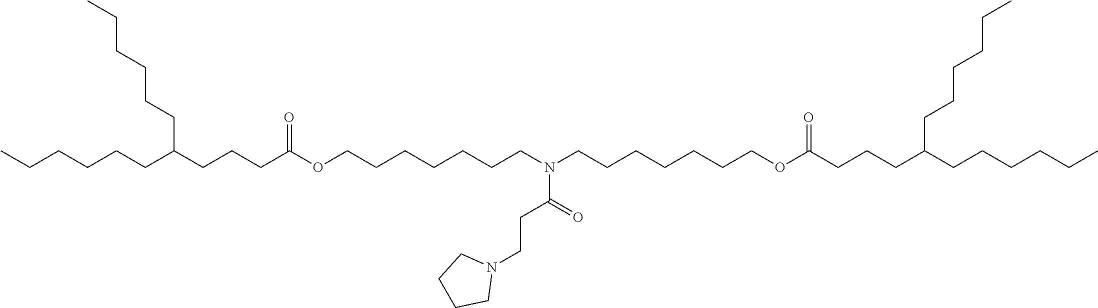 [SMILES: CCCCCCC(CCCCCC)CCCC(=O)OCCCCCCCN(CCCCCCCOC(=O)CCCC(CCCCCC)CCCCCC)C(=O)CCN1CCCC1] |
| 2238 | 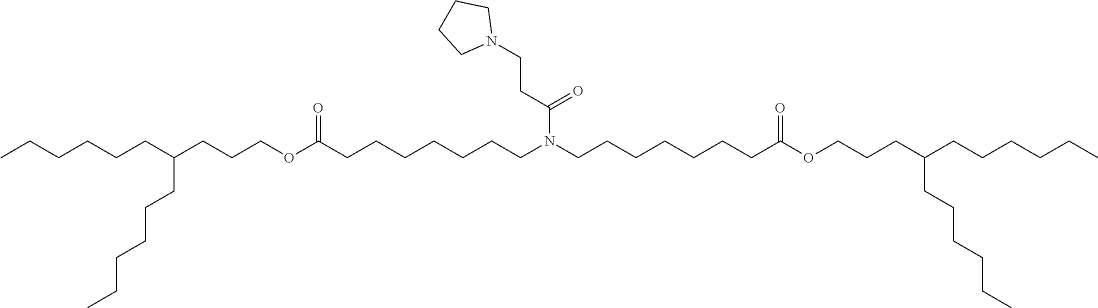 [SMILES: CCCCCCC(CCCCCC)CCCOC(=O)CCCCCCCN(CCCCCCCC(=O)OCCCC(CCCCCC)CCCCCC)C(=O)CCN1CCCC1] |
| 2239 | 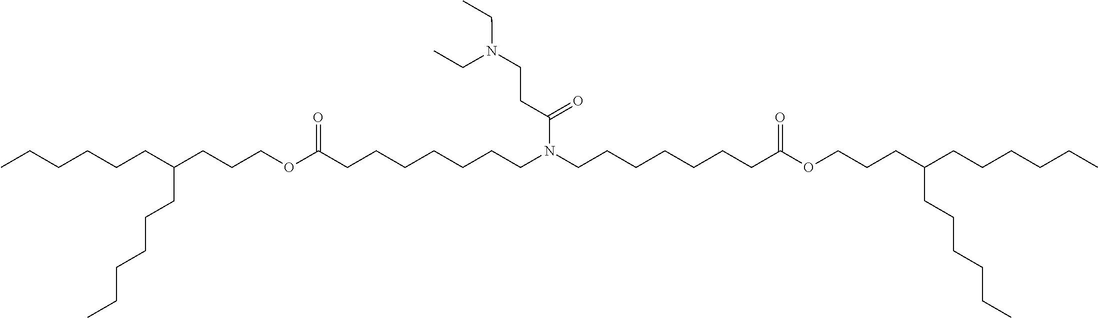 [SMILES: CCCCCCC(CCCCCC)CCCOC(=O)CCCCCCCN(CCCCCCCC(=O)OCCCC(CCCCCC)CCCCCC)C(=O)CCN(CC)CC] |
| 2241 | 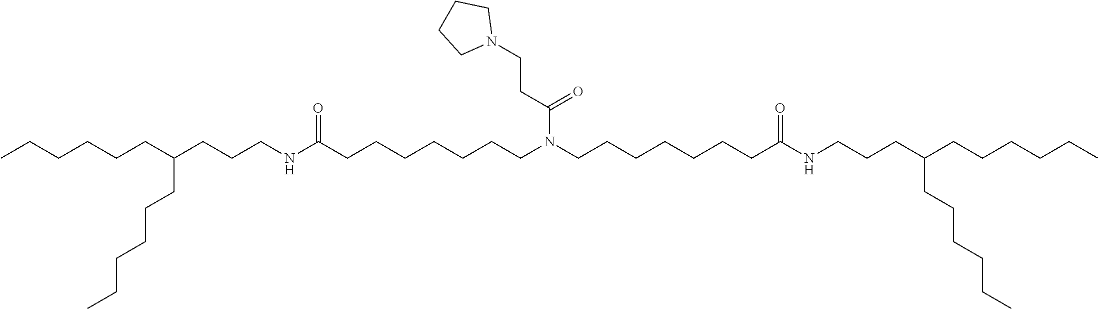 [SMILES: CCCCCCC(CCCCCC)CCCNC(=O)CCCCCCCN(CCCCCCCC(=O)NCCCC(CCCCCC)CCCCCC)C(=O)CCN1CCCC1] |
| 2242 | 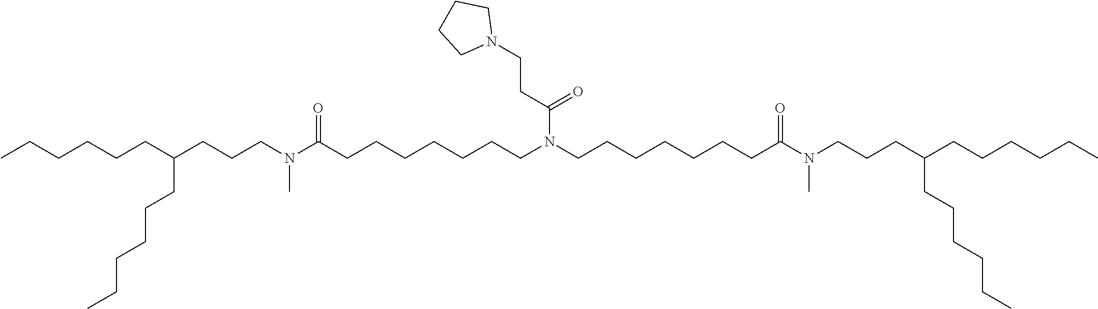 [SMILES: CCCCCCC(CCCCCC)CCCN(C)C(=O)CCCCCCCN(CCCCCCCC(=O)N(C)CCCC(CCCCCC)CCCCCC)C(=O)CCN1CCCC1] |
| 2244 | 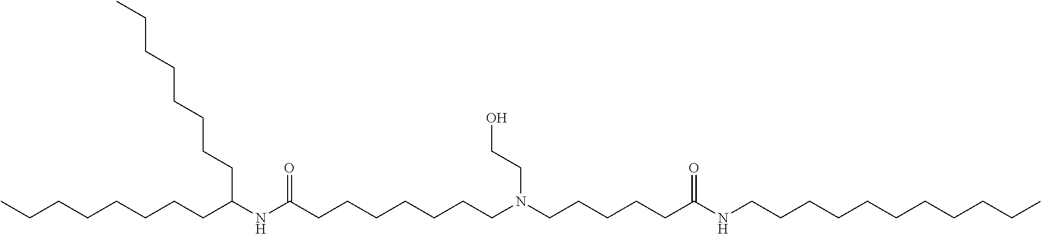 [SMILES: CCCCCCCCCCCNC(=O)CCCCCN(CCO)CCCCCCCC(=O)NC(CCCCCCCC)CCCCCCCC] |
| 2245 | 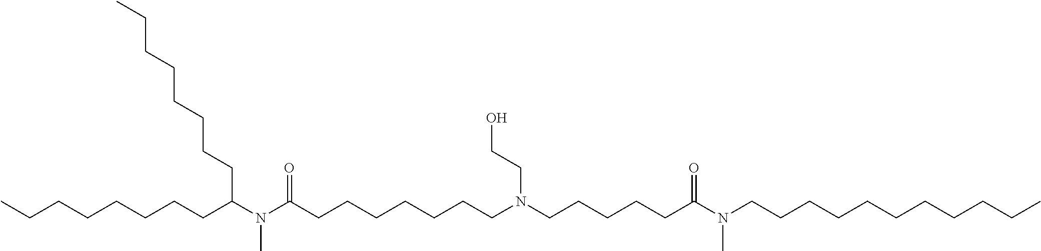 [SMILES: CCCCCCCCCCCN(C)C(=O)CCCCCN(CCO)CCCCCCCC(=O)N(C)C(CCCCCCCC)CCCCCCCC] |
| 2249 | 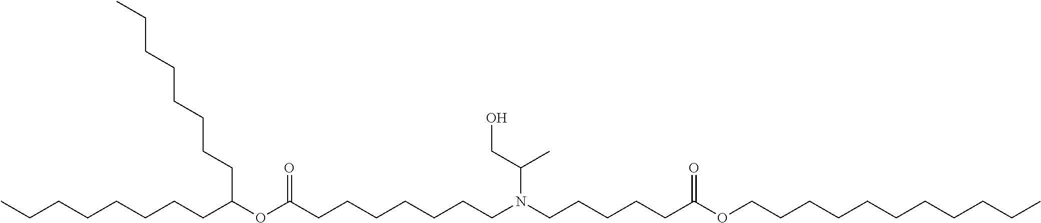 [SMILES: CCCCCCCCCCCOC(=O)CCCCCN(CCCCCCCC(=O)OC(CCCCCCCC)CCCCCCCC)C(C)CO] |
| 2250 | 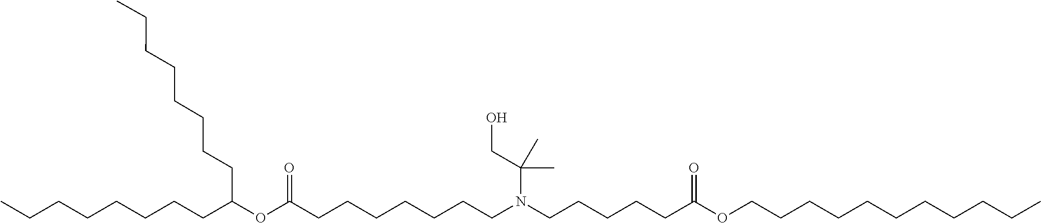 [SMILES: CCCCCCCCCCCOC(=O)CCCCCN(CCCCCCCC(=O)OC(CCCCCCCC)CCCCCCCC)C(C)(C)CO] |
| 2276 | 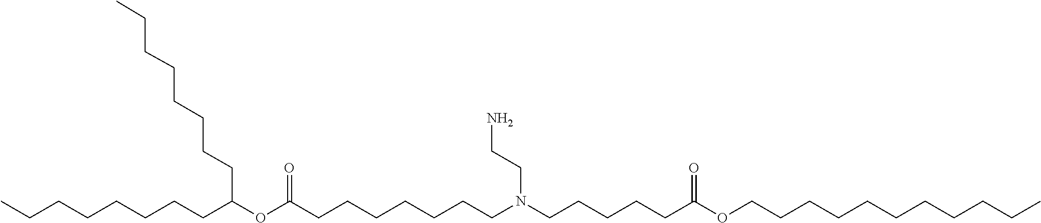 [SMILES: CCCCCCCCCCCOC(=O)CCCCCN(CCN)CCCCCCCC(=O)OC(CCCCCCCC)CCCCCCCC] |
| 2277 | 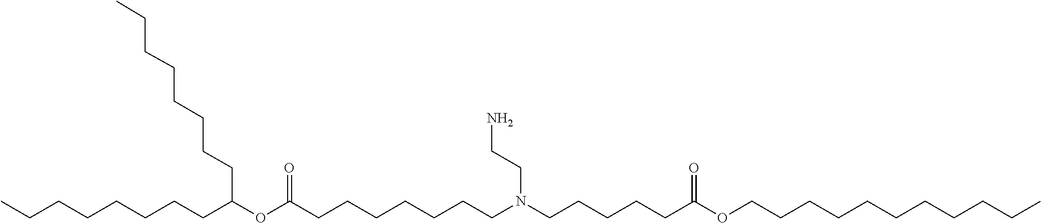 [SMILES: CCCCCCCCCCCOC(=O)CCCCCN(CCN)CCCCCCCC(=O)OC(CCCCCCCC)CCCCCCCC] |
| 2300 | 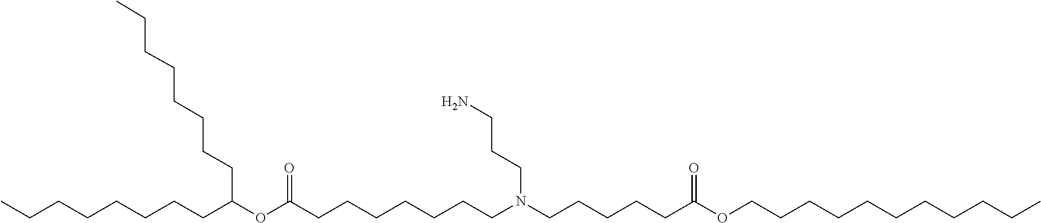 [SMILES: CCCCCCCCCCCOC(=O)CCCCCN(CCCN)CCCCCCCC(=O)OC(CCCCCCCC)CCCCCCCC] |
| 2301 | 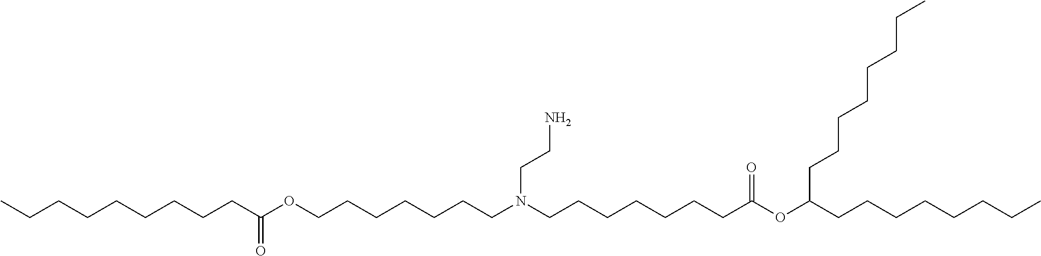 [SMILES: CCCCCCCCCC(=O)OCCCCCCCN(CCN)CCCCCCCC(=O)OC(CCCCCCCC)CCCCCCCC] |
| 2312 | 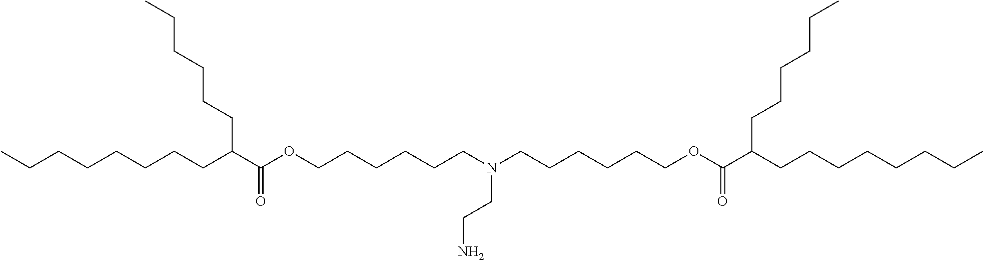 [SMILES: CCCCCCCCC(CCCCCC)C(=O)OCCCCCCN(CCN)CCCCCCOC(=O)C(CCCCCC)CCCCCCCC] |
| 2313 | 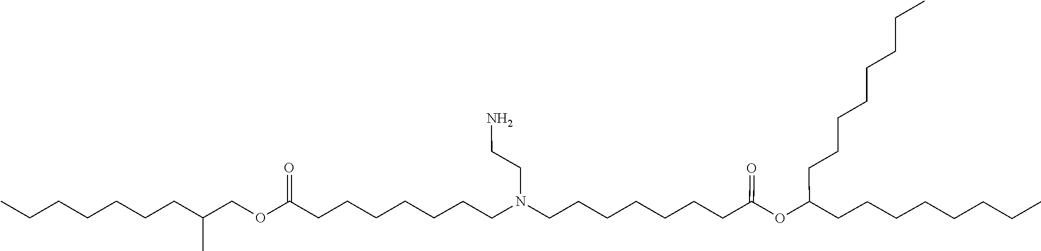 [SMILES: CCCCCCCCC(CCCCCCCC)OC(=O)CCCCCCCN(CCN)CCCCCCCC(=O)OCC(C)CCCCCCC] |
| 2314 | 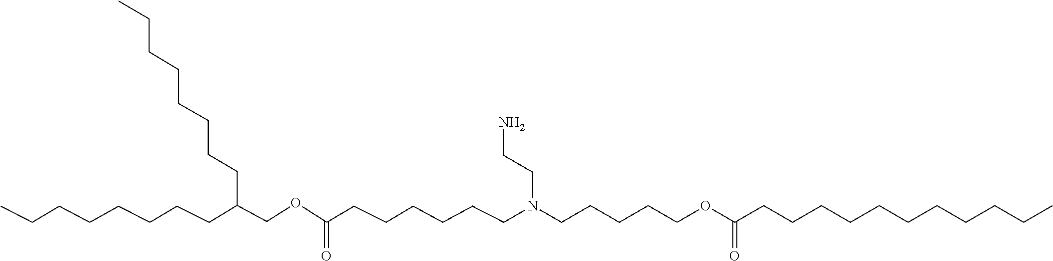 [SMILES: CCCCCCCCCCCC(=O)OCCCCCN(CCN)CCCCCCC(=O)OCC(CCCCCCCC)CCCCCCCC] |
| | 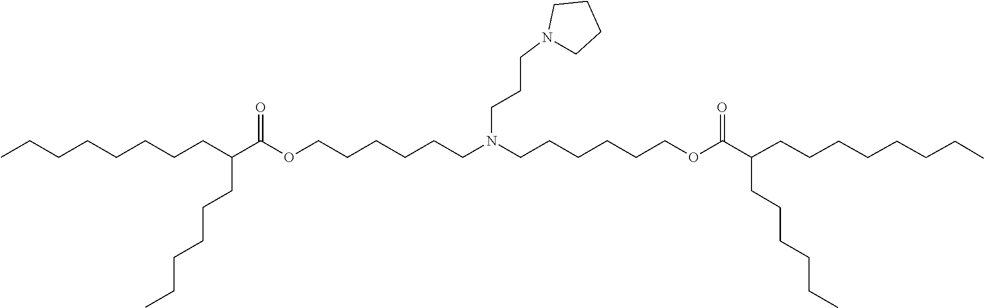 [SMILES: CCCCCCCCC(CCCCCC)C(=O)OCCCCCCN(CCCCCCOC(=O)C(CCCCCC)CCCCCCCC)CCCN1CCCC1] |
| | 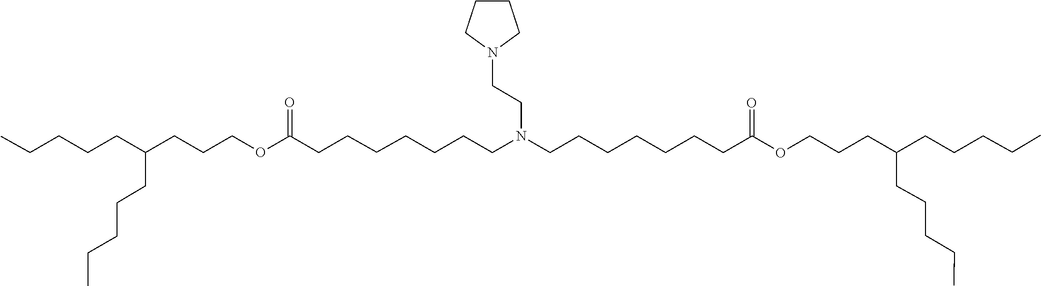 [SMILES: CCCCCC(CCCCC)CCCOC(=O)CCCCCCCN(CCCCCCCC(=O)OCCCC(CCCCC)CCCCC)CCN1CCCC1] |
| | 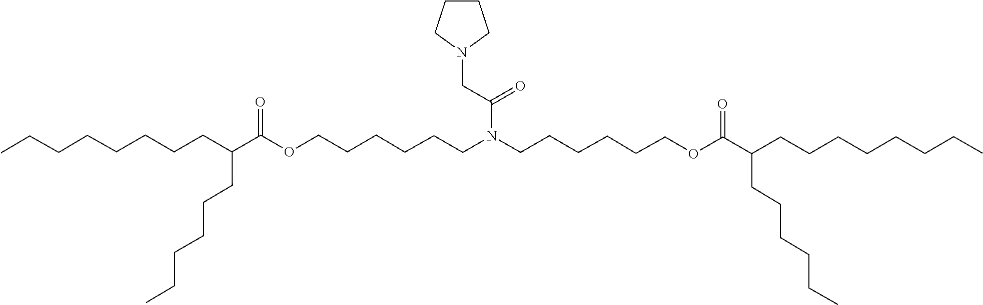 [SMILES: CCCCCCCCC(CCCCCC)C(=O)OCCCCCCN(CCCCCCOC(=O)C(CCCCCC)CCCCCCCC)C(=O)CN1CCCC1] |
| | 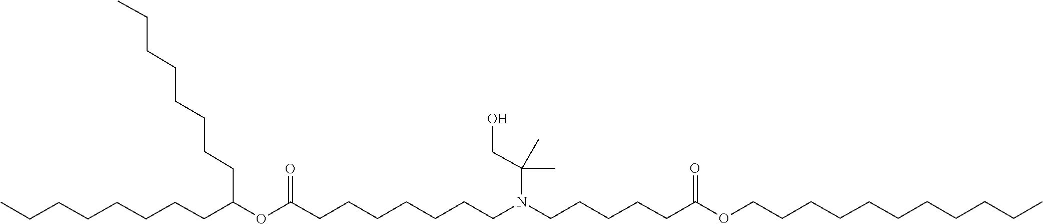 [SMILES: CCCCCCCCCCCOC(=O)CCCCCN(CCCCCCCC(=O)OC(CCCCCCCC)CCCCCCCC)C(C)(C)CO] |
| | 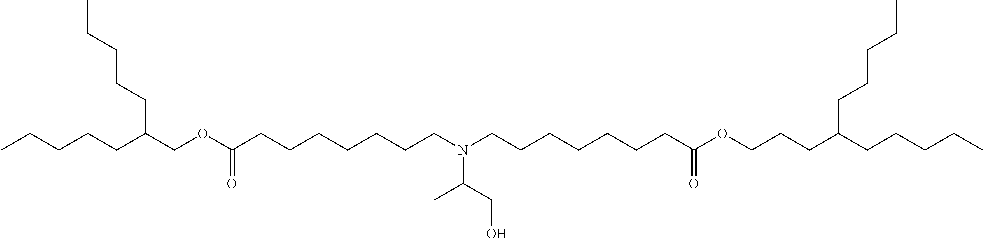 [SMILES: CCCCCC(CCCCC)CCCOC(=O)CCCCCCCN(CCCCCCCC(=O)OCC(CCCCC)CCCCC)C(C)CO] |
| | 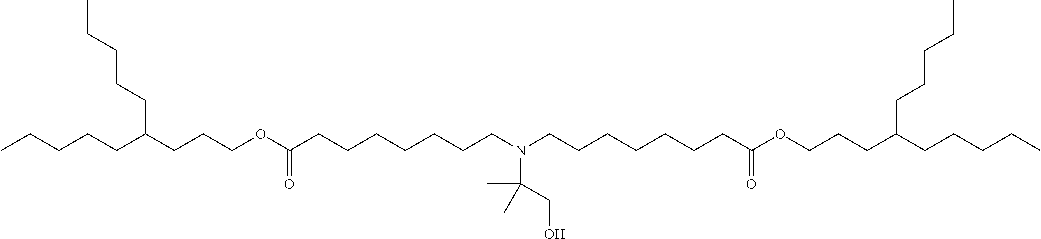 [SMILES: CCCCCC(CCCCC)CCCOC(=O)CCCCCCCN(CCCCCCCC(=O)OCCCC(CCCCC)CCCCC)C(C)(C)CO] |
| | 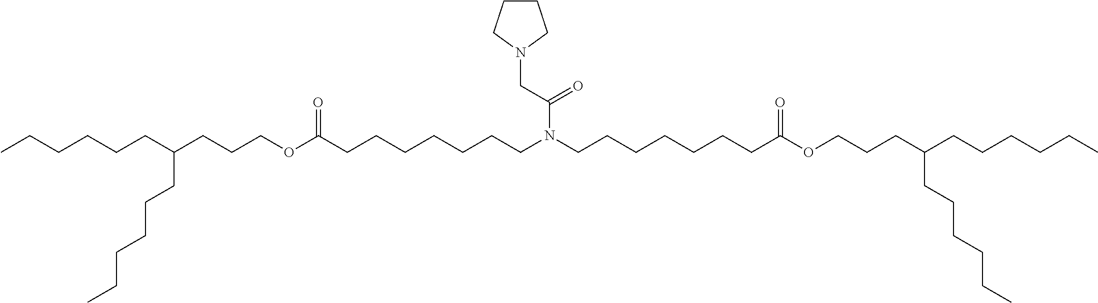 [SMILES: CCCCCCC(CCCCCC)CCCOC(=O)CCCCCCCN(CCCCCCCC(=O)OCCCC(CCCCCC)CCCCCC)C(=O)CN1CCCC1] |
| | 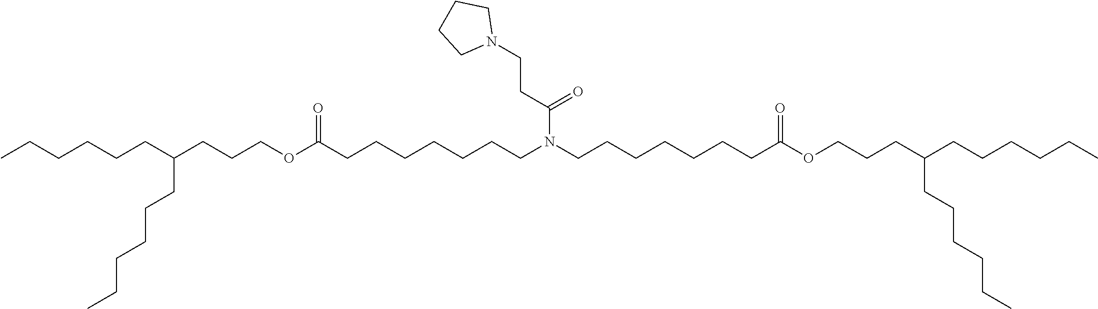 [SMILES: CCCCCCC(CCCCCC)CCCOC(=O)CCCCCCCN(CCCCCCCC(=O)OCCCC(CCCCCC)CCCCCC)C(=O)CCN1CCCC1] |
| | 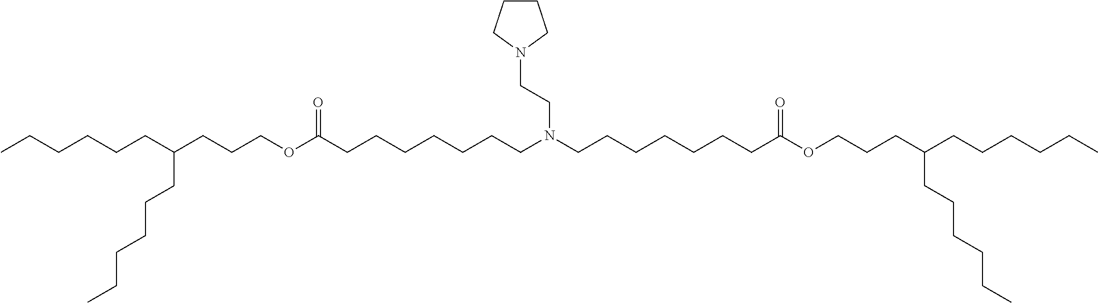 [SMILES: CCCCCCC(CCCCCC)CCCOC(=O)CCCCCCCN(CCCCCCCC(=O)OCCCC(CCCCCC)CCCCCC)CCN1CCCC1] |
| | 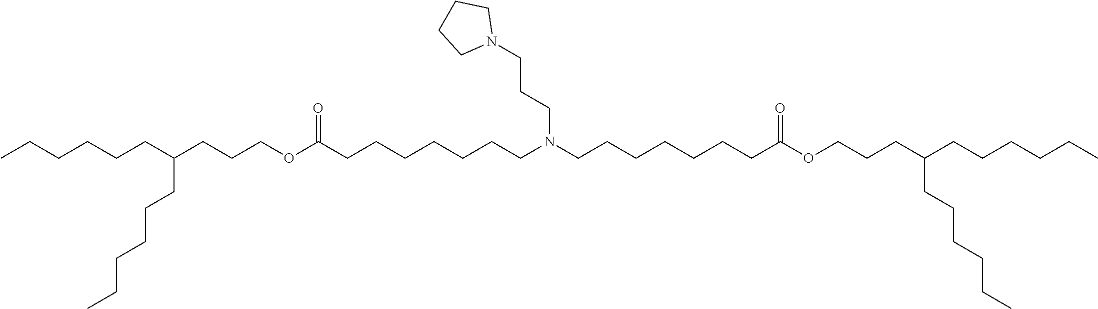 [SMILES: CCCCCCC(CCCCCC)CCCOC(=O)CCCCCCCN(CCCCCCCC(=O)OCCCC(CCCCCC)CCCCCC)CCCN1CCCC1] |
| | 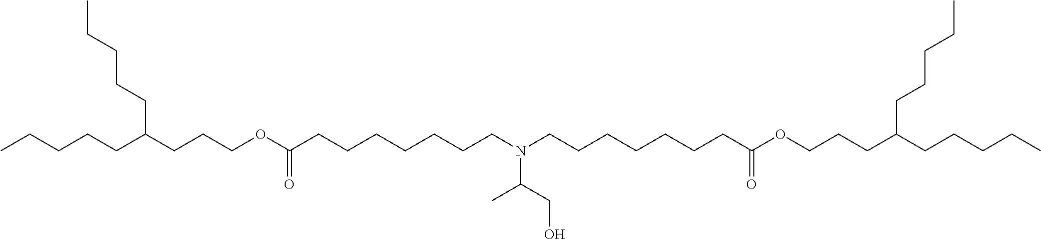 [SMILES: CCCCCC(CCCCC)CCCOC(=O)CCCCCCCN(CCCCCCCC(=O)OCCCC(CCCCC)CCCCC)C(C)CO] |
| | 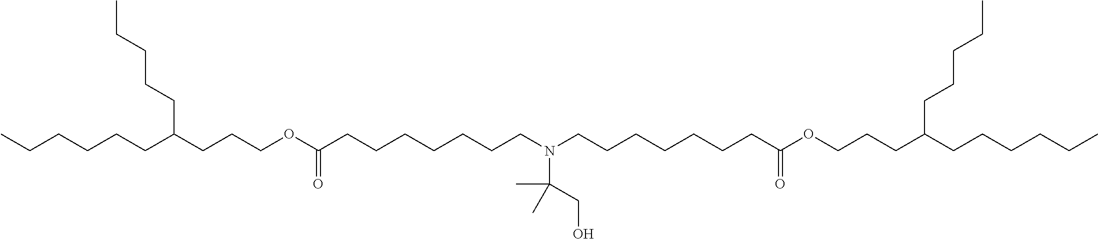 [SMILES: CCCCCCC(CCCCC)CCCOC(=O)CCCCCCCN(CCCCCCCC(=O)OCCCC(CCCCC)CCCCCC)C(C)(C)CO] |
| | 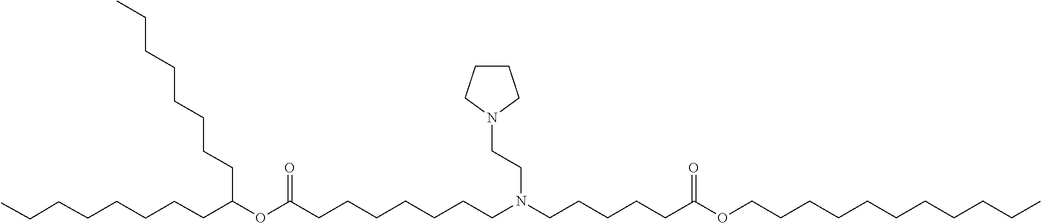 [SMILES: CCCCCCCCCCCOC(=O)CCCCCN(CCCCCCCC(=O)OC(CCCCCCCC)CCCCCCCC)CCN1CCCC1] |
| | 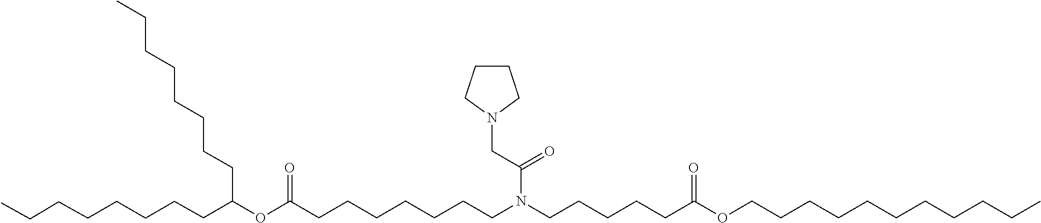 [SMILES: CCCCCCCCCCCOC(=O)CCCCCN(CCCCCCCC(=O)OC(CCCCCCCC)CCCCCCCC)C(=O)CN1CCCC1] |
| | 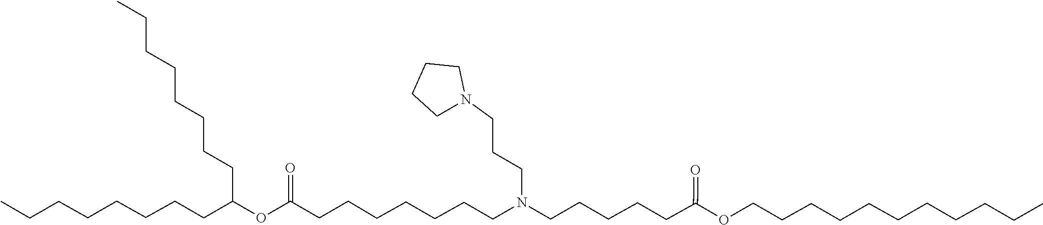 [SMILES: CCCCCCCCCCCOC(=O)CCCCCN(CCCCCCCC(=O)OC(CCCCCCCC)CCCCCCCC)CCCN1CCCC1] |
| | 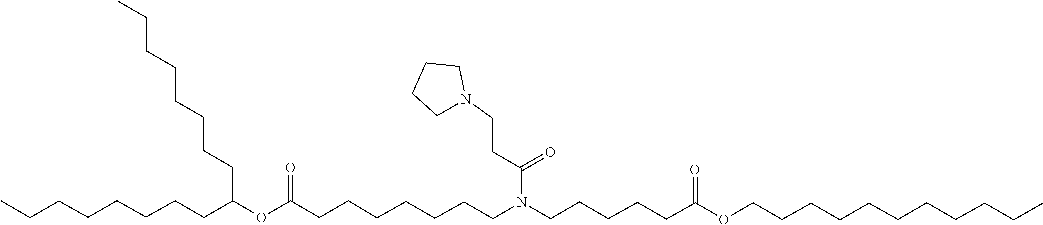 [SMILES: CCCCCCCCCCCOC(=O)CCCCCN(CCCCCCCC(=O)OC(CCCCCCCC)CCCCCCCC)C(=O)CCN1CCCC1] |
| | 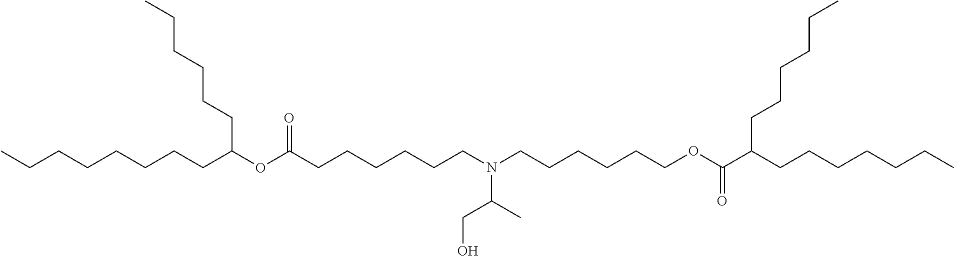 [SMILES: CCCCCCCCC(CCCCCC)OC(=O)CCCCCCN(CCCCCCOC(=O)C(CCCCCC)CCCCCCC)C(C)CO] |
| | 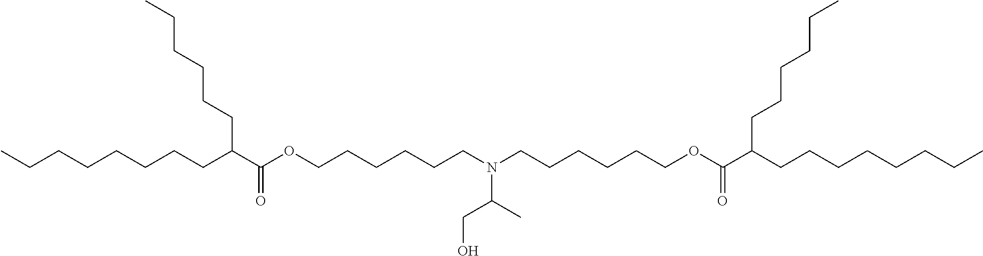 [SMILES: CCCCCCCCC(CCCCCC)C(=O)OCCCCCCN(CCCCCCOC(=O)C(CCCCCC)CCCCCCCC)C(C)CO] |
| | 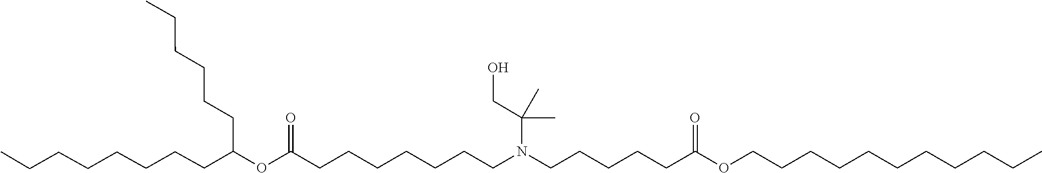 [SMILES: CCCCCCCCCCCOC(=O)CCCCCN(CCCCCCCC(=O)OC(CCCCCC)CCCCCCCC)C(C)(C)CO] |
| | 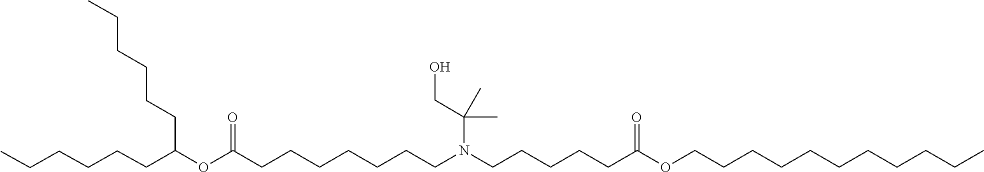 [SMILES: CCCCCCCCCCCOC(=O)CCCCCN(CCCCCCCC(=O)OC(CCCCCC)CCCCCC)C(C)(C)CO] |
| | 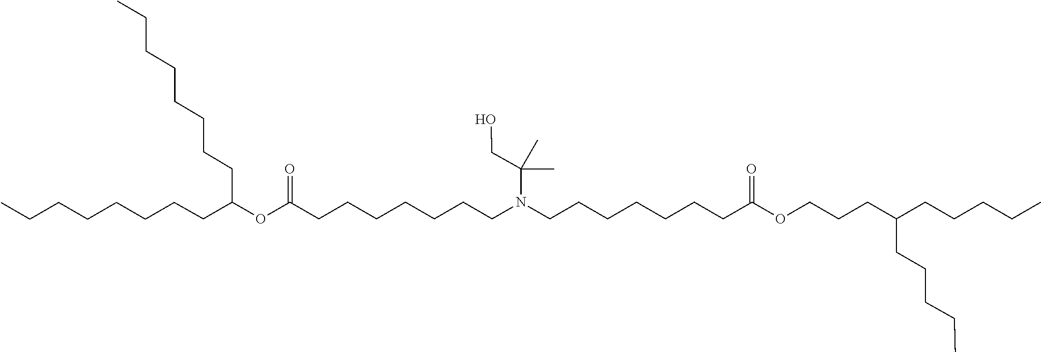 [SMILES: CCCCCCCCC(CCCCCCCC)OC(=O)CCCCCCCN(CCCCCCCC(=O)OCCCC(CCCCC)CCCCC)C(C)(C)CO] |
| | 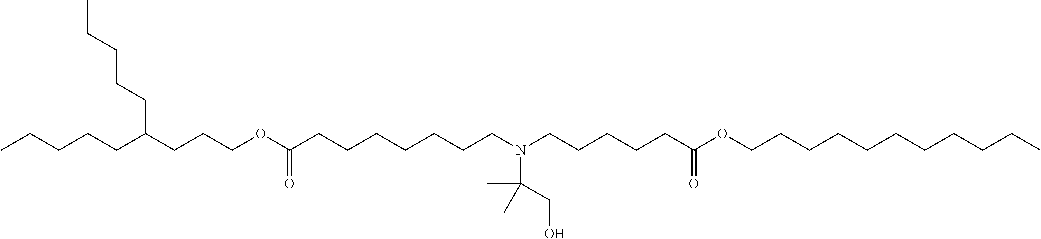 [SMILES: CCCCCCCCCCCOC(=O)CCCCCN(CCCCCCCC(=O)OCCCC(CCCCC)CCCCC)C(C)(C)CO] |
| | 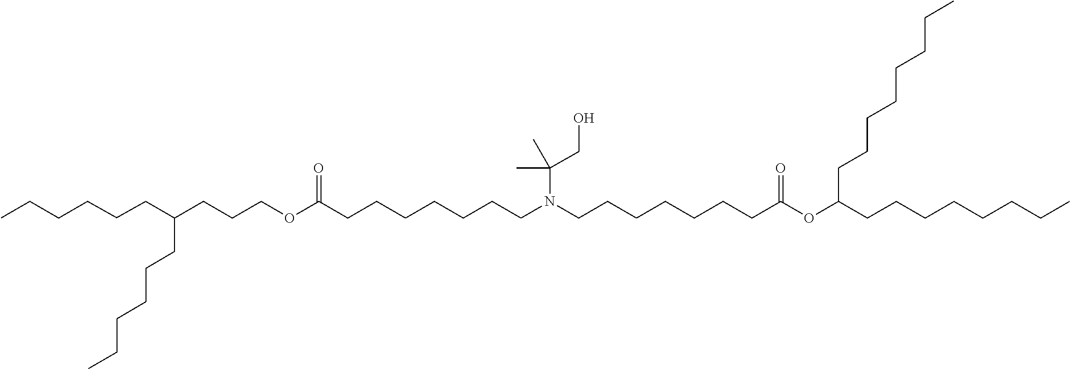 [SMILES: CCCCCCCCC(CCCCCCCC)OC(=O)CCCCCCCN(CCCCCCCC(=O)OCCCC(CCCCCC)CCCCCC)C(C)(C)CO] |
| | 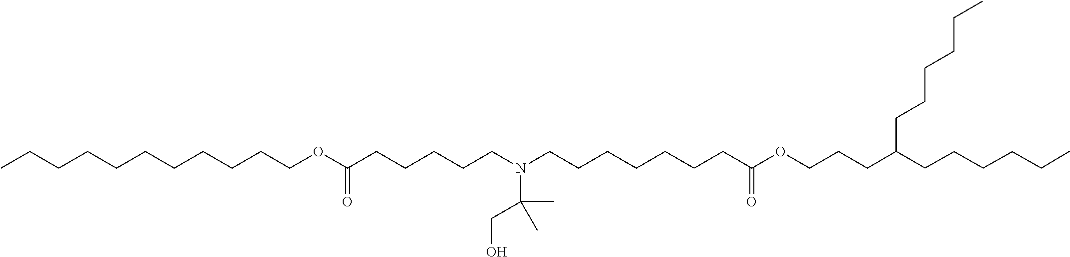 [SMILES: CCCCCCCCCCCOC(=O)CCCCCN(CCCCCCCC(=O)OCCCC(CCCCCC)CCCCCC)C(C)(C)CO] |
| | 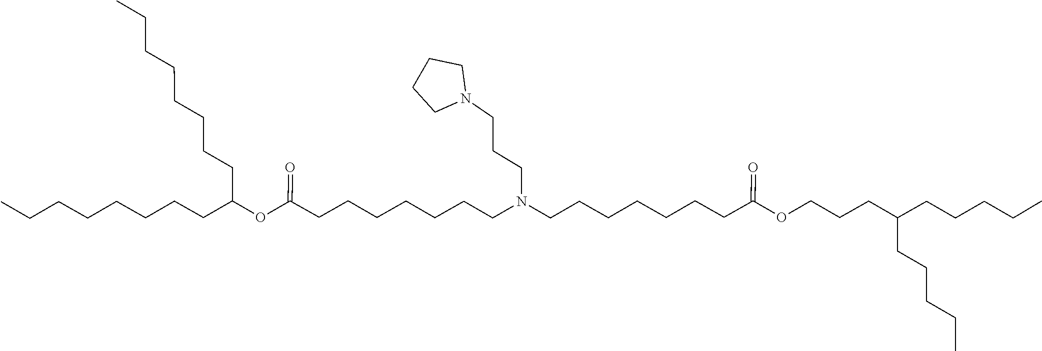 [SMILES: CCCCCCCCC(CCCCCCCC)OC(=O)CCCCCCCN(CCCCCCCC(=O)OCCCC(CCCCC)CCCCC)CCCN1CCCC1] |
| | 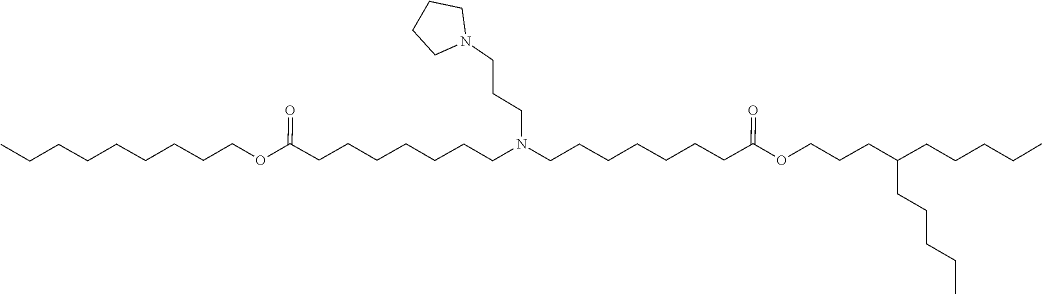 [SMILES: CCCCCCCCCOC(=O)CCCCCCCN(CCCCCCCC(=O)OCCCC(CCCCC)CCCCC)CCCN1CCCC1] |
| | 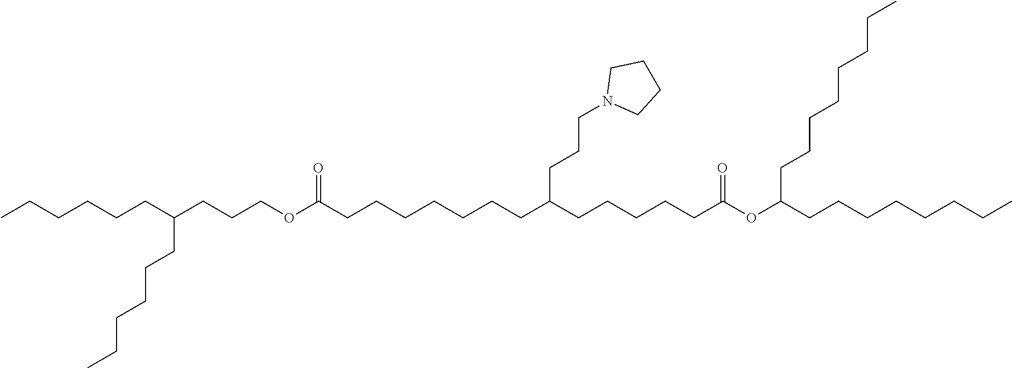 [SMILES: CCCCCCCCC(CCCCCCCC)OC(=O)CCCCCC(CCCCCCCC(=O)OCCCC(CCCCCC)CCCCCC)CCCN1CCCC1] |
| | 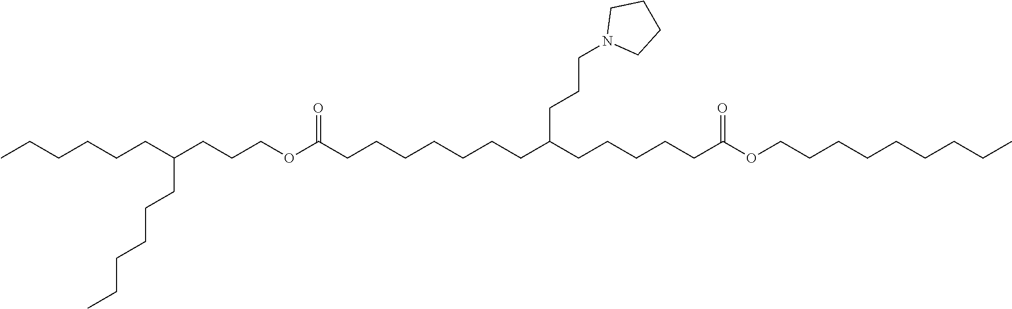 [SMILES: CCCCCCCCCOC(=O)CCCCCC(CCCCCCCC(=O)OCCCC(CCCCCC)CCCCCC)CCCN1CCCC1] |
| | 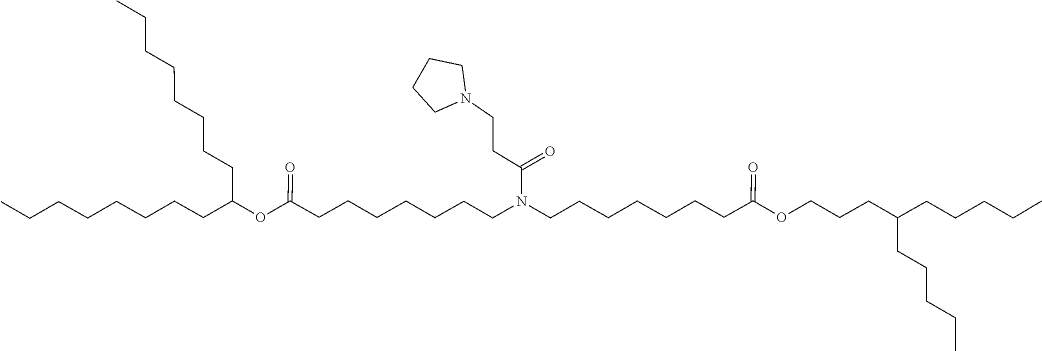 [SMILES: CCCCCCCCC(CCCCCCCC)OC(=O)CCCCCCCN(CCCCCCCC(=O)OCCCC(CCCCC)CCCCC)C(=O)CCN1CCCC1] |
| | 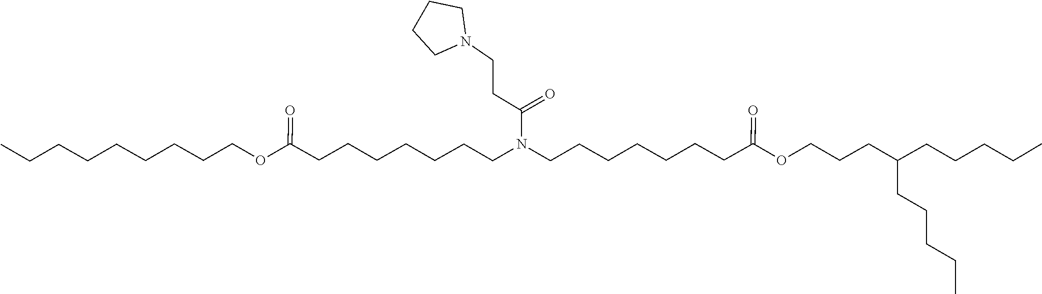 [SMILES: CCCCCCCCCOC(=O)CCCCCCCN(CCCCCCCC(=O)OCCCC(CCCCC)CCCCC)C(=O)CCN1CCCC1] |
| | 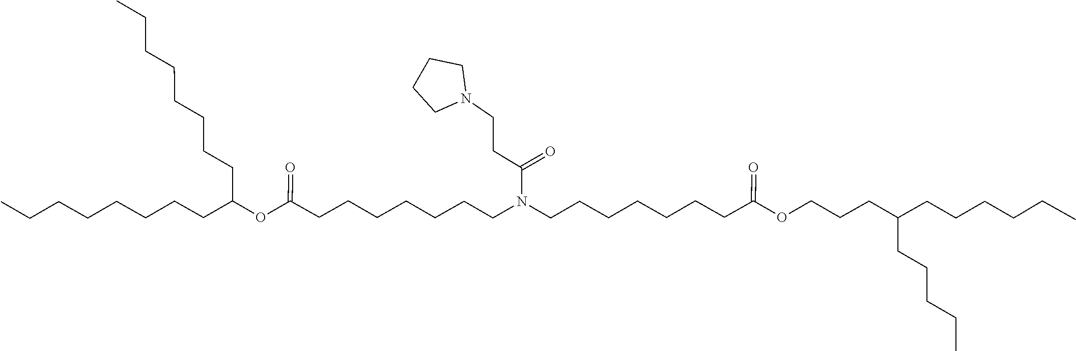 [SMILES: CCCCCCCCC(CCCCCCCC)OC(=O)CCCCCCCN(CCCCCCCC(=O)OCCCC(CCCCC)CCCCCC)C(=O)CCN1CCCC1] |
| | 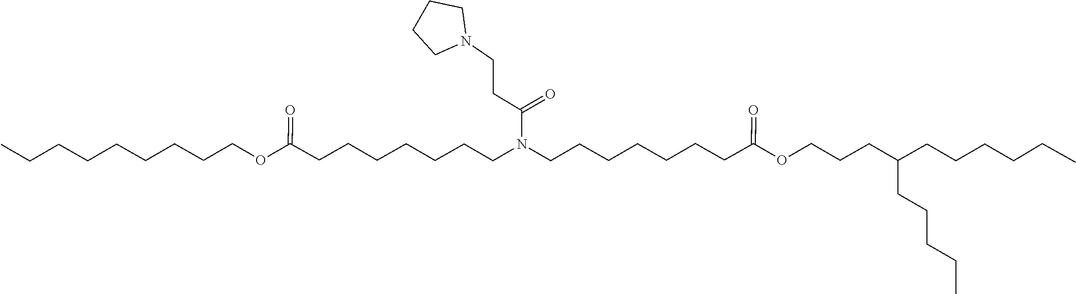 [SMILES: CCCCCCCCCOC(=O)CCCCCCCN(CCCCCCCC(=O)OCCCC(CCCCC)CCCCCC)C(=O)CCN1CCCC1] |
| | 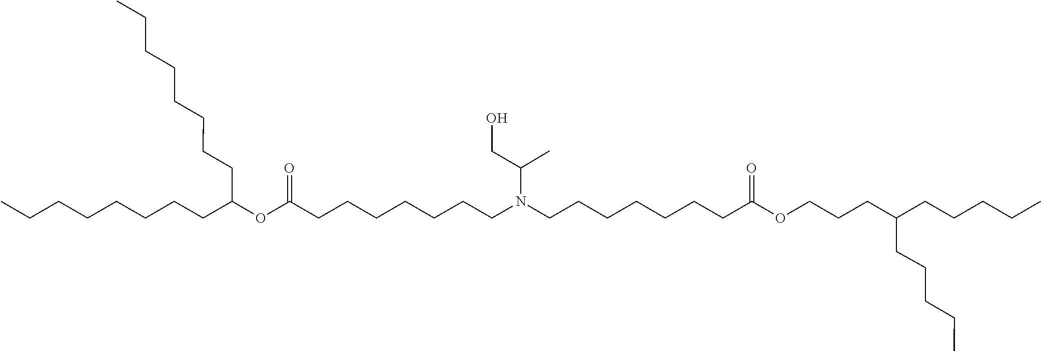 [SMILES: CCCCCCCCC(CCCCCCCC)OC(=O)CCCCCCCN(CCCCCCCC(=O)OCCCC(CCCCC)CCCCC)C(C)CO] |
| | 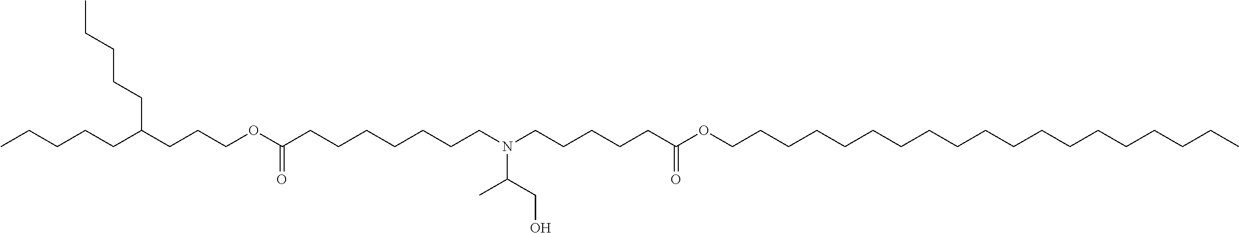 [SMILES: CCCCCCCCCCCCCCCCCCCOC(=O)CCCCCN(CCCCCCCC(=O)OCCCC(CCCCC)CCCCC)C(C)CO] |
| | 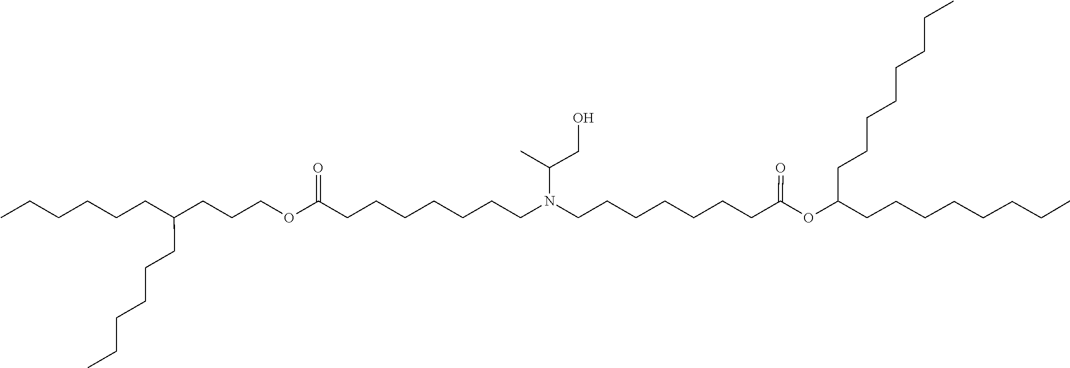 [SMILES: CCCCCCCCC(CCCCCCCC)OC(=O)CCCCCCCN(CCCCCCCC(=O)OCCCC(CCCCCC)CCCCCC)C(C)CO] |
| | 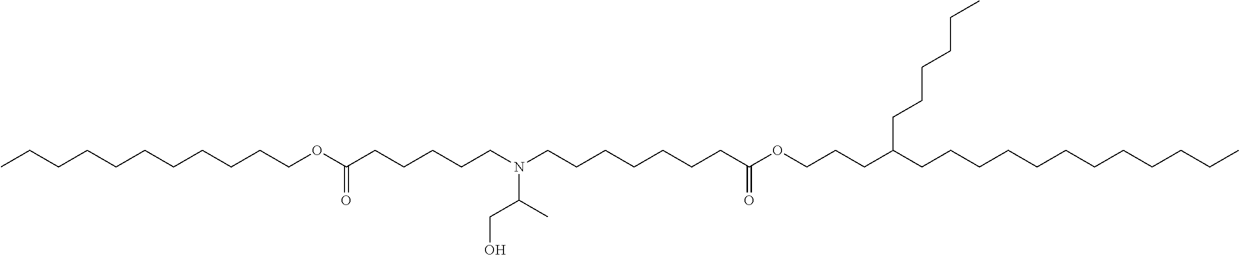 [SMILES: CCCCCCCCCCCCC(CCCCCC)CCCOC(=O)CCCCCCCN(CCCCCC(=O)OCCCCCCCCCCC)C(C)CO] |
| | 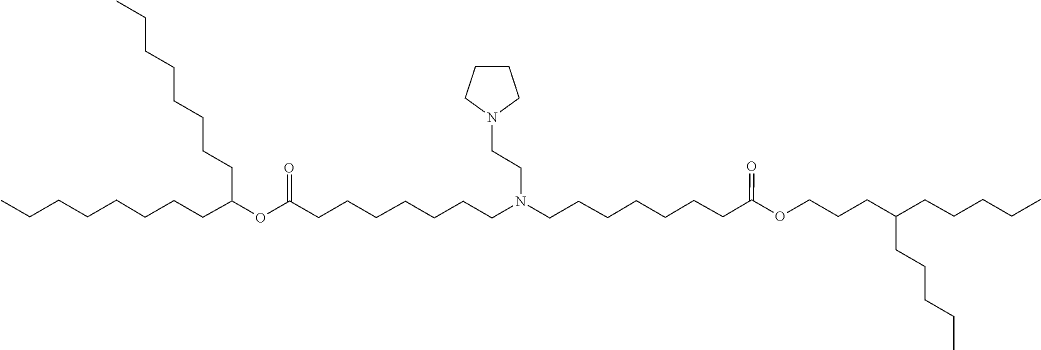 [SMILES: CCCCCCCCC(CCCCCCCC)OC(=O)CCCCCCCN(CCCCCCCC(=O)OCCCC(CCCCC)CCCCC)CCN1CCCC1] |
| | 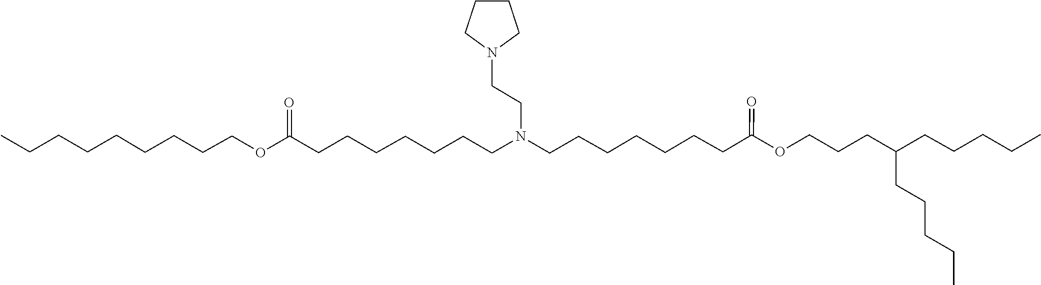 [SMILES: CCCCCCCCCOC(=O)CCCCCCCN(CCCCCCCC(=O)OCCCC(CCCCC)CCCCC)CCN1CCCC1] |
| | 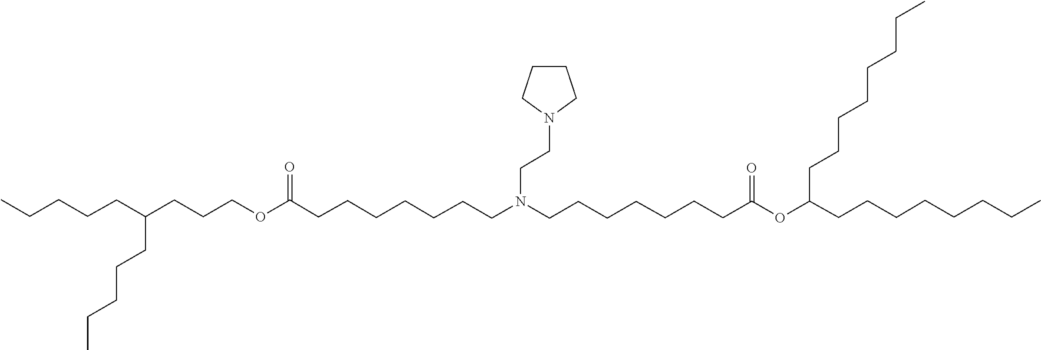 [SMILES: CCCCCCCCC(CCCCCCCC)OC(=O)CCCCCCCN(CCCCCCCC(=O)OCCCC(CCCCC)CCCCC)CCN1CCCC1] |
| | 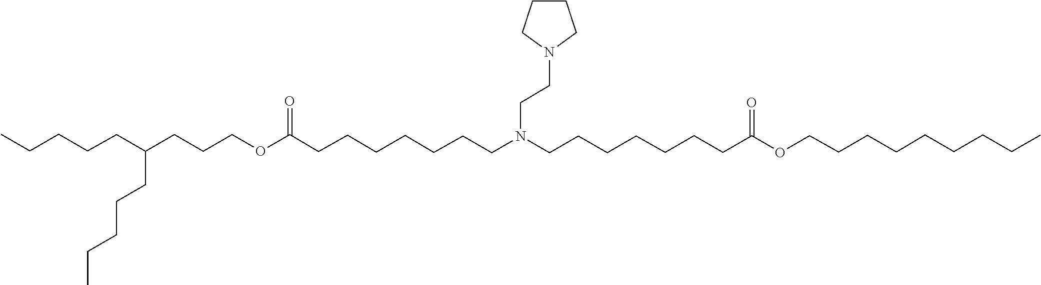 [SMILES: CCCCCCCCCOC(=O)CCCCCCCN(CCCCCCCC(=O)OCCCC(CCCCC)CCCCC)CCN1CCCC1] |
| | 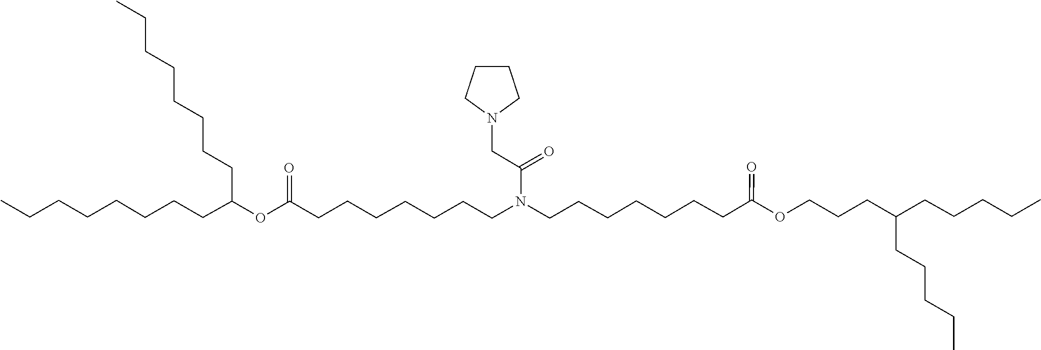 [SMILES: CCCCCCCCC(CCCCCCCC)OC(=O)CCCCCCCN(CCCCCCCC(=O)OCCCC(CCCCC)CCCCC)C(=O)CN1CCCC1] |
| | 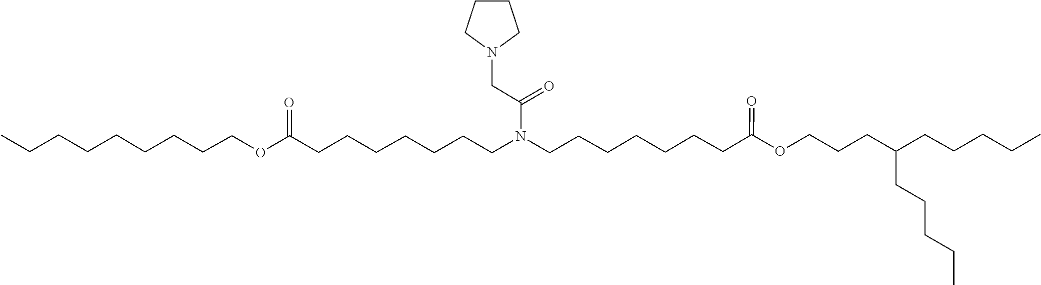 [SMILES: CCCCCCCCCOC(=O)CCCCCCCN(CCCCCCCC(=O)OCCCC(CCCCC)CCCCC)C(=O)CN1CCCC1] |
| | 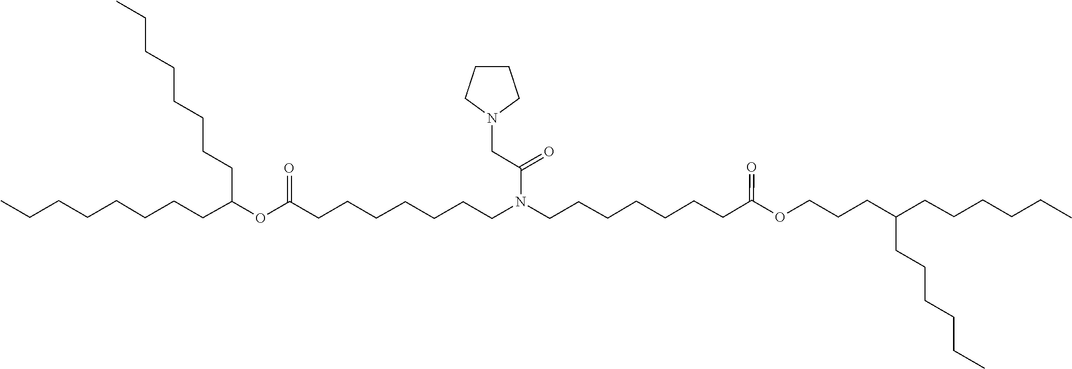 [SMILES: CCCCCCCCC(CCCCCCCC)OC(=O)CCCCCCCN(CCCCCCCC(=O)OCCCC(CCCCCC)CCCCCC)C(=O)CN1CCCC1] |
| | 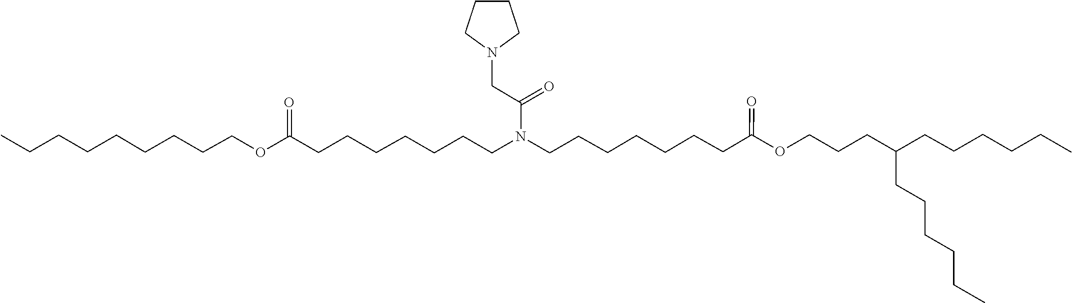 [SMILES: CCCCCCCCCOC(=O)CCCCCCCN(CCCCCCCC(=O)OCCCC(CCCCCC)CCCCCC)C(=O)CN1CCCC1] |
